(19) United States
(12) Patent Application Publication
(10) Pub. No.: US 2024/0226164 A1
(43) Pub. Date: Jul. 11, 2024

(54) HYPOIMMUNOGENIC CELLS COMPRISING ENGINEERED HLA-E OR HLA-G

(71) Applicant: Sana Biotechnology, Inc., Seattle, WA (US)

(72) Inventors: Sonja Schrepfer, San Mateo, CA (US); Edward Rebar, San Francisco, CA (US)

(21) Appl. No.: 18/561,581

(22) PCT Filed: May 25, 2022
(86) PCT No.: PCT/US22/30934

§ 371 Date: Nov. 16, 2023

Related U.S. Application Data
(60) Provisional application No. 63/194,106, filed on May 27, 2021.
(60) Provisional application No. 63/255,912, filed on Oct. 14, 2021.

(51) Int. Cl. A61K 35/17; C07K 14/47; C07K 14/74; C12N 5/0783; C12N 9/22; C12N 15/11; C12N 15/90

(52) U.S. Cl. CPC A61K 35/17; C07K 14/4747; C07K 14/70539; C12N 5/0636; C12N 9/22; C12N 15/11; C12N 15/907; C12N 2310/20; C12N 2510/00; C12N 2800/80

(57) ABSTRACT

Disclosed herein are engineered cells and/or hypoimmunogenic cells including hypoimmunogenic stem cells, hypoimmunogenic cells differentiated therefrom, and hypoimmunogenic CAR-T cells and related methods of their use and generation comprising one or more exogenous receptors selected from the group consisting of a human leukocyte antigen E (HLA-E) variant protein, a human leukocyte antigen G (HLA-G) variant protein, and an exogenous PD-L1 protein. Provided herein are cells further exhibiting reduced expression of MHC I and MHC II human leukocyte antigens and T-cell receptors.

### CROSS REFERENCE TO RELATED APPLICATIONS

This application claims the benefit of U.S. Provisional Application No. 63/194,106, filed May 27, 2021, and U.S. Provisional Application No. 63/255,912, filed Oct. 14, 2021 which are hereby incorporated by reference in their entireties.

### BACKGROUND

Off-the-shelf CAR-T cells and other therapeutic cells can offer advantages over autologous cell-based strategies, including ease of manufacturing, quality control and avoidance of malignant contamination and T cell dysfunction. However, the vigorous host-versus-graft immune response against histoincompatible cells prevents expansion and persistence of allogeneic cells and mitigates the efficacy of this approach.

There is substantial evidence in both animal models and human patients that hypoimmunogenic cell transplantation is a scientifically feasible and clinically promising approach to the treatment of numerous disorders, conditions, and diseases.

There remains a need for novel approaches, compositions and methods for producing cell-based therapies that avoid detection by the recipient's immune system.

### SUMMARY

Provided is an engineered cell comprising one or more exogenous receptors selected from the group consisting of a human leukocyte antigen E (HLA-E) variant protein, a human leukocyte antigen G (HLA-G) variant protein, and an exogenous PD-L1 protein.

In some embodiments, the engineered cell comprises two or more exogenous receptors selected from the group consisting of a human leukocyte antigen E (HLA-E) variant protein, a human leukocyte antigen G (HLA-G) variant protein, and an exogenous PD-L1 protein.

In some embodiments, the engineered cell further comprises reduced expression of MHC class I and/or MHC class II human leukocyte antigens relative to an unaltered or unmodified wild-type cell.

Provided is a hypoimmunogenic cell comprising: (i) reduced expression of MHC class I and/or MHC class II human leukocyte antigens relative to an unaltered or unmodified wild-type cell; and one or more exogenous receptors selected from the group consisting of an HLA-E variant protein, an HLA-G variant protein, and an exogenous PD-L1 protein.

In some embodiments, the engineered cell or the hypoimmunogenic cell further comprises reduced expression and/or no expression of one or more receptors selected from the group consisting of HLA-A, HLA-B, HLA-C, and CD155. In some embodiments, the engineered cell or the hypoimmunogenic cell further comprises no expression of HLA-A and HLA-B.

In some embodiments, the HLA-E variant protein comprises a modification in the antigen binding cleft and/or the HLA-G variant protein comprises a modification in the antigen binding cleft.

In some embodiments, the HLA-E variant protein comprises a modification that increases protein stability compared to a wild-type HLA-E protein and/or the HLA-G variant protein comprises a modification that increases protein stability compared to a wild-type HLA-G protein.

In some embodiments, i) the HLA-E variant protein comprises a modification that increases the recycling rate of the non-antigen bound HLA-E variant protein such that the HLA-E variant protein remains on the cell surface for a longer period of time compared to a wild-type HLA-E protein, and/or ii) the HLA-G variant protein comprises a modification that increases the recycling rate of the non-antigen bound HLA-G variant protein such that the HLA-G variant protein remains on the cell surface for a longer period of time compared to a wild-type HLA-G protein.

In some embodiments, the modification at the antigen binding cleft of the HLA-E variant protein prevents an antigen peptide from binding to the HLA-E variant protein and/or wherein the modification at the antigen binding cleft of the HLA-G variant protein prevents an antigen peptide from binding to the HLA-G variant protein

In some embodiments, the HLA-E variant protein comprises a modification such that the HLA-E variant protein binds a first decoy peptide and/or the HLA-G variant protein comprises a modification such that the HLA-G variant protein binds a second decoy peptide.

In some embodiments, the first decoy peptide of the HLA-E variant protein is tethered to the HLA-E variant protein. In some embodiments, the first decoy peptide of the HLA-E variant protein binds the antigen binding cleft of the HLA-E variant protein.

In some embodiments, the second decoy peptide of the HLA-G variant protein is tethered to the HLA-G variant protein. In some embodiments, the second decoy peptide of the HLA-G variant protein binds the antigen binding cleft of the HLA-G variant protein.

In some embodiments, the first decoy peptide and the second decoy peptide are different peptides.

In some embodiments, the HLA-E variant protein comprises a deletion in one or more of the intracellular domains and/or the HLA-G variant protein comprises a deletion in one or more of the intracellular domains. In some embodiments, the deletion in the one or more of the intracellular domains of HLA-E reduces or eliminates HLA-E signaling and/or the deletion in the one or more of the intracellular domains of HLA-G reduces or eliminates HLA-G signaling.

In some embodiments, i) the HLA-E variant protein comprises a deletion or other modification in the extracellular antigen binding domain region of the variant protein such that when the HLA-E variant protein is bound to an antigen peptide, the variant protein fails to recognize another binding partner, and/or ii) the HLA-G variant protein comprises a deletion or other modification in the extracellular antigen binding domain region of the variant protein such that when the HLA-G variant protein is bound to an antigen peptide, the variant protein fails to recognize another binding partner.

In some embodiments, the HLA-E variant protein comprises an HLA-E single chain dimer comprising an HLA-E heavy chain, a B2M subunit, and a linker, wherein the linker connects the HLA-E heavy chain and the B2M subunit. In some embodiments, the HLA-E variant protein comprises an HLA-E single chain trimer comprising an HLA-E heavy chain, a B2M subunit, an antigen peptide, a first linker, and a second linker, wherein the first linker connects the HLA-E heavy chain and the B2M subunit and the second linker connects the B2M subunit to the antigen peptide.

In some embodiments, the engineered cell or the hypoimmunogenic cell does not express MHC class I and/or MHC class II human leukocyte antigens. In some embodiments, the engineered cell or the hypoimmunogenic cell does not express HLA-DP, HLA-DQ, and/or HLA-DR antigens.

In some embodiments, the engineered cell or the hypoimmunogenic cell comprises reduced expression of beta-2-microglobulin (B2M) and/or MHC class II transactivator (CIITA) relative to an unaltered or unmodified wild-type cell. In some embodiments, the engineered cell or the hypoimmunogenic cell does not express B2M and/or CIITA.

In some embodiments, the engineered cell or the hypoimmunogenic cell comprises one or more exogenous polynucleotides selected from the group consisting of a first polynucleotide encoding the HLA-E variant protein, a second polynucleotide encoding the HLA-G variant protein, and a third polynucleotide encoding the exogenous PD-L1 protein.

In some embodiments, the engineered cell or the hypoimmunogenic cell comprising two or more exogenous polynucleotides selected from the group consisting of a first polynucleotide encoding the HLA-E variant protein, a second polynucleotide encoding the HLA-G variant protein, and a third polynucleotide encoding the exogenous PD-L1 protein.

In some embodiments, the first polynucleotide encoding the HLA-E variant protein is inserted into a first specific locus of at least one allele of the cell.

In some embodiments, the second polynucleotide encoding the HLA-G variant protein is inserted into a second specific locus of at least one allele of the cell.

In some embodiments, the third polynucleotide encoding the exogenous PD-L1 protein is inserted into a third specific locus of at least one allele of the cell.

In some embodiments, the first, second and/or third specific loci are selected from the group consisting of a safe harbor locus, an RHD locus, a B2M locus, a CIITA locus, a TRAC locus, a TRB locus, an HLA-A locus, an HLA-B locus, an HLA-C locus, and a CD155 locus.

In some embodiments, the safe harbor locus is selected from the group consisting of a CCR5 locus, a CXCR4 locus, a PPP1R12C locus, an ALB locus, a SHS231 locus, a CLYBL locus, a Rosa locus, an F3 (CD142) locus, a MICA locus, a MICB locus, a LRP1 (CD91) locus, a HMGB1 locus, an ABO locus, a FUT1 locus, and a KDM5D locus.

In some embodiments, the e any two of the first, second and third loci are the same locus.

In some embodiments, the first, second and third loci are the same locus.

In some embodiments, the first, second and third loci are different loci.

In some embodiments, the engineered cell or the hypoimmunogenic cell further comprises a single bicistronic polynucleotide comprising two polynucleotides selected from the group consisting of the first polynucleotide, the second polynucleotide and the third polynucleotide.

In some embodiments, the first polynucleotide, second polynucleotide and/or third polynucleotide are introduced into the engineered cell or the hypoimmunogenic cell using a lentiviral vector.

In some embodiments, the engineered cell or the hypoimmunogenic cell is derived from a human cell or an animal cell. In some embodiments, the engineered cell or the hypoimmunogenic cell is a differentiated cell derived from an induced pluripotent stem cell or a progeny thereof. In some embodiments, the differentiated cell is selected from the group consisting of a T cell, a natural killer (NK) cell, and an endothelial cell. In some embodiments, the engineered cell or the hypoimmunogenic cell is a primary immune cell or a progeny thereof. In some embodiments, the primary immune cell or a progeny thereof is a T cell or an NK cell.

In some embodiments, the T cell comprises one or more one or more chimeric antigen receptors (CARs). In some embodiments, the one or more CARs are selected from the group consisting of a CD19-specific CAR, such that the T cell is a CD19 CAR T cell, a CD20-specific CAR, such that the T cell is a CD20 CAR T cell, a CD22-specific CAR, such that the T cell is a CD22 CAR T cell, and a BCMA-specific CAR such that the T cell is a BCMA CAR T cell, or a combination thereof. In some embodiments, the T cell comprises a CD19-specific CAR and a CD22-specific CAR such that the cell is a CD19/CD22 CAR T cell.

In some embodiments, the CD19-specific CAR and a CD22-specific CAR are encoded by a single bicistronic polynucleotide. In some embodiments, the CD19-specific CAR and a CD22-specific CAR are encoded by two separate polynucleotides.

In some embodiments, the one or more CARs are introduced to the T cell using a lentiviral vector.

In some embodiments, the one or more CARs are introduced to the T cell in vivo in a recipient patient. In some embodiments, the one or more CARs are introduced to the T cell by contacting the recipient patient with a composition comprising one or more lentiviral vectors comprising (i) a CD4 binding agent or a CD8 binding agent, and (ii) one or more polynucleotides encoding the one or more CARs, wherein the T cell of the recipient patient is transduced with the one or more lentiviral vectors.

In some embodiments, the one or more CARs are introduced the T cell using CRISPR/Cas gene editing. In some embodiments, the CRISPR/Cas gene editing is carried out ex vivo from a donor subject.

In some embodiments, the CRISPR/Cas gene editing is carried out using a lentiviral vector.

In some embodiments, the CRISPR/Cas gene editing is carried out in vivo in a recipient patient. In some embodiments, the CRISPR/Cas gene editing is carried out by contacting the recipient patient with a composition comprising lentiviral vectors comprising (i) a CD4 binding agent or a CD8 binding agent, (ii) polynucleotides encoding CRISPR/Cas gene editing components, and (iii) one or more polynucleotides encoding the one or more CARs, wherein the T cell of the recipient patient is transduced with the lentiviral vectors.

In some embodiments, the differentiated cell or the progeny thereof, or the primary immune cell or the progeny thereof evades NK cell mediated cytotoxicity upon administration to a recipient patient. In some embodiments, the differentiated cell or the progeny thereof, or the primary immune cell or the progeny thereof is protected from cell lysis by mature NK cells upon administration to a recipient patient. In some embodiments, the differentiated cell or the progeny thereof, or the primary immune cell or the progeny thereof does not induce an immune response to the cell upon administration to a recipient patient.

Provided is a pharmaceutical composition comprising a population of any of the engineered cells described or a population of any of the hypoimmunogenic cells described, and a pharmaceutically acceptable additive, carrier, diluent or excipient.

Provided is a method of treating a condition or disease in a patient in need thereof comprising administering a population of any of the differentiated cells described to the patient. In some embodiments, the differentiated cells are selected from the group consisting of T cells, NK cells, and endothelial cells.

In some embodiments, the method further comprises administering a therapeutic agent that binds and/or interacts with one or more receptors on NK cells selected from the group consisting of CD94, KIR2DL4, PD-1, an inhibitory NK cell receptor, and an activating NK receptor. In some embodiments, the therapeutic agent is selected from the group consisting of an antibody and fragments and variants thereof, an antibody mimetic, a small molecule, a blocking peptide, and a receptor antagonist.

In some embodiments, the condition or disease is selected from the group consisting of cancer, cardiovascular disease, stroke, peripheral artery disease (PAD), abdominal aortic aneurysm (AAA), carotid artery disease (CAD), arteriovenous malformation (AVM), critical limb-threatening ischemia (CLTI), pulmonary embolism (blood clots), deep vein thrombosis (DVT), chronic venous insufficiency (CVI), and any another vascular disorder/condition.

In some embodiments, the administration is selected from the group consisting of intravenous injection, intramuscular injection, intravascular injection, and transplantation.

Provided is a method of treating cancer in a patient in need thereof comprising administering a population of any of the primary immune cells described to the patient. In some embodiments, the primary immune cells are selected from the group consisting of T cells and NK cells.

In some embodiments, the present technology relates to the use of a population of engineered T cells for treating a disorder or conditions in a recipient patient, wherein the engineered T cells comprise one or more exogenous receptors selected from the group consisting of an HLA-E variant protein, a HLA-G variant protein, and an exogenous PD-L1 protein and reduced expression of MHC class I and/or MHC class II human leukocyte antigens relative to an unaltered or unmodified wild-type cell, wherein the engineered T cells are propagated from a primary T cell or a progeny thereof, or are derived from an iPSC or a progeny thereof.

In some embodiments, the engineered T cell comprises two or more exogenous receptors selected from the group consisting of a HLA-E variant protein, a HLA-G variant protein, and an exogenous PD-L1 protein.

In some embodiments, the engineered T cell further comprises reduced expression and/or no expression of one or more receptors selected from the group consisting of HLA-A, HLA-B, HLA-C, and CD155. In some embodiments, the engineered T cell further comprises no expression of HLA-A and HLA-B.

In some embodiments, the engineered T cells comprise an HLA-E variant protein and an HLA-G variant protein and reduced expression and/or no expression of one or more receptors selected from the group consisting of HLA-A, HLA-B, HLA-C, and CD155 relative to an unaltered or unmodified wild-type cell.

In some embodiments, the engineered T cells comprise an HLA-E variant protein and an HLA-G variant protein and no expression of HLA-A and HLA-B.

In some embodiments, the engineered T cells comprise an HLA-E variant protein and an exogenous PD-L1 protein and reduced expression and/or no expression of one or more receptors selected from the group consisting of HLA-A, HLA-B, HLA-C, and CD155 relative to an unaltered or unmodified wild-type cell. In some embodiments, the engineered T cells comprise an HLA-E variant protein and an exogenous PD-L1 protein and no expression of HLA-A and HLA-B.

In some embodiments, the engineered T cells comprise an HLA-G variant protein and an exogenous PD-L1 protein and reduced expression and/or no expression of one or more receptors selected from the group consisting of HLA-A, HLA-B, HLA-C, and CD155 relative to an unaltered or unmodified wild-type cell. In some embodiments, the engineered T cells comprise an HLA-G variant protein and an exogenous PD-L1 protein and no expression of HLA-A and HLA-B.

In some embodiments, the engineered T cells comprise an HLA-E variant protein and an HLA-G variant protein and reduced expression of MHC class I and MHC class II human leukocyte antigens relative to an unaltered or unmodified wild-type cell. In some embodiments, the engineered T cells comprise an HLA-E variant protein and an exogenous PD-L1 protein and reduced expression of MHC class I and MHC class II human leukocyte antigens relative to an unaltered or unmodified wild-type cell. In some embodiments, the engineered T cells comprise an HLA-G variant protein and an exogenous PD-L1 protein and reduced expression of MHC class I and MHC class II human leukocyte antigens relative to an unaltered or unmodified wild-type cell.

In some embodiments, the engineered T cells comprise an HLA-E variant protein and an HLA-G variant protein and reduced expression of B2M and/or CIITA relative to an unaltered or unmodified wild-type cell. In some embodiments, the engineered T cells comprise an HLA-E variant protein and an exogenous PD-L1 protein and reduced expression of B2M and/or CIITA relative to an unaltered or unmodified wild-type cell. In some embodiments, the engineered T cells comprise an HLA-G variant protein and an exogenous PD-L1 protein and reduced expression of B2M and/or CIITA relative to an unaltered or unmodified wild-type cell.

In some embodiments, the engineered T cells comprise an HLA-E variant protein and an HLA-G variant protein and reduced expression of B2M and CIITA relative to an unaltered or unmodified wild-type cell. In some embodiments, the engineered T cells comprise an HLA-E variant protein and an exogenous PD-L1 protein and reduced expression of B2M and CIITA relative to an unaltered or unmodified wild-type cell. In some embodiments, the engineered T cells comprise an HLA-G variant protein and an exogenous PD-L1 protein and reduced expression of B2M and CIITA relative to an unaltered or unmodified wild-type cell.

In some embodiments, the engineered T cells do not express MHC class I human leukocyte antigens, do not express MHC class II human leukocyte antigens and comprise an HLA-E variant protein and an HLA-G variant protein. In some embodiments, the engineered T cells do not express MHC class I human leukocyte antigens, do not express MHC class II human leukocyte antigens and comprise an HLA-E variant protein and an exogenous PD-L1 protein.

In some embodiments, the engineered T cells do not express B2M, do not express CIITA and comprise an HLA-G variant protein and an exogenous PD-L1 protein. In some embodiments, the engineered T cells do not express B2M, do not express CIITA and comprise an HLA-E variant protein and an HLA-G variant protein. In some embodiments, the engineered T cells do not express B2M, do not express CIITA and comprise an HLA-E variant protein and an exogenous PD-L1 protein. In some embodiments, the engineered T cells do not express B2M, do not express CIITA and comprise an HLA-G variant protein and an exogenous PD-L1 protein.

In some embodiments, the HLA-E variant protein comprises a modification in the antigen binding cleft and/or the HLA-G variant protein comprises a modification in the antigen binding cleft.

In some embodiments, the modification at the antigen binding cleft of the HLA-E variant protein prevents an antigen peptide from binding to the HLA-E variant protein and/or wherein the modification at the antigen binding cleft of the HLA-G variant protein prevents an antigen peptide from binding to the HLA-G variant protein.

In some embodiments, the HLA-E variant protein comprises a modification such that the HLA-E variant protein binds a first decoy peptide and/or the HLA-G variant protein comprises a modification such that the HLA-G variant protein binds a second decoy peptide. In some embodiments, the first decoy peptide of the HLA-E variant protein is tethered to the HLA-E variant protein. In some embodiments, the first decoy peptide of the HLA-E variant protein binds the antigen binding cleft of the HLA-E variant protein. In some embodiments, the second decoy peptide of the HLA-G variant protein is tethered to the HLA-G variant protein. In some embodiments, the second decoy peptide of the HLA-G variant protein binds the antigen binding cleft of the HLA-G variant protein. In some embodiments, the first decoy peptide and the second decoy peptide are different peptides

In some embodiments, the HLA-E variant protein comprises a deletion in one or more of the intracellular domains and/or the HLA-G variant protein comprises a deletion in one or more of the intracellular domains.

In some embodiments, the deletion in the one or more of the intracellular domains of HLA-E reduces or eliminates HLA-E signaling and/or the deletion in the one or more of the intracellular domains of HLA-G reduces or eliminates HLA-G signaling.

In some embodiments, i) the HLA-E variant protein comprises a deletion or other modification in the extracellular antigen binding domain region of the variant protein such that when the HLA-E variant protein is bound to an antigen peptide, the variant protein fails to recognize another binding partner, and/or ii) the HLA-G variant protein comprises a deletion or other modification in the extracellular antigen binding domain region of the variant protein such that when the HLA-G variant protein is bound to an antigen peptide, the variant protein fails to recognize another binding partner.

In some embodiments, the HLA-E variant protein comprises an HLA-E single chain dimer comprising an HLA-E heavy chain, a B2M subunit, and a linker wherein the linker connects the HLA-E heavy chain and the B2M subunit.

In some embodiments, the HLA-E variant protein comprises an HLA-E single chain trimer comprising an HLA-E heavy chain, a B2M subunit, an antigen peptide, a first linker, and a second linker, wherein the first linker connects the HLA-E heavy chain and the B2M subunit and the second linker connects the B2M subunit to the antigen peptide.

In some embodiments, the engineered T cells comprise one or more exogenous polynucleotides selected from the group consisting of a first polynucleotide encoding the HLA-E variant protein, a second polynucleotide encoding the HLA-G variant protein, and a third polynucleotide encoding the exogenous PD-L1 protein. In some embodiments, the engineered T cells comprise two or more exogenous polynucleotides selected from the group consisting of a first polynucleotide encoding the HLA-E variant protein, a second polynucleotide encoding the HLA-G variant protein, and a third polynucleotide encoding the exogenous PD-L1 protein.

In some embodiments, the first polynucleotide encoding the HLA-E variant protein is inserted into a first specific locus of at least one allele of the cell, the second polynucleotide encoding the HLA-G variant protein is inserted into a second specific locus of at least one allele of the cell, and/or the third polynucleotide encoding the exogenous PD-L1 protein is inserted into a third specific locus of at least one allele of the cell.

In some embodiments, the first, second and/or third specific loci are selected from the group consisting of a safe harbor locus, an RHD locus, a B2M locus, a CIITA locus, a TRAC locus, a TRB locus, an HLA-A locus, an HLA-B locus, an HLA-C locus, and a CD155 locus.

In some embodiments, the safe harbor locus is selected from the group consisting of a CCR5 locus, a CXCR4 locus, a PPP1R12C locus, an ALB locus, a SHS231 locus, a CLYBL locus, a Rosa locus, an F3 (CD142) locus, a MICA locus, a MICB locus, a LRP1 (CD91) locus, a HMGB1 locus, an ABO locus, a FUT1 locus, and a KDM5D locus.

In some embodiments, the any two of the first, second and third loci are the same locus. In some embodiments, the first, second and third loci are the same locus. In some embodiments, the first, second and third loci are different loci.

In some embodiments, the engineered T cells further comprise a single bicistronic polynucleotide comprising two polynucleotides selected from the group consisting of the first polynucleotide, the second polynucleotide and the third polynucleotide.

In some embodiments, the first polynucleotide, the second polynucleotide and/or the third polynucleotide are introduced into the engineered T cell using CRISPR/Cas gene editing.

In some embodiments, the first polynucleotide, second polynucleotide and/or third polynucleotide are introduced into the engineered T cell using a lentiviral vector.

In some embodiments, the engineered T cell comprises one or more one or more chimeric antigen receptors (CARs).

In some embodiments, the one or more CARs are selected from the group consisting of a CD19-specific CAR, such that the engineered T cell is a CD19 CAR T cell, a CD20-specific CAR, such that the engineered T cell is a CD20 CAR T cell, a CD22-specific CAR, such that the engineered T cell is a CD22 CAR T cell, and a BCMA-specific CAR such that the engineered T cell is a BCMA CAR T cell, or a combination thereof. In some embodiments, the engineered T cell comprises a CD19-specific CAR and a CD22-specific CAR such that the cell is a CD19/CD22 CAR T cell.

In some embodiments, the CD19-specific CAR and a CD22-specific CAR are encoded by a single bicistronic polynucleotide. In some embodiments, the CD19-specific CAR and a CD22-specific CAR are encoded by a two separate polynucleotides.

In some embodiments, the one or more CARs are introduced to the engineered T cell using a lentiviral vector.

In some embodiments, the one or more CARs are introduced to the engineered T cell in vivo in the recipient patient. In some embodiments, the one or more CARs are introduced to the engineered T cell by contacting the recipient patient with a composition comprising one or more lentiviral vectors comprising (i) a CD4 binding agent or a CD8 binding agent, and (ii) one or more polynucleotides encoding the one or more CARs, wherein the engineered T cell of the recipient patient is transduced with the one or more lentiviral vectors.

In some embodiments, the one or more CARs are introduced the engineered T cell using CRISPR/Cas gene editing. In some embodiments, the CRISPR/Cas gene editing is carried out ex vivo from a donor subject.

In some embodiments, the CRISPR/Cas gene editing is carried out using a lentiviral vector.

In some embodiments, the CRISPR/Cas gene editing is carried out in vivo in the recipient patient. In some embodiments, the CRISPR/Cas gene editing is carried out by contacting the recipient patient with a composition comprising one or more lentiviral vectors comprising (i) a CD4 binding agent or a CD8 binding agent, (ii) polynucleotides encoding CRISPR/Cas gene editing components, and (iii) one or more polynucleotides encoding the one or more CARs, wherein the T cell of the recipient patient is transduced with the one or more lentiviral vectors.

In some embodiments, the present technology relates to the use of a population of engineered differentiated cells for treating a disorder or conditions in a recipient patient, wherein the engineered differentiated cells comprise one or more exogenous receptors selected from the group consisting of an HLA-E variant protein, a HLA-G variant protein, and an exogenous PD-L1 protein and reduced expression of MHC class I and/or MHC class II human leukocyte antigens relative to an unaltered or unmodified wild-type cell, wherein the engineered differentiated cells are derived an iPSC or a progeny thereof.

In some embodiments, the engineered differentiated cells comprise two or more exogenous receptors selected from the group consisting of a HLA-E variant protein, a HLA-G variant protein, and an exogenous PD-L1 protein.

In some embodiments, the engineered differentiated cell further comprises reduced expression and/or no expression of one or more receptors selected from the group consisting of HLA-A, HLA-B, HLA-C, and CD155. In some embodiments, the engineered differentiated cell further comprises no expression of HLA-A and HLA-B.

In some embodiments, the engineered differentiated cells comprise an HLA-E variant protein and an HLA-G variant protein and reduced expression and/or no expression of one or more receptors selected from the group consisting of HLA-A, HLA-B, HLA-C, and CD155 relative to an unaltered or unmodified wild-type cell. In some embodiments, the engineered differentiated cells comprise an HLA-E variant protein and an HLA-G variant protein and no expression of HLA-A and HLA-B.

In some embodiments, the engineered differentiated cells comprise an HLA-E variant protein and an exogenous PD-L1 protein and reduced expression and/or no expression of one or more receptors selected from the group consisting of HLA-A, HLA-B, HLA-C, and CD155 relative to an unaltered or unmodified wild-type cell. In some embodiments, the engineered differentiated cells comprise an HLA-E variant protein and an exogenous PD-L1 protein and no expression of HLA-A and HLA-B.

In some embodiments, the engineered differentiated cells comprise an HLA-G variant protein and an exogenous PD-L1 protein and reduced expression and/or no expression of one or more receptors selected from the group consisting of HLA-A, HLA-B, HLA-C, and CD155 relative to an unaltered or unmodified wild-type cell. In some embodiments, the engineered differentiated cells comprise an HLA-G variant protein and an exogenous PD-L1 protein and no expression of HLA-A and HLA-B.

In some embodiments, the engineered differentiated cells comprise an HLA-E variant protein and an HLA-G variant protein and reduced expression of MHC class I and MHC class II human leukocyte antigens relative to an unaltered or unmodified wild-type cell. In some embodiments, the engineered differentiated cells comprise an HLA-E variant protein and an exogenous PD-L1 protein and reduced expression of MHC class I and MHC class II human leukocyte antigens relative to an unaltered or unmodified wild-type cell. In some embodiments, the engineered differentiated cells comprise an HLA-G variant protein and an exogenous PD-L1 protein and reduced expression of MHC class I and MHC class II human leukocyte antigens relative to an unaltered or unmodified wild-type cell.

In some embodiments, the engineered differentiated cells comprise an HLA-E variant protein and an HLA-G variant protein and reduced expression of B2M and/or CIITA relative to an unaltered or unmodified wild-type cell. In some embodiments, the engineered differentiated cells comprise an HLA-E variant protein and an exogenous PD-L1 protein and reduced expression of B2M and/or CIITA relative to an unaltered or unmodified wild-type cell. In some embodiments, the engineered differentiated cells comprise an HLA-G variant protein and an exogenous PD-L1 protein and reduced expression of B2M and/or CIITA relative to an unaltered or unmodified wild-type cell.

In some embodiments, the engineered differentiated cells comprise an HLA-E variant protein and an HLA-G variant protein and reduced expression of B2M and CIITA relative to an unaltered or unmodified wild-type cell. T In some embodiments, the engineered differentiated cells comprise an HLA-E variant protein and an exogenous PD-L1 protein and reduced expression of B2M and CIITA relative to an unaltered or unmodified wild-type cell. In some embodiments, the engineered differentiated cells comprise an HLA-G variant protein and an exogenous PD-L1 protein and reduced expression of B2M and CIITA relative to an unaltered or unmodified wild-type cell.

In some embodiments, the engineered differentiated cells do not express MHC class I human leukocyte antigens, do not express MHC class II human leukocyte antigens and comprise an HLA-E variant protein and an HLA-G variant protein. In some embodiments, the engineered differentiated cells do not express MHC class I human leukocyte antigens, do not express MHC class II human leukocyte antigens and comprise an HLA-E variant protein and an exogenous PD-L1 protein.

In some embodiments, the engineered differentiated cells do not express B2M, do not express CIITA and comprise an HLA-G variant protein and an exogenous PD-L1 protein. In some embodiments, the engineered differentiated cells do not express B2M, do not express CIITA and comprise an HLA-E variant protein and an HLA-G variant protein. In some embodiments, the engineered differentiated cells do not express B2M, do not express CIITA and comprise an HLA-E variant protein and an exogenous PD-L1 protein. In some embodiments, the engineered T cells do not express B2M, do not express CIITA and comprise an HLA-G variant protein and an exogenous PD-L1 protein.

In some embodiments, the HLA-E variant protein comprises a modification in the antigen binding cleft and/or the HLA-G variant protein comprises a modification in the antigen binding cleft.

In some embodiments, the modification at the antigen binding cleft of the HLA-E variant protein prevents an antigen peptide from binding to the HLA-E variant protein and/or wherein the modification at the antigen binding cleft of the HLA-G variant protein prevents an antigen peptide from binding to the HLA-G variant protein

In some embodiments, the HLA-E variant protein comprises a modification such that the HLA-E variant protein binds a first decoy peptide and/or the HLA-G variant protein comprises a modification such that the HLA-G variant protein binds a second decoy peptide. In some embodiments, the first decoy peptide of the HLA-E variant protein is tethered to the HLA-E variant protein. In some embodiments, the first decoy peptide of the HLA-E variant protein binds the antigen binding cleft of the HLA-E variant protein. In some embodiments, the second decoy peptide of the HLA-G variant protein is tethered to the HLA-G variant protein. In some embodiments, the second decoy peptide of the HLA-G variant protein binds the antigen binding cleft of the HLA-G variant protein. In some embodiments, the first decoy peptide and the second decoy peptide are different peptides.

In some embodiments, the HLA-E variant protein comprises a deletion in one or more of the intracellular domains and/or the HLA-G variant protein comprises a deletion in one or more of the intracellular domains. In some embodiments, the deletion in the one or more of the intracellular domains of HLA-E reduces or eliminates HLA-E signaling and/or the deletion in the one or more of the intracellular domains of HLA-G reduces or eliminates HLA-G signaling.

In some embodiments, i) the HLA-E variant protein comprises a deletion or other modification in the extracellular antigen binding domain region of the variant protein such that when the HLA-E variant protein is bound to an antigen peptide, the variant protein fails to recognize another binding partner, and/or ii) the HLA-G variant protein comprises a deletion or other modification in the extracellular antigen binding domain region of the variant protein such that when the HLA-G variant protein is bound to an antigen peptide, the variant protein fails to recognize another binding partner.

In some embodiments, the HLA-E variant protein comprises an HLA-E single chain dimer comprising an HLA-E heavy chain, a B2M subunit, and a linker wherein the linker connects the HLA-E heavy chain and the B2M subunit. In some embodiments, the HLA-E variant protein comprises an HLA-E single chain trimer comprising an HLA-E heavy chain, a B2M subunit, an antigen peptide, a first linker, and a second linker, wherein the first linker connects the HLA-E heavy chain and the B2M subunit and the second linker connects the B2M subunit to the antigen peptide.

In some embodiments, the engineered differentiated cells comprise one or more exogenous polynucleotides selected from the group consisting of a first polynucleotide encoding the HLA-E variant protein, a second polynucleotide encoding the HLA-G variant protein, and a third polynucleotide encoding the exogenous PD-L1 protein. In some embodiments, the engineered differentiated cells comprise two or more exogenous polynucleotides selected from the group consisting of a first polynucleotide encoding the HLA-E variant protein, a second polynucleotide encoding the HLA-G variant protein, and a third polynucleotide encoding the exogenous PD-L1 protein.

In some embodiments, the first polynucleotide encoding the HLA-E variant protein is inserted into a first specific locus of at least one allele of the cell, the second polynucleotide encoding the HLA-G variant protein is inserted into a second specific locus of at least one allele of the cell, and/or the third polynucleotide encoding the exogenous PD-L1 protein is inserted into a third specific locus of at least one allele of the cell.

In some embodiments, the first, second and/or third specific loci are selected from the group consisting of a safe harbor locus, an RHD locus, a B2M locus, a CIITA locus, a TRAC locus, a TRB locus, an HLA-A locus, an HLA-B locus, an HLA-C locus, and a CD155 locus. In some embodiments, the safe harbor locus is selected from the group consisting of a CCR5 locus, a CXCR4 locus, a PPP1R12C locus, an ALB locus, a SHS231 locus, a CLYBL locus, a Rosa locus, an F3 (CD142) locus, a MICA locus, a MICB locus, a LRP1 (CD91) locus, a HMGB1 locus, an ABO locus, a FUT1 locus, and a KDM5D locus.

In some embodiments, any two of the first, second and third loci are the same locus. In some embodiments, the first, second and third loci are the same locus. In some embodiments, the first, second and third loci are different loci.

In some embodiments, the engineered differentiated cells further comprise a single bicistronic polynucleotide comprising two polynucleotides selected from the group consisting of the first polynucleotide, the second polynucleotide and the third polynucleotide.

In some embodiments, the first polynucleotide, the second polynucleotide and/or the third polynucleotide are introduced the engineered differentiated cell using CRISPR/Cas gene editing.

In some embodiments, the first polynucleotide, second polynucleotide and/or third polynucleotide are introduced into the engineered differentiated cell using a lentiviral vector.

Provided is a human leukocyte antigen E (HLA-E) variant protein comprising a modification at the antigen binding cleft.

In some embodiments, the modification at the antigen binding cleft of the HLA-E variant protein prevents an antigen peptide from binding to the variant protein.

In some embodiments, the HLA-E variant protein binds a decoy peptide. In some embodiments, the decoy peptide of the HLA-E variant protein is tethered to the HLA-E variant protein.

In some embodiments, the decoy peptide of the HLA-E variant protein binds the antigen binding cleft of the HLA-E variant protein.

In some embodiments, the HLA-E variant protein comprises a deletion in one or more of the intracellular domains.

In some embodiments, the HLA-E variant protein comprises an HLA-E single chain dimer comprising an HLA-E heavy chain, a B2M subunit, and a linker wherein the linker connects the HLA-E heavy chain and the B2M subunit.

In some embodiments, the HLA-E variant protein comprises an HLA-E single chain trimer comprising an HLA-E heavy chain, a B2M subunit, an antigen peptide, a first linker, and a second linker, wherein the first linker connects the HLA-E heavy chain and the B2M subunit and the second linker connects the B2M subunit to the antigen peptide.

In some embodiments, provided herein is a human leukocyte antigen G (HLA-G) variant protein comprising a modification in the antigen binding cleft. In some embodiments, the modification at the antigen binding cleft of the HLA-G variant protein prevents an antigen peptide from binding to the variant protein.

In some embodiments, the HLA-G variant protein binds a decoy peptide. In some embodiments, the decoy peptide of the HLA-G variant protein is tethered to the HLA-G variant protein.

In some embodiments, the decoy peptide of the HLA-G variant protein binds the antigen binding cleft of the HLA-G variant protein.

In some embodiments, the HLA-G variant protein comprises a deletion in one or more of the intracellular domains.

Provided herein is a polynucleotide construct comprising a polynucleotide encoding any of the HLA-E variant proteins described. Provided herein is a polynucleotide construct comprising a polynucleotide encoding any of the HLA-G variant proteins described.

In some embodiments, the polynucleotide construct further comprises one or more polynucleotides for CRISPR/Cas gene editing. In some embodiments, the polynucleotide construct further comprises one or more polynucleotides for CRISPR/Cas gene editing to insert the polynucleotide encoding the HLA-E variant protein into a specific locus of at least one allele of a cell. In some embodiments, the polynucleotide construct further comprises one or more polynucleotides for CRISPR/Cas gene editing to insert the polynucleotide encoding the HLA-G variant protein into a specific locus of at least one allele of a cell. In some embodiments, the specific locus is selected from the group consisting of a safe harbor locus, an RHD locus, a B2M locus, a CIITA locus, a TRAC locus, a TRB locus, an HLA-A locus, an HLA-B locus, an HLA-C locus, and a CD155 locus. In some embodiments, the safe harbor locus is selected from the group consisting of a CCR5 locus, a CXCR4 locus, a PPP1R12C locus, an ALB locus, a SHS231 locus, a CLYBL locus, a Rosa locus, an F3 (CD142) locus, a MICA locus, a MICB locus, a LRP1 (CD91) locus, a HMGB1 locus, an ABO locus, a FUT1 locus, and a KDM5D locus.

Provided is a single bicistronic polynucleotide construct comprising a first polynucleotide encoding any of the HLA-E variant protein described and a second polynucleotide encoding any of the HLA-G variant protein described. Provided herein is a single bicistronic polynucleotide construct comprising a first polynucleotide encoding any of the HLA-E variant proteins described and a second polynucleotide encoding an PD-L1 protein. In some embodiments, provided is a single bicistronic polynucleotide construct comprising a first polynucleotide encoding the HLA-G variant protein and a second polynucleotide encoding an PD-L1 protein.

In some embodiments, the construct further comprises a promoter. In some embodiments, the promoter is a constitutive promoter. In some embodiments, the promoter is a tissue-type specific promoter.

Detailed descriptions of hypoimmunogenic cells, methods of producing thereof, and methods of using thereof are found in U.S. Provisional Application No. 63/065,342 filed on Aug. 13, 2020, U.S. Provisional Application No. 63/136,152 filed on Dec. 31, 2020, U.S. Provisional Application No. 63/175,030 filed on Apr. 14, 2021, U.S. Provisional Application No. 63/175,003 filed on Apr. 14, 2021, and U.S. Provisional Application filed on Jan. 11, 2021 (Attorney Docket No. 18615-30046.00), WO2016/183041 filed May 9, 2015, WO2018/132783 filed Jan. 14, 2018, WO2020/018615 filed Jul. 17, 2019, WO2020/018620 filed Jul. 17, 2019, WO2020/168317 filed Feb. 16, 2020, PCT/US2021/029443 filed Apr. 27, 2021, the disclosures of which including the examples, sequence listings and figures are incorporated herein by reference in their entireties.

### BRIEF DESCRIPTION OF THE DRAWINGS

FIG. 1 is a schematic diagram depicting exemplary molecules that can mediate NK cell evasion. Overexpression of various molecules such as HLA-E, HLA-G, PD-L1 and CD47 in K562 cells which lack expression of HLA-I and HLA-II may prevent activation of an NK cell mediated innate immune response.

FIGS. 2A-2D show flow cytometry data measuring HLA-A/B/C and HLA-II levels on K562 cells in vitro and in vivo, compared to an isotype control. No expression of HLA-I and HLA-II was detected on K562 cells in vitro and in vivo.

FIGS. 3A-3B show flow cytometry data measuring HLA-E levels on unmodified K562 cells and modified K562 cells that express exogenous HLA-E proteins, compared to an isotype control.

FIGS. 4A-4B show flow cytometry data measuring HLA-G levels on unmodified K562 cells and modified K562 cells that express exogenous HLA-G proteins, compared to an isotype control.

FIGS. 5A-5B depict flow cytometry data measuring PD-L1 levels on unmodified K562 cells and modified K562 cells that express exogenous PD-L1 proteins, compared to an isotype control.

FIGS. 6A-6C show flow cytometry data measuring KIR2DL levels on unsorted NK cells, CD56 high NK cells (also referred to as “immature NK cells”), and CD56 dim NK cells (also referred to as “mature NK cells”), compared to an isotype control.

FIGS. 7A-7G depict flow cytometry data measuring CD56 and CD94 levels on unsorted NK cells. FIG. 7A shows the FACS plot of CD94 vs. CD56. FIG. 7B shows the percentage of CD56 high immature NK cells. FIG. 7C shows the percentage of CD56 high/CD94 high immature NK cells. FIG. 7D shows the percentage of CD94 high NK cells. FIG. 7E shows the percentages of CD56 dim mature NK cells. FIG. 7E shows the percentage of CD56 dim/CD94 dim mature NK cells. FIG. 7F shows the percentage of CD94 dim NK cells.

FIGS. 8A-8J depict cell killing data of K562+HLA-E^{KI} cells by various NK cell subpopulations including unsorted NK cells, CD56 high/CD94 high immature NK cells, CD56 dim/CD94 dim mature NK cells, CD94 high NK cells, and CD94 dim NK cells.

FIGS. 9A-9G show flow cytometry data measuring CD56 and KIR2DL4 levels on unsorted NK cells. FIG. 9A shows the FACS plot of KIR2DL4 vs. CD56. FIG. 9B shows the percentage of CD56 high NK cells. FIG. 9C shows the percentage of CD56 high/KIR2DL4 high NK cells. FIG. 9D shows the percentage of KIR2DL4 high NK cells. FIG. 9E shows the percentage of CD56 dim NK cells. FIG. 9F shows the percentage of CD56 dim/KIR2DL4 dim NK cells. FIG. 9G shows the percentage of KIR2DL4 dim NK cells.

FIGS. 10A-10J depict cell killing data of K562+HLA-G^{KI} cells by various NK cell subpopulations including unsorted NK cells, CD56 high/KIR2DL4 high NK cells, CD56 dim/KIR2DL4 dim NK cells, KIR3DL4 high NK cells, and KIR3DL4 dim NK cells.

FIGS. 11A-11G show flow cytometry data measuring CD56 and PD-1 levels on unsorted NK cells. FIG. 11A shows the FACS plot of PD-1 vs. CD56. FIG. 11B shows the percentages of CD56 high NK cells. FIG. 11C shows the percentage of CD56 high/PD-1 high NK cells. FIG. 11D shows the percentage of PD-1 high NK cells. FIG. 11E shows the percentages of CD56 dim NK cells. FIG. 11F shows the percentage of CD56 dim/PD-1 dim NK cells. FIG. 11G shows the percentage of PD-1 dim NK cells.

FIGS. 12A-12J show from cell killing data of K562+PD-L1^{KI} cells by various NK cell subpopulations including unsorted NK cells, CD56 high/PD-1 high NK cells, CD56 dim/PD-1 dim NK cells, PD-1 high NK cells, and PD-1 dim NK cells.

FIGS. 13A-13H show granzyme B and perforin release levels by NK cells as determined by a standard ELISA assay. Levels were evaluated from unsorted NK cells and specific NK cell subpopulations exposed to unmodified K562 cells (FIG. 13A), HLA-E knock-in K562 cells (FIG. 13B), HLA-G knock-in K562 cells (FIG. 13C), and PD-L1 knock-in K562 cells (FIG. 13D).

FIGS. 14A-14H depict expression levels of NK cell inhibitory ligands and NK cell activation ligands on unstimulated and stimulated cells including unmodified K562 cells (FIG. 14A), HLA-E knock-in K562 cells (FIG. 14B), HLA-G knock-in K562 cells (FIG. 14C), and PD-L1 knock-in K562 cells (FIG. 14D).

FIGS. 15A-15G show data of immune evasion in vivo following adoptive transfer of human NK cell into immunodeficient NSG mice along with either (i) a mixture of human mock T cells and HLA-I/II deficient cells (FIG. 15A) or (ii) a mixture of human mock T cells and MHC I/II deficient cells overexpressing either HLA-E (FIG. 15B), HLA-G (FIG. 15C), or PD-L1 (FIG. 15D).

FIGS. 16A-16B show levels of T cell activation and donor-specific antibody binding detected in samples from humanized mice injected with either human T cells, HLA-I/II deficient cells, or MHC I/II deficient cells overexpressing either HLA-E, HLA-G, or PD-L1. FIG. 16A depicts data from an IFNg (TH1) ELISPOT assay. FIG. 16B depicts data from an IgM antibody binding.

Other objects, advantages and embodiments of the present technology will be apparent from the detailed description following.

### DETAILED DESCRIPTION

### I. Introduction

Described herein are engineered or modified human immune evasive cells based, in part, on the hypoimmune editing platform described in WO2018132783. To overcome the problem of a subject's immune rejection of these stem cell-derived transplants, the inventors have developed and describe herein hypoimmunogenic cells (e.g., hypoimmunogenic pluripotent cells, differentiated cells derived from such and primary cells) that represent a viable source for any transplantable cell type. Such cells are protected from adaptive and/or innate immune rejection upon administration to a recipient subject. Advantageously, the cells disclosed herein are not rejected by the recipient subject's immune system, regardless of the subject's genetic make-up. Such cells are protected from adaptive and innate immune rejection upon administration to a recipient subject. In some embodiments, the hypoimmunogenic cells do not express MHC I and/or II antigens and/or T-cell receptors. In many embodiments, the hypoimmunogenic cells do not express MHC I and II antigens and/or T-cell receptors and overexpress a HLA-E variant protein, a HLA-G variant protein, and/or an exogenous PD-L1 protein. In many embodiments, the hypoimmunogenic cells such as hypoimmunogenic T cells including those derived from hypoimmunogenic iPSCs or primary T cells do not express MHC I and II antigens and/or T-cell receptors, overexpress a HLA-E variant protein, a HLA-G variant protein, and/or an exogenous PD-L1 protein and express exogenous CARs.

In some embodiments, hypoimmunogenic cells outlined herein are not subject to an innate immune cell rejection. In some instances, hypoimmunogenic cells are not susceptible to NK cell-mediated lysis. In some instances, hypoimmunogenic cells are not susceptible to macrophage engulfment. In some embodiments, hypoimmunogenic cells are useful as a source of universally compatible cells or tissues (e.g., universal donor cells or tissues) that are transplanted into a recipient subject with little to no immunosuppressant agent needed. Such hypoimmunogenic cells retain cell-specific characteristics and features upon transplantation.

The technology disclosed herein utilizes expression of tolerogenic factors and modulation (e.g., reduction or elimination) of MHC I, MHC II, and/or TCR expression in human cells. In some embodiments, genome editing technologies utilizing rare-cutting endonucleases (e.g., the CRISPR/Cas, TALEN, zinc finger nuclease, meganuclease, and homing endonuclease systems) are also used to reduce or eliminate expression of critical immune genes (e.g., by deleting genomic DNA of critical immune genes) in the cells. In some embodiments, genome editing technologies or other gene modulation technologies are used to insert tolerance-inducing (tolerogenic) factors in human cells, rendering the cells and their progeny (include any differentiated cells prepared therefrom) able to evade immune recognition upon engrafting into a recipient subject. As such, the cells described herein exhibit modulated expression of one or more genes and factors that affect MHC I, MHC II, and/or TCR expression and evade the recipient subject's immune system.

The genome editing techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

The practice of the numerous embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

### II. Definitions

As used herein to characterize a cell, the term “hypoimmunogenic” generally means that such cell is less prone to innate or adaptive immune rejection by a subject into which such cells are transplanted, e.g., the cell is less prone to allorejection by a subject into which such cells are transplanted. For example, relative to an unaltered or unmodified wild-type cell, such a hypoimmunogenic cell may be about 2.5%, 5%, 10%, 20%, 30%, 40%, 50%, 60%, 70%, 80%, 90%, 95%, 97.5%, 99% or more less prone to immune rejection by a subject into which such cells are transplanted. In some embodiments, genome editing technologies are used to modulate the expression of MHC I and MHC II genes, and thus, generate a hypoimmunogenic cell. In some embodiments, a hypoimmunogenic cell evades immune rejection in an MHC-mismatched allogenic recipient. In some instance, differentiated cells produced from the hypoimmunogenic stem cells outlined herein evade immune rejection when administered (e.g., transplanted or grafted) to an MHC-mismatched allogenic recipient. In some embodiments, a hypoimmunogenic cell is protected from T cell-mediated adaptive immune rejection and/or innate immune cell rejection. Detailed descriptions of hypoimmunogenic cells, methods of producing thereof, and methods of using thereof are found in WO2016183041 filed May 9, 2015; WO2018132783 filed Jan. 14, 2018; WO2018176390 filed Mar. 20, 2018; WO2020018615 filed Jul. 17, 2019; WO2020018620 filed Jul. 17, 2019; PCT/US2020/44635 filed Jul. 31, 2020; U.S. 62/881,840 filed Aug. 1, 2019; U.S. 62/891,180 filed Aug. 23, 2019; U.S. 63/016,190, filed Apr. 27, 2020; and U.S. 63/052,360 filed Jul. 15, 2020, the disclosures including the examples, sequence listings and figures are incorporated herein by reference in their entirety.

Hypoimmunogencity of a cell can be determined by evaluating the immunogenicity of the cell such as the cell's ability to elicit adaptive and innate immune responses. Such immune response can be measured using assays recognized by those skilled in the art. In some embodiments, an immune response assay measures the effect of a hypoimmunogenic cell on T cell proliferation, T cell activation, T cell killing, NK cell proliferation, NK cell activation, and macrophage activity. In some cases, hypoimmunogenic cells and derivatives thereof undergo decreased killing by T cells and/or NK cells upon administration to a subject. In some instances, the cells and derivatives thereof show decreased macrophage engulfment compared to an unmodified or wildtype cell. In some embodiments, a hypoimmunogenic cell elicits a reduced or diminished immune response in a recipient subject compared to a corresponding unmodified wild-type cell. In some embodiments, a hypoimmunogenic cell is nonimmunogenic or fails to elicit an immune response in a recipient subject.

“Immunosuppressive factor” or “immune regulatory factor” or “tolerogenic factor” as used herein include hypoimmunity factors, complement inhibitors, and other factors that modulate or affect the ability of a cell to be recognized by the immune system of a host or recipient subject upon administration, transplantation, or engraftment.

“Immune signaling factor” as used herein refers to, in some cases, a molecule, protein, peptide and the like that activates immune signaling pathways.

“Safe harbor locus” as used herein refers to a gene locus that allows safe expression of a transgene or an exogenous gene. Exemplary “safe harbor” loci include, but are not limited to, a CCR5 gene, a CXCR4 gene, a PPP1R12C (also known as AAVS1) gene, an albumin gene, a SHS231 locus, a CLYBL gene, a Rosa gene (e.g., ROSA26), an F3 gene (also known as CD142), a MICA gene, a MICB gene, a LRP1 gene (also known as CD91), a HMGB1 gene, an ABO gene, a RHD gene, a FUT1 gene, and a KDM5D gene (also known as HY). The exogenous gene can be inserted in the CDS region for B2M, CIITA, TRAC, TRBC, CCR5, F3 (i.e., CD142), MICA, MICB, LRP1, HMGB1, ABO, RHD, FUT1, or KDM5D (i.e., HY). The exogenous gene can be inserted in introns 1 or 2 for PPP1R12C (i.e., AAVS1) or CCR5. The exogenous gene can be inserted in exons 1 or 2 or 3 for CCR5. The exogenous gene can be inserted in intron 2 for CLYBL. The exogenous gene can be inserted in a 500 bp window in Ch-4:58, 976, 613 (i.e., SHS231). The exogenous gene can be insert in any suitable region of the aforementioned safe harbor loci that allows for expression of the exogenous, including, for example, an intron, an exon or a coding sequence region in a safe harbor locus.

A “gene” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation.

The term “genetic modification” and its grammatical equivalents as used herein can refer to one or more alterations of a nucleic acid, e.g., the nucleic acid within an organism's genome. For example, genetic modification can refer to alterations, additions, and/or deletion of genes or portions of genes or other nucleic acid sequences. A genetically modified cell can also refer to a cell with an added, deleted and/or altered gene or portion of a gene. A genetically modified cell can also refer to a cell with an added nucleic acid sequence that is not a gene or gene portion. Genetic modifications include, for example, both transient knock-in or knock-down mechanisms, and mechanisms that result in permanent knock-in, knock-down, or knock-out of target genes or portions of genes or nucleic acid sequences Genetic modifications include, for example, both transient knock-in and mechanisms that result in permanent knock-in of nucleic acids seqeunces Genetic modifications also include, for example, reduced or increased transcription, reduced or increased mRNA stability, reduced or increased translation, and reduced or increased protein stability.

“Modulation” of gene expression refers to a change in the expression level of a gene. Modulation of expression can include, but is not limited to, gene activation and gene repression. Modulation may also be complete, i.e. wherein gene expression is totally inactivated or is activated to wildtype levels or beyond; or it may be partial, wherein gene expression is partially reduced, or partially activated to some fraction of wildtype levels.

The term “operatively linked” or “operably linked” are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

A “vector” or “construct” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors. Methods for the introduction of vectors or constructs into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

“Pluripotent stem cells” as used herein have the potential to differentiate into any of the three germ layers: endoderm (e.g., the stomach linking, gastrointestinal tract, lungs, etc.), mesoderm (e.g., muscle, bone, blood, urogenital tissue, etc) or ectoderm (e.g., epidermal tissues and nervous system tissues). The term “pluripotent stem cells,” as used herein, also encompasses “induced pluripotent stem cells”, or “iPSCs”, “embryonic stem cells”, or “ESCs”, a type of pluripotent stem cell derived from a non-pluripotent cell. In some embodiments, a pluripotent stem cell is produced or generated from a cell that is not a pluripotent cell. In other words, pluripotent stem cells can be direct or indirect progeny of a non-pluripotent cell. Examples of parent cells include somatic cells that have been reprogrammed to induce a pluripotent, undifferentiated phenotype by various means. Such “ESC”, “ESC”, “iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009); Huangfu et al, Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) The generation of induced pluripotent stem cells (iPSCs) is outlined below. As used herein, “hiPSCs” are human induced pluripotent stem cells. In some embodiments, “pluripotent stem cells,” as used herein, also encompasses mesenchymal stem cells (MSCs), and/or embryonic stem cells (ESCs).

In some embodiments, the cells are engineered to have reduced or increased expression of one or more targets relative to an unaltered or unmodified wild-type cell. In some embodiments, the cells are engineered to have constitutive reduced or increased expression of one or more targets relative to an unaltered or unmodified wild-type cell. In some embodiments, the cells are engineered to have regulatable reduced or increased expression of one or more targets relative to an unaltered or unmodified wild-type cell. By “wild-type” or “wt” or “control” in the context of a cell means any cell found in nature. Examples of wild-type or control cells include primary cells and T cells found in nature.

By “HLA” or “human leukocyte antigen” complex is a gene complex encoding the major histocompatibility complex (MHC) proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A, HLA-B and HLA-C, which present peptides from the inside of the cell, and antigens presented by the HLA-I complex attract killer T-cells (also known as CD8+ T-cells or cytotoxic T cells). The HLA-I proteins are associated with β-2 microglobulin (B2M). HLA-II includes five proteins, HLA-DP, HLA-DM, HLA-DOB, HLA-DQ and HLA-DR, which present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

As used herein, the terms “protein variant” or “variant protein,” as well as grammatical variations thereof are used interchangeably to refer to a protein that differs from a parent protein by virtue of at least one amino acid alteration, including modification, substitution, insertion, or deletion. The terms “amino acid modification” or “modification” or “amino acid substitution” or “substitution,” as used herein refers to an amino acid substitution, insertion, and/or deletion in a polypeptide sequence. An “amino acid substitution” or “substitution” as used herein, refers to replacement of an amino acid at a particular position in a parent polypeptide sequence with another (e.g., different) amino acid. An “amino acid insertion” or “insertion” as used herein refers to an addition of an amino acid at a particular position in a parent polypeptide sequence. An “amino acid deletion” or “deletion,” as used herein refers to removal of an amino acid at a particular position in a parent polypeptide sequence.

As used herein, the terms “grafting”, “administering,” “introducing,” “implanting” and “transplanting” as well as grammatical variations thereof are used interchangeably in the context of the placement of cells (e.g., cells described herein) into a subject, by a method or route which results in at least partial localization of the introduced cells at a desired site. The cells can be implanted directly to the desired site, or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years. In some embodiments, the cells can also be administered (e.g., injected) a location other than the desired site, such as in the brain or subcutaneously, for example, in a capsule to maintain the implanted cells at the implant location and avoid migration of the implanted cells.

As used herein, the terms “treating” and “treatment” includes administering to a subject a therapeutically or clinically effective amount of cells described herein so that the subject has a reduction in at least one symptom of the disease or an improvement in the disease, for example, beneficial or desired therapeutic or clinical results. For purposes of this technology, beneficial or desired therapeutic or clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. In some embodiments, one or more symptoms of a condition, disease or disorder are alleviated by at least 5%, at least 10%, at least 20%, at least 30%, at least 40%, or at least 50% upon treatment of the condition, disease or disorder.

For purposes of this technology, beneficial or desired therapeutic or clinical results of disease treatment include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable.

The term “cancer” as used herein is defined as a hyperproliferation of cells whose unique trait (e.g., loss of normal controls) results in unregulated growth, lack of differentiation, local tissue invasion, and metastasis. With respect to the inventive methods, the cancer can be any cancer, including any of acute lymphocytic cancer, acute myeloid leukemia, alveolar rhabdomyosarcoma, bladder cancer, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and urinary bladder cancer. As used herein, the term “tumor” refers to an abnormal growth of cells or tissues of the malignant type, unless otherwise specifically indicated and does not include a benign type tissue.

The term “chronic infectious disease” refers to a disease caused by an infectious agent wherein the infection has persisted. Such a disease may include hepatitis (A, B, or C), herpes virus (e.g., VZV, HSV-1, HSV-6, HSV-II, CMV, and EBV), and HIV/AIDS. Non-viral examples may include chronic fungal diseases such Aspergillosis, Candidiasis, Coccidioidomycosis, and diseases associated with Cryptococcus and Histoplasmosis. None limiting examples of chronic bacterial infectious agents may be Chlamydia pneumoniae, Listeria monocytogenes, and Mycobacterium tuberculosis. In some embodiments, the disorder is human immunodeficiency virus (HIV) infection. In some embodiments, the disorder is acquired immunodeficiency syndrome (AIDS).

The term “autoimmune disease” refers to any disease or disorder in which the subject mounts a destructive immune response against its own tissues. Autoimmune disorders can affect almost every organ system in the subject (e.g., human), including, but not limited to, diseases of the nervous, gastrointestinal, and endocrine systems, as well as skin and other connective tissues, eyes, blood and blood vessels. Examples of autoimmune diseases include, but are not limited to Hashimoto's thyroiditis, Systemic lupus erythematosus, Sjogren's syndrome, Graves' disease, Scleroderma, Rheumatoid arthritis, Multiple sclerosis, Myasthenia gravis and Diabetes.

In additional or alternative embodiments, the present technology contemplates altering target polynucleotide sequences in any manner which is available to the skilled artisan, e.g., utilizing a nuclease system such as a TAL effector nuclease (TALEN), zinc finger nuclease (ZFN) system, or RNA-guided transposases. It should be understood that although examples of methods utilizing CRISPR/Cas (e.g., Cas9 and Cas12a) and TALEN are described in detail herein, the present technology is not limited to the use of these methods/systems. Other methods of targeting, to reduce or ablate expression in target cells known to the skilled artisan can be utilized herein. The methods provided herein can be used to alter a target polynucleotide sequence in a cell. The present technology contemplates altering target polynucleotide sequences in a cell for any purpose. In some embodiments, the target polynucleotide sequence in a cell is altered to produce a mutant cell. As used herein, a “mutant cell” refers to a cell with a resulting genotype that differs from its original genotype. In some instances, a “mutant cell” exhibits a mutant phenotype, for example when a normally functioning gene is altered using the gene editing systems (e.g., CRISPR/Cas systems) of the present disclosure. In other instances, a “mutant cell” exhibits a wild-type phenotype, for example when a gene editing system (e.g., CRISPR/Cas systems) of the present disclosure is used to correct a mutant genotype. In some embodiments, the target polynucleotide sequence in a cell is altered to correct or repair a genetic mutation (e.g., to restore a normal phenotype to the cell). In some embodiments, the target polynucleotide sequence in a cell is altered to induce a genetic mutation (e.g., to disrupt the function of a gene or genomic element).

The methods of the present technology can be used to alter a target polynucleotide sequence in a cell. The present technology contemplates altering target polynucleotide sequences in a cell for any purpose. In some embodiments, the target polynucleotide sequence in a cell is altered to produce a mutant cell. As used herein, a “mutant cell” refers to a cell with a resulting genotype that differs from its original genotype. In some instances, a “mutant cell” exhibits a mutant phenotype, for example when a normally functioning gene is altered using the CRISPR/Cas systems of the present technology. In other instances, a “mutant cell” exhibits a wild-type phenotype, for example when a CRISPR/Cas system of the present technology is used to correct a mutant genotype. In some embodiments, the target polynucleotide sequence in a cell is altered to correct or repair a genetic mutation (e.g., to restore a normal phenotype to the cell). In some embodiments, the target polynucleotide sequence in a cell is altered to induce a genetic mutation (e.g., to disrupt the function of a gene or genomic element).

In some embodiments, the alteration is an indel. As used herein, “indel” refers to a mutation resulting from an insertion, deletion, or a combination thereof. As will be appreciated by those skilled in the art, an indel in a coding region of a genomic sequence will result in a frameshift mutation, unless the length of the indel is a multiple of three. In some embodiments, the alteration is a point mutation. As used herein, “point mutation” refers to a substitution that replaces one of the nucleotides. A CRISPR/Cas system of the present technology can be used to induce an indel of any length or a point mutation in a target polynucleotide sequence.

As used herein, “knock out” or “knock-out” includes deleting all or a portion of the target polynucleotide sequence in a way that interferes with the function of the target polynucleotide sequence. For example, a knock out can be achieved by altering a target polynucleotide sequence by inducing an insertion or a deletion (“indel”) in the target polynucleotide sequence in a functional domain of the target polynucleotide sequence (e.g., a DNA binding domain). Those skilled in the art will readily appreciate how to use the CRISPR/Cas systems of the present technology to knock out a target polynucleotide sequence or a portion thereof based upon the details described herein.

In some embodiments, the alteration results in a knock out or knock down of the target polynucleotide sequence or a portion thereof. Knocking out a target polynucleotide sequence or a portion thereof using a gene editing system (e.g., CRISPR/Cas) of the present technology can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject) or for changing the genotype or phenotype of a cell.

By “knock in” or “knock-in” herein is meant a process that adds a genetic function to a host cell as well as a genetic modification resulting from the insertion of a DNA sequence into a chromosomal locus in a host cell. This causes increased levels of expression of the knocked in gene, portion of gene, or nucleic acid sequence inserted product, e.g., an increase in RNA transcript levels and/or encoded protein levels. As will be appreciated by those in the art, this can be accomplished in several ways, including adding one or more additional copies of the gene to the host cell or altering a regulatory component of the endogenous gene increasing expression of the protein is made or inserting a specific nucleic acid sequence whose expression is desired. This may be accomplished by modifying the promoter, adding a different promoter, adding an enhancer, or modifying other gene expression sequences.

In some embodiments, the alteration results in reduced expression or decreased expression of the target polynucleotide sequence and/or the target polypeptide sequence. The terms “decrease,” “reduced,” “reduction,” and “decrease” are all used herein generally to mean a decrease by a statistically significant amount. However, for avoidance of doubt, decrease,” “reduced,” “reduction,” or “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level. The reduced expression or decreased expression can result from reduced gene expression, reduced protein/polypeptide expression, reduced mRNA translation, reduced mRNA stability, reduced surface expression of the protein/polypeptide, as well as reduced functional expression, for example due to a reduction in protein/polypeptide activity, function, and/or stability.

The terms “increased,” “increase,” “enhance,” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “exogenous” in intended to mean that the referenced molecule or the referenced polypeptide is introduced into the cell of interest. The polypeptide can be introduced, for example, by introduction of an encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell.

The term “endogenous” refers to a referenced molecule or polypeptide that is present in the cell. Similarly, the term when used in reference to expression of an encoding nucleic acid refers to expression of an encoding nucleic acid contained within the cell and not exogenously introduced.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al, infra).

One example of an algorithm that is suitable for determining percent sequence identity and sequence similarity is the BLAST algorithm, which is described in Altschul et al, J. Mol. Biol. 215:403-410 (1990). Software for performing BLAST analyses is publicly available through the National Center for Biotechnology Information.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states, which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g., dog, cat, horse, and the like, or production mammal, e.g., cow, sheep, pig, and the like.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features readily separated from or combined with the features of any of the other several embodiments without departing from the scope or spirit of the present technology. Any recited method may be carried out in the order of events recited or in any other order that is logically possible. Although any methods and materials similar or equivalent to those described herein may also be used in the practice or testing of the present technology, representative illustrative methods and materials are now described.

As described in the present technology, the following terms will be employed, and are defined as indicated below.

Before the present technology is further described, it is to be understood that this technology is not limited to numerous embodiments described, as such may, of course, vary. It is also to be understood that the terminology used herein is for the purpose of describing some embodiments only, and is not intended to be limiting, since the scope of the present technology will be limited only by the appended claims.

Unless defined otherwise, all technical and scientific terms used herein have the same meaning as commonly understood by one of ordinary skill in the art to which this technology belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the present technology. The upper and lower limits of these smaller ranges may independently be included in the smaller ranges and are also encompassed within the present technology, subject to any specifically excluded limit in the stated range. Where the stated range includes one or both of the limits, ranges excluding either or both of those included limits are also included in the present technology. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context presented, provides the substantial equivalent of the specifically recited number. The term about is used herein to mean plus or minus ten percent (10%) of a value. For example, “about 100” refers to any number between 90 and 110.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the technology described herein is not entitled to antedate such publication by virtue of prior technology. Further, the dates of publication provided might be different from the actual publication dates, which may need to be independently confirmed.

### III. DETAILED DESCRIPTION OF THE EMBODIMENTS

### A. Hypoimmunogenic Cells

In some embodiments, the present technology provides engineered (e.g., modified and genetically modified) cells that express one or more exogenous receptors that enable the cells to evade activating NK cell mediated immune responses. In some embodiments, the exogenous receptors include, but are not limited to, an HLA-E variant protein, an HLA-G variant protein, and an exogenous PD-L1 protein. In some instances, the exogenous PD-L1 protein is a wild-type PD-L1 protein or a variant thereof.

In some embodiments, the cells are induced pluripotent stem cells, any type of differentiated cells thereof, primary immune cells and other primary cells of any tissue. In some embodiments, the differentiated cells are T cells and subpopulations thereof, NK cells and subpopulations thereof, and endothelial cells and subpopulations thereof. In some embodiments, the primary immune cells are T cells and subpopulations thereof and NK cells and subpopulations thereof. In some embodiments, the primary tissue cells include primary endothelial cells and subpopulations thereof.

In some embodiments, cells described herein express one or more exogenous receptors selected from the group consisting of an HLA-E variant protein, an HLA-G variant protein, and an exogenous PD-L1 protein such that polynucleotide(s) encoding the exogenous receptor(s) are inserted into (e.g., knocked into) a gene locus selected from the group consisting of an HLA-A locus, an HLA-B locus, an HLA-C locus, a CD155 locus, a B2M locus, a CIITA locus, an RHD locus, a TRAC locus, a TRB locus and a safe harbor locus.

In some embodiments, an HLA-E variant polynucleotide is knocked into a gene locus selected from the group consisting of an HLA-A locus, an HLA-B locus, an HLA-C locus, a CD155 locus, a B2M locus, a CIITA locus, an RHD locus, a TRAC locus, a TRB locus and a safe harbor locus. In some embodiments, an HLA-G variant polynucleotide is knocked into a gene locus selected from the group consisting of an HLA-A locus, an HLA-B locus, an HLA-C locus, a CD155 locus, a B2M locus, a CIITA locus, an RHD locus, a TRAC locus, a TRB locus and a safe harbor locus. In some embodiments, a PD-L1 polynucleotide is knocked into a gene locus selected from the group consisting of an HLA-A locus, an HLA-B locus, an HLA-C locus, a CD155 locus, a B2M locus, a CIITA locus, an RHD locus, a TRAC locus, a TRB locus and a safe harbor locus.

In some embodiments, an HLA-E variant polynucleotide and an HLA-G variant polynucleotide are knocked into a gene locus selected from the group consisting of an HLA-A locus, an HLA-B locus, an HLA-C locus, a CD155 locus, a B2M locus, a CIITA locus, an RHD locus, a TRAC locus, a TRB locus and a safe harbor locus. In some embodiments, an HLA-E variant polynucleotide and a PD-L1 polynucleotide are knocked into a gene locus selected from the group consisting of an HLA-A locus, an HLA-B locus, an HLA-C locus, a CD155 locus, a B2M locus, a CIITA locus, an RHD locus, a TRAC locus, a TRB locus and a safe harbor locus. In some embodiments, an HLA-G variant polynucleotide and a PD-L1 polynucleotide are knocked into a gene locus selected from the group consisting of an HLA-A locus, an HLA-B locus, an HLA-C locus, a CD155 locus, a B2M locus, a CIITA locus, an RHD locus, a TRAC locus, a TRB locus and a safe harbor locus.

In some embodiments, an HLA-E variant polynucleotide is inserted into an HLA-A locus, disrupting one or both alleles of the HLA-A gene. In some embodiments, an HLA-E variant polynucleotide is inserted into an HLA-B locus, disrupting one or both alleles of the HLA-B gene. In some embodiments, an HLA-E variant polynucleotide is inserted into an HLA-C locus, disrupting one or both alleles of the HLA-C gene. In some embodiments, an HLA-E variant polynucleotide t is inserted into a CD155 locus, disrupting one or both alleles of the CD155 gene. In some embodiments, an HLA-E variant polynucleotide is inserted into a B2M locus, disrupting one or both alleles of the B2M gene. In some embodiments, an HLA-E variant polynucleotide is inserted into a CIITA locus, disrupting one or both alleles of the CIITA gene. In some embodiments, an HLA-E variant polynucleotide is inserted into an RHD locus, disrupting one or both alleles of the RHD gene. In some embodiments, an HLA-E variant polynucleotide is inserted into a TRAC locus, disrupting one or both alleles of the TRAC gene. In some embodiments, an HLA-E variant polynucleotide t is inserted into a TRBC locus, disrupting one or both alleles of the TRB gene. In some embodiments, an HLA-E variant polynucleotide is inserted into a safe harbor locus, disrupting one or both alleles of the safe harbor gene.

In some embodiments, an HLA-G variant polynucleotide is inserted into an HLA-A locus, disrupting one or both alleles of the HLA-A gene. In some embodiments, an HLA-G variant polynucleotide is inserted into an HLA-B locus, disrupting one or both alleles of the HLA-B gene. In some embodiments, an HLA-G variant polynucleotide is inserted into an HLA-C locus, disrupting one or both alleles of the HLA-C gene. In some embodiments, an HLA-G variant polynucleotide is inserted into a CD155 locus, disrupting one or both alleles of the CD155 gene. In some embodiments, an HLA-G variant polynucleotide is inserted into a B2M locus, disrupting one or both alleles of the B2M gene. In some embodiments, an HLA-G variant polynucleotide is inserted into a CIITA locus, disrupting one or both alleles of the CIITA gene. In some embodiments, an HLA-G variant polynucleotide is inserted into an RHD locus, disrupting one or both alleles of the RHD gene. In some embodiments, an HLA-G variant polynucleotide is inserted into a TRAC locus, disrupting one or both alleles of the TRAC gene. In some embodiments, an HLA-G variant is inserted into a TRBC locus, disrupting one or both alleles of the TRB gene. In some embodiments, an HLA-G variant polynucleotide is inserted into a safe harbor locus, disrupting one or both alleles of the safe harbor gene.

In some embodiments, an exogenous PD-L1 polynucleotide is inserted into an HLA-A locus, disrupting one or both alleles of the HLA-A gene. In some embodiments, a PD-L1 variant is inserted into an HLA-B locus, disrupting one or both alleles of the HLA-B gene. In some embodiments, an exogenous PD-L1 polynucleotide is inserted into an HLA-C locus, disrupting one or both alleles of the HLA-C gene. In some embodiments, a PD-L1 variant is inserted into a CD155 locus, disrupting one or both alleles of the CD155 gene. In some embodiments, an exogenous PD-L1 polynucleotide is inserted into a B2M locus, disrupting one or both alleles of the B2M gene. In some embodiments, a PD-L1 variant is inserted into a CIITA locus, disrupting one or both alleles of the CIITA gene. In some embodiments, an exogenous PD-L1 polynucleotide is inserted into an RHD locus, disrupting one or both alleles of the RHD gene. In some embodiments, a PD-L1 variant is inserted into a TRAC locus, disrupting one or both alleles of the TRAC gene. In some embodiments, an exogenous PD-L1 polynucleotide is inserted into a TRBC locus, disrupting one or both alleles of the TRB gene. In some embodiments, a PD-L1 variant is inserted into a safe harbor locus, disrupting one or both alleles of the safe harbor gene.

In some embodiments, an HLA-E variant and an HLA-G variant are inserted into an HLA-A locus, disrupting one or both alleles of the HLA-A gene. In some embodiments, an HLA-E variant and an HLA-G variant are inserted into an HLA-B locus, disrupting one or both alleles of the HLA-B gene. In some embodiments, an HLA-E variant and an HLA-G variant are inserted into an HLA-C locus, disrupting one or both alleles of the HLA-C gene. In some embodiments, an HLA-E variant and an HLA-G variant are inserted into a CD155 locus, disrupting one or both alleles of the CD155 gene. In some embodiments, an HLA-E variant and an HLA-G variant are inserted into a B2M locus, disrupting one or both alleles of the B2M gene. In some embodiments, an HLA-E variant and an HLA-G variant are inserted into a CIITA locus, disrupting one or both alleles of the CIITA gene. In some embodiments, an HLA-E variant and an HLA-G variant are inserted into an RHD locus, disrupting one or both alleles of the RHD gene. In some embodiments, an HLA-E variant and an HLA-G variant are inserted into a TRAC locus, disrupting one or both alleles of the TRAC gene. In some embodiments, an HLA-E variant and an HLA-G variant are inserted into a TRBC locus, disrupting one or both alleles of the TRB gene. In some embodiments, an HLA-E variant and an HLA-G variant are inserted into a safe harbor locus, disrupting one or both alleles of the safe harbor gene.

In some embodiments, an HLA-E variant and an exogenous PD-L1 are inserted into an HLA-A locus, disrupting one or both alleles of the HLA-A gene. In some embodiments, an HLA-E variant and an exogenous PD-L1 are inserted into an HLA-B locus, disrupting one or both alleles of the HLA-B gene. In some embodiments, an HLA-E variant and an exogenous PD-L1 are inserted into an HLA-C locus, disrupting one or both alleles of the HLA-C gene. In some embodiments, an HLA-E variant and an exogenous PD-L1 are inserted into a CD155 locus, disrupting one or both alleles of the CD155 gene. In some embodiments, an HLA-E variant and an exogenous PD-L1 are inserted into a B2M locus, disrupting one or both alleles of the B2M gene. In some embodiments, an HLA-E variant and an exogenous PD-L1 are inserted into a CIITA locus, disrupting one or both alleles of the CIITA gene. In some embodiments, an HLA-E variant and an exogenous PD-L1 are inserted into an RHD locus, disrupting one or both alleles of the RHD gene. In some embodiments, an HLA-E variant and an exogenous PD-L1 are inserted into a TRAC locus, disrupting one or both alleles of the TRAC gene. In some embodiments, an HLA-E variant and an exogenous PD-L1 are inserted into a TRBC locus, disrupting one or both alleles of the TRB gene. In some embodiments, an HLA-E variant and an exogenous PD-L1 are inserted into a safe harbor locus, disrupting one or both alleles of the safe harbor gene.

In some embodiments, an HLA-G variant and an exogenous PD-L1 are inserted into an HLA-A locus, disrupting one or both alleles of the HLA-A gene. In some embodiments, an HLA-G variant and an exogenous PD-L1 are inserted into an HLA-B locus, disrupting one or both alleles of the HLA-B gene. In some embodiments, an HLA-G variant and an exogenous PD-L1 are inserted into an HLA-C locus, disrupting one or both alleles of the HLA-C gene. In some embodiments, an HLA-G variant and an exogenous PD-L1 are inserted into a CD155 locus, disrupting one or both alleles of the CD155 gene. In some embodiments, an HLA-G variant and an exogenous PD-L1 are inserted into a B2M locus, disrupting one or both alleles of the B2M gene. In some embodiments, an HLA-G variant and an exogenous PD-L1 are inserted into a CIITA locus, disrupting one or both alleles of the CIITA gene. In some embodiments, an HLA-G variant and an exogenous PD-L1 are inserted into an RHD locus, disrupting one or both alleles of the RHD gene. In some embodiments, an HLA-G variant and an exogenous PD-L1 are inserted into a TRAC locus, disrupting one or both alleles of the TRAC gene. In some embodiments, an HLA-G variant and an exogenous PD-L1 are inserted into a TRBC locus, disrupting one or both alleles of the TRB gene. In some embodiments, an HLA-G variant and an exogenous PD-L1 are inserted into a safe harbor locus, disrupting one or both alleles of the safe harbor gene.

In some embodiments, the present technology is directed to pluripotent stem cells, (e.g., pluripotent stem cells and induced pluripotent stem cells (iPSCs)), differentiated cells derived from such pluripotent stem cells (such as but not limited to T cells and NK cells), and primary cells (such as, but not limited to, primary T cells and primary NK cells). In some embodiments, the pluripotent stem cells, differentiated cells derived therefrom, and primary cells such as primary T cells and primary NK cells are engineered for reduced expression or no expression of MHC class I and/or MHC class II human leukocyte antigens, and in some instances, for reduced expression or lack of expression of a T-cell receptor (TCR) complex. In some embodiments, the hypoimmune T cells and primary T cells overexpress a HLA-E variant protein, a HLA-G variant protein, and/or an exogenous PD-L1 protein and a chimeric antigen receptor (CAR), as well as exhibit (i) reduced expression or no expression of MHC class I and/or MHC class II human leukocyte antigens, and (ii) reduced expression or no expression of a T-cell receptor (TCR) complex. In some embodiments, the CAR comprises an antigen binding domain that binds to any one selected from the group consisting of CD19, CD22, CD38, CD123, CD138, and BCMA. In some embodiments, the CAR is a CD19-specific CAR. In some embodiments, the CAR is a CD22-specific CAR. In some instances, the CAR is a CD38-specific CAR. In some embodiments, the CAR is a CD123-specific CAR. In some embodiments, the CAR is a CD138-specific CAR. In some instances, the CAR is a BCMA-specific CAR. In some embodiments, the CAR is a bispecific CAR. In some embodiments, the bispecific CAR is a CD19/CD22-bispecific CAR. In some embodiments, the bispecific CAR is a BCMA/CD38-bispecific CAR. In some embodiments, the cells described express a CD19-specific CAR and a different CAR, such as, but not limited to a CD22-specific CAR, a CD38-specific CAR, a CD123-specific CAR, a CD138-specific CAR, and a BCMA-specific CAR. In some embodiments, the cells described express a CD22-specific CAR and a different CAR, such as, but not limited to a CD19-specific CAR, a CD38-specific CAR, a CD123-specific CAR, a CD138-specific CAR, and a BCMA-specific CAR. In some embodiments, the cells described express a CD38-specific CAR and a different CAR, such as, but not limited to a CD22-specific CAR, a CD18-specific CAR, a CD123-specific CAR, a CD138-specific CAR, and a BCMA-specific CAR. In some embodiments, the cells described express a CD123-specific CAR and a different CAR, such as, but not limited to a CD22-specific CAR, a CD38-specific CAR, a CD19-specific CAR, a CD138-specific CAR, and a BCMA-specific CAR. In some embodiments, the cells described express a CD138-specific CAR and a different CAR, such as, but not limited to a CD22-specific CAR, a CD38-specific CAR, a CD123-specific CAR, a CD19-specific CAR, and a BCMA-specific CAR. In some embodiments, the cells described express a BCMA-specific CAR and a different CAR, such as, but not limited to a CD22-specific CAR, a CD38-specific CAR, a CD123-specific CAR, a CD138-specific CAR, and a CD19-specific CAR.

In some embodiments, hypoimmune T cells derived from iPSCs and primary T cells overexpress a HLA-E variant protein, a HLA-G variant protein, and/or an exogenous PD-L1 protein and a chimeric antigen receptor (CAR), and include a genomic modification of the HLA-A gene. In some embodiments, hypoimmune T cells derived from iPSCs and primary T cells overexpress a HLA-E variant protein, a HLA-G variant protein, and/or an exogenous PD-L1 protein and a chimeric antigen receptor (CAR), and include a genomic modification of the HLA-B gene. In some embodiments, hypoimmune T cells derived from iPSCs and primary T cells overexpress a HLA-E variant protein, a HLA-G variant protein, and/or an exogenous PD-L1 protein and a chimeric antigen receptor (CAR), and include a genomic modification of the HLA-C gene. In some embodiments, hypoimmune T cells derived from iPSCs and primary T cells overexpress a HLA-E variant protein, a HLA-G variant protein, and/or an exogenous PD-L1 protein and a chimeric antigen receptor (CAR), and include a genomic modification of the CD155 gene. In some embodiments, hypoimmune T cells derived from iPSCs and primary T cells overexpress a HLA-E variant protein, a HLA-G variant protein, and/or an exogenous PD-L1 protein and a chimeric antigen receptor (CAR), and include a genomic modification of the B2M gene. In some embodiments, hypoimmune T cells derived from iPSCs and primary T cells overexpress a HLA-E variant protein, a HLA-G variant protein, and/or an exogenous PD-L1 protein and include a genomic modification of the CIITA gene. In some embodiments, hypoimmune T cells derived from iPSCs and primary T cells overexpress a HLA-E variant protein, a HLA-G variant protein, and/or an exogenous PD-L1 protein and a CAR, and include a genomic modification of the TRAC gene. In some embodiments, hypoimmune T cells derived from iPSCs and primary T cells overexpress a HLA-E variant protein, a HLA-G variant protein, and/or an exogenous PD-L1 protein and a CAR, and include a genomic modification of the TRB gene. In some embodiments, hypoimmune T cells derived from iPSCs and primary T cells overexpress a HLA-E variant protein, a HLA-G variant protein, and/or an exogenous PD-L1 protein and a CAR, and include one or more genomic modifications selected from the group consisting of the HLA-A, HLA-B, HLA-C, CD155, B2M, CIITA, TRAC, and TRB genes. In some embodiments, hypoimmune T cells derived from iPSCs and primary T cells overexpress a HLA-E variant protein, a HLA-G variant protein, and/or an exogenous PD-L1 protein and a CAR, and include genomic modifications of the HLA-A, HLA-B, HLA-C, CD155, B2M, CIITA, TRAC, and TRB genes. In some embodiments, the cells are HLA-A^{-/-} cells that also express HLA-E variant proteins, HLA-G variant proteins, and/or an exogenous PD-L1 proteins as well as CARs. In some embodiments, the cells are HLA-B^{-/-} cells that also express HLA-E variant proteins, HLA-G variant proteins, and/or an exogenous PD-L1 proteins as well as CARs. In some embodiments, the cells are HLA-C^{-/-} cells that also express HLA-E variant proteins, HLA-G variant proteins, and/or an exogenous PD-L1 proteins as well as CARs. In some embodiments, the cells are CD155^{-/-} cells that also express HLA-E variant proteins, HLA-G variant proteins, and/or an exogenous PD-L1 proteins as well as CARs. In some embodiments, the cells are HLA-A^{-/-}, HLA-B^{-/-} cells that also express HLA-E variant proteins, HLA-G variant proteins, and/or an exogenous PD-L1 proteins as well as CARs. In some embodiments, the cells are HLA-A^{-/-}, HLA-C^{-/-}-cells that also express HLA-E variant proteins, HLA-G variant proteins, and/or an exogenous PD-L1 proteins as well as CARs. In some embodiments, the cells are HLA-A^{-/-}, CD155^{-/-} cells that also express HLA-E variant proteins, HLA-G variant proteins, and/or an exogenous PD-L1 proteins as well as CARs. In some embodiments, the cells are HLA-B^{-/-}, HLA-C^{-/-}-cells that also express HLA-E variant proteins, HLA-G variant proteins, and/or an exogenous PD-L1 proteins as well as CARs. In some embodiments, the cells are HLA-C^{-/-}, CD155^{-/-}-cells that also express HLA-E variant proteins, HLA-G variant proteins, and/or an exogenous PD-L1 proteins as well as CARs. In some embodiments, the cells are HLA-B^{-/-}, CD155^{-/-} cells that also express HLA-E variant proteins, HLA-G variant proteins, and/or an exogenous PD-L1 proteins as well as CARs. In some embodiments, the cells are HLA-A^{-/-}, HLA-B^{-/-}, HLA-C^{-/-} cells that also express HLA-E variant proteins, HLA-G variant proteins, and/or an exogenous PD-L1 proteins as well as CARs. In some embodiments, the cells are HLA-A^{-/-}, HLA-C^{-/-}, CD155^{-/-} cells that also express HLA-E variant proteins, HLA-G variant proteins, and/or an exogenous PD-L1 proteins as well as CARs.

In some embodiments, the cells are B2M^{-/-}, CIITA^{-/-}, TRAC^{-/-}, cells that also express HLA-E variant proteins, HLA-G variant proteins, and/or an exogenous PD-L1 proteins as well as CARs. In some embodiments, hypoimmune T cells are produced by differentiating induced pluripotent stem cells such as hypoimmunogenic induced pluripotent stem cells. In some embodiments, the hypoimmune T cells derived from iPSCs and primary T cells are B2M^{-/-}, CIITA^{-/-}, TRB^{-/-}, cells that also express HLA-E variant proteins, HLA-G variant proteins, and/or exogenous PD-L1 proteins as well as CARs. In some embodiments, the cells are B2M^{-/-}, CIITA^{-/-}, TRAC^{-/-}, TRB^{-/-}, cells that also express HLA-E variant proteins, HLA-G variant proteins, and/or exogenous PD-L1 proteins CARs. In many embodiments, the cells are B2M^{indel/indel} CIITA^{indel/indel}, TRAC^{indel/indel}, cells that also express HLA-E variant proteins, HLA-G variant proteins, and/or exogenous PD-L1 proteins CARs. In many embodiments, the cells are B2M^{indel/indel}, CIITA^{indel/indel}, TRB^{indel/indel}, cells that also express HLA-E variant proteins, HLA-G variant proteins, and/or exogenous PD-L1 proteins CARs. In many embodiments, the cells are B2M^{indel/indel}, CIITA^{indel/indel}, TRAC^{indel/indel}, TRB^{indel/indel}, cells that also express HLA-E variant proteins, HLA-G variant proteins, and/or exogenous PD-L1 proteins CARs.

In some embodiments, the engineered or modified cells described are pluripotent stem cells, induced pluripotent stem cells, NK cells differentiated from such pluripotent stem cells and induced pluripotent stem cells, T cells differentiated from such pluripotent stem cells and induced pluripotent stem cells, primary T cells or primary T cells. Non-limiting examples of T cells and primary T cells include CD3+ T cells, CD4+ T cells, CD8+ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), γδ T cells, and any other subtype of T cells. In some embodiments, the primary T cells are selected from a group that includes cytotoxic T-cells, helper T-cells, memory T-cells, regulatory T-cells, tumor infiltrating lymphocytes, and combinations thereof. Non-limiting examples of NK cells and primary NK cells include immature NK cells and mature NK cells.

In some embodiments, the primary T cells are from a pool of primary T cells from one or more donor subjects that are different than the recipient subject (e.g., the patient administered the cells). The primary T cells can be obtained from 1, 2, 3, 4, 5, 6, 7, 8, 9, 10, 20, 50, 100 or more donor subjects and pooled together. The primary T cells can be obtained from 1 or more, 2 or more, 3 or more, 4 or more, 5 or more, 6 or more, 7 or more, 8 or more, 9 or more, 10, or more 20 or more, 50 or more, or 100 or more donor subjects and pooled together. In some embodiments, the primary T cells are harvested from one or a plurality of individuals, and in some instances, the primary T cells or the pool of primary T cells are cultured in vitro. In some embodiments, the primary T cells or the pool of primary T cells are engineered to exogenously express a HLA-E variant protein, a HLA-G variant protein, and/or an exogenous PD-L1 protein and cultured in vitro.

In many embodiments, the primary T cells or the pool of primary T cells are engineered to express a chimeric antigen receptor (CAR). The CAR can be any known to those skilled in the art. Useful CARs include those that bind an antigen selected from a group that includes CD19, CD22, CD38, CD123, CD138, and BCMA. In some cases, the CAR is the same or equivalent to those used in FDA-approved CAR-T cell therapies such as, but not limited to, those used in tisagenlecleucel and axicabtagene ciloleucel, or others under investigation in clinical trials.

In some embodiments, the primary T cells or the pool of primary T cells are engineered to exhibit reduced expression of an endogenous T cell receptor compared to unmodified primary T cells. In many embodiments, the primary T cells or the pool of primary T cells are engineered to exhibit reduced expression of CTLA-4, PD-1, or both CTLA-4 and PD-1, as compared to unmodified primary T cells. Methods of genetically modifying a cell including a T cell are described in detail, for example, in WO2020/018620 and WO2016/183041, the disclosures of which are herein incorporated by reference in their entireties, including the tables, appendices, sequence listing and figures.

In some embodiments, the CAR-T cells comprise a CAR selected from a group including: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transmembrane domain, three or four signaling domains, and a domain which upon successful signaling of the CAR induces expression of a cytokine gene.

In some embodiments, the antigen binding domain of the CAR is selected from a group including, but not limited to, (a) an antigen binding domain targets an antigen characteristic of a neoplastic cell; (b) an antigen binding domain that targets an antigen characteristic of a T cell; (c) an antigen binding domain targets an antigen characteristic of an autoimmune or inflammatory disorder; (d) an antigen binding domain that targets an antigen characteristic of senescent cells; (e) an antigen binding domain that targets an antigen characteristic of an infectious disease; and (f) an antigen binding domain that binds to a cell surface antigen of a cell.

In some embodiments, the antigen binding domain is selected from a group that includes an antibody, an antigen-binding portion or fragment thereof, an scFv, and a Fab. In some embodiments, the antigen binding domain binds to CD19, CD22, CD38, CD123, CD138, or BCMA. In some embodiments, the antigen binding domain is an anti-CD19 scFv such as but not limited to FMC63.

In some embodiments, the transmembrane domain comprises one selected from a group that includes a transmembrane region of TCRα, TCRβ, TCRζ, CD3ε, CD3γ, CD3δ, CD3ζ, CD4, CD5, CD8a, CD8B, CD9, CD16, CD28, CD45, CD22, CD33, CD34, CD37, CD40, CD40L/CD154, CD45, CD64, CD80, CD86, OX40/CD134, 4-1BB/CD137, CD154, FcεRIγ, VEGFR2, FAS, FGFR2B, and functional variant thereof.

In some embodiments, the signaling domain(s) of the CAR comprises a costimulatory domain(s). For instance, a signaling domain can contain a costimulatory domain. Or, a signaling domain can contain one or more costimulatory domains. In many embodiments, the signaling domain comprises a costimulatory domain. In other embodiments, the signaling domains comprise costimulatory domains. In some cases, when the CAR comprises two or more costimulatory domains, two costimulatory domains are not the same. In some embodiments, the costimulatory domains comprise two costimulatory domains that are not the same. In some embodiments, the costimulatory domain enhances cytokine production, CAR-T cell proliferation, and/or CAR-T cell persistence during T cell activation. In some embodiments, the costimulatory domains enhance cytokine production, CAR-T cell proliferation, and/or CAR-T cell persistence during T cell activation.

As described herein, a fourth generation CAR can contain an antigen binding domain, a transmembrane domain, three or four signaling domains, and a domain which upon successful signaling of the CAR induces expression of a cytokine gene. In some instances, the cytokine gene is an endogenous or exogenous cytokine gene of the hypoimmunogenic cells. In some cases, the cytokine gene encodes a pro-inflammatory cytokine. In some embodiments, the pro-inflammatory cytokine is selected from a group that includes IL-1, IL-2, IL-9, IL-12, IL-18, TNF, IFN-gamma, and a functional fragment thereof. In some embodiments, the domain which upon successful signaling of the CAR induces expression of the cytokine gene comprises a transcription factor or functional domain or fragment thereof.

In some embodiments, the CAR comprises a CD3 zeta (CD3ζ) domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In other embodiments, the CAR comprises (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In many embodiments, the CAR comprises (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene. In some embodiments, the CAR comprises a (i) an anti-CD19 scFv; (ii) a CD8a hinge and transmembrane domain or functional variant thereof; (iii) a 4-1BB costimulatory domain or functional variant thereof; and (iv) a CD3ζ signaling domain or functional variant thereof.

Methods for introducing a CAR construct or producing a CAR-T cells are well known to those skilled in the art. Detailed descriptions are found, for example, in Vormittag et al., Curr Opin Biotechnol, 2018, 53, 162-181; and Eyquem et al., Nature, 2017, 543, 113-117.

In some embodiments, the cells derived from primary T cells comprise reduced expression of an endogenous T cell receptor, for example by disruption of an endogenous T cell receptor gene (e.g., T cell receptor alpha constant region (TRAC) or T cell receptor beta constant region (TRB)). In some embodiments, an exogenous nucleic acid encoding a polypeptide as disclosed herein (e.g., a chimeric antigen receptor, a HLA-E variant protein, a HLA-G variant protein, and/or an exogenous PD-L1 protein, or another tolerogenic factor disclosed herein) is inserted at the disrupted T cell receptor gene. In some embodiments, an exogenous nucleic acid encoding a polypeptide is inserted at a TRAC or a TRB gene locus.

In some embodiments, a HLA-E variant transgene, a HLA-G variant transgene, and/or an exogenous PD-L1 transgene is inserted into a pre-selected locus of the cell. In some embodiments, a transgene encoding a CAR is inserted into a pre-selected locus of the cell. In many embodiments, a HLA-E variant transgene, a HLA-G variant transgene, and/or an exogenous PD-L1 transgene and a transgene encoding a CAR are inserted into a pre-selected locus of the cell. The pre-selected locus can be a safe harbor locus. Non-limiting examples of a safe harbor locus include, but are not limited to, a CCR5 gene locus, a CXCR4 gene locus, a PPP1R12C (also known as AAVS1) gene locus, an albumin gene locus, a SHS231 gene locus, a CLYBL gene locus, a Rosa gene locus (e.g., ROSA26 gene locus), an F3 gene locus (also known as CD142), a MICA gene locus, a MICB gene locus, a LRP1 gene locus (also known as a CD91 gene locus), a HMGB1 gene locus, an ABO gene locus, ad RHD gene locus, a FUT1 locus, and a KDM5D gene locus. The HLA-E variant transgene, a HLA-G variant transgene, and/or an exogenous PD-L1 transgene can be inserted in Introns 1 or 2 for PPP1R12C (i.e., AAVS1) or CCR5. The HLA-E variant transgene, a HLA-G variant transgene, and/or an exogenous PD-L1 transgene can be inserted in Exons 1 or 2 or 3 for CCR5. The HLA-E variant transgene, a HLA-G variant transgene, and/or an exogenous PD-L1 transgene can be inserted in intron 2 for CLYBL. The HLA-E variant transgene, a HLA-G variant transgene, and/or an exogenous PD-L1 transgene can be inserted in a 500 bp window in Ch-4:58, 976, 613 (i.e., SHS231). The HLA-E variant transgene, a HLA-G variant transgene, and/or an exogenous PD-L1 transgene can be insert in any suitable region of the aforementioned safe harbor loci that allows for expression of the exogenous, including, for example, an intron, an exon or a coding sequence region in a safe harbor locus. In some embodiments, the pre-selected locus is selected from the group consisting of the HLA-A locus, the HLA-B locus, the HLA-C locus, the CD155 locus, the B2M locus, the CIITA locus, the TRAC locus, and the TRB locus. In some embodiments, the pre-selected locus is the HLA-A locus. In some embodiments, the pre-selected locus is the HLA-B locus. In some embodiments, the pre-selected locus is the HLA-C locus. In some embodiments, the pre-selected locus is the CD155 locus. In some embodiments, the pre-selected locus is the B2M locus. In some embodiments, the pre-selected locus is the CIITA locus. In some embodiments, the pre-selected locus is the TRAC locus. In some embodiments, the pre-selected locus is the TRB locus.

In some embodiments, a HLA-E variant transgene, a HLA-G variant transgene, and/or an exogenous PD-L1 transgene and a transgene encoding a CAR are inserted into the same locus. In some embodiments, a HLA-E variant transgene, a HLA-G variant transgene, and/or an exogenous PD-L1 transgene and a transgene encoding a CAR are inserted into different loci. In many instances, a HLA-E variant transgene, a HLA-G variant transgene, and/or an exogenous PD-L1 transgene is inserted into a safe harbor locus. In many instances, a transgene encoding a CAR is inserted into a safe harbor locus. In some instances, a HLA-E variant transgene, a HLA-G variant transgene, and/or an exogenous PD-L1 transgene is inserted into an HLA-A locus. In some instances, a transgene encoding a CAR is inserted into an HLA-A locus. In some instances, a HLA-E variant transgene, a HLA-G variant transgene, and/or an exogenous PD-L1 transgene is inserted into an HLA-B locus. In some instances, a transgene encoding a CAR is inserted into an HLA-B locus. In some instances, a HLA-E variant transgene, a HLA-G variant transgene, and/or an exogenous PD-L1 transgene is inserted into an HLA-B locus. In some instances, a transgene encoding a CAR is inserted into an HLA-B locus. In some instances, a HLA-E variant transgene, a HLA-G variant transgene, and/or an exogenous PD-L1 transgene is inserted into a CD155 locus. In some instances, a transgene encoding a CAR is inserted into a CD155 locus. In some instances, a HLA-E variant transgene, a HLA-G variant transgene, and/or an exogenous PD-L1 transgene is inserted into a B2M locus. In some instances, a transgene encoding a CAR is inserted into a B2M locus. In certain instances, a HLA-E variant transgene, a HLA-G variant transgene, and/or an exogenous PD-L1 transgene is inserted into a CIITA locus. In certain instances, a transgene encoding a CAR is inserted into a CIITA locus. In particular instances, a HLA-E variant transgene, a HLA-G variant transgene, and/or an exogenous PD-L1 transgene is inserted into a TRAC locus. In particular instances, a transgene encoding a CAR is inserted into a TRAC locus. In many other instances, a HLA-E variant transgene, a HLA-G variant transgene, and/or an exogenous PD-L1 transgene is inserted into a TRB locus. In many other instances, a transgene encoding a CAR is inserted into a TRB locus. In some embodiments, a HLA-E variant transgene, a HLA-G variant transgene, and/or an exogenous PD-L1 transgene and a transgene encoding a CAR are inserted into a safe harbor locus (e.g., a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, ad RHD gene locus, a FUT1 locus, and a KDM5D gene locus.

In many embodiments, a HLA-E variant transgene, a HLA-G variant transgene, and/or an exogenous PD-L1 transgene and a transgene encoding a CAR are inserted into a safe harbor locus. In many embodiments, a HLA-E variant transgene, a HLA-G variant transgene, and/or an exogenous PD-L1 transgene and a transgene encoding a CAR are controlled by a single promoter and are inserted into a safe harbor locus. In many embodiments, a HLA-E variant transgene, a HLA-G variant transgene, and/or an exogenous PD-L1 transgene and a transgene encoding a CAR are controlled by their own promoters and are inserted into a safe harbor locus. In many embodiments, a HLA-E variant transgene, a HLA-G variant transgene, and/or an exogenous PD-L1 transgene and a transgene encoding a CAR are inserted into a TRAC locus. In many embodiments, a HLA-E variant transgene, a HLA-G variant transgene, and/or an exogenous PD-L1 transgene and a transgene encoding a CAR are controlled by a single promoter and are inserted into a TRAC locus. In many embodiments, a HLA-E variant transgene, a HLA-G variant transgene, and/or an exogenous PD-L1 transgene and a transgene encoding a CAR are controlled by their own promoters and are inserted into a TRAC locus. In some embodiments, a HLA-E variant transgene, a HLA-G variant transgene, and/or an exogenous PD-L1 transgene and a transgene encoding a CAR are inserted into a TRB locus. In some embodiments, a HLA-E variant transgene, a HLA-G variant transgene, and/or an exogenous PD-L1 transgene and a transgene encoding a CAR are controlled by a single promoter and are inserted into a TRB locus. In some embodiments, a HLA-E variant transgene, a HLA-G variant transgene, and/or an exogenous PD-L1 transgene and a transgene encoding a CAR are controlled by their own promoters and are inserted into a TRB locus. In other embodiments, a HLA-E variant transgene, a HLA-G variant transgene, and/or an exogenous PD-L1 transgene and a transgene encoding a CAR are inserted into a B2M locus. In other embodiments, a HLA-E variant transgene, a HLA-G variant transgene, and/or an exogenous PD-L1 transgene and a transgene encoding a CAR are controlled by a single promoter and are inserted into a B2M locus. In other embodiments, a HLA-E variant transgene, a HLA-G variant transgene, and/or an exogenous PD-L1 transgene and a transgene encoding a CAR are controlled by their own promoters and are inserted into a B2M locus. In various embodiments, a HLA-E variant transgene, a HLA-G variant transgene, and/or an exogenous PD-L1 transgene and a transgene encoding a CAR are inserted into a CIITA locus. In various embodiments, a HLA-E variant transgene, a HLA-G variant transgene, and/or an exogenous PD-L1 transgene and a transgene encoding a CAR are controlled by a single promoter and are inserted into a CIITA locus. In various embodiments, a HLA-E variant transgene, a HLA-G variant transgene, and/or an exogenous PD-L1 transgene and a transgene encoding a CAR are controlled by their own promoters and are inserted into a CIITA locus. In some embodiments, a HLA-E variant transgene, a HLA-G variant transgene, and/or an exogenous PD-L1 transgene and a transgene encoding a CAR are inserted into an HLA-A locus. In various embodiments, a HLA-E variant transgene, a HLA-G variant transgene, and/or an exogenous PD-L1 transgene and a transgene encoding a CAR are controlled by a single promoter and are inserted into an HLA-A locus. In various embodiments, a HLA-E variant transgene, a HLA-G variant transgene, and/or an exogenous PD-L1 transgene and a transgene encoding a CAR are controlled by their own promoters and are inserted into an HLA-A locus. In some embodiments, a HLA-E variant transgene, a HLA-G variant transgene, and/or an exogenous PD-L1 transgene and a transgene encoding a CAR are inserted into an HLA-B locus. In various embodiments, a HLA-E variant transgene, a HLA-G variant transgene, and/or an exogenous PD-L1 transgene and a transgene encoding a CAR are controlled by a single promoter and are inserted into an HLA-B locus. In various embodiments, a HLA-E variant transgene, a HLA-G variant transgene, and/or an exogenous PD-L1 transgene and a transgene encoding a CAR are controlled by their own promoters and are inserted into an HLA-B locus. In some embodiments, a HLA-E variant transgene, a HLA-G variant transgene, and/or an exogenous PD-L1 transgene and a transgene encoding a CAR are inserted into an HLA-C locus. In various embodiments, a HLA-E variant transgene, a HLA-G variant transgene, and/or an exogenous PD-L1 transgene and a transgene encoding a CAR are controlled by a single promoter and are inserted into an HLA-C locus. In various embodiments, a HLA-E variant transgene, a HLA-G variant transgene, and/or an exogenous PD-L1 transgene and a transgene encoding a CAR are controlled by their own promoters and are inserted into an HLA-C locus. In various embodiments, a HLA-E variant transgene, a HLA-G variant transgene, and/or an exogenous PD-L1 transgene and a transgene encoding a CAR are inserted into a CD155 locus. In various embodiments, a HLA-E variant transgene, a HLA-G variant transgene, and/or an exogenous PD-L1 transgene and a transgene encoding a CAR are controlled by a single promoter and are inserted into a CD155 locus. In various embodiments, a HLA-E variant transgene, a HLA-G variant transgene, and/or an exogenous PD-L1 transgene and a transgene encoding a CAR are controlled by their own promoters and are inserted into a CD155 locus.

In some instances, the promoter controlling expression of any transgene described is a constitutive promoter. In other instances, the promoter for any transgene described is an inducible promoter. In some embodiments, the promoter is an EF1α promoter. In some embodiments, the promoter is CAG promoter. In some embodiments, a HLA-E variant transgene, a HLA-G variant transgene, and/or an exogenous PD-L1 transgene and a transgene encoding a CAR are both controlled by a constitutive promoter. In some embodiments, a HLA-E variant transgene, a HLA-G variant transgene, and/or an exogenous PD-L1 transgene and a transgene encoding a CAR are both controlled by an inducible promoter. In some embodiments, a HLA-E variant transgene, a HLA-G variant transgene, and/or an exogenous PD-L1 transgene is controlled by a constitutive promoter and a transgene encoding a CAR is controlled by an inducible promoter. In some embodiments, a HLA-E variant transgene, a HLA-G variant transgene, and/or an exogenous PD-L1 transgene is controlled by an inducible promoter and a transgene encoding a CAR is controlled by a constitutive promoter. In various embodiments, a HLA-E variant transgene, a HLA-G variant transgene, and/or an exogenous PD-L1 transgene is controlled by an EF1α promoter and a transgene encoding a CAR is controlled by an EF1α promoter. In some embodiments, a HLA-E variant transgene, a HLA-G variant transgene, and/or an exogenous PD-L1 transgene is controlled by a CAG promoter and a transgene encoding a CAR is controlled by a CAG promoter. In some embodiments, a HLA-E variant transgene, a HLA-G variant transgene, and/or an exogenous PD-L1 transgene is controlled by a CAG promoter and a transgene encoding a CAR is controlled by an EF1α promoter. In some embodiments, a HLA-E variant transgene, a HLA-G variant transgene, and/or an exogenous PD-L1 transgene is controlled by an EF1α promoter and a transgene encoding a CAR is controlled by a CAG promoter. In some embodiments, expression of both a HLA-E variant transgene, a HLA-G variant transgene, and/or an exogenous PD-L1 transgene and a transgene encoding a CAR is controlled by a single EF1α promoter. In some embodiments, expression of both a HLA-E variant transgene, a HLA-G variant transgene, and/or an exogenous PD-L1 transgene and a transgene encoding a CAR is controlled by a single CAG promoter.

In another embodiment, the present technology disclosed herein is directed to pluripotent stem cells, (e.g., pluripotent stem cells and induced pluripotent stem cells (iPSCs)), differentiated cells derived from such pluripotent stem cells (e.g., hypoimmune T cells), and primary T cells that overexpress a HLA-E variant, a HLA-G variant, and/or an exogenous PD-L1 (such as exogenously express HLA-E variant, HLA-G variant, and/or exogenous PD-L1 proteins), have reduced expression or lack expression of MHC class I and/or MHC class II human leukocyte antigens, and have reduced expression or lack expression of a T-cell receptor (TCR) complex. In some embodiments, the hypoimmune T cells and primary T cells overexpress a HLA-E variant, a HLA-G variant, and/or an exogenous PD-L1 (such as exogenously express HLA-E variant, HLA-G variant, and/or exogenous PD-L1 proteins), have reduced expression or lack expression of MHC class I and/or MHC class II human leukocyte antigens, and have reduced expression or lack expression of a T-cell receptor (TCR) complex.

In some embodiments, pluripotent stem cells, (e.g., pluripotent stem cells and induced pluripotent stem cells (iPSCs)), differentiated cells derived from such pluripotent stem cells (e.g., hypoimmune T cells), and primary T cells overexpress an HLA-E variant, an HLA-G variant, and/or an exogenous PD-L1 proteins and include a genomic modification of the HLA-A gene. In some embodiments, pluripotent stem cells, (e.g., pluripotent stem cells and induced pluripotent stem cells (iPSCs)), differentiated cells derived from such pluripotent stem cells (e.g., hypoimmune T cells), and primary T cells overexpress an HLA-E variant, an HLA-G variant, and/or an exogenous PD-L1 proteins and include a genomic modification of the HLA-B gene. In some embodiments, pluripotent stem cells, (e.g., pluripotent stem cells and induced pluripotent stem cells (iPSCs)), differentiated cells derived from such pluripotent stem cells (e.g., hypoimmune T cells), and primary T cells overexpress an HLA-E variant, an HLA-G variant, and/or an exogenous PD-L1 proteins and include a genomic modification of the HLA-C gene. In some embodiments, pluripotent stem cells, (e.g., pluripotent stem cells and induced pluripotent stem cells (iPSCs)), differentiated cells derived from such pluripotent stem cells (e.g., hypoimmune T cells), and primary T cells overexpress an HLA-E variant, an HLA-G variant, and/or an exogenous PD-L1 proteins and include a genomic modification of the CD155 gene. In some embodiments, pluripotent stem cells, (e.g., pluripotent stem cells and induced pluripotent stem cells (iPSCs)), differentiated cells derived from such pluripotent stem cells (e.g., hypoimmune T cells), and primary T cells overexpress an HLA-E variant, an HLA-G variant, and/or an exogenous PD-L1 proteins and include a genomic modification of the B2M gene. In some embodiments, pluripotent stem cells, differentiated cell derived from such pluripotent stem cells and primary T cells overexpress an HLA-E variant, an HLA-G variant, and/or an exogenous PD-L1 proteins and include a genomic modification of the CIITA gene. In some embodiments, pluripotent stem cells, T cells differentiated from such pluripotent stem cells and primary T cells overexpress an HLA-E variant, an HLA-G variant, and/or an exogenous PD-L1 proteins and include a genomic modification of the TRAC gene. In some embodiments, pluripotent stem cells, T cells differentiated from such pluripotent stem cells and primary T cells overexpress an HLA-E variant, an HLA-G variant, and/or an exogenous PD-L1 proteins and include a genomic modification of the TRB gene. In some embodiments, pluripotent stem cells, T cells differentiated from such pluripotent stem cells and primary T cells overexpress an HLA-E variant, an HLA-G variant, and/or an exogenous PD-L1 proteins and include one or more genomic modifications selected from the group consisting of the HLA-A, HLA-B, HLA-C, CD155, B2M, CIITA, TRAC and TRB genes. In some embodiments, pluripotent stem cells, T cells differentiated from such pluripotent stem cells and primary T cells overexpress an HLA-E variant, an HLA-G variant, and/or an exogenous PD-L1 proteins and include genomic modifications of the B2M, CIITA and TRAC genes. In some embodiments, pluripotent stem cells, T cells differentiated from such pluripotent stem cells and primary T cells overexpress an HLA-E variant, an HLA-G variant, and/or an exogenous PD-L1 proteins and include genomic modifications of the HLA-A, HLA-B, HLA-C, CD155, B2M, CIITA and TRB genes. In some embodiments, pluripotent stem cells, T cells differentiated from such pluripotent stem cells and primary T cells overexpress an HLA-E variant, an HLA-G variant, and/or an exogenous PD-L1 proteins and include genomic modifications of the HLA-A, HLA-B, HLA-C, CD155, B2M, CIITA, TRAC and TRB genes. In some embodiments, the cells are HLA-A^{-/-} as well as HLA-E variant, HLA-G variant, and/or PD-L1 cells. In some embodiments, the cells are HLA-C^{-/-} as well as HLA-E variant, HLA-G variant, and/or PD-L1 cells. In some embodiments, the cells are HLA-B^{-/-} as well as HLA-E variant, HLA-G variant, and/or PD-L1 cells. In some embodiments, the cells are CD155^{-/-} as well as HLA-E variant, HLA-G variant, and/or PD-L1 cells. In many embodiments, the cells are HLA-A^{-/-}, HLA-C^{-/-} as well as HLA-E variant, HLA-G variant, and/or PD-L1 cells. In many embodiments, the cells are HLA-A^{-/-}, HLA-B^{-/-} as well as HLA-E variant, HLA-G variant, and/or PD-L1 cells. In many embodiments, the pluripotent stem cells, differentiated cell derived from such pluripotent stem cells and primary T cells are HLA-A^{-/-}, CD155^{-/-}, as well as HLA-E variant, HLA-G variant, and/or PD-L1 cells. In many embodiments, the cells are HLA-B^{-/-}, HLA-C^{-/-} as well as HLA-E variant, HLA-G variant, and/or PD-L1 cells. In many embodiments, the cells are HLA-B^{-/-}, CD155^{-/-} as well as HLA-E variant, HLA-G variant, and/or PD-L1 cells. In many embodiments, the cells are HLA-C^{-/-}, CD155^{-/-} as well as HLA-E variant, HLA-G variant, and/or PD-L1 cells. In many embodiments, the pluripotent stem cells, differentiated cell derived from such pluripotent stem cells and primary T cells are HLA-A^{-/-}, HLA-C^{-/-}, CD155^{-/-}, as well as HLA-E variant, HLA-G variant, and/or PD-L1 cells. In many embodiments, the pluripotent stem cells, differentiated cell derived from such pluripotent stem cells and primary T cells are HLA-A^{-/-}, HLA-B^{-/-}, HLA-C^{-/-}, as well as HLA-E variant, HLA-G variant, and/or PD-L1 cells. In many embodiments, the pluripotent stem cells, differentiated cell derived from such pluripotent stem cells and primary T cells are HLA-A^{-/-}, HLA-B^{-/-}, HLA-C^{-/-}, CD155^{-/-}, as well as HLA-E variant, HLA-G variant, and/or PD-L1 cells.

In many embodiments, the pluripotent stem cells, differentiated cell derived from such pluripotent stem cells and primary T cells are B2M^{-/-}, CIITA^{-/-}, TRAC^{-/-}, as well as HLA-E variant, HLA-G variant, and/or PD-L1 cells. In many embodiments, the cells are B2M^{-/-}, CIITA^{-/-}, TRB^{-/-}, as well as HLA-E variant, HLA-G variant, and/or PD-L1 cells. In many embodiments, the cells are B2M^{-/-}, CIITA^{-/-}, TRAC^{-/-}, TRB^{-/-}, as well as HLA-E variant, HLA-G variant, and/or PD-L1 cells. In some embodiments, the cells are B2M^{indel/indel}, CIITA^{indel/indel} TRAC^{indel/indel}, as well as HLA-E variant, HLA-G variant, and/or PD-L1 cells. In some embodiments, the cells are B2M^{indel/indel}, CIITA^{indel/indel}, TRB^{indel/indel}, as well as HLA-E variant, HLA-G variant, and/or PD-L1 cells. In some embodiments, the cells are B2M^{indel/indel} CIITA^{indel/indel}, TRAC^{indel/indel}, TRB^{indel/indel}, as well as HLA-E variant⁺, HLA-G variant⁺, and/or PD-L1⁺ cells. In some embodiments, the engineered or modified cells described are pluripotent stem cells, T cells differentiated from such pluripotent stem cells or primary T cells. Non-limiting examples of primary T cells include CD3+ T cells, CD4+ T cells, CD8+ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), γδ T cells, and any other subtype of T cells.

In some embodiments, a HLA-E variant transgene, a HLA-G variant transgene, and/or an exogenous PD-L1 transgene is inserted into a pre-selected locus of the cell. The pre-selected locus can be a safe harbor locus. Non-limiting examples of a safe harbor locus includes a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, ad RHD gene locus, a FUT1 locus, and a KDM5D gene locus. In some embodiments, the pre-selected locus is the TRAC locus. In some embodiments, a HLA-E variant transgene, a HLA-G variant transgene, and/or an exogenous PD-L1 transgene is inserted into a safe harbor locus (e.g., a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, ad RHD gene locus, a FUT1 locus, and a KDM5D gene locus. In many embodiments, a CD47 transgene is inserted into the B2M locus. In many embodiments, a HLA-E variant transgene, a HLA-G variant transgene, and/or an exogenous PD-L1 transgene is inserted into the B2M locus. In many embodiments, a HLA-E variant transgene, a HLA-G variant transgene, and/or an exogenous PD-L1 transgene is inserted into the TRAC locus. In many embodiments, a HLA-E variant transgene, a HLA-G variant transgene, and/or an exogenous PD-L1 transgene is inserted into the TRB locus.

In some instances, expression of a HLA-E variant transgene, a HLA-G variant transgene, and/or an exogenous PD-L1 transgene is controlled by a constitutive promoter. In other instances, expression of a HLA-E variant transgene, a HLA-G variant transgene, and/or an exogenous PD-L1 transgene is controlled by an inducible promoter. In some embodiments, the promoter is an EF1alpha (EF1a) promoter. In some embodiments, the promoter a CAG promoter.

In yet another embodiment, the present technology disclosed herein is directed to pluripotent stem cells, (e.g., pluripotent stem cells and induced pluripotent stem cells (iPSCs)), T cells derived from such pluripotent stem cells (e.g., hypoimmune T cells), and primary T cells that have reduced expression or lack expression of MHC class I and/or MHC class II human leukocyte antigens and have reduced expression or lack expression of a T-cell receptor (TCR) complex. In some embodiments, the cells have reduced or lack expression of MHC class I antigens, MHC class II antigens, and TCR complexes.

In some embodiments, pluripotent stem cells (e.g., iPSCs), differentiated cells derived from such (e.g., T cells differentiated from such), and primary T cells include a genomic modification of the B2M gene. In some embodiments, pluripotent stem cells (e.g., iPSCs), differentiated cells derived from such (e.g., T cells differentiated from such), and primary T cells include a genomic modification of the CIITA gene. In some embodiments, pluripotent stem cells (e.g., iPSCs), T cells differentiated from such, and primary T cells include a genomic modification of the TRAC gene. In some embodiments, pluripotent stem cells (e.g., iPSCs), T cells differentiated from such, and primary T cells include a genomic modification of the TRB gene. In some embodiments, pluripotent stem cells (e.g., iPSCs), T cells differentiated from such, and primary T cells include one or more genomic modifications selected from the group consisting of the B2M, CIITA and TRAC genes. In some embodiments, pluripotent stem cells (e.g., iPSCs), T cells differentiated from such, and primary T cells include one or more genomic modifications selected from the group consisting of the B2M, CIITA and TRB genes. In some embodiments, pluripotent stem cells (e.g., iPSCs), T cells differentiated from such, and primary T cells include one or more genomic modifications selected from the group consisting of the B2M, CIITA, TRAC and TRB genes. In many embodiments, the cells including iPSCs, T cells differentiated from such, and primary T cells are B2M^{-/-}, CIITA^{-/-}, TRAC^{-/-} cells. In many embodiments, the cells including iPSCs, T cells differentiated from such, and primary T cells are B2M^{-/-}, CIITA^{-/-}, TRB-cells. In some embodiments, the cells including iPSCs, T cells differentiated from such, and primary T cells are B2M^{indel/indel}, CIITA^{indel/indel}, TRAC^{indel/indel} cells. In some embodiments, the cells including iPSCs, T cells differentiated from such, and primary T cells are B2M^{indel/indel}, CIITA^{indel/indel}, TRB^{indel/indel} cells. In some embodiments, the cells including iPSCs, T cells differentiated from such, and primary T cells are B2M^{indel/indel}, CIITA^{indel/indel}, TRAC^{indel/indel}, TRB^{indel/indel} cells. In some embodiments, the modified cells described are pluripotent stem cells, induced pluripotent stem cells, T cells differentiated from such pluripotent stem cells and induced pluripotent stem cells, or primary T cells. Non-limiting examples of primary T cells include CD3+ T cells, CD4+ T cells, CD8+ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), γδ T cells, and any other subtype of T cells.

Cells of the present technology exhibit reduced or lack expression of MHC class I antigens, MHC class II antigens, and/or TCR complexes. Reduction of MHC I and/or MHC II expression can be accomplished, for example, by one or more of the following: (1) targeting the polymorphic HLA alleles (HLA-A, HLA-B, HLA-C) and MHC-II genes directly; (2) removal of B2M, which will prevent surface trafficking of all MHC-I molecules; (3) removal of CIITA, which will prevent surface trafficking of all MHC-II molecules; and/or (4) deletion of components of the MHC enhanceosomes, such as LRC5, RFX5, RFXANK, RFXAP, IRF1, NF-Y (including NFY-A, NFY-B, NFY-C), and CIITA that are critical for HLA expression.

In some embodiments, HLA expression is interfered with by targeting individual HLAs (e.g., knocking out expression of HLA-A, HLA-B, HLA-C, HLA-DP, HLA-DQ, and/or HLA-DR), targeting transcriptional regulators of HLA expression (e.g., knocking out expression of NLRC5, CIITA, RFX5, RFXAP, RFXANK, NFY-A, NFY-B, NFY-C and/or IRF-1), blocking surface trafficking of MHC class I molecules (e.g., knocking out expression of B2M and/or TAP1), and/or targeting with HLA-Razor (see, e.g., WO2016183041).

In some embodiments, the cells disclosed herein including, but not limited to, pluripotent stem cells, induced pluripotent stem cells, differentiated cells derived from such stem cells, and primary T cells do not express one or more human leukocyte antigens (e.g., HLA-A, HLA-B, HLA-C, HLA-DP, HLA-DQ, and/or HLA-DR) corresponding to MHC-I and/or MHC-II and are thus characterized as being hypoimmunogenic. For example, in many embodiments, the pluripotent stem cells and induced pluripotent stem cells disclosed have been modified such that the stem cell or a differentiated stem cell prepared therefrom do not express or exhibit reduced expression of one or more of the following MHC-I molecules: HLA-A, HLA-B and HLA-C. In some embodiments, one or more of HLA-A, HLA-B and HLA-C may be “knocked-out” of a cell. A cell that has a knocked-out HLA-A gene, HLA-B gene, and/or HLA-C gene may exhibit reduced or eliminated expression of each knocked-out gene.

In some embodiments, guide RNAs that allow simultaneous deletion of all MHC class I alleles by targeting a conserved region in the HLA genes are identified as HLA Razors. In some embodiments, the gRNAs are part of a CRISPR system. In alternative embodiments, the gRNAs are part of a TALEN system. In some embodiments, an HLA Razor targeting an identified conserved region in HLAs is described in WO2016183041. In some embodiments, multiple HLA Razors targeting identified conserved regions are utilized. It is generally understood that any guide that targets a conserved region in HLAs can act as an HLA Razor.

Methods provided are useful for inactivation or ablation of MHC class I expression and/or MHC class II expression in cells such as but not limited to pluripotent stem cells, differentiated cells, and primary T cells. In some embodiments, genome editing technologies utilizing rare-cutting endonucleases (e.g., the CRISPR/Cas, TALEN, zinc finger nuclease, meganuclease, and homing endonuclease systems) are also used to reduce or eliminate expression of critical immune genes (e.g., by deleting genomic DNA of critical immune genes) in cells. In many embodiments, genome editing technologies or other gene modulation technologies are used to insert tolerance-inducing factors in human cells, rendering them and the differentiated cells prepared therefrom hypoimmunogenic cells. As such, the hypoimmunogenic cells have reduced or eliminated expression of MHC I and MHC II expression. In some embodiments, the cells are nonimmunogenic (e.g., do not induce an immune response) in a recipient subject.

In some embodiments, the cell includes a modification to increase expression of a HLA-E variant protein, a HLA-G variant protein, and/or an exogenous PD-L1 protein and one or more factors selected from the group consisting of DUX4, CD24, CD27, CD46, CD55, CD59, CD200, HLA-C, HLA-E, HLA-E heavy chain, HLA-G, PD-L1, IDO1, CTLA4-Ig, C1-Inhibitor, IL-10, IL-35, IL-39, FasL, CCL21, CCL22, Mfge8, and Serpinb9.

In some embodiments, the cell comprises a genomic modification of one or more target polynucleotide sequences that regulate the expression of either MHC class I molecules, MHC class II molecules, or MHC class I and MHC class II molecules. In some embodiments, a genetic editing system is used to modify one or more target polynucleotide sequences. In some embodiments, the targeted polynucleotide sequence is one or more selected from the group including B2M, CIITA, and NLRC5. In some embodiments, the cell comprises a genetic editing modification to the B2M gene. In some embodiments, the cell comprises a genetic editing modification to the CIITA gene. In some embodiments, the cell comprises a genetic editing modification to the NLRC5 gene. In some embodiments, the cell comprises genetic editing modifications to the B2M and CIITA genes. In some embodiments, the cell comprises genetic editing modifications to the B2M and NLRC5 genes. In some embodiments, the cell comprises genetic editing modifications to the CIITA and NLRC5 genes. In numerous embodiments, the cell comprises genetic editing modifications to the B2M, CIITA and NLRC5 genes. In many embodiments, the genome of the cell has been altered to reduce or delete critical components of HLA expression.

In some embodiments, the present disclosure provides a cell (e.g., stem cell, induced pluripotent stem cell, differentiated cell, hematopoietic stem cell, primary NK cell, CAR-NK cell, primary T cell or CAR-T cell) or population thereof comprising a genome in which a gene has been edited to delete a contiguous stretch of genomic DNA, thereby reducing or eliminating surface expression of MHC class I molecules in the cell or population thereof. In certain embodiments, the present disclosure provides a cell (e.g., stem cell, induced pluripotent stem cell, differentiated cell, hematopoietic stem cell, primary NK cell, CAR-NK cell, primary T cell or CAR-T cell) or population thereof comprising a genome in which a gene has been edited to delete a contiguous stretch of genomic DNA, thereby reducing or eliminating surface expression of MHC class II molecules in the cell or population thereof. In numerous embodiments, the present disclosure provides a cell (e.g., stem cell, induced pluripotent stem cell, differentiated cell, hematopoietic stem cell, primary NK cell, CAR-NK cell, primary T cell or CAR-T cell) or population thereof comprising a genome in which one or more genes has been edited to delete a contiguous stretch of genomic DNA, thereby reducing or eliminating surface expression of MHC class I and II molecules in the cell or population thereof.

In many embodiments, the expression of MHC I molecules and/or MHC II molecules is modulated by targeting and deleting a contiguous stretch of genomic DNA, thereby reducing or eliminating expression of a target gene selected from the group consisting of B2M, CIITA, and NLRC5. In some embodiments, described herein are genetically edited cells (e.g., modified human cells) comprising exogenous HLA-E variant proteins, HLA-G variant proteins, and/or exogenous PD-L1 proteins and inactivated or modified CIITA gene sequences, and in some instances, additional gene modifications that inactivate or modify B2M gene sequences. In some embodiments, described herein are genetically edited cells comprising exogenous HLA-E variant proteins, HLA-G variant proteins, and/or exogenous PD-L1 proteins and inactivated or modified CIITA gene sequences, and in some instances, additional gene modifications that inactivate or modify NLRC5 gene sequences. In some embodiments, described herein are genetically edited cells comprising exogenous HLA-E variant proteins, HLA-G variant proteins, and/or exogenous PD-L1 proteins and inactivated or modified B2M gene sequences, and in some instances, additional gene modifications that inactivate or modify NLRC5 gene sequences. In some embodiments, described herein are genetically edited cells comprising exogenous HLA-E variant proteins, HLA-G variant proteins, and/or exogenous PD-L1 proteins and inactivated or modified B2M gene sequences, and in some instances, additional gene modifications that inactivate or modify CIITA gene sequences and NLRC5 gene sequences.

Provided herein are cells exhibiting a modification of one or more targeted polynucleotide sequences that regulates the expression of any one of the following: (a) MHC I antigens, (b) MHC II antigens, (c) TCR complexes, (d) both MHC I and II antigens, and (e) MHC I and II antigens and TCR complexes. In certain embodiments, the modification includes increasing expression of a HLA-E variant protein, a HLA-G variant protein, and/or an exogenous PD-L1 protein. In some embodiments, the cells include an exogenous or recombinant HLA-E variant polypeptide, a HLA-G variant polypeptide, and/or an exogenous PD-L1 polypeptide. In many embodiments, the modification includes expression of a chimeric antigen receptor. In some embodiments, the cells comprise an exogenous or recombinant chimeric antigen receptor polypeptide.

In some embodiments, the cell includes a genomic modification of one or more targeted polynucleotide sequences that regulates the expression of MHC I antigens, MHC II antigens and/or TCR complexes. In some embodiments, a genetic editing system is used to modify one or more targeted polynucleotide sequences. In some embodiments, the polynucleotide sequence targets one or more genes selected from the group consisting of B2M, CIITA, TRAC, and TRB. In many embodiments, the genome of a T cell (e.g., a T cell differentiated from hypoimmunogenic iPSCs and a primary T cell) has been altered to reduce or delete critical components of HLA and TCR expression, e.g., HLA-A antigen, HLA-B antigen, HLA-C antigen, HLA-DP antigen, HLA-DQ antigen, HLA-DR antigens, TCR-alpha and TCR-beta.

In some embodiments, the present disclosure provides a cell or population thereof comprising a genome in which a gene has been edited to delete a contiguous stretch of genomic DNA, thereby reducing or eliminating surface expression of MHC class I molecules in the cell or population thereof. In certain embodiments, the present disclosure provides a cell or population thereof comprising a genome in which a gene has been edited to delete a contiguous stretch of genomic DNA, thereby reducing or eliminating surface expression of MHC class II molecules in the cell or population thereof. In many embodiments, the present disclosure provides a cell or population thereof comprising a genome in which a gene has been edited to delete a contiguous stretch of genomic DNA, thereby reducing or eliminating surface expression of TCR molecules in the cell or population thereof. In numerous embodiments, the present disclosure provides a cell or population thereof comprising a genome in which one or more genes has been edited to delete a contiguous stretch of genomic DNA, thereby reducing or eliminating surface expression of MHC class I and II molecules and TCR complex molecules in the cell or population thereof.

In some embodiments, the cells and methods described herein include genomically editing human cells to cleave CIITA gene sequences as well as editing the genome of such cells to alter one or more additional target polynucleotide sequences such as, but not limited to, B2M TRAC, and TRB. In some embodiments, the cells and methods described herein include genomically editing human cells to cleave B2M gene sequences as well as editing the genome of such cells to alter one or more additional target polynucleotide sequences such as, but not limited to, CIITA, TRAC, and TRB. In some embodiments, the cells and methods described herein include genomically editing human cells to cleave TRAC gene sequences as well as editing the genome of such cells to alter one or more additional target polynucleotide sequences such as, but not limited to, B2M, CIITA, and TRB. In some embodiments, the cells and methods described herein include genomically editing human cells to cleave TRB gene sequences as well as editing the genome of such cells to alter one or more additional target polynucleotide sequences such as, but not limited to, B2M, CIITA, and TRAC.

Provided herein are hypoimmunogenic stem cells comprising reduced expression of HLA-A, HLA-B, HLA-C, CIITA, TCR-alpha, and TCR-beta relative to a wild-type stem cell. In some embodiments, the hypoimmunogenic stem cell further comprise a set of exogenous genes comprising a first gene encoding an HLA-E variant, an HLA-G variant, and/or an exogenous PD-L1 and a second gene encoding a chimeric antigen receptor (CAR), wherein the first and/or second genes are inserted into a specific locus of at least one allele of the cell. Also provided herein are hypoimmunogenic primary T cells including any subtype of primary T cells comprising reduced expression of HLA-A, HLA-B, HLA-C, CIITA, TCR-alpha, and TCR-beta relative to a wild-type primary T cell. In some embodiments, the hypoimmunogenic stem cell further comprises a set of exogenous genes comprising a first gene encoding an HLA-E variant, an HLA-G variant, and/or an exogenous PD-L1 and a second gene encoding a chimeric antigen receptor (CAR), wherein the first and/or second genes are inserted into a specific locus of at least one allele of the cell. Further provided herein are hypoimmunogenic T cells differentiated from hypoimmunogenic induced pluripotent stem cells comprising reduced expression of HLA-A, HLA-B, HLA-C, CIITA, TCR-alpha, and TCR-beta relative to a wild-type primary T cell. In some embodiments, the hypoimmunogenic stem cell further comprises a set of exogenous genes comprising a first gene encoding an HLA-E variant, an HLA-G variant, and/or exogenous PD-L1 and a second gene encoding a chimeric antigen receptor (CAR), wherein the first and/or second genes are inserted into a specific locus of at least one allele of the cell.

In some embodiments, the population of engineered cells described evades NK cell mediated cytotoxicity upon administration to a recipient patient. In some embodiments, the population of engineered cells evades NK cell mediated cytotoxicity by one or more subpopulations of NK cells. In some embodiments, the population of engineered is protected from cell lysis by NK cells, including immature and/or mature NK cells upon administration to a recipient patient. In some embodiments, the population of engineered cells does not induce an immune response to the cell upon administration to a recipient patient.

In some embodiments, the population of engineered cells described elicits a reduced level of immune activation or no immune activation upon administration to a recipient subject. In some embodiments, the cells elicit a reduced level of systemic TH1 activation or no systemic TH1 activation in a recipient subject. In some embodiments, the cells elicit a reduced level of immune activation of peripheral blood mononuclear cells (PBMCs) or no immune activation of PBMCs in a recipient subject. In some embodiments, the cells elicit a reduced level of donor-specific IgG antibodies or no donor specific IgG antibodies against the cells upon administration to a recipient subject. In some embodiments, the cells elicit a reduced level of IgM and IgG antibody production or no IgM and IgG antibody production against the cells in a recipient subject. In some embodiments, the cells elicit a reduced level of cytotoxic T cell killing of the cells upon administration to a recipient subject.

### B. HLA-E Variants

In some embodiments, an HLA-E variant protein has a modification (e.g., one or more deletions, truncations, insertions and/or substitutions) at its antigen binding cleft. In some embodiments, the HLA-E variant protein has a modification at its antigen binding cleft such that the variant has reduced binding affinity or no binding affinity for an antigen peptide compared to an unmodified HLA-E protein. In some embodiments, the modification can alter the characteristics and/or properties of the variant protein compared its wild-type equivalent. In some instances, the modification increases the protein's stability compared to a wild-type HLA-E protein. In some embodiments, HLA-E protein stability is related to cell surface expression of the protein. In other words, an HLA-E variant protein is present at the surface of a cell at a higher level, at a higher frequency, and the like as compared to an unmodified HLA-E protein. In some embodiments, the modification increases the recycling rate (e.g., turnover rate or endocytic recycling rate) of a non-antigen peptide bound HLA-E variant protein. In some instances, the increased recycling rate corresponds to increased receptor endocytosis and recycling back to the cell surface, compared to a wild-type HLA-E protein.

In some embodiments, the modification at the antigen binding cleft inhibits an antigen peptide from binding to the HLA-E variant protein. The modification allows a decoy peptide to bind to the HLA-E variant, such as at the antigen binding cleft. In some embodiments, the decoy peptide is not covalently linked to the HLA-E variant protein. In some embodiments, the decoy peptide is linked to the HLA-E variant. In some instances, the decoy peptide is attached to the variant protein by a flexible linker. In some embodiments, the HLA-E variant protein includes one or more deletions (including truncations) in an intracellular domain of the protein. In some embodiments, the HLA-E variant protein includes one or more deletions (including truncations) in a plurality of intracellular domains of the protein. In some instances, such a deletion reduces HLA-E signaling. In some embodiments, the HLA-E variant protein includes a modification (e.g., one or more deletions, truncation, insertions and/or substitutions) in the extracellular domain such that the HLA-E variant protein cannot bind to another protein (e.g., a binding partner) when the HLA-E variant protein binds to an antigen peptide.

In some embodiments, the HLA-E variant protein is substantially similar to the HLA-E single chain dimer or the HLA-E single chain trimer as described in Gornalusse et al., Nat Biotech, 2017, 35, 765-772, the contents are herein incorporated by reference in its entirely. In some embodiments, the HLA-E single chain dimer comprises an HLA-E single chain (heavy chain), a B2M protein or a fragment thereof, and optionally, a linker linking the HLA-E single chain to the B2M protein. In some embodiments, the HLA-E single chain trimer comprises an HLA-E single chain, a B2M protein or a fragment thereof, and an antigen peptide such that the HLA-E single chain is linked to the B2M protein (by way of an optional linker) and the antigen peptide is linked to the B2M protein (by way of an optional linker).

In some embodiments, provided herein is an HLA-E polynucleotide or a variant of the HLA-E polynucleotide. In some embodiments, the HLA-E polynucleotide sequence is a homolog of HLA-E. In some embodiments, the polynucleotide sequence is an ortholog of HLA-E.

In some embodiments, the cells outlined herein comprise a genetic modification targeting the gene encoding the HLA-E polypeptide. In many embodiments, cells of the present technology, such as but not limited to, primary T cells, primary NK cells, primary endothelial cells, T cells derived from iPSCs, NK cells derived from iPSCs, and endothelial cells derived from iPSCs comprise a genetic modification targeting the HLA-E gene. The genetic modification can induce expression of HLA-E polynucleotides and HLA-E polypeptides in T cells including primary T cells, T cells derived from iPSCs, and CAR-T cells. The genetic modification can induce expression of HLA-E polynucleotides and HLA-E polypeptides in NK cells including primary NK cells, NK cells derived from iPSCs, and CAR-NK cells.

Assays to test whether the HLA-E gene has been activated or inactivated are known and described herein. In some embodiments, the resulting genetic modification of the HLA-E gene by PCR and the reduction or the enhancement of HLA-E expression can be assays by FACS analysis. In another embodiment, HLA-E protein expression is detected using a Western blot of cells lysates probed with antibodies to the HLA-E protein. In another embodiment, reverse transcriptase polymerase chain reactions (RT-PCR) are used to confirm the presence of the activating or inactivating genetic modification.

Disruption/elimination of both alleles of the B2M gene in a cell can eliminate surface expression of all MHC class I molecules and leave the cell vulnerable to NK cell mediated lysis. This response has been termed a “missing-self” response (see, Gornalusse et al., supra) and in some embodiments, the response can be prevented by overexpression of an HLA-E variant protein.

### C. HLA-G Variants

In some embodiments, an HLA-G variant protein has a modification (e.g., one or more deletions, truncations, insertions and/or substitutions) in its antigen binding cleft. In some embodiments, the HLA-G variant protein has a modification at its antigen binding cleft such that the variant has reduced binding affinity or no binding affinity for an antigen peptide compared to an unmodified HLA-G protein.

In some embodiments, the modification can alter the characteristics and/or properties of the HLA-G variant protein compared its wild-type equivalent. In some instances, the modification increases the protein's stability compared to a wild-type HLA-G protein. In some embodiments, HLA-G protein stability is related to cell surface expression of the protein. In other words, an HLA-G variant protein is present at the surface of a cell at a higher level, at a higher frequency, and the like as compared to an unmodified HLA-G protein. In some embodiments, the modification increases the recycling rate (e.g., turnover rate or endocytic recycling rate) of a non-antigen peptide bound HLA-G variant protein. In some instances, the increased recycling rate corresponds to increased receptor endocytosis and recycling back to the cell surface, compared to a wild-type HLA-G protein.

In some embodiments, the modification at the antigen binding cleft inhibits an antigen peptide from binding to the HLA-G variant protein. The modification allows a decoy peptide to bind to the HLA-G variant, such as at the antigen binding cleft. In some embodiments, the decoy peptide is not covalently linked to the HLA-G variant protein. In some embodiments, the decoy peptide is linked to the HLA-G variant. In some instances, the decoy peptide is attached to the variant protein by a flexible linker. In some embodiments, the HLA-G variant protein includes one or more deletions (including truncations) in an intracellular domain of the protein. In some embodiments, the HLA-G variant protein includes one or more deletions (including truncations) in a plurality of intracellular domains of the protein. In some instances, such a deletion or truncation reduces HLA-G signaling. In some embodiments, the HLA-E variant protein includes a modification (e.g., one or more deletions, truncations, insertions and/or substitutions) in the extracellular domain such that the HLA-G variant protein cannot bind to another protein (e.g., a binding partner) when the HLA-G variant protein binds to an antigen peptide.

In some embodiments, provided herein is an HLA-G polynucleotide or a variant of the HLA-G polynucleotide. In some embodiments, the HLA-G polynucleotide sequence is a homolog of HLA-E. In some embodiments, the polynucleotide sequence is an ortholog of HLA-G.

In some embodiments, the cells outlined herein comprise a genetic modification targeting the gene encoding the HLA-G polypeptide. In many embodiments, cells of the present technology, such as but not limited to, primary T cells, primary NK cells, primary endothelial cells, T cells derived from iPSCs, NK cells derived from iPSCs, and endothelial cells derived from iPSCs comprise a genetic modification targeting the HLA-G gene. The genetic modification can induce expression of HLA-G polynucleotides and HLA-G polypeptides in T cells including primary T cells, T cells derived from iPSCs, and CAR-T cells. The genetic modification can induce expression of HLA-G polynucleotides and HLA-G polypeptides in NK cells including primary NK cells, NK cells derived from iPSCs, and CAR-NK cells.

Assays to test whether the HLA-G gene has been activated or inactivated are known and described herein. In some embodiments, the resulting genetic modification of the HLA-G gene by PCR and the reduction or the enhancement of HLA-G expression can be assays by FACS analysis. In another embodiment, HLA-G protein expression is detected using a Western blot of cells lysates probed with antibodies to the HLA-G protein. In another embodiment, reverse transcriptase polymerase chain reactions (RT-PCR) are used to confirm the presence of the activating or inactivating genetic modification.

### D. PD-L1

In some embodiments, the target polynucleotide sequence is PD-L1 or a variant of PD-L1. In some embodiments, the target polynucleotide sequence is a homolog of PD-L1. In some embodiments, the target polynucleotide sequence is an ortholog of PD-L1.

In some embodiments, the cells outlined herein comprise a genetic modification targeting the gene encoding the PD-L1 polypeptide. In many embodiments, cells of the present technology, such as but not limited to, primary T cells, primary NK cells, primary endothelial cells, T cells derived from iPSCs, NK cells derived from iPSCs, and endothelial cells derived from iPSCs comprise a genetic modification targeting the PD-L1 gene. The genetic modification can induce expression of PD-L1 polynucleotides and PD-L1 polypeptides in T cells including primary T cells, T cells derived from iPSCs, and CAR-T cells. The genetic modification can induce expression of PD-L1 polynucleotides and PD-L1 polypeptides in NK cells including primary NK cells, NK cells derived from iPSCs, and CAR-NK cells.

Assays to test whether the CD274 (also known as B7-H, B7H1, PD-L1, PDCD1L1, PDCD1LG1, PDL1, and hPD-L1) gene has been activated or inactivated are known and described herein. In some embodiments, the resulting genetic modification of the PDCD1 gene by PCR and the reduction of PD-1 expression can be assays by FACS analysis. In another embodiment, PD-1 protein expression is detected using a Western blot of cells lysates probed with antibodies to the PD-1 protein. In another embodiment, reverse transcriptase polymerase chain reactions (RT-PCR) are used to confirm the presence of the inactivating genetic modification.

Useful genomic, polynucleotide and polypeptide information about human PD-L1 including the CD274 gene are provided in, for example, the GeneCard Identifier GC09P005450, HGNC 17635, NCBI Entrez Gene 29126, Ensembl ENSG00000120217, OMIMR 605402, UniProtKB/Swiss-Prot Q9NZQ7, NP_054862.1, and NM_014143.4.

### E. HLA-A

In some embodiments, the present technology modulates (e.g., reduces or eliminates) the expression of MHC I genes by targeting and modulating (e.g., reducing or eliminating) HLA-I expression. In some embodiments, the modulation occurs using a CRISPR/Cas system. HLA-A is one of three major types of MHC class I transmembrane proteins. HLA-A protein binds beta2-microglobulin and antigen peptides.

In some embodiments, the cells described herein comprise gene modifications at the gene locus encoding the HLA-A protein. In other words, the cells comprise a genetic modification at the HLA-A locus. In some instances, the nucleotide sequence encoding the HLA-A protein is set forth in RefSeq. Nos. NM_001242758.1 and NM_002116.7, and NCBI Genbank No. U03862.1. In some instances, the HLA-A gene locus is described in NCBI Gene ID No. 3105. In some cases, the amino acid sequence of HLA-A is set forth in RefSeq. Nos. NP 001229687.1 and NP_002107.3. Additional descriptions of the HLA-A protein and gene locus can be found in Uniprot No. P04439, HGNC Ref. No. 4931, and OMIM Ref. No. 142800.

In some embodiments, the hypoimmunogenic cells outlined herein comprise a genetic modification targeting the HLA-A gene. In some embodiments, the genetic modification targeting the HLA-A gene by the rare-cutting endonuclease comprises a Cas protein or a polynucleotide encoding a Cas protein, and at least one guide ribonucleic acid sequence for specifically targeting the HLA-A gene. In some embodiments, the at least one guide ribonucleic acid sequence for specifically targeting the HLA-A gene is selected from the group consisting of SEQ ID NOS:2-1418 and in Table 8 and Appendix 1 of WO2016183041, which is herein incorporated by reference. In some embodiments, the cell has a reduced ability to induce an immune response in a recipient subject. In some embodiments, an exogenous nucleic acid encoding a polypeptide as disclosed herein (e.g., a chimeric antigen receptor, a HLA-E variant protein, a HLA-G variant protein, and/or an exogenous PD-L1 protein, or another tolerogenic factor disclosed herein) is inserted at the HLA-A gene.

Assays to test whether the HLA-A gene has been inactivated are known and described herein. In some embodiments, the resulting genetic modification of the HLA-A gene by PCR and the reduction of HLA-I expression can be assays by FACS analysis. In another embodiment, HLA-A protein expression is detected using a Western blot of cells lysates probed with antibodies to the HLA-A protein. In another embodiment, reverse transcriptase polymerase chain reactions (RT-PCR) are used to confirm the presence of the inactivating genetic modification.

### F. HLA-B

In some embodiments, the present technology modulates (e.g., reduces or eliminates) the expression of MHC I genes by targeting and modulating (e.g., reducing or eliminating) HLA-I expression. In some embodiments, the modulation occurs using a CRISPR/Cas system. HLA-B is another of the three major types of MHC class I transmembrane proteins. In a MHC class I heterodimeric molecule, HLA-B protein serves as a heavy chain binds beta2-microglobulin which can be referred to as a light chain. The HLA-B protein is about 45 kDa and is encoded by 8 exons. Exon 1 encodes the leader peptide; exon 2 and 3 encode the alpha1 and alpha2 domains, which both bind an antigen peptide; exon 4 encodes the alpha3 domain; exon 5 encodes the transmembrane region; and exons 6 and 7 encode the cytoplasmic tail.

In some embodiments, the cells described herein comprise gene modifications at the gene locus encoding the HLA-B protein. In other words, the cells comprise a genetic modification at the HLA-B locus. In some instances, the nucleotide sequence encoding the HLA-B protein is set forth in RefSeq. No. NM_005514, and NCBI Genbank No. U03698.1.

In some instances, the HLA-B gene locus is described in NCBI Gene ID No. 3106. In some cases, the amino acid sequence of HLA-B is set forth in RefSeq. No. NP 005505.2.

Additional descriptions of the HLA-B protein and gene locus can be found in Uniprot No. P01889, HGNC Ref. No. 4932, and OMIM Ref. No. 142830.

In some embodiments, the hypoimmunogenic cells outlined herein comprise a genetic modification targeting the HLA-B gene. In some embodiments, the genetic modification targeting the HLA-B gene by the rare-cutting endonuclease comprises a Cas protein or a polynucleotide encoding a Cas protein, and at least one guide ribonucleic acid sequence for specifically targeting the HLA-B gene. In some embodiments, the at least one guide ribonucleic acid sequence for specifically targeting the HLA-B gene is selected from the group consisting of SEQ ID NOS: 1419-3277 and in Table 9 and Appendix 2 of WO2016183041, which is herein incorporated by reference. In some embodiments, the cell has a reduced ability to induce an immune response in a recipient subject. In some embodiments, an exogenous nucleic acid encoding a polypeptide as disclosed herein (e.g., a chimeric antigen receptor, a HLA-E variant protein, a HLA-G variant protein, and/or an exogenous PD-L1 protein, or another tolerogenic factor disclosed herein) is inserted at the HLA-B gene.

Assays to test whether the HLA-B gene has been inactivated are known and described herein. In some embodiments, the resulting genetic modification of the HLA-B gene by PCR and the reduction of HLA-I expression can be assays by FACS analysis. In another embodiment, HLA-B protein expression is detected using a Western blot of cells lysates probed with antibodies to the HLA-B protein. In another embodiment, reverse transcriptase polymerase chain reactions (RT-PCR) are used to confirm the presence of the inactivating genetic modification.

### G. HLA-C

In some embodiments, the present technology modulates (e.g., reduces or eliminates) the expression of MHC I genes by targeting and modulating (e.g., reducing or eliminating) HLA-I expression. In some embodiments, the modulation occurs using a CRISPR/Cas system. HLA-C is another of the three major types of MHC class I transmembrane proteins. In a MHC class I heterodimeric molecule, HLA-C protein serves as a heavy chain binds beta2-microglobulin which can be referred to as a light chain. The HLA-C protein is about 45 kDa and is encoded by 8 exons. Exon 1 encodes the leader peptide; exon 2 and 3 encode the alpha1 and alpha2 domains, which both bind an antigen peptide; exon 4 encodes the alpha3 domain; exon 5 encodes the transmembrane region; and exons 6 and 7 encode the cytoplasmic tail.

In some embodiments, the cells described herein comprise gene modifications at the gene locus encoding the HLA-C protein. In other words, the cells comprise a genetic modification at the HLA-C locus. In some instances, the nucleotide sequence encoding the HLA-C protein is set forth in RefSeq. No. NM_002117.5, and NCBI Genbank No. M24097.1

In some instances, the HLA-C gene locus is described in NCBI Gene ID No. 3107. In some cases, the amino acid sequence of HLA-C is set forth in RefSeq. No. NP_002108.4.

Additional descriptions of the HLA-C protein and gene locus can be found in Uniprot No. P10321, HGNC Ref. No. 4933, and OMIM Ref. No. 142840.

In some embodiments, the hypoimmunogenic cells outlined herein comprise a genetic modification targeting the HLA-C gene. In some embodiments, the genetic modification targeting the HLA-C gene by the rare-cutting endonuclease comprises a Cas protein or a polynucleotide encoding a Cas protein, and at least one guide ribonucleic acid sequence for specifically targeting the HLA-C gene. In some embodiments, the at least one guide ribonucleic acid sequence for specifically targeting the HLA-C gene is selected from the group consisting of SEQ ID NOS:3278-5183 and in Table 10 and Appendix 3 of WO2016183041, which is herein incorporated by reference. In some embodiments, the cell has a reduced ability to induce an immune response in a recipient subject. In some embodiments, an exogenous nucleic acid encoding a polypeptide as disclosed herein (e.g., a chimeric antigen receptor, a HLA-E variant protein, a HLA-G variant protein, and/or an exogenous PD-L1 protein, or another tolerogenic factor disclosed herein) is inserted at the HLA-C gene.

Assays to test whether the HLA-C gene has been inactivated are known and described herein. In some embodiments, the resulting genetic modification of the HLA-C gene by PCR and the reduction of HLA-I expression can be assays by FACS analysis. In another embodiment, HLA-C protein expression is detected using a Western blot of cells lysates probed with antibodies to the HLA-C protein. In another embodiment, reverse transcriptase polymerase chain reactions (RT-PCR) are used to confirm the presence of the inactivating genetic modification.

### H. CD155

In some embodiments, the present technology modulates (e.g., reduces or eliminates) the expression of CD155. In some embodiments, the modulation occurs using a CRISPR/Cas system. CD155 is a transmembrane glycoproteins belonging to the immunoglobulin superfamily. It is recognized in the art that CD155 mediates NK cell adhesion and triggers NK cell effector functions. CD155 can binds two different NK cell receptors, such as CD96 and CD22.

In some embodiments, the cells described herein comprise gene modifications at the gene locus encoding the CD155 protein. In other words, the cells comprise a genetic modification at the CD155 locus. In some instances, the nucleotide sequence encoding the CD155 protein is set forth in RefSeq. Nos. NM_001135768.2, NM_001135769.2, and NM 001135770.3 and NCBI Genbank No. M24097.1. In some instances, the CD155 gene locus is described in NCBI Gene ID No. 5817. In some cases, the amino acid sequence of CD155 is set forth in RefSeq. No. NP_1129240.1, NP_1129241.1 and NP_1129242.1. Additional descriptions of the CD155 protein and gene locus can be found in Uniprot No. P15151, HGNC Ref. No. 9705, and OMIM Ref. No. 173850.

In some embodiments, the hypoimmunogenic cells outlined herein comprise a genetic modification targeting the CD155 gene. In some embodiments, the genetic modification targeting the CD155 gene by the rare-cutting endonuclease comprises a Cas protein or a polynucleotide encoding a Cas protein, and at least one guide ribonucleic acid sequence for specifically targeting the CD155 gene. In some embodiments, the at least one guide ribonucleic acid sequence for specifically targeting the CD155 gene is selected from the group consisting of those described in WO2016183041, which is herein incorporated by reference. In some embodiments, the cell has a reduced ability to induce an immune response in a recipient subject. In some embodiments, an exogenous nucleic acid encoding a polypeptide as disclosed herein (e.g., a chimeric antigen receptor, a HLA-E variant protein, a HLA-G variant protein, and/or an exogenous PD-L1 protein, or another tolerogenic factor disclosed herein) is inserted at the CD155 gene.

Assays to test whether the CD155 gene has been inactivated are known and described herein. In some embodiments, the resulting genetic modification of the CD155 gene by PCR and the reduction of HLA-I expression can be assays by FACS analysis. In another embodiment, CD155 protein expression is detected using a Western blot of cells lysates probed with antibodies to the CD155 protein. In another embodiment, reverse transcriptase polymerase chain reactions (RT-PCR) are used to confirm the presence of the inactivating genetic modification.

### I. CIITA

In some embodiments, the present technology modulates (e.g., reduces or eliminates) the expression of MHC II genes by targeting and modulating (e.g., reducing or eliminating) Class II transactivator (CIITA) expression. In some embodiments, the modulation occurs using a CRISPR/Cas system. CIITA is a member of the LR or nucleotide binding domain (NBD) leucine-rich repeat (LRR) family of proteins and regulates the transcription of MHC II by associating with the MHC enhanceosome.

In some embodiments, the target polynucleotide sequence of the present technology is a variant of CIITA. In some embodiments, the target polynucleotide sequence is a homolog of CIITA. In some embodiments, the target polynucleotide sequence is an ortholog of CIITA.

In some embodiments, reduced or eliminated expression of CIITA reduces or eliminates expression of one or more of the following MHC class II are HLA-DP, HLA-DM, HLA-DOA, HLA-DOB, HLA-DQ, and HLA-DR.

In some embodiments, the cells described herein comprise gene modifications at the gene locus encoding the CIITA protein. In other words, the cells comprise a genetic modification at the CIITA locus. In some instances, the nucleotide sequence encoding the CIITA protein is set forth in RefSeq. No. NM_000246.4 and NCBI Genbank No. U18259. In some instances, the CIITA gene locus is described in NCBI Gene ID No. 4261. In certain cases, the amino acid sequence of CIITA is depicted as NCBI GenBank No. AAA88861.1. Additional descriptions of the CIITA protein and gene locus can be found in Uniprot No. P33076, HGNC Ref. No. 7067, and OMIM Ref. No. 600005.

In some embodiments, the hypoimmunogenic cells outlined herein comprise a genetic modification targeting the CIITA gene. In some embodiments, the genetic modification targeting the CIITA gene by the rare-cutting endonuclease comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from the group consisting of SEQ ID NOS:5184-36352 of Table 12 of WO2016183041, which is herein incorporated by reference. In some embodiments, the cell has a reduced ability to induce an immune response in a recipient subject. In some embodiments, an exogenous nucleic acid encoding a polypeptide as disclosed herein (e.g., a chimeric antigen receptor, a HLA-E variant protein, a HLA-G variant protein, and/or an exogenous PD-L1 protein, or another tolerogenic factor disclosed herein) is inserted at the CIITA gene.

Assays to test whether the CIITA gene has been inactivated are known and described herein. In some embodiments, the resulting genetic modification of the CIITA gene by PCR and the reduction of HLA-II expression can be assays by FACS analysis. In another embodiment, CIITA protein expression is detected using a Western blot of cells lysates probed with antibodies to the CIITA protein. In another embodiment, reverse transcriptase polymerase chain reactions (RT-PCR) are used to confirm the presence of the inactivating genetic modification.

### J. B2M

In some embodiments, the technology disclosed herein modulates (e.g., reduces or eliminates) the expression of MHC-I genes by targeting and modulating (e.g., reducing or eliminating) expression of the accessory chain B2M. In some embodiments, the modulation occurs using a CRISPR/Cas system. By modulating (e.g., reducing or deleting) expression of B2M, surface trafficking of MHC-I molecules is blocked and the cell rendered hypoimmunogenic. In some embodiments, the cell has a reduced ability to induce an immune response in a recipient subject.

In some embodiments, the target polynucleotide sequence of the present technology is a variant of B2M. In some embodiments, the target polynucleotide sequence is a homolog of B2M. In some embodiments, the target polynucleotide sequence is an ortholog of B2M.

In some embodiments, decreased or eliminated expression of B2M reduces or eliminates expression of one or more of the following MHC I molecules: HLA-A, HLA-B, and HLA-C.

In some embodiments, the cells described herein comprise gene modifications at the gene locus encoding the B2M protein. In other words, the cells comprise a genetic modification at the B2M locus. In some instances, the nucleotide sequence encoding the B2M protein is set forth in RefSeq. No. NM_004048.4 and Genbank No. AB021288.1. In some instances, the B2M gene locus is described in NCBI Gene ID No. 567. In certain cases, the amino acid sequence of B2M is depicted as NCBI GenBank No. BAA35182.1. Additional descriptions of the B2M protein and gene locus can be found in Uniprot No. P61769, HGNC Ref. No. 914, and OMIM Ref. No. 109700.

In some embodiments, the hypoimmunogenic cells outlined herein comprise a genetic modification targeting the B2M gene. In some embodiments, the genetic modification targeting the B2M gene by the rare-cutting endonuclease comprises a Cas protein or a polynucleotide encoding a Cas protein, and at least one guide ribonucleic acid sequence for specifically targeting the B2M gene. In some embodiments, the at least one guide ribonucleic acid sequence for specifically targeting the B2M gene is selected from the group consisting of SEQ ID NOS:81240-85644 of Table 15 of WO2016183041, which is herein incorporated by reference. In some embodiments, an exogenous nucleic acid encoding a polypeptide as disclosed herein (e.g., a chimeric antigen receptor, a HLA-E variant protein, a HLA-G variant protein, and/or an exogenous PD-L1 protein, or another tolerogenic factor disclosed herein) is inserted at the B2M gene.

Assays to test whether the B2M gene has been inactivated are known and described herein. In some embodiments, the resulting genetic modification of the B2M gene by PCR and the reduction of HLA-I expression can be assays by FACS analysis. In another embodiment, B2M protein expression is detected using a Western blot of cells lysates probed with antibodies to the B2M protein. In another embodiment, reverse transcriptase polymerase chain reactions (RT-PCR) are used to confirm the presence of the inactivating genetic modification.

### K. NLRC5

In many embodiments, the technology disclosed herein modulate (e.g., reduce or eliminate) the expression of MHC-I genes by targeting and modulating (e.g., reducing or eliminating) expression of the NLR family, CARD domain containing 5/NOD27/CLR16.1 (NLRC5). In some embodiments, the modulation occurs using a CRISPR/Cas system. NLRC5 is a critical regulator of MHC-I-mediated immune responses and, similar to CIITA, NLRC5 is highly inducible by IFN-γ and can translocate into the nucleus. NLRC5 activates the promoters of MHC-I genes and induces the transcription of MHC-I as well as related genes involved in MHC-I antigen presentation.

In some embodiments, the target polynucleotide sequence is a variant of NLRC5. In some embodiments, the target polynucleotide sequence is a homolog of NLRC5. In some embodiments, the target polynucleotide sequence is an ortholog of NLRC5.

In some embodiments, decreased or eliminated expression of NLRC5 reduces or eliminates expression of one or more of the following MHC I molecules—HLA-A, HLA-B, and HLA-C.

In some embodiments, the cells outlined herein comprise a genetic modification targeting the NLRC5 gene. In some embodiments, the genetic modification targeting the NLRC5 gene by the rare-cutting endonuclease comprises a Cas protein or a polynucleotide encoding a Cas protein, and at least one guide ribonucleic acid sequence for specifically targeting the NLRC5 gene. In some embodiments, the at least one guide ribonucleic acid sequence for specifically targeting the NLRC5 gene is selected from the group consisting of SEQ ID NOS:36353-81239 of Appendix 3 or Table 14 of WO2016183041, the disclosure is incorporated by reference in its entirety.

Assays to test whether the NLRC5 gene has been inactivated are known and described herein. In some embodiments, the resulting genetic modification of the NLRC5 gene by PCR and the reduction of HLA-I expression can be assays by FACS analysis. In another embodiment, NLRC5 protein expression is detected using a Western blot of cells lysates probed with antibodies to the NLRC5 protein. In another embodiment, reverse transcriptase polymerase chain reactions (RT-PCR) are used to confirm the presence of the inactivating genetic modification.

### L. TRAC

In many embodiments, the technologies disclosed herein modulate (e.g., reduce or eliminate) the expression of TCR genes including the TRAC gene by targeting and modulating (e.g., reducing or eliminating) expression of the constant region of the T cell receptor alpha chain. In some embodiments, the modulation occurs using a CRISPR/Cas system. By modulating (e.g., reducing or deleting) expression of TRAC, surface trafficking of TCR molecules is blocked. In some embodiments, the cell also has a reduced ability to induce an immune response in a recipient subject.

In some embodiments, the target polynucleotide sequence of the present technology is a variant of TRAC. In some embodiments, the target polynucleotide sequence is a homolog of TRAC. In some embodiments, the target polynucleotide sequence is an ortholog of TRAC.

In some embodiments, decreased or eliminated expression of TRAC reduces or eliminates TCR surface expression.

In some embodiments, the cells, such as, but not limited to, pluripotent stem cells, induced pluripotent stem cells, T cells differentiated from induced pluripotent stem cells, primary T cells, and cells derived from primary T cells comprise gene modifications at the gene locus encoding the TRAC protein. In other words, the cells comprise a genetic modification at the TRAC locus. In some instances, the nucleotide sequence encoding the TRAC protein is set forth in Genbank No. X02592.1. In some instances, the TRAC gene locus is described in RefSeq. No. NG 001332.3 and NCBI Gene ID No. 28755. In certain cases, the amino acid sequence of TRAC is depicted as Uniprot No. P01848. Additional descriptions of the TRAC protein and gene locus can be found in Uniprot No. P01848, HGNC Ref. No. 12029, and OMIM Ref. No. 186880.

In some embodiments, the hypoimmunogenic cells outlined herein comprise a genetic modification targeting the TRAC gene. In some embodiments, the genetic modification targeting the TRAC gene by the rare-cutting endonuclease comprises a Cas protein or a polynucleotide encoding a Cas protein, and at least one guide ribonucleic acid sequence for specifically targeting the TRAC gene. In some embodiments, the at least one guide ribonucleic acid sequence for specifically targeting the TRAC gene is selected from the group consisting of SEQ ID NOS:532-609 and 9102-9797 of US20160348073, which is herein incorporated by reference.

Assays to test whether the TRAC gene has been inactivated are known and described herein. In some embodiments, the resulting genetic modification of the TRAC gene by PCR and the reduction of TCR expression can be assays by FACS analysis. In another embodiment, TRAC protein expression is detected using a Western blot of cells lysates probed with antibodies to the TRAC protein. In another embodiment, reverse transcriptase polymerase chain reactions (RT-PCR) are used to confirm the presence of the inactivating genetic modification.

### M. TRB

In many embodiments, the technologies disclosed herein modulate (e.g., reduce or eliminate) the expression of TCR genes including the gene encoding T cell antigen receptor, beta chain (e.g., the TRB, TRBC, or TCRB gene) by targeting and modulating (e.g., reducing or eliminating) expression of the constant region of the T cell receptor beta chain. In some embodiments, the modulation occurs using a CRISPR/Cas system. By modulating (e.g., reducing or deleting) expression of TRB, surface trafficking of TCR molecules is blocked. In some embodiments, the cell also has a reduced ability to induce an immune response in a recipient subject.

In some embodiments, the target polynucleotide sequence of the present technology is a variant of TRB. In some embodiments, the target polynucleotide sequence is a homolog of TRB. In some embodiments, the target polynucleotide sequence is an ortholog of TRB.

In some embodiments, decreased or eliminated expression of TRB reduces or eliminates TCR surface expression.

In some embodiments, the cells, such as, but not limited to, pluripotent stem cells, induced pluripotent stem cells, T cells differentiated from induced pluripotent stem cells, primary T cells, and cells derived from primary T cells comprise gene modifications at the gene locus encoding the TRB protein. In other words, the cells comprise a genetic modification at the TRB gene locus. In some instances, the nucleotide sequence encoding the TRB protein is set forth in UniProt No. PODSE2. In some instances, the TRB gene locus is described in RefSeq. No. NG_001333.2 and NCBI Gene ID No. 6957. In certain cases, the amino acid sequence of TRB is depicted as Uniprot No. P01848. Additional descriptions of the TRB protein and gene locus can be found in GenBank No. L36092.2, Uniprot No. PODSE2, and HGNC Ref. No. 12155.

In some embodiments, the hypoimmunogenic cells outlined herein comprise a genetic modification targeting the TRB gene. In some embodiments, the genetic modification targeting the TRB gene by the rare-cutting endonuclease comprises a Cas protein or a polynucleotide encoding a Cas protein, and at least one guide ribonucleic acid sequence for specifically targeting the TRB gene. In some embodiments, the at least one guide ribonucleic acid sequence for specifically targeting the TRB gene is selected from the group consisting of SEQ ID NOS:610-765 and 9798-10532 of US20160348073, which is herein incorporated by reference.

Assays to test whether the TRB gene has been inactivated are known and described herein. In some embodiments, the resulting genetic modification of the TRB gene by PCR and the reduction of TCR expression can be assays by FACS analysis. In another embodiment, TRB protein expression is detected using a Western blot of cells lysates probed with antibodies to the TRB protein. In another embodiment, reverse transcriptase polymerase chain reactions (RT-PCR) are used to confirm the presence of the inactivating genetic modification.

### N. Additional Tolerogenic Factors

In many embodiments, one or more tolerogenic factors can be inserted or reinserted into genome-edited cells to create immune-privileged universal donor cells, such as universal donor stem cells, universal donor T cells, or universal donor cells. In many embodiments, the hypoimmunogenic cells disclosed herein have been further modified to express one or more tolerogenic factors. Exemplary tolerogenic factors include, without limitation, one or more of CD47, DUX4, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FasL, CCL21, CCL22, Mfge8, and Serpinb9. In some embodiments, the tolerogenic factors are selected from the group consisting of CD200, HLA-G, HLA-E, HLA-C, HLA-E heavy chain, PD-L1, IDO1, CTLA4-Ig, IL-10, IL-35, FasL, Serpinb9, CCL21, CCL22, and Mfge8. In some embodiments, the tolerogenic factors are selected from the group consisting of DUX4, HLA-C, HLA-E, HLA-F, HLA-G, PD-L1, CTLA-4-Ig, C1-inhibitor, and IL-35. In some embodiments, the tolerogenic factors are selected from the group consisting of HLA-C, HLA-E, HLA-F, HLA-G, PD-L1, CTLA-4-Ig, C1-inhibitor, and IL-35. In some embodiments, the tolerogenic factors are selected from a group including CD47, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FasL, CCL21, CCL22, Mfge8, and Serpinb9.

Useful genomic, polynucleotide and polypeptide information about human CD27 (which is also known as CD27L receptor, Tumor Necrosis Factor Receptor Superfamily Member 7, TNFSF7, T Cell Activation Antigen S152, Tp55, and T14) are provided in, for example, the GeneCard Identifier GC12P008144, HGNC No. 11922, NCBI Gene ID 939, Uniprot No. P26842, and NCBI RefSeq Nos. NM_001242.4 and NP_001233.1.

Useful genomic, polynucleotide and polypeptide information about human CD46 are provided in, for example, the GeneCard Identifier GC01P207752, HGNC No. 6953, NCBI Gene ID 4179, Uniprot No. P15529, and NCBI RefSeq Nos. NM_002389.4, NM_153826.3, NM_172350.2, NM_172351.2, NM_172352.2 NP_758860.1, NM_172353.2, NM_172359.2, NM_172361.2, NP_002380.3, NP_722548.1, NP_758860.1, NP_758861.1, NP_758862.1, NP_758863.1, NP_758869.1, and NP_758871.1.

Useful genomic, polynucleotide and polypeptide information about human CD55 (also known as complement decay-accelerating factor) are provided in, for example, the GeneCard Identifier GC01P207321, HGNC No. 2665, NCBI Gene ID 1604, Uniprot No. P08174, and NCBI RefSeq Nos. NM_000574.4, NM_001114752.2, NM_001300903.1, NM_001300904.1, NP_000565.1, NP_001108224.1, NP_001287832.1, and NP_001287833.1.

Useful genomic, polynucleotide and polypeptide information about human CD59 are provided in, for example, the GeneCard Identifier GC11M033704, HGNC No. 1689, NCBI Gene ID 966, Uniprot No. P13987, and NCBI RefSeq Nos. NP_000602.1, NM_000611.5, NP_001120695.1, NM_001127223.1, NP_001120697.1, NM_001127225.1, NP_001120698.1, NM 001127226.1, NP_001120699.1, NM_001127227.1, NP_976074.1, NM_203329.2, NP_976075.1, NM_203330.2, NP_976076.1, and NM_203331.2.

Useful genomic, polynucleotide and polypeptide information about human CD200 are provided in, for example, the GeneCard Identifier GC03P112332, HGNC No. 7203, NCBI Gene ID 4345, Uniprot No. P41217, and NCBI RefSeq Nos. NP_001004196.2, NM_001004196.3, NP_001305757.1, NM_001318828.1, NP_005935.4, NM_005944.6, XP_005247539.1, and XM_005247482.2.

Useful genomic, polynucleotide and polypeptide information about human HLA-C are provided in, for example, the GeneCard Identifier GC06M031272, HGNC No. 4933, NCBI Gene ID 3107, Uniprot No. P10321, and NCBI RefSeq Nos. NP_002108.4 and NM_002117.5.

Useful genomic, polynucleotide and polypeptide information about human HLA-E are provided in, for example, the GeneCard Identifier GC06P047281, HGNC No. 4962, NCBI Gene ID 3133, Uniprot No. P13747, and NCBI RefSeq Nos. NP_005507.3 and NM_005516.5.

Useful genomic, polynucleotide and polypeptide information about human HLA-G are provided in, for example, the GeneCard Identifier GC06P047256, HGNC No. 4964, NCBI Gene ID 3135, Uniprot No. P17693, and NCBI RefSeq Nos. NP 002118.1 and NM 002127.5.

Useful genomic, polynucleotide and polypeptide information about human PD-L1 or CD274 are provided in, for example, the GeneCard Identifier GC09P005450, HGNC No. 17635, NCBI Gene ID 29126, Uniprot No. Q9NZQ7, and NCBI RefSeq Nos. NP_001254635.1, NM_001267706.1, NP_054862.1, and NM_014143.3.

Useful genomic, polynucleotide and polypeptide information about human IDO1 are provided in, for example, the GeneCard Identifier GC08P039891, HGNC No. 6059, NCBI Gene ID 3620, Uniprot No. P14902, and NCBI RefSeq Nos. NP_002155.1 and NM_002164.5.

Useful genomic, polynucleotide and polypeptide information about human IL-10 are provided in, for example, the GeneCard Identifier GC01M206767, HGNC No. 5962, NCBI Gene ID 3586, Uniprot No. P22301, and NCBI RefSeq Nos. NP_000563.1 and NM_000572.2.

Useful genomic, polynucleotide and polypeptide information about human Fas ligand (which is known as FasL, FASLG, CD178, TNFSF6, and the like) are provided in, for example, the GeneCard Identifier GC01P172628, HGNC No. 11936, NCBI Gene ID 356, Uniprot No. P48023, and NCBI RefSeq Nos. NP_000630.1, NM_000639.2, NP_001289675.1, and NM 001302746.1.

Useful genomic, polynucleotide and polypeptide information about human CCL21 are provided in, for example, the GeneCard Identifier GC09M034709, HGNC No. 10620, NCBI Gene ID 6366, Uniprot No. 000585, and NCBI RefSeq Nos. NP_002980.1 and NM_002989.3.

Useful genomic, polynucleotide and polypeptide information about human CCL22 are provided in, for example, the GeneCard Identifier GC16P057359, HGNC No. 10621, NCBI Gene ID 6367, Uniprot No. 000626, and NCBI RefSeq Nos. NP_002981.2, NM_002990.4, XP_016879020.1, and XM_017023531.1.

Useful genomic, polynucleotide and polypeptide information about human Mfge8 are provided in, for example, the GeneCard Identifier GC15M088898, HGNC No. 7036, NCBI Gene ID 4240, Uniprot No. Q08431, and NCBI RefSeq Nos. NP_001108086.1, NM_001114614.2, NP_001297248.1, NM_001310319.1, NP_001297249.1, NM_001310320.1, NP_001297250.1, NM_001310321.1, NP_005919.2, and NM_005928.3.

Useful genomic, polynucleotide and polypeptide information about human SerpinB9 are provided in, for example, the GeneCard Identifier GC06M002887, HGNC No. 8955, NCBI Gene ID 5272, Uniprot No. P50453, and NCBI RefSeq Nos. NP_004146.1, NM_004155.5, XP_005249241.1, and XM_005249184.4.

Methods for modulating expression of genes and factors (proteins) include genome editing technologies, and RNA or protein expression technologies and the like. For all of these technologies, well known recombinant techniques are used, to generate recombinant nucleic acids as outlined herein.

In some instances, a gene editing system such as the CRISPR/Cas system is used to facilitate the insertion of tolerogenic factors, such as the tolerogenic factors into a safe harbor locus, such as the AAVS1 locus, to actively inhibit immune rejection. In some instances, the tolerogenic factors are inserted into a safe harbor locus using an expression vector. In some embodiments, the safe harbor locus is an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus.

In some embodiments, expression of a target gene (e.g., an HLA-E variant, an HLA-G variant, and/or exogenous PD-L1, or another tolerogenic factor gene) is increased by expression of fusion protein or a protein complex containing (1) a site-specific binding domain specific for the endogenous target gene (e.g., an HLA-E variant, an HLA-G variant, and/or exogenous PD-L1, or another tolerogenic factor gene) and (2) a transcriptional activator.

In some embodiments, the regulatory factor is comprised of a site-specific DNA-binding nucleic acid molecule, such as a guide RNA (gRNA). In some embodiments, the method is achieved by site specific DNA-binding targeted proteins, such as zinc finger proteins (ZFP) or fusion proteins containing ZFP, which are also known as zinc finger nucleases (ZFNs).

In some embodiments, the regulatory factor comprises a site-specific binding domain, such as using a DNA binding protein or DNA-binding nucleic acid, which specifically binds to or hybridizes to the gene at a targeted region. In some embodiments, the provided polynucleotides or polypeptides are coupled to or complexed with a site-specific nuclease, such as a modified nuclease. For example, in some embodiments, the administration is effected using a fusion comprising a DNA-targeting protein of a modified nuclease, such as a meganuclease or an RNA-guided nuclease such as a clustered regularly interspersed short palindromic nucleic acid (CRISPR)-Cas system, such as CRISPR-Cas9 system. In some embodiments, the nuclease is modified to lack nuclease activity. In some embodiments, the modified nuclease is a catalytically dead dCas9.

In some embodiments, the site-specific binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. See also U.S. Pat. Nos. 5,420,032; 6,833,252; Belfort et al., (1997) Nucleic Acids Res. 25:3379-3388; Dujon et al., (1989) Gene 82:115-118; Perler et al, (1994) Nucleic Acids Res. 22, 1125-1127; Jasin (1996) Trends Genet. 12:224-228; Gimble et al., (1996) J. Mol. Biol. 263: 163-180; Argast et al, (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al, (2002) Molec. Cell 10:895-905; Epinat et al, (2003) Nucleic Acids Res. 31:2952-2962; Ashworth et al, (2006) Nature 441:656-659; Paques et al, (2007) Current Gene Therapy 7:49-66; U.S. Patent Publication No. 2007/0117128.

Zinc finger, TALE, and CRISPR system binding domains can be “engineered” to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some embodiments, the site-specific binding domain comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion.

Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed.

In some embodiments, the site-specific binding domain comprises a naturally occurring or engineered (non-naturally occurring) transcription activator-like protein (TAL) DNA binding domain, such as in a transcription activator-like protein effector (TALE) protein, See, e.g., U.S. Patent Publication No. 20110301073, incorporated by reference in its entirety herein.

In some embodiments, the site-specific binding domain is derived from the CRISPR/Cas system. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system, or a “targeting sequence”), and/or other sequences and transcripts from a CRISPR locus.

In general, a guide sequence includes a targeting domain comprising a polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some examples, the targeting domain of the gRNA is complementary, e.g., at least 80, 85, 90, 95, 98 or 99% complementary, e.g., fully complementary, to the target sequence on the target nucleic acid.

In some embodiments, the target site is upstream of a transcription initiation site of the target gene. In some embodiments, the target site is adjacent to a transcription initiation site of the gene. In some embodiments, the target site is adjacent to an RNA polymerase pause site downstream of a transcription initiation site of the gene.

In some embodiments, the targeting domain is configured to target the promoter region of the target gene to promote transcription initiation, binding of one or more transcription enhancers or activators, and/or RNA polymerase. One or more gRNA can be used to target the promoter region of the gene. In some embodiments, one or more regions of the gene can be targeted. In certain aspects, the target sites are within 600 base pairs on either side of a transcription start site (TSS) of the gene.

It is within the level of a skilled artisan to design or identify a gRNA sequence that is or comprises a sequence targeting a gene, including the exon sequence and sequences of regulatory regions, including promoters and activators. A genome-wide gRNA database for CRISPR genome editing is publicly available, which contains exemplary single guide RNA (sgRNA) target sequences in constitutive exons of genes in the human genome or mouse genome (see e.g., genescript.com/gRNA-database.html; see also, Sanjana et al. (2014) Nat. Methods, 11:783-4; www.e-crisp.org/E-CRISP/; crispr.mit.edu/). In some embodiments, the gRNA sequence is or comprises a sequence with minimal off-target binding to a non-target gene.

In some embodiments, the regulatory factor further comprises a functional domain, e.g., a transcriptional activator.

In some embodiments, the transcriptional activator is or contains one or more regulatory elements, such as one or more transcriptional control elements of a target gene, whereby a site-specific domain as provided above is recognized to drive expression of such gene. In some embodiments, the transcriptional activator drives expression of the target gene. In some cases, the transcriptional activator, can be or contain all or a portion of a heterologous transactivation domain. For example, in some embodiments, the transcriptional activator is selected from Herpes simplex-derived transactivation domain, Dnmt3a methyltransferase domain, p65, VP16, and VP64.

In some embodiments, the regulatory factor is a zinc finger transcription factor (ZF-TF). In some embodiments, the regulatory factor is VP64-p65-Rta (VPR).

In certain embodiments, the regulatory factor further comprises a transcriptional regulatory domain. Common domains include, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g. kinases, acetylases and deacetylases); and DNA modifying enzymes (e.g., methyltransferases such as members of the DNMT family (e.g., DNMT1, DNMT3A, DNMT3B, DNMT3L, etc., topoisomerases, helicases, ligases, kinases, phosphatases, polymerases, endonucleases) and their associated factors and modifiers. See, e.g., U.S. Publication No. 2013/0253040, incorporated by reference in its entirety herein.

Suitable domains for achieving activation include the HSV VP 16 activation domain (see, e.g., Hagmann et al, J. Virol. 71, 5952-5962 (1 97)) nuclear hormone receptors (see, e.g., Torchia et al., Curr. Opin. Cell. Biol. 10:373-383 (1998)); the p65 subunit of nuclear factor kappa B (Bitko & Bank, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Beerli et al., (1998) Proc. Natl. Acad. Sci. USA 95:14623-33), and degron (Molinari et al., (1999) EMBO J. 18, 6439-6447). Additional exemplary activation domains include, Oct 1, Oct-2A, Spl, AP-2, and CTF1 (Seipel et al, EMBOJ. 11, 4961-4968 (1992) as well as p300, CBP, PCAF, SRC1 PvALF, AtHD2A and ERF-2. See, for example, Robyr et al, (2000) Mol. Endocrinol. 14:329-347; Collingwood et al, (1999) J. Mol. Endocrinol 23:255-275; Leo et al, (2000) Gene 245:1-11; Manteuffel-Cymborowska (1999) Acta Biochim. Pol. 46:77-89; Mckenna et al, (1999) J. Steroid Biochem. Mol. Biol. 69:3-12; Malik et al, (2000) Trends Biochem. Sci. 25:277-283; and Lemon et al, (1999) Curr. Opin. Genet. Dev. 9:499-504. Additional exemplary activation domains include, but are not limited to, OsGAI, HALF-1, C1, AP1, ARF-5, -6, -1, and -8, CPRF1, CPRF4, MYC-RP/GP, and TRAB1, See, for example, Ogawa et al, (2000) Gene 245:21-29; Okanami et al, (1996) Genes Cells 1:87-99; Goff et al, (1991) Genes Dev. 5:298-309; Cho et al, (1999) Plant Mol Biol 40:419-429; Ulmason et al, (1999) Proc. Natl. Acad. Sci. USA 96:5844-5849; Sprenger-Haussels et al, (2000) Plant J. 22:1-8; Gong et al, (1999) Plant Mol. Biol. 41:33-44; and Hobo et al., (1999) Proc. Natl. Acad. Sci. USA 96:15,348-15,353.

Exemplary repression domains that can be used to make genetic repressors include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B, DNMT3L, etc.), Rb, and MeCP2. See, for example, Bird et al, (1999) Cell 99:451-454; Tyler et al, (1999) Cell 99:443-446; Knoepfler et al, (1999) Cell 99:447-450; and Robertson et al, (2000) Nature Genet. 25:338-342. Additional exemplary repression domains include, but are not limited to, ROM2 and AtHD2A. See, for example, Chem et al, (1996) Plant Cell 8:305-321; and Wu et al, (2000) Plant J. 22:19-27.

In some instances, the domain is involved in epigenetic regulation of a chromosome. In some embodiments, the domain is a histone acetyltransferase (HAT), e.g. type-A, nuclear localized such as MYST family members MOZ, Ybf2/Sas3, MOF, and Tip60, GNAT family members Gcn5 or pCAF, the p300 family members CBP, p300 or Rttl09 (Bemdsen and Denu (2008) Curr Opin Struct Biol 18(6):682-689). In other instances the domain is a histone deacetylase (HD AC) such as the class I (HDAC-1, 2, 3, and 8), class II (HDAC IIA (HDAC-4, 5, 7 and 9), HD AC IIB (HDAC 6 and 10)), class IV (HDAC-1 1), class III (also known as sirtuins (SIRTs); SIRT1-7) (see Mottamal et al., (2015) Molecules 20(3):3898-3941). Another domain that is used in some embodiments is a histone phosphorylase or kinase, where examples include MSK1, MSK2, ATR, ATM, DNA-PK, Bubl, VprBP, IKK-a, PKCpi, Dik/Zip, JAK2, PKC5, WSTF and CK2. In some embodiments, a methylation domain is used and may be chosen from groups such as Ezh2, PRMT1/6, PRMT5/7, PRMT 2/6, CARMI, set7/9, MLL, ALL-1, Suv 39h, G9a, SETDB1, Ezh2, Set2, Dotl, PRMT 1/6, PRMT 5/7, PR-Set7 and Suv4-20h, Domains involved in sumoylation and biotinylation (Lys9, 13, 4, 18 and 12) may also be used in some embodiments (review see Kousarides (2007) Cell 128:693-705).

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, IL) Catalogue. Methods and compositions for making fusions between a minor groove binder and a polypeptide have been described. Mapp et al, (2000) Proc. Natl. Acad. Sci. USA 97:3930-3935. Likewise, CRISPR/Cas TFs and nucleases comprising a sgRNA nucleic acid component in association with a polypeptide component function domain are also known to those of skill in the art and detailed herein.

In some embodiments, the present disclosure provides a cell (e.g., a primary T cell and a hypoimmunogenic stem cell and derivative thereof) or population thereof comprising a genome in which the cell genome has been modified to express an HLA-E variant, an HLA-G variant, and/or an exogenous PD-L1. In some embodiments, the present disclosure provides a method for altering a cell genome to express an HLA-E variant, an HLA-G variant, and/or an exogenous PD-L1. In many embodiments, at least one ribonucleic acid or at least one pair of ribonucleic acids may be utilized to facilitate the insertion of an HLA-E variant, an HLA-G variant, and/or an exogenous PD-L1 into a cell line. In many embodiments, the at least one ribonucleic acid or the at least one pair of ribonucleic acids is selected from the group consisting of SEQ ID NOS:200784-231885 of Table 29 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-C. In some embodiments, the present disclosure provides a method for altering a cell genome to express HLA-C. In many embodiments, at least one ribonucleic acid or at least one pair of ribonucleic acids may be utilized to facilitate the insertion of HLA-C into a cell line. In many embodiments, the at least one ribonucleic acid or the at least one pair of ribonucleic acids is selected from the group consisting of SEQ ID NOS:3278-5183 of Table 10 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-E. In some embodiments, the present disclosure provides a method for altering a cell genome to express HLA-E. In many embodiments, at least one ribonucleic acid or at least one pair of ribonucleic acids may be utilized to facilitate the insertion of HLA-E into a cell line. In many embodiments, the at least one ribonucleic acid or the at least one pair of ribonucleic acids is selected from the group consisting of SEQ ID NOS: 189859-193183 of Table 19 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-F. In some embodiments, the present disclosure provides a method for altering a cell genome to express HLA-F. In many embodiments, at least one ribonucleic acid or at least one pair of ribonucleic acids may be utilized to facilitate the insertion of HLA-F into a cell line. In many embodiments, the at least one ribonucleic acid or the at least one pair of ribonucleic acids is selected from the group consisting of SEQ ID NOS: 688808-399754 of Table 45 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-G. In some embodiments, the present disclosure provides a method for altering a cell genome to express HLA-G. In many embodiments, at least one ribonucleic acid or at least one pair of ribonucleic acids may be utilized to facilitate the insertion of HLA-G into a stem cell line. In many embodiments, the at least one ribonucleic acid or the at least one pair of ribonucleic acids is selected from the group consisting of SEQ ID NOS: 188372-189858 of Table 18 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express PD-L1. In some embodiments, the present disclosure provides a method for altering a cell genome to express PD-L1. In many embodiments, at least one ribonucleic acid or at least one pair of ribonucleic acids may be utilized to facilitate the insertion of PD-L1 into a stem cell line. In many embodiments, the at least one ribonucleic acid or the at least one pair of ribonucleic acids is selected from the group consisting of SEQ ID NOS: 193184-200783 of Table 21 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express CTLA4-Ig. In some embodiments, the present disclosure provides a method for altering a cell genome to express CTLA4-Ig. In many embodiments, at least one ribonucleic acid or at least one pair of ribonucleic acids may be utilized to facilitate the insertion of CTLA4-Ig into a stem cell line. In many embodiments, the at least one ribonucleic acid or the at least one pair of ribonucleic acids is selected from any one disclosed in WO2016183041, including the sequence listing.

In some embodiments, the present disclosure provides a cell (e.g., a primary T cell and a hypoimmunogenic stem cell and derivative thereof) or population thereof comprising a genome in which the cell genome has been modified to express CI-inhibitor. In some embodiments, the present disclosure provides a method for altering a cell genome to express CI-inhibitor. In many embodiments, at least one ribonucleic acid or at least one pair of ribonucleic acids may be utilized to facilitate the insertion of CI-inhibitor into a stem cell line. In many embodiments, the at least one ribonucleic acid or the at least one pair of ribonucleic acids is selected from any one disclosed in WO2016183041, including the sequence listing.

In some embodiments, the present disclosure provides a cell (e.g., a primary T cell and a hypoimmunogenic stem cell and derivative thereof) or population thereof comprising a genome in which the cell genome has been modified to express IL-35. In some embodiments, the present disclosure provides a method for altering a cell genome to express IL-35. In many embodiments, at least one ribonucleic acid or at least one pair of ribonucleic acids may be utilized to facilitate the insertion of IL-35 into a stem cell line. In many embodiments, the at least one ribonucleic acid or the at least one pair of ribonucleic acids is selected from any one disclosed in WO2016183041, including the sequence listing.

In some embodiments, the tolerogenic factors are expressed in a cell using an expression vector. For example, the expression vector for expressing an HLA-E variant, an HLA-G variant, and/or an exogenous PD-L1 in a cell comprises a polynucleotide sequence encoding an HLA-E variant, an HLA-G variant, and/or an exogenous PD-L1. The expression vector can be an inducible expression vector. The expression vector can be a viral vector, such as but not limited to, a lentiviral vector.

In some embodiments, a suitable gene editing system (e.g., CRISPR/Cas system or any of the gene editing systems described herein) is used to facilitate the insertion of a polynucleotide encoding a tolerogenic factor, into a genomic locus of the hypoimmunogenic cell. In some cases, the polynucleotide encoding the tolerogenic factor is inserted into a safe harbor locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding the tolerogenic factor is inserted into an HLA-A gene locus, an HLA-B gene locus, an HLA-C gene locus, a CD155 gene locus, a B2M gene locus, a CIITA gene locus, a TRAC gene locus, or a TRB gene locus. In some embodiments, the polynucleotide encoding the tolerogenic factor is inserted into any one of the gene loci depicted in Table 1 provided herein. In many embodiments, the polynucleotide encoding the tolerogenic factor is operably linked to a promoter.

### O. Chimeric Antigen Receptors

Provided herein are hypoimmunogenic cells comprising a chimeric antigen receptor (CAR). In some embodiments, the CAR is binds to CD19. In some embodiments, the CAR is binds to CD22. In some embodiments, the CAR is binds to CD19. In some embodiments, the CAR is binds to CD19 and CD22. In some embodiments, the CAR is selected from the group consisting of a first-generation CAR, a second generation CAR, a third generation CAR, and a fourth generation CAR. In some embodiments, the CAR includes a single binding domain that binds to a single target antigen. In some embodiments, the CAR includes a single binding domain that binds to more than one target antigen, e.g., 2, 3, or more target antigens. In some embodiments, the CAR includes two binding domains such that each binding domain binds to different target antigens. In some embodiments, the CAR includes two binding domains such that each binding domain binds to the same target antigen. Detailed descriptions of exemplary CARs including CD19-specific, CD22-specific and CD19/CD22-bispecific CARs can be found in WO2012/079000, WO2016/149578 and WO2020/014482, the disclosures including the sequence listings and figures are incorporated herein by reference in their entirety.

In some embodiments, the CD19 specific CAR includes an anti-CD19 single-chain antibody fragment (scFv), a transmembrane domain such as one derived from human CD8α, a 4-1BB (CD137) co-stimulatory signaling domain, and a CD3ζ signaling domain. In some embodiments, the CD22 specific CAR includes an anti-CD22 scFv, a transmembrane domain such as one derived from human CD8α, a 4-1BB (CD137) co-stimulatory signaling domain, and a CD3ζ signaling domain. In some embodiments, the CD19/CD22-bispecific CAR includes an anti-CD19 scFv, an anti-CD22 scFv, a transmembrane domain such as one derived from human CD8α, a 4-1BB (CD137) co-stimulatory signaling domain, and a CD3ζ signaling domain.

In some embodiments, a hypoimmunogenic cell described herein comprises a polynucleotide encoding a chimeric antigen receptor (CAR) comprising an antigen binding domain. In some embodiments, a hypoimmunogenic cell described herein comprises a chimeric antigen receptor (CAR) comprising an antigen binding domain. In some embodiments, the polynucleotide is or comprises a chimeric antigen receptor (CAR) comprising an antigen binding domain. In some embodiments, the CAR is or comprises a first-generation CAR comprising an antigen binding domain, a transmembrane domain, and at least one signaling domain (e.g., one, two or three signaling domains). In some embodiments, the CAR comprises a second-generation CAR comprising an antigen binding domain, a transmembrane domain, and at least two signaling domains. In some embodiments, the CAR comprises a third generation CAR comprising an antigen binding domain, a transmembrane domain, and at least three signaling domains. In some embodiments, a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an antibody, an antibody fragment, an scFv or a Fab.

### 1. Antigen Binding Domain (ABD) Targets an Antigen Characteristic of a Neoplastic or Cancer Cell

In some embodiments, the antigen binding domain (ABD) targets an antigen characteristic of a neoplastic cell. In other words, the antigen binding domain targets an antigen expressed by a neoplastic or cancer cell. In some embodiments, the ABD binds a tumor associated antigen. In some embodiments, the antigen characteristic of a neoplastic cell (e.g., antigen associated with a neoplastic or cancer cell) or a tumor associated antigen is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, epidermal growth factor receptors (EGFR) (including ErbB1/EGFR, ErbB2/HER2, ErbB3/HER3, and ErbB4/HER4), fibroblast growth factor receptors (FGFR) (including FGF1, FGF2, FGF3, FGF4, FGF5, FGF6, FGF7, FGF18, and FGF21), vascular endothelial growth factor receptors (VEGFR) (including VEGF-A, VEGF-B, VEGF-C, VEGF-D, and PIGF), RET Receptor and the Eph Receptor Family (including EphA1, EphA2, EphA3, EphA4, EphA5, EphA6, EphA7, EphA8, EphA9, EphA10, EphB1, EphB2. EphB3, EphB4, and EphB6), CXCR1, CXCR2, CXCR3, CXCR4, CXCR6, CCR1, CCR2, CCR3, CCR4, CCR5, CCR6, CCR8, CFTR, CIC-1, CIC-2, CIC-4, CIC-5, CIC-7, CIC-Ka, CIC-Kb, Bestrophins, TMEM16A, GABA receptor, glycin receptor, ABC transporters, NAV1.1, NAV1.2, NAV1.3, NAV1.4, NAV1.5, NAV1.6, NAV1.7, NAV1.8, NAV1.9, sphingosin-1-phosphate receptor (S1P1R), NMDA channel, transmembrane protein, multispan transmembrane protein, T-cell receptor motifs, T-cell alpha chains, T-cell β chains, T-cell γ chains, T-cell δ chains, CCR7, CD3, CD4, CD5, CD7, CD8, CD11b, CD11c, CD16, CD19, CD20, CD21, CD22, CD25, CD28, CD34, CD35, CD40, CD45RA, CD45RO, CD52, CD56, CD62L, CD68, CD80, CD95, CD117, CD127, CD133, CD137 (4-1BB), CD163, F4/80, IL-4Ra, Sca-1, CTLA-4, GITR, GARP, LAP, granzyme B, LFA-1, transferrin receptor, NKp46, perforin, CD4+, Th1, Th2, Th17, Th40, Th22, Th9, Tfh, canonical Treg. FoxP3+, Tr1, Th3, Treg17, T_{RE}G; CDCP, NT5E, EpCAM, CEA, gpA33, mucins, TAG-72, carbonic anhydrase IX, PSMA, folate binding protein, gangliosides (e.g., CD2, CD3, GM2), Lewis-γ², VEGF, VEGFR 1/2/3, αVβ3, α5β1, ErbB1/EGFR, ErbB1/HER2, ErB3, c-MET, IGF1R, EphA3, TRAIL-R1, TRAIL-R2, RANKL, FAP, Tenascin, PDL-1, BAFF, HDAC, ABL, FLT3, KIT, MET, RET, IL-1β, ALK, RANKL, mTOR, CTLA-4, IL-6, IL-6R, JAK3, BRAF, PTCH, Smoothened, PIGF, ANPEP, TIMP1, PLAUR, PTPRJ, LTBR, ANTXR1, folate receptor alpha (FRa), ERBB2 (Her2/neu), EphA2, IL-13Ra2, epidermal growth factor receptor (EGFR), mesothelin, TSHR, CD19, CD123, CD22, CD30, CD171, CS-1, CLL-1, CD33, EGFRvIII, GD2, GD3, BCMA, MUC16 (CA125), L1CAM, LeY, MSLN, IL13Rα1, L1-CAM, Tn Ag, prostate specific membrane antigen (PSMA), ROR1, FLT3, FAP, TAG72, CD38, CD44v6, CEA, EPCAM, B7H3, KIT, interleukin-11 receptor a (IL-11Ra), PSCA, PRSS21, VEGFR2, LewisY, CD24, platelet-derived growth factor receptor-beta (PDGFR-beta), SSEA-4, CD20, MUC1, NCAM, Prostase, PAP, ELF2M, Ephrin B2, IGF-1 receptor, CAIX, LMP2, gp100, bcr-abl, tyrosinase, Fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLACI, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, ETV6-AML, sperm protein 17, XAGE1, Tie 2, MAD-CT-1, MAD-CT-2, major histocompatibility complex class I-related gene protein (MR1), urokinase-type plasminogen activator receptor (uPAR), Fos-related antigen 1, p53, p53 mutant, prostein, survivin, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYPIB I, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, a neoantigen, CD133, CD15, CD184, CD24, CD56, CD26, CD29, CD44, HLA-A, HLA-B, HLA-C, (HLA-A,B,C) CD49f, CD151 CD340, CD200, tkrA, trkB, or trkC, or an antigenic fragment or antigenic portion thereof.

### 2. ABD Targets an Antigen Characteristic of a T Cell

In some embodiments, the antigen binding domain targets an antigen characteristic of a T cell. In some embodiments, the ABD binds an antigen associated with a T cell. In some instances, such an antigen is expressed by a T cell or is located on the surface of a T cell. In some embodiments, the antigen characteristic of a T cell or the T cell associated antigen is selected from a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, AKT1; AKT2; AKT3; ATF2; BCL10; CALM1; CD3D (CD3δ); CD3E (CD3ε); CD3G (CD3γ); CD4; CD8; CD28; CD45; CD80 (B7-1); CD86 (B7-2); CD247 (CD35); CTLA-4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p38B); MAPK12 (p38γ); MAPK13 (p388); MAPK14 (p38a); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70.

### 3. ABD Targets an Antigen Characteristic of an Autoimmune or Inflammatory Disorder

In some embodiments, the antigen binding domain targets an antigen characteristic of an autoimmune or inflammatory disorder. In some embodiments, the ABD binds an antigen associated with an autoimmune or inflammatory disorder. In some instances, the antigen is expressed by a cell associated with an autoimmune or inflammatory disorder. In some embodiments, the autoimmune or inflammatory disorder is selected from chronic graft-vs-host disease (GVHD), lupus, arthritis, immune complex glomerulonephritis, goodpasture syndrome, uveitis, hepatitis, systemic sclerosis or scleroderma, type I diabetes, multiple sclerosis, cold agglutinin disease, Pemphigus vulgaris, Grave's disease, autoimmune hemolytic anemia, Hemophilia A, Primary Sjogren's Syndrome, thrombotic thrombocytopenia purrpura, neuromyelits optica, Evan's syndrome, IgM mediated neuropathy, cryoglobulinemia, dermatomyositis, idiopathic thrombocytopenia, ankylosing spondylitis, bullous pemphigoid, acquired angioedema, chronic urticarial, antiphospholipid demyelinating polyneuropathy, and autoimmune thrombocytopenia or neutropenia or pure red cell aplasias, while exemplary non-limiting examples of alloimmune diseases include allosensitization (see, for example, Blazar et al., 2015, Am. J. Transplant, 15(4):931-41) or xenosensitization from hematopoietic or solid organ transplantation, blood transfusions, pregnancy with fetal allosensitization, neonatal alloimmune thrombocytopenia, hemolytic disease of the newborn, sensitization to foreign antigens such as can occur with replacement of inherited or acquired deficiency disorders treated with enzyme or protein replacement therapy, blood products, and gene therapy. In some embodiments, the antigen characteristic of an autoimmune or inflammatory disorder is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, an antigen binding domain of a CAR binds to a ligand expressed on B cells, plasma cells, or plasmablasts. In some embodiments, an antigen binding domain of a CAR binds to CD10, CD19, CD20, CD22, CD24, CD27, CD38, CD45R, CD138, CD319, BCMA, CD28, TNF, interferon receptors, GM-CSF, ZAP-70, LFA-1, CD3 gamma, CD5 or CD2. See, e.g., US 2003/0077249; WO 2017/058753; WO 2017/058850, the contents of which are herein incorporated by reference.

### 4. ABD Targets an Antigen Characteristic of Senescent Cells

In some embodiments, the antigen binding domain targets an antigen characteristic of senescent cells, e.g., urokinase-type plasminogen activator receptor (uPAR). In some embodiments, the ABD binds an antigen associated with a senescent cell. In some instances, the antigen is expressed by a senescent cell. In some embodiments, the CAR may be used for treatment or prophylaxis of disorders characterized by the aberrant accumulation of senescent cells, e.g., liver and lung fibrosis, atherosclerosis, diabetes and osteoarthritis.

### 5. ABD Targets an Antigen Characteristic of an Infectious Disease

In some embodiments, the antigen binding domain targets an antigen characteristic of an infectious disease. In some embodiments, the ABD binds an antigen associated with an infectious disease. In some instances, the antigen is expressed by a cell affected by an infectious disease. In some embodiments, wherein the infectious disease is selected from HIV, hepatitis B virus, hepatitis C virus, Human herpes virus, Human herpes virus 8 (HHV-8, Kaposi sarcoma-associated herpes virus (KSHV)), Human T-lymphotrophic virus-1 (HTLV-1), Merkel cell polyomavirus (MCV), Simian virus 40 (SV40), Epstein-Barr virus, CMV, human papillomavirus. In some embodiments, the antigen characteristic of an infectious disease is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, HIV Env, gp120, or CD4-induced epitope on HIV-1 Env.

### 6. ABD Binds to a Cell Surface Antigen of a Cell

In some embodiments, an antigen binding domain binds to a cell surface antigen of a cell. In some embodiments, a cell surface antigen is characteristic of (e.g., expressed by) a particular or specific cell type. In some embodiments, a cell surface antigen is characteristic of more than one type of cell.

In some embodiments, a CAR antigen binding domain binds a cell surface antigen characteristic of a T cell, such as a cell surface antigen on a T cell. In some embodiments, an antigen characteristic of a T cell may be a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, an antigen binding domain of a CAR binds a T cell receptor. In some embodiments, a T cell receptor may be AKT1; AKT2; AKT3; ATF2; BCL10; CALM1; CD3D (CD3δ); CD3E (CD3ε); CD3G (CD3γ); CD4; CD8; CD28; CD45; CD80 (B7-1); CD86 (B7-2); CD247 (CD3ζ); CTLA-4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p38β); MAPK12 (p38γ); MAPK13 (p38δ); MAPK14 (p38α); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70.

### 7. Transmembrane Domain

In some embodiments, the CAR transmembrane domain comprises at least a transmembrane region of the alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments, the transmembrane domain comprises at least a transmembrane region(s) of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or functional variant thereof, antigen binding domain binds

### 8. Signaling Domain or Plurality of Signaling Domains

In some embodiments, a CAR described herein comprises one or at least one signaling domain selected from one or more of B7-1/CD80; B7-2/CD86; B7-H1/PD-L1; B7-H2; B7-H3; B7-H4; B7-H6; B7-H7; BTLA/CD272; CD28; CTLA-4; Gi24/VISTA/B7-H5; ICOS/CD278; PD-1; PD-L2/B7-DC; PDCD6); 4-1BB/TNFSF9/CD137; 4-1BB Ligand/TNFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNFSF4; RELT/TNFRSF19L; TACI/TNFRSF13B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thy1; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, or functional fragment thereof.

In some embodiments, the at least one signaling domain comprises a CD3 zeta domain or an immunoreceptor tyrosine-based activation motif (ITAM), or functional variant thereof. In other embodiments, the at least one signaling domain comprises (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least one signaling domain comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least two signaling domains comprise a CD3 zeta domain or an immunoreceptor tyrosine-based activation motif (ITAM), or functional variant thereof. In other embodiments, the at least two signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least two signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least three signaling domains comprise a CD3 zeta domain or an immunoreceptor tyrosine-based activation motif (ITAM), or functional variant thereof. In other embodiments, the at least three signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the least three signaling domains comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least three signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof.

In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof.

In some embodiments, the CAR comprises (i) a CD3 zeta domain, or an immunoreceptor tyrosine-based activation motif (ITAM), or functional variant thereof; (ii) a CD28 domain, or a 4-1BB domain, or functional variant thereof, and/or (iii) a 4-1BB domain, or a CD134 domain, or functional variant thereof.

In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

9. Domain which Upon Successful Signaling of the CAR Induces Expression of a Cytokine Gene

In some embodiments, a first, second, third, or fourth generation CAR further comprises a domain which upon successful signaling of the CAR induces expression of a cytokine gene. In some embodiments, a cytokine gene is endogenous or exogenous to a target cell comprising a CAR which comprises a domain which upon successful signaling of the CAR induces expression of a cytokine gene. In some embodiments, a cytokine gene encodes a pro-inflammatory cytokine. In some embodiments, a cytokine gene encodes IL-1, IL-2, IL-9, IL-12, IL-18, TNF, or IFN-gamma, or functional fragment thereof. In some embodiments, a domain which upon successful signaling of the CAR induces expression of a cytokine gene is or comprises a transcription factor or functional domain or fragment thereof. In some embodiments, a domain which upon successful signaling of the CAR induces expression of a cytokine gene is or comprises a transcription factor or functional domain or fragment thereof. In some embodiments, a transcription factor or functional domain or fragment thereof is or comprises a nuclear factor of activated T cells (NFAT), an NF-κB, or functional domain or fragment thereof. See, e.g., Zhang. C. et al., Engineering CAR-T cells. Biomarker Research. 5:22 (2017); WO 2016126608; Sha, H. et al. Chimaeric antigen receptor T-cell therapy for tumour immunotherapy. Bioscience Reports Jan. 27, 2017, 37 (1).

In some embodiments, the CAR further comprises one or more spacers, e.g., wherein the spacer is a first spacer between the antigen binding domain and the transmembrane domain. In some embodiments, the first spacer includes at least a portion of an immunoglobulin constant region or variant or modified version thereof. In some embodiments, the spacer is a second spacer between the transmembrane domain and a signaling domain. In some embodiments, the second spacer is an oligopeptide, e.g., wherein the oligopeptide comprises glycine and serine residues such as but not limited to glycine-serine doublets. In some embodiments, the CAR comprises two or more spacers, e.g., a spacer between the antigen binding domain and the transmembrane domain and a spacer between the transmembrane domain and a signaling domain.

In some embodiments, any one of the cells described herein comprises a nucleic acid encoding a CAR or a first-generation CAR. In some embodiments, a first-generation CAR comprises an antigen binding domain, a transmembrane domain, and signaling domain. In some embodiments, a signaling domain mediates downstream signaling during T cell activation.

In some embodiments, any one of the cells described herein comprises a nucleic acid encoding a CAR or a second-generation CAR. In some embodiments, a second-generation CAR comprises an antigen binding domain, a transmembrane domain, and two signaling domains. In some embodiments, a signaling domain mediates downstream signaling during T cell activation. In some embodiments, a signaling domain is a costimulatory domain. In some embodiments, a costimulatory domain enhances cytokine production, CAR-T cell proliferation, and/or CAR-T cell persistence during T cell activation.

In some embodiments, any one of the cells described herein comprises a nucleic acid encoding a CAR or a third generation CAR. In some embodiments, a third generation CAR comprises an antigen binding domain, a transmembrane domain, and at least three signaling domains. In some embodiments, a signaling domain mediates downstream signaling during T cell activation. In some embodiments, a signaling domain is a costimulatory domain. In some embodiments, a costimulatory domain enhances cytokine production, CAR-T cell proliferation, and or CAR-T cell persistence during T cell activation. In some embodiments, a third generation CAR comprises at least two costimulatory domains. In some embodiments, the at least two costimulatory domains are not the same.

In some embodiments, any one of the cells described herein comprises a nucleic acid encoding a CAR or a fourth generation CAR. In some embodiments, a fourth generation CAR comprises an antigen binding domain, a transmembrane domain, and at least two, three, or four signaling domains. In some embodiments, a signaling domain mediates downstream signaling during T cell activation. In some embodiments, a signaling domain is a costimulatory domain. In some embodiments, a costimulatory domain enhances cytokine production, CAR-T cell proliferation, and or CAR-T cell persistence during T cell activation.

### 10. ABD Comprising an Antibody or Antigen-Binding Portion Thereof

In some embodiments, a CAR antigen binding domain is or comprises an antibody or antigen-binding portion thereof. In some embodiments, a CAR antigen binding domain is or comprises an scFv or Fab. In some embodiments, a CAR antigen binding domain comprises an scFv or Fab fragment of a CD19 antibody; CD22 antibody; T-cell alpha chain antibody; T-cell β chain antibody; T-cell γ chain antibody; T-cell δ chain antibody; CCR7 antibody; CD3 antibody; CD4 antibody; CD5 antibody; CD7 antibody; CD8 antibody; CD11b antibody; CD11c antibody; CD16 antibody; CD20 antibody; CD21 antibody; CD25 antibody; CD28 antibody; CD34 antibody; CD35 antibody; CD40 antibody; CD45RA antibody; CD45RO antibody; CD52 antibody; CD56 antibody; CD62L antibody; CD68 antibody; CD80 antibody; CD95 antibody; CD117 antibody; CD127 antibody; CD133 antibody; CD137 (4-1 BB) antibody; CD163 antibody; F4/80 antibody; IL-4Ra antibody; Sca-1 antibody; CTLA-4 antibody; GITR antibody GARP antibody; LAP antibody; granzyme B antibody; LFA-1 antibody; MR1 antibody; uPAR antibody; or transferrin receptor antibody.

In some embodiments, a CAR comprises a signaling domain which is a costimulatory domain. In some embodiments, a CAR comprises a second costimulatory domain. In some embodiments, a CAR comprises at least two costimulatory domains. In some embodiments, a CAR comprises at least three costimulatory domains. In some embodiments, a CAR comprises a costimulatory domain selected from one or more of CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83. In some embodiments, if a CAR comprises two or more costimulatory domains, two costimulatory domains are different. In some embodiments, if a CAR comprises two or more costimulatory domains, two costimulatory domains are the same.

In addition to the CARs described herein, various chimeric antigen receptors and nucleotide sequences encoding the same are known in the art and would be suitable for fusosomal delivery and reprogramming of target cells in vivo and in vitro as described herein. See, e.g., WO2013040557; WO2012079000; WO2016030414; Smith T, et al., Nature Nanotechnology. 2017. DOI: 10.1038/NNANO.2017.57, the disclosures of which are herein incorporated by reference.

### 11. CAR

In certain embodiments, the cell may comprise an exogenous gene encoding a CAR. CARs (also known as chimeric immunoreceptors, chimeric T cell receptors, or artificial T cell receptors) are receptor proteins that have been engineered to give host cells (e.g., T cells) the new ability to target a specific protein. The receptors are chimeric because they combine both antigen-binding and T cell activating functions into a single receptor. The polycistronic vector of the present technology may be used to express one or more CARs in a host cell (e.g., a T cell) for use in cell-based therapies against various target antigens. The CARs expressed by the one or more expression cassettes may be the same or different. In these embodiments, the CAR may comprise an extracellular binding domain (also referred to as a “binder”) that specifically binds a target antigen, a transmembrane domain, and an intracellular signaling domain. In certain embodiments, the CAR may further comprise one or more additional elements, including one or more signal peptides, one or more extracellular hinge domains, and/or one or more intracellular costimulatory domains. Domains may be directly adjacent to one another, or there may be one or more amino acids linking the domains. The nucleotide sequence encoding a CAR may be derived from a mammalian sequence, for example, a mouse sequence, a primate sequence, a human sequence, or combinations thereof. In the cases where the nucleotide sequence encoding a CAR is non-human, the sequence of the CAR may be humanized. The nucleotide sequence encoding a CAR may also be codon-optimized for expression in a mammalian cell, for example, a human cell. In any of these embodiments, the nucleotide sequence encoding a CAR may be at least 80% identical (e.g., at least 80%, at least 85%, at least 90%, at least 95%, at least 96%, at least 97%, at least 98%, at least 99%, or 100% identical) to any of the nucleotide sequences disclosed herein. The sequence variations may be due to codon-optimalization, humanization, restriction enzyme-based cloning scars, and/or additional amino acid residues linking the functional domains, etc.

In certain embodiments, the CAR may comprise a signal peptide at the N-terminus. Non-limiting examples of signal peptides include CD8a signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 2 below.

TABLE 2
Exemplary sequences of signal peptides

| SEQ ID | | |
|---|---|---|
| NO: | Sequence | Description |
| 6 | MALPVTALLLPLALLLHAARP | CD8α signal |
| | | peptide |
| 7 | METDTLLLWVLLLWVPGSTG | IgK signal |
| | | peptide |
| 8 | MLLLVTSLLLCELPHPAFLLIP | GMCSFR-α |
| | | (CSF2RA) |
| | | signal |
| | | peptide |

In certain embodiments, the extracellular binding domain of the CAR may comprise one or more antibodies specific to one target antigen or multiple target antigens. The antibody may be an antibody fragment, for example, an scFv, or a single-domain antibody fragment, for example, a VHH. In certain embodiments, the scFv may comprise a heavy chain variable region (V_{H}) and a light chain variable region (V_{L}) of an antibody connected by a linker. The V_{H} and the V_{L} may be connected in either order, i.e., V_{H}-linker-V_{L} or V_{L}-linker-V_{H}. Non-limiting examples of linkers include Whitlow linker, (G₄S)ₙ (n can be a positive integer, e.g., 1, 2, 3, 4, 5, 6, etc.) linker, and variants thereof. In certain embodiments, the antigen may be an antigen that is exclusively or preferentially expressed on tumor cells, or an antigen that is characteristic of an autoimmune or inflammatory disease. Exemplary target antigens include, but are not limited to, CD5, CD19, CD20, CD22, CD23, CD30, CD70, Kappa, Lambda, and B cell maturation agent (BCMA), G-protein coupled receptor family C group 5 member D (GPRC5D) (associated with leukemias); CS1/SLAMF7, CD38, CD138, GPRC5D, TACI, and BCMA (associated with myelomas); GD2, HER2, EGFR, EGFRVIII, B7H3, PSMA, PSCA, CAIX, CD171, CEA, CSPG4, EPHA2, FAP, FRα, IL-13Rα, Mesothelin, MUC1, MUC16, and ROR1 (associated with solid tumors). In any of these embodiments, the extracellular binding domain of the CAR can be codon-optimized for expression in a host cell or have variant sequences to increase functions of the extracellular binding domain.

In certain embodiments, the CAR may comprise a hinge domain, also referred to as a spacer. The terms “hinge” and “spacer” may be used interchangeably in the present disclosure. Non-limiting examples of hinge domains include CD8a hinge domain, CD28 hinge domain, IgG4 hinge domain, IgG4 hinge-CH2-CH3 domain, and variants thereof, the amino acid sequences of which are provided in Table 3 below.

TABLE 3
Exemplary sequences of hinge domains

| SEQ ID | | |
|---|---|---|
| NO: | Sequence | Description |
| 9 | TTTPAPRPPTPAPTIASQPLSLRPEACRP | CD8α hinge |
| | AAGGAVHTRGLDFACD | domain |
| 10 | IEVMYPPPYLDNEKSNGTIIHVKGKHLCP | CD28 hinge |
| | SPLFPGPSKP | domain |
| 113 | AAAIEVMYPPPYLDNEKSNGTIIHVKGKH | CD28 hinge |
| | LCPSPLFPGPSKP | domain |
| 11 | ESKYGPPCPPCP | IgG4 hinge |
| | | domain |
| 12 | ESKYGPPCPSCP | IgG4 hinge |
| | | domain |
| 13 | ESKYGPPCPPCPAPEFLGGPSVFLFPPKP | IgG4 hinge- |
| | KDTLMISRTPEVTCVVVDVSQEDPEVQFN | CH2-CH3 |
| | WYVDGVEVHNAKTKPREEQFNSTYRVVSV | domain |
| | LTVLHQDWLNGKEYKCKVSNKGLPSSIEK | |
| | TISKAKGQPREPQVYTLPPSQEEMTKNQV | |
| | SLTCLVKGFYPSDIAVEWESNGQPENNYK | |
| | TTPPVLDSDGSFFLYSRLTVDKSRWQEGN | |
| | VFSCSVMHEALHNHYTQKSLSLSLGK | |

In certain embodiments, the transmembrane domain of the CAR may comprise a transmembrane region of the alpha, beta, or zeta chain of a T cell receptor, CD28, CD38, CD45, CD4, CD5, CD8, CD9, CD16, CD22, CD33, CD37, CD64, CD80, CD86, CD134, CD137, CD154, or a functional variant thereof, including the human versions of each of these sequences. In other embodiments, the transmembrane domain may comprise a transmembrane region of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or a functional variant thereof, including the human versions of each of these sequences. Table 4 provides the amino acid sequences of a few exemplary transmembrane domains.

TABLE 4
Exemplary sequences of
transmembrane domains

| SEQ ID | | |
|---|---|---|
| NO: | Sequence | Description |
| 14 | IYIWAPLAGTCGVLLL | CD8α |
| | SLVITLYC | transmembrane |
| | | domain |
| 15 | FWVLVVVGGVLACYSL | CD28 |
| | LVTVAFIIFWV | transmembrane |
| | | domain |
| 114 | MFWVLVVVGGVLACYS | CD28 |
| | LLVTVAFIIFWV | transmembrane |
| | | domain |

In certain embodiments, the intracellular signaling domain and/or intracellular costimulatory domain of the CAR may comprise one or more signaling domains selected from B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFβ, OX40/TNFRSF4, OX40 Ligand/TNFSF4, RELT/TNFRSF19L, TACI/TNFRSF13B, TL1A/TNFSF15, TNFα, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD32, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and a functional variant thereof including the human versions of each of these sequences. In some embodiments, the intracellular signaling domain and/or intracellular costimulatory domain comprises one or more signaling domains selected from a CD3ζ domain, an ITAM, a CD28 domain, 4-1BB domain, or a functional variant thereof. Table 5 provides the amino acid sequences of a few exemplary intracellular costimulatory and/or signaling domains. In certain embodiments, as in the case of tisagenlecleucel as described below, the CD3ζ signaling domain of SEQ ID NO:18 may have a mutation, e.g., a glutamine (Q) to lysine (K) mutation, at amino acid position 14 (see SEQ ID NO:115).

TABLE 5
Exemplary sequences of intracellular
costimulatory and/or signaling domains

| SEQ | | |
|---|---|---|
| ID | | |
| NO: | Sequence | Description |
| 16 | KRGRKKLLYIFKQPFMRPVQTTQEED | 4-1BB |
| | GCSCRFPEEEEGGCEL | costimulatory |
| | | domain |
| 17 | RSKRSRLLHSDYMNMTPRRPGPTRKH | CD28 |
| | YQPYAPPRDFAAYRS | costimulatory |
| | | domain |
| 18 | RVKFSRSADAPAYQQGQNQLYNELNL | CD3ζ signaling |
| | GRREEYDVLDKRRGRDPEMGGKPRRK | domain |
| | NPQEGLYNELQKDKMAEAYSEIGMKG | |
| | ERRRGKGHDGLYQGLSTATKDTYDAL | |
| | HMQALPPR | |
| 115 | RVKFSRSADAPAYKQGQNQLYNELNL | CD3ζ signaling |
| | GRREEYDVLDKRRGRDPEMGGKPRRK | domain (with |
| | NPQEGLYNELQKDKMAEAYSEIGMKG | Q to K mutation |
| | ERRRGKGHDGLYQGLSTATKDTYDAL | at position 14) |
| | HMQALPPR | |

In certain embodiments where the polycistronic vector encodes two or more CARs, the two or more CARs may comprise the same functional domains, or one or more different functional domains, as described. For example, the two or more CARs may comprise different signal peptides, extracellular binding domains, hinge domains, transmembrane domains, costimulatory domains, and/or intracellular signaling domains, in order to minimize the risk of recombination due to sequence similarities. Or, alternatively, the two or more CARs may comprise the same domains. In the cases where the same domain(s) and/or backbone are used, it is optional to introduce codon divergence at the nucleotide sequence level to minimize the risk of recombination.

### CD19 CAR

In some embodiments, the CAR is a CD19 CAR, and in these embodiments, the polycistronic vector comprises an expression cassette that contains a nucleotide sequence encoding a CD19 CAR. In some embodiments, the CD19 CAR may comprise a signal peptide, an extracellular binding domain that specifically binds CD19, a hinge domain, a transmembrane domain, an intracellular costimulatory domain, and/or an intracellular signaling domain in tandem.

In some embodiments, the signal peptide of the CD19 CAR comprises a CD8a signal peptide. In some embodiments, the CD8a signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD19 CAR is specific to CD19, for example, human CD19. The extracellular binding domain of the CD19 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD19 CAR comprises an scFv derived from the FMC63 monoclonal antibody (FMC63), which comprises the heavy chain variable region (V_{H}) and the light chain variable region (V_{L}) of FMC63 connected by a linker. FMC63 and the derived scFv have been described in Nicholson et al., Mol. Immun. 34(16-17): 1157-1165 (1997) and PCT Application Publication No. WO2018/213337, the entire contents of each of which are incorporated by reference herein. In some embodiments, the amino acid sequences of the entire FMC63-derived scFv (also referred to as FMC63 scFv) and its different portions are provided in Table 6 below. In some embodiments, the CD19-specific scFv comprises or consists of an amino acid sequence set forth in SEQ ID NO: 19, 20, or 25, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:19, 20, or 25. In some embodiments, the CD19-specific scFv may comprise one or more CDRs having amino acid sequences set forth in SEQ ID NOs: 21-23 and 26-28. In some embodiments, the CD19-specific scFv may comprise a light chain with one or more CDRs having amino acid sequences set forth in SEQ ID NOs: 21-23. In some embodiments, the CD19-specific scFv may comprise a heavy chain with one or more CDRs having amino acid sequences set forth in SEQ ID NOs: 26-28. In any of these embodiments, the CD19-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD19 CAR comprises or consists of the one or more CDRs as described herein.

In some embodiments, the linker linking the V_{H} and the V_{L} portions of the scFv is a Whitlow linker having an amino acid sequence set forth in SEQ ID NO:24. In some embodiments, the Whitlow linker may be replaced by a different linker, for example, a 3xG₄S linker having an amino acid sequence set forth in SEQ ID NO:30, which gives rise to a different FMC63-derived scFv having an amino acid sequence set forth in SEQ ID NO:29. In certain of these embodiments, the CD19-specific scFv comprises or consists of an amino acid sequence set forth in SEQ ID NO:29 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:29.

TABLE 6
Exemplary sequences of anti-CD19
scFv and components

| SEQ | | |
|---|---|---|
| ID | | |
| NO: | Amino Acid Sequence | Description |
| 19 | DIQMTQTTSSLSASLGDRVTISCRAS | Anti-CD19 FMC63 scFv |
| | QDISKYLNWYQQKPDGTVKLLIYHTS | entire sequence, |
| | RLHSGVPSRFSGSGSGTDYSLTISNL | with Whitlow linker |
| | EQEDIATYFCQQGNTLPYTFGGGTKL | |
| | EITGSTSGSGKPGSGEGSTKGEVKLQ | |
| | ESGPGLVAPSQSLSVTCTVSGVSLPD | |
| | YGVSWIRQPPRKGLEWLGVIWGSETT | |
| | YYNSALKSRLTIIKDNSKSQVFLKMN | |
| | SLQTDDTAIYYCAKHYYYGGSYAMDY | |
| | WGQGTSVTVSS | |
| 20 | DIQMTQTTSSLSASLGDRVTISCRAS | Anti-CD19 FMC63 scFv |
| | QDISKYLNWYQQKPDGTVKLLIYHTS | light chain variable |
| | RLHSGVPSRFSGSGSGTDYSLTISNL | region |
| | EQEDIATYFCQQGNTLPYTFGGGTKL | |
| | EIT | |
| 21 | QDISKY | Anti-CD19 FMC63 scFv |
| | | light chain CDR1 |
| 22 | HTS | Anti-CD19 FMC63 scFv |
| | | light chain CDR2 |
| 23 | QQGNTLPYT | Anti-CD19 FMC63 scFv |
| | | light chain CDR3 |
| 24 | GSTSGSGKPGSGEGSTKG | Whitlow linker |
| 25 | EVKLQESGPGLVAPSQSLSVTCTVSG | Anti-CD19 FMC63 scFv |
| | VSLPDYGVSWIRQPPRKGLEWLGVIW | heavy chain variable |
| | GSETTYYNSALKSRLTIIKDNSKSQV | region |
| | FLKMNSLQTDDTAIYYCAKHYYYGGS | |
| | YAMDYWGQGTSVTVSS | |
| 26 | GVSLPDYG | Anti-CD19 FMC63 scFv |
| | | heavy chain CDR1 |
| 27 | IWGSETT | Anti-CD19 FMC63 scFv |
| | | heavy chain CDR2 |
| 28 | AKHYYYGGSYAMDY | Anti-CD19 FMC63 scFv |
| | | heavy chain CDR3 |
| 29 | DIQMTQTTSSLSASLGDRVTISCRAS | Anti-CD19 FMC63 scFv |
| | QDISKYLNWYQQKPDGTVKLLIYHTS | entire sequence, |
| | RLHSGVPSRFSGSGSGTDYSLTISNL | with 3xG4S linker |
| | EQEDIATYFCQQGNTLPYTFGGGTKL | |
| | EITGGGGSGGGGSGGGGSEVKLQESG | |
| | PGLVAPSQSLSVTCTVSGVSLPDYGV | |
| | SWIRQPPRKGLEWLGVIWGSETTYYN | |
| | SALKSRLTIIKDNSKSQVFLKMNSLQ | |
| | TDDTAIYYCAKHYYYGGSYAMDYWGQ | |
| | GTSVTVSS | |
| 30 | GGGGSGGGGSGGGGS | 3xG4S linker |

In some embodiments, the extracellular binding domain of the CD19 CAR is derived from an antibody specific to CD19, including, for example, SJ25C1 (Bejcek et al., Cancer Res. 55:2346-2351 (1995)), HD37 (Pezutto et al., J. Immunol. 138(9): 2793-2799 (1987)), 4G7 (Meeker et al., Hybridoma 3:305-320 (1984)), B43 (Bejcek (1995)), BLY3 (Bejcek (1995)), B4 (Freedman et al., 70:418-427 (1987)), B4 HB12b (Kansas & Tedder, J. Immunol. 147:4094-4102 (1991); Yazawa et al., Proc. Natl. Acad. Sci. USA 102:15178-15183 (2005); Herbst et al., J. Pharmacol. Exp. Ther. 335:213-222 (2010)), BU12 (Callard et al., J. Immunology, 148(10): 2983-2987 (1992)), and CLB-CD19 (De Rie Cell. Immunol. 118:368-381(1989)). In any of these embodiments, the extracellular binding domain of the CD19 CAR can comprise or consist of the V_{H}, the V_{L}, and/or one or more CDRs of any of the antibodies.

In some embodiments, the hinge domain of the CD19 CAR comprises a CD8a hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO: 12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the CD19 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the CD19 CAR comprises a 4-1BB costimulatory domain. 4-1BB, also known as CD137, transmits a potent costimulatory signal to T cells, promoting differentiation and enhancing long-term survival of T lymphocytes. In some embodiments, the 4-1BB costimulatory domain is human. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain. CD28 is another co-stimulatory molecule on T cells. In some embodiments, the CD28 costimulatory domain is human. In some embodiments, the CD28 costimulatory domain comprises or consists of an amino acid sequence set forth in SEQ ID NO: 17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17. In some embodiments, the intracellular costimulatory domain of the CD19 CAR comprises a 4-1BB costimulatory domain and a CD28 costimulatory domain as described.

In some embodiments, the intracellular signaling domain of the CD19 CAR comprises a CD3 zeta (ζ) signaling domain. CD3ζ associates with T cell receptors (TCRs) to produce a signal and contains immunoreceptor tyrosine-based activation motifs (ITAMs). The CD3ζ signaling domain refers to amino acid residues from the cytoplasmic domain of the zeta chain that are sufficient to functionally transmit an initial signal necessary for T cell activation. In some embodiments, the CD3ζ signaling domain is human. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO: 19 or SEQ ID NO:29, the CD8α hinge domain of SEQ ID NO:9, the CD8α transmembrane domain of SEQ ID NO:14, the 4-1BB costimulatory domain of SEQ ID NO: 16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO: 19 or SEQ ID NO:29, the IgG4 hinge domain of SEQ ID NO: 11 or SEQ ID NO: 12, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO: 19 or SEQ ID NO:29, the CD28 hinge domain of SEQ ID NO:10, the CD28 transmembrane domain of SEQ ID NO:15, the CD28 costimulatory domain of SEQ ID NO: 17, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR as set forth in SEQ ID NO: 116 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:116 (see Table 7). The encoded CD19 CAR has a corresponding amino acid sequence set forth in SEQ ID NO:117 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:117, with the following components: CD8α signal peptide, FMC63 scFv (V_{L}-Whitlow linker-V_{H}), CD8α hinge domain, CD8α transmembrane domain, 4-1BB costimulatory domain, and CD3ζ signaling domain.

In some embodiments, the polycistronic vector comprises an expression cassette that contains a nucleotide sequence encoding a commercially available embodiment of CD19 CAR. Non-limiting examples of commercially available embodiments of CD19 CARs expressed and/or encoded by T cells include tisagenlecleucel, lisocabtagene maraleucel, axicabtagene ciloleucel, and brexucabtagene autoleucel.

In some embodiments, the polycistronic vector comprises an expression cassette that contains a nucleotide sequence encoding tisagenlecleucel or portions thereof. Tisagenlecleucel comprises a CD19 CAR with the following components: CD8α signal peptide, FMC63 scFv (V_{L}-3xG₄S linker-V_{H}), CD8α hinge domain, CD8α transmembrane domain, 4-1BB costimulatory domain, and CD3 signaling domain. The nucleotide and amino acid sequence of the CD19 CAR in tisagenlecleucel are provided in Table 7, with annotations of the sequences provided in Table 8.

In some embodiments, the polycistronic vector comprises an expression cassette that contains a nucleotide sequence encoding lisocabtagene maraleucel or portions thereof. Lisocabtagene maraleucel comprises a CD19 CAR with the following components: GMCSFR-α or CSF2RA signal peptide, FMC63 scFv (V_{L}-Whitlow linker-V_{H}), IgG4 hinge domain, CD28 transmembrane domain, 4-1BB costimulatory domain, and CD3ζ signaling domain. The nucleotide and amino acid sequence of the CD19 CAR in lisocabtagene maraleucel are provided in Table 7, with annotations of the sequences provided in Table 9.

In some embodiments, the polycistronic vector comprises an expression cassette that contains a nucleotide sequence encoding axicabtagene ciloleucel or portions thereof. Axicabtagene ciloleucel comprises a CD19 CAR with the following components: GMCSFR-α or CSF2RA signal peptide, FMC63 scFv (V_{L}-Whitlow linker-V_{H}), CD28 hinge domain, CD28 transmembrane domain, CD28 costimulatory domain, and CD3ζ signaling domain. The nucleotide and amino acid sequence of the CD19 CAR in axicabtagene ciloleucel are provided in Table 7, with annotations of the sequences provided in Table 10.

In some embodiments, the polycistronic vector comprises an expression cassette that contains a nucleotide sequence encoding brexucabtagene autoleucel or portions thereof. Brexucabtagene autoleucel comprises a CD19 CAR with the following components: GMCSFR-α signal peptide, FMC63 scFv, CD28 hinge domain, CD28 transmembrane domain, CD28 costimulatory domain, and CD3ζ signaling domain.

In some embodiments, the polycistronic vector comprises an expression cassette that contains a nucleotide sequence encoding a CD19 CAR as set forth in SEQ ID NO: 31, 33, or 35, or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 31, 33, or 35. The encoded CD19 CAR has a corresponding amino acid sequence set forth in SEQ ID NO: 32, 34, or 36, respectively, or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 32, 34, or 36, respectively.

TABLE 7
Exemplary sequences of CD19 CARs

| SEQ ID | | |
|---|---|---|
| NO: | Sequence | Description |
| 116 | atggccttaccagtgaccgccttgctcctgccgctggccttgctgct | Exemplary CD19 |
| | ccacgccgccaggccggacatccagatgacacagactacatcctccc | CAR nucleotide |
| | tgtctgcctctctgggagacagagtcaccatcagttgcagggcaagt | sequence |
| | caggacattagtaaatatttaaattggtatcagcagaaaccagatgg | |
| | aactgttaaactcctgatctaccatacatcaagattacactcaggag | |
| | tcccatcaaggttcagtggcagtgggtctggaacagattattctctc | |
| | accattagcaacctggagcaagaagatattgccacttacttttgcca | |
| | acagggtaatacgcttccgtacacgttcggaggggggaccaagctgg | |
| | agatcacaggctccacctctggatccggcaagcccggatctggcgag | |
| | ggatccaccaagggcgaggtgaaactgcaggagtcaggacctggcct | |
| | ggtggcgccctcacagagcctgtccgtcacatgcactgtctcagggg | |
| | tctcattacccgactatggtgtaagctggattcgccagcctccacga | |
| | aagggtctggagtggctgggagtaatatggggtagtgaaaccacata | |
| | ctataattcagctctcaaatccagactgaccatcatcaaggacaact | |
| | ccaagagccaagttttcttaaaaatgaacagtctgcaaactgatgac | |
| | acagccatttactactgtgccaaacattattactacggtggtagcta | |
| | tgctatggactactggggccaaggaacctcagtcaccgtctcctcaa | |
| | ccacgacgccagcgccgcgaccaccaacaccggcgcccaccatcgcg | |
| | tcgcagcccctgtccctgcgcccagaggcgtgccggccagcgggggg | |
| | ggcgcagtgcacacgagggggctggacttcgcctgtgatatctacat | |
| | ctgggcgcccttggccgggacttgtggggtccttctcctgtcactgg | |
| | ttatcaccctttactgcaaacggggcagaaagaaactcctgtatata | |
| | ttcaaacaaccatttatgagaccagtacaaactactcaagaggaaga | |
| | tggctgtagctgccgatttccagaagaagaagaaggaggatgtgaac | |
| | tgagagtgaagttcagcaggagcgcagacgcccccgcgtaccagcag | |
| | ggccagaaccagctctataacgagctcaatctaggacgaagagagga | |
| | gtacgatgttttggacaagagacgtggccgggaccctgagatggggg | |
| | gaaagccgagaaggaagaaccctcaggaaggcctgtacaatgaactg | |
| | cagaaagataagatggcggaggcctacagtgagattgggatgaaagg | |
| | cgagcgccggaggggcaaggggcacgatggcctttaccagggtctca | |
| | gtacagccaccaaggacacctacgacgcccttcacatgcaggccctg | |
| | ccccctcgc | |
| 117 | MALPVTALLLPLALLLHAARPDIQMTQTTSSLSASLGDRVTISCRAS | Exemplary CD19 |
| | QDISKYLNWYQQKPDGTVKLLIYHTSRLHSGVPSRFSGSGSGTDYSL | CAR amino acid |
| | TISNLEQEDIATYFCQQGNTLPYTFGGGTKLEITGSTSGSGKPGSGE | sequence |
| | GSTKGEVKLQESGPGLVAPSQSLSVTCTVSGVSLPDYGVSWIRQPPR | |
| | KGLEWLGVIWGSETTYYNSALKSRLTIIKDNSKSQVFLKMNSLQTDD | |
| | TAIYYCAKHYYYGGSYAMDYWGQGTSVTVSSTTTPAPRPPTPAPTIA | |
| | SQPLSLRPEACRPAAGGAVHTRGLDFACDIYIWAPLAGTCGVLLLSL | |
| | VITLYCKRGRKKLLYIFKQPFMRPVQTTQEEDGCSCRFPEEEEGGCE | |
| | LRVKFSRSADAPAYQQGQNQLYNELNLGRREEYDVLDKRRGRDPEMG | |
| | GKPRRKNPQEGLYNELQKDKMAEAYSEIGMKGERRRGKGHDGLYQGL | |
| | STATKDTYDALHMQALPPR | |
| 31 | atggccttaccagtgaccgccttgctcctgccgctggccttgctgct | Tisagenlecleucel |
| | ccacgccgccaggccggacatccagatgacacagactacatcctccc | CD19 CAR |
| | tgtctgcctctctgggagacagagtcaccatcagttgcagggcaagt | nucleotide |
| | caggacattagtaaatatttaaattggtatcagcagaaaccagatgg | sequence |
| | aactgttaaactcctgatctaccatacatcaagattacactcaggag | |
| | tcccatcaaggttcagtggcagtgggtctggaacagattattctctc | |
| | accattagcaacctggagcaagaagatattgccacttacttttgcca | |
| | acagggtaatacgcttccgtacacgttcggaggggggaccaagctgg | |
| | agatcacaggtggcggtggctcgggcggtggtgggtcgggtggcggc | |
| | ggatctgaggtgaaactgcaggagtcaggacctggcctggtggcgcc | |
| | ctcacagagcctgtccgtcacatgcactgtctcaggggtctcattac | |
| | ccgactatggtgtaagctggattcgccagcctccacgaaagggtctg | |
| | gagtggctgggagtaatatggggtagtgaaaccacatactataattc | |
| | agctctcaaatccagactgaccatcatcaaggacaactccaagagcc | |
| | aagttttcttaaaaatgaacagtctgcaaactgatgacacagccatt | |
| | tactactgtgccaaacattattactacggtggtagctatgctatgga | |
| | ctactggggccaaggaacctcagtcaccgtctcctcaaccacgacgc | |
| | cagcgccgcgaccaccaacaceggegcccaccatcgcgtcgcagccc | |
| | ctgtccctgcgcccagaggcgtgccggccagcggcggggggcgcagt | |
| | gcacacgagggggctggacttcgcctgtgatatctacatctgggcgc | |
| | ccttggccgggacttgtggggtccttctcctgtcactggttatcacc | |
| | ctttactgcaaacggggcagaaagaaactcctgtatatattcaaaca | |
| | accatttatgagaccagtacaaactactcaagaggaagatggctgta | |
| | gctgccgatttccagaagaagaagaaggaggatgtgaactgagagtg | |
| | aagttcagcaggagcgcagacgcccccgcgtacaagcagggccagaa | |
| | ccagctctataacgagctcaatctaggacgaagagaggagtacgatg | |
| | ttttggacaagagacgtggccgggaccctgagatggggggaaagccg | |
| | agaaggaagaaccctcaggaaggcctgtacaatgaactgcagaaaga | |
| | taagatggcggaggcctacagtgagattgggatgaaaggcgagcgcc | |
| | ggaggggcaaggggcacgatggcctttaccagggtctcagtacagcc | |
| | accaaggacacctacgacgcccttcacatgcaggccctgccccctcg | |
| | c | |
| 32 | MALPVTALLLPLALLLHAARPDIQMTQTTSSLSASLGDRVTISCRAS | Tisagenlecleucel |
| | QDISKYLNWYQQKPDGTVKLLIYHTSRLHSGVPSRFSGSGSGTDYSL | CD19 CAR amino |
| | TISNLEQEDIATYFCQQGNTLPYTFGGGTKLEITGGGGSGGGGSGGG | acid sequence |
| | GSEVKLQESGPGLVAPSQSLSVTCTVSGVSLPDYGVSWIRQPPRKGL | |
| | EWLGVIWGSETTYYNSALKSRLTIIKDNSKSQVFLKMNSLQTDDTAI | |
| | YYCAKHYYYGGSYAMDYWGQGTSVTVSSTTTPAPRPPTPAPTIASQP | |
| | LSLRPEACRPAAGGAVHTRGLDFACDIYIWAPLAGTCGVLLLSLVIT | |
| | LYCKRGRKKLLYIFKQPFMRPVQTTQEEDGCSCRFPEEEEGGCELRV | |
| | KFSRSADAPAYKQGQNQLYNELNLGRREEYDVLDKRRGRDPEMGGKP | |
| | RRKNPQEGLYNELQKDKMAEAYSEIGMKGERRRGKGHDGLYQGLSTA | |
| | TKDTYDALHMQALPPR | |
| 33 | atgctgctgctggtgaccagcctgctgctgtgcgagctgccccaccc | Lisocabtagene |
| | cgcctttctgctgatccccgacatccagatgacccagaccacctcca | maraleucel CD19 |
| | gcctgagcgccagcctgggcgaccgggtgaccatcagctgccgggcc | CAR nucleotide |
| | agccaggacatcagcaagtacctgaactggtatcagcagaagcccga | sequence |
| | cggcaccgtcaagctgctgatctaccacaccagccggctgcacagcg | |
| | gcgtgcccagccggtttagcggcagcggctccggcaccgactacagc | |
| | ctgaccatctccaacctggaacaggaagatatcgccacctacttttg | |
| | ccagcagggcaacacactgccctacacctttggcggcggaacaaagc | |
| | tggaaatcaccggcagcacctccggcagcggcaagcctggcagcggc | |
| | gagggcagcaccaagggcgaggtgaagctgcaggaaagcggccctgg | |
| | cctggtggcccccagccagagcctgagcgtgacctgcaccgtgagcg | |
| | gcgtgagcctgcccgactacggcgtgagctggatccggcagcccccc | |
| | aggaagggcctggaatggctgggcgtgatctggggcagcgagaccac | |
| | ctactacaacagcgccctgaagagccggctgaccatcatcaaggaca | |
| | acagcaagagccaggtgttcctgaagatgaacagcctgcagaccgac | |
| | gacaccgccatctactactgcgccaagcactactactacggcggcag | |
| | ctacgccatggactactggggccagggcaccagcgtgaccgtgagca | |
| | gcgaatctaagtacggaccgccctgccccccttgccctatgttctgg | |
| | gtgctggtggtggtcggaggcgtgctggcctgctacagcctgctggt | |
| | caccgtggccttcatcatcttttgggtgaaacggggcagaaagaaac | |
| | tcctgtatatattcaaacaaccatttatgagaccagtacaaactact | |
| | caagaggaagatggctgtagctgccgatttccagaagaagaagaagg | |
| | aggatgtgaactgcgggtgaagttcagcagaagcgccgacgcccctg | |
| | cctaccagcagggccagaatcagctgtacaacgagctgaacctgggc | |
| | agaagggaagagtacgacgtcctggataagcggagaggccgggaccc | |
| | tgagatgggggcaagcctcggcggaagaacccccaggaaggcctgta | |
| | taacgaactgcagaaagacaagatggccgaggcctacagcgagatcg | |
| | gcatgaagggcgagcggaggcggggcaagggccacgacggcctgtat | |
| | cagggcctgtccaccgccaccaaggatacctacgacgccctgcacat | |
| | gcaggccctgcccccaagg | |
| 34 | MLLLVTSLLLCELPHPAFLLIPDIQMTQTTSSLSASLGDRVTISCRA | Lisocabtagene |
| | SQDISKYLNWYQQKPDGTVKLLIYHTSRLHSGVPSRFSGSGSGTDYS | maraleucel CD19 |
| | LTISNLEQEDIATYFCQQGNTLPYTFGGGTKLEITGSTSGSGKPGSG | CAR amino acid |
| | EGSTKGEVKLQESGPGLVAPSQSLSVTCTVSGVSLPDYGVSWIRQPP | sequence |
| | RKGLEWLGVIWGSETTYYNSALKSRLTIIKDNSKSQVFLKMNSLQTD | |
| | DTAIYYCAKHYYYGGSYAMDYWGQGTSVTVSSESKYGPPCPPCPMFW | |
| | VLVVVGGVLACYSLLVTVAFIIFWVKRGRKKLLYIFKQPFMRPVQTT | |
| | QEEDGCSCRFPEEEEGGCELRVKFSRSADAPAYQQGQNQLYNELNLG | |
| | RREEYDVLDKRRGRDPEMGGKPRRKNPQEGLYNELQKDKMAEAYSEI | |
| | GMKGERRRGKGHDGLYQGLSTATKDTYDALHMQALPPR | |
| 35 | atgcttctcctggtgacaagccttctgctctgtgagttaccacaccc | Axicabtagene |
| | agcattcctcctgatcccagacatccagatgacacagactacatcct | ciloleucel CD19 |
| | ccctgtctgcctctctgggagacagagtcaccatcagttgcagggca | CAR nucleotide |
| | agtcaggacattagtaaatatttaaattggtatcagcagaaaccaga | sequence |
| | tggaactgttaaactcctgatctaccatacatcaagattacactcag | |
| | gagtcccatcaaggttcagtggcagtgggtctggaacagattattct | |
| | ctcaccattagcaacctggagcaagaagatattgccacttacttttg | |
| | ccaacagggtaatacgcttccgtacacgttcggaggggggactaagt | |
| | tggaaataacaggctccacctctggatccggcaagcccggatctggc | |
| | gagggatccaccaagggcgaggtgaaactgcaggagtcaggacctgg | |
| | cctggtggcgccctcacagagcctgtccgtcacatgcactgtctcag | |
| | gggtctcattacccgactatggtgtaagctggattcgccagcctcca | |
| | cgaaagggtctggagtggctgggagtaatatggggtagtgaaaccac | |
| | atactataattcagctctcaaatccagactgaccatcatcaaggaca | |
| | actccaagagccaagttttcttaaaaatgaacagtctgcaaactgat | |
| | gacacagccatttactactgtgccaaacattattactacggtggtag | |
| | ctatgctatggactactggggtcaaggaacctcagtcaccgtctcct | |
| | cagcggccgcaattgaagttatgtatcctcctccttacctagacaat | |
| | gagaagagcaatggaaccattatccatgtgaaagggaaacacctttg | |
| | tccaagtcccctatttcccggaccttctaagcccttttgggtgctgg | |
| | tggtggttgggggagtcctggcttgctatagcttgctagtaacagtg | |
| | gcctttattattttctgggtgaggagtaagaggagcaggctcctgca | |
| | cagtgactacatgaacatgactccccgccgccccgggcccacccgca | |
| | agcattaccagccctatgccccaccacgcgacttcgcagcctatcgc | |
| | tccagagtgaagttcagcaggagcgcagacgcccccgcgtaccagca | |
| | gggccagaaccagctctataacgagctcaatctaggacgaagagagg | |
| | agtacgatgttttggacaagagacgtggccgggaccctgagatgggg | |
| | ggaaagccgagaaggaagaaccctcaggaaggcctgtacaatgaact | |
| | gcagaaagataagatggcggaggcctacagtgagattgggatgaaag | |
| | gcgagcgccggaggggcaaggggcacgatggcctttaccagggtctc | |
| | agtacagccaccaaggacacctacgacgcccttcacatgcaggccct | |
| | gccccctcgc | |
| 36 | MLLLVTSLLLCELPHPAFLLIPDIQMTQTTSSLSASLGDRVTISCRA | Axicabtagene |
| | SQDISKYLNWYQQKPDGTVKLLIYHTSRLHSGVPSRFSGSGSGTDYS | ciloleucel CD19 |
| | LTISNLEQEDIATYFCQQGNTLPYTFGGGTKLEITGSTSGSGKPGSG | CAR amino acid |
| | EGSTKGEVKLQESGPGLVAPSQSLSVTCTVSGVSLPDYGVSWIRQPP | sequence |
| | RKGLEWLGVIWGSETTYYNSALKSRLTIIKDNSKSQVFLKMNSLQTD | |
| | DTAIYYCAKHYYYGGSYAMDYWGQGTSVTVSSAAAIEVMYPPPYLDN | |
| | EKSNGTIIHVKGKHLCPSPLFPGPSKPFWVLVVVGGVLACYSLLVTV | |
| | AFIIFWVRSKRSRLLHSDYMNMTPRRPGPTRKHYQPYAPPRDFAAYR | |
| | SRVKFSRSADAPAYQQGQNQLYNELNLGRREEYDVLDKRRGRDPEMG | |
| | GKPRRKNPQEGLYNELQKDKMAEAYSEIGMKGERRRGKGHDGLYQGL | |
| | STATKDTYDALHMQALPPR | |

TABLE 8
Annotation of tisagenlecleucel CD19 CAR sequences

| | Nucleotide | Amino Acid |
|---|---|---|
| | Sequence | Sequence |
| Feature | Position | Position |
| CD8α signal peptide | 1-63 | 1-21 |
| FMC63 scFv (V_{L}-3xG₄S linker-V_{H}) | 64-789 | 22-263 |
| CD8α hinge domain | 790-924 | 264-308 |
| CD8α transmembrane domain | 925-996 | 309-332 |
| 4-1BB costimulatory domain | 997-1122 | 333-374 |
| CD3ζ signaling domain | 1123-1458 | 375-486 |

TABLE 9
Annotation of lisocabtagene maraleucel CD19 CAR sequences

| | Nucleotide | Amino Acid |
|---|---|---|
| | Sequence | Sequence |
| Feature | Position | Position |
| GMCSFR-α signal peptide | 1-66 | 1-22 |
| FMC63 scFv (V_{L}-Whitlow linker-V_{H}) | 67-801 | 23-267 |
| IgG4 hinge domain | 802-837 | 268-279 |
| CD28 transmembrane domain | 838-921 | 280-307 |
| 4-1BB costimulatory domain | 922-1047 | 308-349 |
| CD3ζ signaling domain | 1048-1383 | 350-461 |

TABLE 10
Annotation of axicabtagene ciloleucel CD19 CAR sequences

| | Nucleotide | Amino Acid |
|---|---|---|
| | Sequence | Sequence |
| Feature | Position | Position |
| CSF2RA signal peptide | 1-66 | 1-22 |
| FMC63 scFv (V_{L}-Whitlow linker-V_{H}) | 67-801 | 23-267 |
| CD28 hinge domain | 802-927 | 268-309 |
| CD28 transmembrane domain | 928-1008 | 310-336 |
| CD28 costimulatory domain | 1009-1131 | 337-377 |
| CD3ζ signaling domain | 1132-1467 | 378-489 |

In some embodiments, the polycistronic vector comprises an expression cassette that contains a nucleotide sequence encoding CD19 CAR as set forth in SEQ ID NO: 31, 33, or 35, or at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 31, 33, or 35. The encoded CD19 CAR has a corresponding amino acid sequence set forth in SEQ ID NO: 32, 34, or 36, respectively, is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 32, 34, or 36, respectively.

### CD20 CAR

In some embodiments, the CAR is a CD20 CAR, and in these embodiments, the polycistronic vector comprises an expression cassette that contains a nucleotide sequence encoding a CD20 CAR. CD20 is an antigen found on the surface of B cells as early at the pro-B phase and progressively at increasing levels until B cell maturity, as well as on the cells of most B-cell neoplasms. CD20 positive cells are also sometimes found in cases of Hodgkins disease, myeloma, and thymoma. In some embodiments, the CD20 CAR may comprise a signal peptide, an extracellular binding domain that specifically binds CD20, a hinge domain, a transmembrane domain, an intracellular costimulatory domain, and/or an intracellular signaling domain in tandem.

In some embodiments, the signal peptide of the CD20 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD20 CAR is specific to CD20, for example, human CD20. The extracellular binding domain of the CD20 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD20 CAR is derived from an antibody specific to CD20, including, for example, Leu16, IF5, 1.5.3, rituximab, obinutuzumab, ibritumomab, ofatumumab, tositumumab, odronextamab, veltuzumab, ublituximab, and ocrelizumab. In any of these embodiments, the extracellular binding domain of the CD20 CAR can comprise or consist of the V_{H}, the V_{L}, and/or one or more CDRs of any of the antibodies.

In some embodiments, the extracellular binding domain of the CD20 CAR comprises an scFv derived from the Leu16 monoclonal antibody, which comprises the heavy chain variable region (V_{H}) and the light chain variable region (V_{L}) of Leu16 connected by a linker. See Wu et al., Protein Engineering. 14(12): 1025-1033 (2001). In some embodiments, the linker is a 3xG₄S linker. In other embodiments, the linker is a Whitlow linker as described herein. In some embodiments, the amino acid sequences of different portions of the entire Leu16-derived scFv (also referred to as Leu16 scFv) and its different portions are provided in Table 11 below. In some embodiments, the CD20-specific scFv comprises or consists of an amino acid sequence set forth in SEQ ID NO:37, 38, or 42, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:37, 38, or 42. In some embodiments, the CD20-specific scFv may comprise one or more CDRs having amino acid sequences set forth in SEQ ID NOs: 39-41, 43 and 44. In some embodiments, the CD20-specific scFv may comprise a light chain with one or more CDRs having amino acid sequences set forth in SEQ ID NOs: 39-41. In some embodiments, the CD20-specific scFv may comprise a heavy chain with one or more CDRs having amino acid sequences set forth in SEQ ID NOs: 43-44. In any of these embodiments, the CD20-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD20 CAR comprises or consists of the one or more CDRs as described herein.

TABLE 11
Exemplary sequences of anti-CD20
scFv and components

| SEQ | | |
|---|---|---|
| ID | | |
| NO: | Amino Acid Sequence | Description |
| 37 | DIVLTQSPAILSASPGEKVTMTCRA | Anti-CD20 Leu16 scFv |
| | SSSVNYMDWYQKKPGSSPKPWIYAT | entire sequence, |
| | SNLASGVPARFSGSGSGTSYSLTIS | with Whitlow linker |
| | RVEAEDAATYYCQQWSFNPPTFGGG | |
| | TKLEIKGSTSGSGKPGSGEGSTKGE | |
| | VQLQQSGAELVKPGASVKMSCKASG | |
| | YTFTSYNMHWVKQTPGQGLEWIGAI | |
| | YPGNGDTSYNQKFKGKATLTADKSS | |
| | STAYMQLSSLTSEDSADYYCARSNY | |
| | YGSSYWFFDVWGAGTTVTVSS | |
| 38 | DIVLTQSPAILSASPGEKVTMTCRA | Anti-CD20 Leu16 scFv |
| | SSSVNYMDWYQKKPGSSPKPWIYAT | light chain variable |
| | SNLASGVPARFSGSGSGTSYSLTIS | region |
| | RVEAEDAATYYCQQWSFNPPTFGGG | |
| | TKLEIK | |
| 39 | RASSSVNYMD | Anti-CD20 Leu16 scFv |
| | | light chain CDR1 |
| 40 | ATSNLAS | Anti-CD20 Leu16 scFv |
| | | light chain CDR2 |
| 41 | QQWSFNPPT | Anti-CD20 Leu16 scFv |
| | | light chain CDR3 |
| 42 | EVQLQQSGAELVKPGASVKMSCKAS | Anti-CD20 Leu16 scFv |
| | GYTFTSYNMHWVKQTPGQGLEWIGA | heavy chain |
| | IYPGNGDTSYNQKFKGKATLTADKS | |
| | SSTAYMQLSSLTSEDSADYYCARSN | |
| | YYGSSYWFFDVWGAGTTVTVSS | |
| 43 | SYNMH | Anti-CD20 Leu16 scFv |
| | | heavy chain CDR1 |
| 44 | AIYPGNGDTSYNQKFKG | Anti-CD20 Leu16 scFv |
| | | heavy chain CDR2 |

In some embodiments, the hinge domain of the CD20 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the CD20 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the CD20 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the CD20 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the CD8α hinge domain of SEQ ID NO:9, the CD8α transmembrane domain of SEQ ID NO:14, the 4-1BB costimulatory domain of SEQ ID NO: 16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the CD28 hinge domain of SEQ ID NO: 10, the CD8α transmembrane domain of SEQ ID NO: 14, the 4-1BB costimulatory domain of SEQ ID NO: 16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the IgG4 hinge domain of SEQ ID NO: 11 or SEQ ID NO: 12, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the CD8α hinge domain of SEQ ID NO:9, the CD28 transmembrane domain of SEQ ID NO: 15, the 4-1BB costimulatory domain of SEQ ID NO: 16, the CD3 signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the CD28 hinge domain of SEQ ID NO: 10, the CD28 transmembrane domain of SEQ ID NO: 15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the IgG4 hinge domain of SEQ ID NO:11 or SEQ ID NO:1, the CD28 transmembrane domain of SEQ ID NO:15, the 4-1BB costimulatory domain of SEQ ID NO: 16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### CD22 CAR

In some embodiments, the CAR is a CD22 CAR, and in these embodiments, the polycistronic vector comprises an expression cassette that contains a nucleotide sequence encoding a CD22 CAR. CD22, which is a transmembrane protein found mostly on the surface of mature B cells that functions as an inhibitory receptor for B cell receptor (BCR) signaling. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. In some embodiments, the CD22 CAR may comprise a signal peptide, an extracellular binding domain that specifically binds CD22, a hinge domain, a transmembrane domain, an intracellular costimulatory domain, and/or an intracellular signaling domain in tandem.

In some embodiments, the signal peptide of the CD22 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD22 CAR is specific to CD22, for example, human CD22. The extracellular binding domain of the CD22 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD22 CAR is derived from an antibody specific to CD22, including, for example, SM03, inotuzumab, epratuzumab, moxetumomab, and pinatuzumab. In any of these embodiments, the extracellular binding domain of the CD22 CAR can comprise or consist of the V_{H}, the V_{L}, and/or one or more CDRs of any of the antibodies.

In some embodiments, the extracellular binding domain of the CD22 CAR comprises an scFv derived from the m971 monoclonal antibody (m971), which comprises the heavy chain variable region (V_{H}) and the light chain variable region (V_{L}) of m971 connected by a linker. In some embodiments, the linker is a 3xG₄S linker. In other embodiments, the Whitlow linker may be used instead. In some embodiments, the amino acid sequences of the entire m971-derived scFv (also referred to as m971 scFv) and its different portions are provided in Table 12 below. In some embodiments, the CD22-specific scFv comprises or consists of an amino acid sequence set forth in SEQ ID NO:45, 46, or 50, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:45, 46, or 50. In some embodiments, the CD22-specific scFv may comprise one or more CDRs having amino acid sequences set forth in SEQ ID NOs: 47-49 and 51-53. In some embodiments, the CD22-specific scFv may comprise a heavy chain with one or more CDRs having amino acid sequences set forth in SEQ ID NOs: 47-49. In some embodiments, the CD22-specific scFv may comprise a light chain with one or more CDRs having amino acid sequences set forth in SEQ ID NOs: 51-53. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

In some embodiments, the extracellular binding domain of the CD22 CAR comprises an scFv derived from m971-L7, which is an affinity matured variant of m971 with significantly improved CD22 binding affinity compared to the parental antibody m971 (improved from about 2 nM to less than 50 pM). In some embodiments, the scFv derived from m971-L7 comprises the V_{H} and the V_{L} of m971-L7 connected by a 3xG₄S linker. In other embodiments, the Whitlow linker may be used instead. In some embodiments, the amino acid sequences of the entire m971-L7-derived scFv (also referred to as m971-L7 scFv) and its different portions are provided in Table 12 below. In some embodiments, the CD22-specific scFv comprises or consists of an amino acid sequence set forth in SEQ ID NO:54, 55, or 59, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:54, 55, or 59. In some embodiments, the CD22-specific scFv may comprise one or more CDRs having amino acid sequences set forth in SEQ ID NOs: 56-58 and 60-62. In some embodiments, the CD22-specific scFv may comprise a heavy chain with one or more CDRs having amino acid sequences set forth in SEQ ID NOs: 56-58. In some embodiments, the CD22-specific scFv may comprise a light chain with one or more CDRs having amino acid sequences set forth in SEQ ID NOs: 60-62. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

TABLE 12
Exemplary sequences of anti-CD22
scFv and components

| SEQ | | |
|---|---|---|
| ID | | |
| NO: | Amino Acid Sequence | Description |
| 45 | QVQLQQSGPGLVKPSQTLSLTCAIS | Anti-CD22 m971 scFv |
| | GDSVSSNSAAWNWIRQSPSRGLEWL | entire sequence, with |
| | GRTYYRSKWYNDYAVSVKSRITINP | 3xG₄S linker |
| | DTSKNQFSLQLNSVTPEDTAVYYCA | |
| | REVTGDLEDAFDIWGQGTMVTVSSG | |
| | GGGSGGGGSGGGGSDIQMTQSPSSL | |
| | SASVGDRVTITCRASQTIWSYLNWY | |
| | QQRPGKAPNLLIYAASSLQSGVPSR | |
| | FSGRGSGTDFTLTISSLQAEDFATY | |
| | YCQQSYSIPQTFGQGTKLEIK | |
| 46 | QVQLQQSGPGLVKPSQTLSLTCAIS | Anti-CD22 m971 scFv |
| | GDSVSSNSAAWNWIRQSPSRGLEWL | heavy chain variable |
| | GRTYYRSKWYNDYAVSVKSRITINP | region |
| | DTSKNQFSLQLNSVTPEDTAVYYCA | |
| | REVTGDLEDAFDIWGQGTMVTVSS | |
| 47 | GDSVSSNSAA | Anti-CD22 m971 scFv |
| | | heavy chain CDR1 |
| 48 | TYYRSKWYN | Anti-CD22 m971 scFv |
| | | heavy chain CDR2 |
| 49 | AREVTGDLEDAFDI | Anti-CD22 m971 scFv |
| | | heavy chain CDR3 |
| 50 | DIQMTQSPSSLSASVGDRVTITCRA | Anti-CD22 m971 scFv |
| | SQTIWSYLNWYQQRPGKAPNLLIYA | light chain |
| | ASSLQSGVPSRFSGRGSGTDFTLTI | |
| | SSLQAEDFATYYCQQSYSIPQTFGQ | |
| | GTKLEIK | |
| 51 | QTIWSY | Anti-CD22 m971 scFv |
| | | light chain CDR1 |
| 52 | AAS | Anti-CD22 m971 scFv |
| | | light chain CDR2 |
| 53 | QQSYSIPQT | Anti-CD22 m971 scFv |
| | | light chain CDR3 |
| 54 | QVQLQQSGPGMVKPSQTLSLTCAIS | Anti-CD22 m971-L7 |
| | GDSVSSNSVAWNWIRQSPSRGLEWL | scFv entire sequence, |
| | GRTYYRSTWYNDYAVSMKSRITINP | with 3xG₄S linker |
| | DTNKNQFSLQLNSVTPEDTAVYYCA | |
| | REVTGDLEDAFDIWGQGTMVTVSSG | |
| | GGGSGGGGSGGGGSDIQMIQSPSSL | |
| | SASVGDRVTITCRASQTIWSYLNWY | |
| | RQRPGEAPNLLIYAASSLQSGVPSR | |
| | FSGRGSGTDFTLTISSLQAEDFATY | |
| | YCQQSYSIPQTFGQGTKLEIK | |
| 55 | QVQLQQSGPGMVKPSQTLSLTCAIS | Anti-CD22 m971-L7 |
| | GDSVSSNSVAWNWIRQSPSRGLEWL | scFv heavy chain |
| | GRTYYRSTWYNDYAVSMKSRITINP | variable region |
| | DTNKNQFSLQLNSVTPEDTAVYYCA | |
| | REVTGDLEDAFDIWGQGTMVTVSS | |
| 56 | GDSVSSNSVA | Anti-CD22 m971-L7 |
| | | scFv heavy chain CDR1 |
| 57 | TYYRSTWYN | Anti-CD22 m971-L7 |
| | | scFv heavy chain CDR2 |
| 58 | AREVTGDLEDAFDI | Anti-CD22 m971-L7 |
| | | scFv heavy chain CDR3 |
| 59 | DIQMIQSPSSLSASVGDRVTITCRA | Anti-CD22 m971-L7 |
| | SQTIWSYLNWYRQRPGEAPNLLIYA | scFv light chain |
| | ASSLQSGVPSRFSGRGSGTDFTLTI | variable region |
| | SSLQAEDFATYYCQQSYSIPQTFGQ | |
| | GTKLEIK | |
| 60 | QTIWSY | Anti-CD22 m971-L7 |
| | | scFv light chain CDR1 |
| 61 | AAS | Anti-CD22 m971-L7 |
| | | scFv light chain CDR2 |
| 62 | QQSYSIPQT | Anti-CD22 m971-L7 |
| | | scFv light chain CDR3 |

In some embodiments, the extracellular binding domain of the CD22 CAR comprises immunotoxins HA22 or BL22. Immunotoxins BL22 and HA22 are therapeutic agents that comprise an scFv specific for CD22 fused to a bacterial toxin, and thus can bind to the surface of the cancer cells that express CD22 and kill the cancer cells. BL22 comprises a dsFv of an anti-CD22 antibody, RFB4, fused to a 38-kDa truncated form of Pseudomonas exotoxin A (Bang et al., Clin. Cancer Res., 11:1545-50 (2005)). HA22 (CAT8015, moxetumomab pasudotox) is a mutated, higher affinity version of BL22 (Ho et al., J. Biol. Chem., 280(1): 607-17 (2005)). Suitable sequences of antigen binding domains of HA22 and BL22 specific to CD22 are disclosed in, for example, U.S. Pat. Nos. 7,541,034; 7,355,012; and 7,982,011, which are hereby incorporated by reference in their entirety.

In some embodiments, the hinge domain of the CD22 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 13.

In some embodiments, the transmembrane domain of the CD22 CAR comprises a CD8α transmembrane domain, for example, a human CD8α transmembrane domain. In some embodiments, the CD8α transmembrane domain comprises or consists of an amino acid sequence set forth in SEQ ID NO: 14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the CD22 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the CD22 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the CD8α hinge domain of SEQ ID NO:9, the CD8α transmembrane domain of SEQ ID NO:14, the 4-1BB costimulatory domain of SEQ ID NO: 16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the CD28 hinge domain of SEQ ID NO: 10, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the IgG4 hinge domain of SEQ ID NO:11 or SEQ ID NO: 12, the CD8α transmembrane domain of SEQ ID NO: 14, the 4-1BB costimulatory domain of SEQ ID NO: 16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the CD8α hinge domain of SEQ ID NO:9, the CD28 transmembrane domain of SEQ ID NO: 15, the 4-1BB costimulatory domain of SEQ ID NO: 16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the CD28 hinge domain of SEQ ID NO: 10, the CD28 transmembrane domain of SEQ ID NO: 15, the 4-1BB costimulatory domain of SEQ ID NO: 16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the IgG4 hinge domain of SEQ ID NO:11 or SEQ ID NO:12, the CD28 transmembrane domain of SEQ ID NO: 15, the 4-1BB costimulatory domain of SEQ ID NO: 16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### BCMA CAR

In some embodiments, the CAR is a BCMA CAR, and in these embodiments, the polycistronic vector comprises an expression cassette that contains a nucleotide sequence encoding a BCMA CAR. BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of BCMA has been recently linked to a number of cancers, such as multiple myeloma, Hodgkin's and non-Hodgkin's lymphoma, various leukemias, and glioblastoma. In some embodiments, the BCMA CAR may comprise a signal peptide, an extracellular binding domain that specifically binds BCMA, a hinge domain, a transmembrane domain, an intracellular costimulatory domain, and/or an intracellular signaling domain in tandem.

In some embodiments, the signal peptide of the BCMA CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the BCMA CAR is specific to BCMA, for example, human BCMA. The extracellular binding domain of the BCMA CAR can be codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the BCMA CAR is derived from an antibody specific to BCMA, including, for example, belantamab, erlanatamab, teclistamab, LCAR-B38M, and ciltacabtagene. In any of these embodiments, the extracellular binding domain of the BCMA CAR can comprise or consist of the V_{H}, the V_{L}, and/or one or more CDRs of any of the antibodies.

In some embodiments, the extracellular binding domain of the BCMA CAR comprises an scFv derived from C11D5.3, a murine monoclonal antibody as described in Carpenter et al., Clin. Cancer Res. 19(8):2048-2060 (2013). See also PCT Application Publication No. WO2010/104949. The C11D5.3-derived scFv may comprise the heavy chain variable region (V_{H}) and the light chain variable region (V_{L}) of C11D5.3 connected by the Whitlow linker, the amino acid sequences of which is provided in Table 13 below. In some embodiments, the BCMA-specific extracellular binding domain comprises or consists of an amino acid sequence set forth in SEQ ID NO:63, 64, or 68, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:63, 64, or 68. In some embodiments, the BCMA-specific extracellular binding domain may comprise one or more CDRs having amino acid sequences set forth in SEQ ID NOs: 65-67 and 69-71. In some embodiments, the BCMA-specific extracellular binding domain may comprise a light chain with one or more CDRs having amino acid sequences set forth in SEQ ID NOs: 65-67. In some embodiments, the BCMA-specific extracellular binding domain may comprise a heavy chain with one or more CDRs having amino acid sequences set forth in SEQ ID NOs: 69-71. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from another murine monoclonal antibody, C12A3.2, as described in Carpenter et al., Clin. Cancer Res. 19(8):2048-2060 (2013) and PCT Application Publication No. WO2010/104949, the amino acid sequence of which is also provided in Table 13 below. In some embodiments, the BCMA-specific extracellular binding domain comprises or consists of an amino acid sequence set forth in SEQ ID NO:72, 73, or 77, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:72, 73, or 77. In some embodiments, the BCMA-specific extracellular binding domain may comprise one or more CDRs having amino acid sequences set forth in SEQ ID NOs: 74-76 and 78-80. In some embodiments, the BCMA-specific extracellular binding domain may comprise a light chain with one or more CDRs having amino acid sequences set forth in SEQ ID NOs: 74-76. In some embodiments, the BCMA-specific extracellular binding domain may comprise a heavy chain with one or more CDRs having amino acid sequences set forth in SEQ ID NOs: 78-80. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises a murine monoclonal antibody with high specificity to human BCMA, referred to as BB2121 in Friedman et al., Hum. Gene Ther. 29(5):585-601 (2018)). See also, PCT Application Publication No. WO2012163805.

In some embodiments, the extracellular binding domain of the BCMA CAR comprises single variable fragments of two heavy chains (VHH) that can bind to two epitopes of BCMA as described in Zhao et al., J. Hematol. Oncol. 11(1): 141 (2018), also referred to as LCAR-B38M. See also, PCT Application Publication No. WO2018/028647.

In some embodiments, the extracellular binding domain of the BCMA CAR comprises a fully human heavy-chain variable domain (FHVH) as described in Lam et al., Nat. Commun. 11(1):283 (2020), also referred to as FHVH33. See also, PCT Application Publication No. WO2019/006072. The amino acid sequences of FHVH33 and its CDRs are provided in Table 13 below. In some embodiments, the BCMA-specific extracellular binding domain comprises or consists of an amino acid sequence set forth in SEQ ID NO:81 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:81. In some embodiments, the BCMA-specific extracellular binding domain may comprise one or more CDRs having amino acid sequences set forth in SEQ ID NOs: 82-84. In any of these embodiments, the BCMA-specific extracellular binding domain may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from CT103A (or CAR0085) as described in U.S. Pat. No. 11,026,975 B2, the amino acid sequence of which is provided in Table 13 below. In some embodiments, the BCMA-specific extracellular binding domain comprises or consists of an amino acid sequence set forth in SEQ ID NO:118, 119, or 123, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 118, 119, or 123. In some embodiments, the BCMA-specific extracellular binding domain may comprise one or more CDRs having amino acid sequences set forth in SEQ ID NOs: 120-122 and 124-126. In some embodiments, the BCMA-specific extracellular binding domain may comprise a light chain with one or more CDRs having amino acid sequences set forth in SEQ ID NOs: 120-122. In some embodiments, the BCMA-specific extracellular binding domain may comprise a heavy chain with one or more CDRs having amino acid sequences set forth in SEQ ID NOs: 124-126. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

Additionally, CARs and binders directed to BCMA have been described in U.S. Application Publication Nos. 2020/0246381 A1 and 2020/0339699 A1, the entire contents of each of which are incorporated by reference herein.

TABLE 13
Exemplary sequences of anti-BCMA
binder and components

| SEQ | | |
|---|---|---|
| ID | | |
| NO: | Amino Acid Sequence | Description |
| 63 | DIVLTQSPASLAMSLGKRATISCRAS | Anti-BCMA C11D5.3 |
| | ESVSVIGAHLIHWYQQKPGQPPKLLI | scFv entire sequence, |
| | YLASNLETGVPARFSGSGSGTDFTLT | with Whitlow linker |
| | IDPVEEDDVAIYSCLQSRIFPRTFGG | |
| | GTKLEIKGSTSGSGKPGSGEGSTKGQ | |
| | IQLVQSGPELKKPGETVKISCKASGY | |
| | TFTDYSINWVKRAPGKGLKWMGWINT | |
| | ETREPAYAYDFRGRFAFSLETSASTA | |
| | YLQINNLKYEDTATYFCALDYSYAMD | |
| | YWGQGTSVTVSS | |
| 64 | DIVLTQSPASLAMSLGKRATISCRAS | Anti-BCMA C11D5.3 |
| | ESVSVIGAHLIHWYQQKPGQPPKLLI | scFv light chain |
| | YLASNLETGVPARFSGSGSGTDFTLT | variable region |
| | IDPVEEDDVAIYSCLQSRIFPRTFGG | |
| | GTKLEIK | |
| 65 | RASESVSVIGAHLIH | Anti-BCMA C11D5.3 |
| | | scFv light chain CDR1 |
| 66 | LASNLET | Anti-BCMA C11D5.3 |
| | | scFv light chain CDR2 |
| 67 | LQSRIFPRT | Anti-BCMA C11D5.3 |
| | | scFv light chain CDR3 |
| 68 | QIQLVQSGPELKKPGETVKISCKASG | Anti-BCMA C11D5.3 |
| | YTFTDYSINWVKRAPGKGLKWMGWIN | scFv heavy chain |
| | TETREPAYAYDFRGRFAFSLETSAST | variable region |
| | AYLQINNLKYEDTATYFCALDYSYAM | |
| | DYWGQGTSVTVSS | |
| 69 | DYSIN | Anti-BCMA C11D5.3 |
| | | scFv heavy chain CDR1 |
| 70 | WINTETREPAYAYDFRG | Anti-BCMA C11D5.3 |
| | | scFv heavy chain CDR2 |
| 71 | DYSYAMDY | Anti-BCMA C11D5.3 |
| | | scFv heavy chain CDR3 |
| 72 | DIVLTQSPPSLAMSLGKRATISCRAS | Anti-BCMA C12A3.2 |
| | ESVTILGSHLIYWYQQKPGQPPTLLI | scFv entire sequence, |
| | QLASNVQTGVPARFSGSGSRTDFTLT | with Whitlow linker |
| | IDPVEEDDVAVYYCLQSRTIPRTFGG | |
| | GTKLEIKGSTSGSGKPGSGEGSTKGQ | |
| | IQLVQSGPELKKPGETVKISCKASGY | |
| | TFRHYSMNWVKQAPGKGLKWMGRINT | |
| | ESGVPIYADDFKGRFAFSVETSASTA | |
| | YLVINNLKDEDTASYFCSNDYLYSLD | |
| | FWGQGTALTVSS | |
| 73 | DIVLTQSPPSLAMSLGKRATISCRAS | Anti-BCMA C12A3.2 |
| | ESVTILGSHLIYWYQQKPGQPPTLLI | scFv light chain |
| | QLASNVQTGVPARFSGSGSRTDFTLT | variable region |
| | IDPVEEDDVAVYYCLQSRTIPRTFGG | |
| | GTKLEIK | |
| 74 | RASESVTILGSHLIY | Anti-BCMA C12A3.2 |
| | | scFv light chain CDR1 |
| 75 | LASNVQT | Anti-BCMA C12A3.2 |
| | | scFv light chain CDR2 |
| 76 | LQSRTIPRT | Anti-BCMA C12A3.2 |
| | | scFv light chain CDR3 |
| 77 | QIQLVQSGPELKKPGETVKISCKASG | Anti-BCMA C12A3.2 |
| | YTFRHYSMNWVKQAPGKGLKWMGRIN | scFv heavy chain |
| | TESGVPIYADDFKGRFAFSVETSAST | variable region |
| | AYLVINNLKDEDTASYFCSNDYLYSL | |
| | DFWGQGTALTVSS | |
| 78 | HYSMN | Anti-BCMA C12A3.2 |
| | | scFv heavy chain CDR1 |
| 79 | RINTESGVPIYADDFKG | Anti-BCMA C12A3.2 |
| | | scFv heavy chain CDR2 |
| 80 | DYLYSLDF | Anti-BCMA C12A3.2 |
| | | scFv heavy chain CDR3 |
| 81 | EVQLLESGGGLVQPGGSLRLSCAASG | Anti-BCMA FHVH33 |
| | FTFSSYAMSWVRQAPGKGLEWVSSIS | entire sequence |
| | GSGDYIYYADSVKGRFTISRDISKNT | |
| | LYLQMNSLRAEDTAVYYCAKEGTGAN | |
| | SSLADYRGQGTLVTVSS | |
| 82 | GFTFSSYA | Anti-BCMA FHVH33 |
| | | CDR1 |
| 83 | ISGSGDYI | Anti-BCMA FHVH33 |
| | | CDR2 |
| 84 | AKEGTGANSSLADY | Anti-BCMA FHVH33 |
| | | CDR3 |
| 118 | DIQMTQSPSSLSASVGDRVTITCRAS | Anti-BCMA CT103A |
| | QSISSYLNWYQQKPGKAPKLLIYAAS | scFv entire sequence, |
| | SLQSGVPSRFSGSGSGTDFTLTISSL | with Whitlow linker |
| | QPEDFATYYCQQKYDLLTFGGGTKVE | |
| | IKGSTSGSGKPGSGEGSTKGQLQLQE | |
| | SGPGLVKPSETLSLTCTVSGGSISSS | |
| | SYYWGWIRQPPGKGLEWIGSISYSGS | |
| | TYYNPSLKSRVTISVDTSKNQFSLKL | |
| | SSVTAADTAVYYCARDRGDTILDVWG | |
| | QGTMVTVSS | |
| 119 | DIQMTQSPSSLSASVGDRVTITCRAS | Anti-BCMA CT103A |
| | QSISSYLNWYQQKPGKAPKLLIYAAS | scFv light chain |
| | SLQSGVPSRFSGSGSGTDFTLTISSL | variable region |
| | QPEDFATYYCQQKYDLLTFGGGTKVE | |
| | IK | |
| 120 | QSISSY | Anti-BCMA CT103A |
| | | scFv light chain CDR1 |
| 121 | AAS | Anti-BCMA CT103A |
| | | scFv light chain CDR2 |
| 122 | QQKYDLLT | Anti-BCMA CT103A |
| | | scFv light chain CDR3 |
| 123 | QLQLQESGPGLVKPSETLSLTCTVSG | Anti-BCMA CT103A |
| | GSISSSSYYWGWIRQPPGKGLEWIGS | scFv heavy chain |
| | ISYSGSTYYNPSLKSRVTISVDTSKN | variable region |
| | QFSLKLSSVTAADTAVYYCARDRGDT | |
| | ILDVWGQGTMVTVSS | |
| 124 | GGSISSSSYY | Anti-BCMA CT103A |
| | | scFv heavy chain CDR1 |
| 125 | ISYSGST | Anti-BCMA CT103A |
| | | scFv heavy chain CDR2 |
| 126 | ARDRGDTILDV | Anti-BCMA CT103A |
| | | scFv heavy chain CDR3 |

In some embodiments, the hinge domain of the BCMA CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO: 12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the BCMA CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the BCMA CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the BCMA CAR comprises a CD3 zeta (ζ) signaling domain, for example, a human CD3ζ signaling domain. In some embodiments, the CD3ζ signaling domain comprises or consists of an amino acid sequence set forth in SEQ ID NO: 18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8α hinge domain of SEQ ID NO:9, the CD8α transmembrane domain of SEQ ID NO: 14, the 4-1BB costimulatory domain of SEQ ID NO: 16, the CD3 signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide (e.g., a CD8α signal peptide) as described.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8α hinge domain of SEQ ID NO:9, the CD8α transmembrane domain of SEQ ID NO:14, the CD28 costimulatory domain of SEQ ID NO:17, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide as described.

In some embodiments, the polycistronic vector comprises an expression cassette that contains a nucleotide sequence encoding a BCMA CAR as set forth in SEQ ID NO: 127 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:127 (see Table 14). The encoded BCMA CAR has a corresponding amino acid sequence set forth in SEQ ID NO:128 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 128, with the following components: CD8α signal peptide, CT103A scFv (V_{L}-Whitlow linker-V_{H}), CD8α hinge domain, CD8α transmembrane domain, 4-1BB costimulatory domain, and CD3ζ signaling domain.

In some embodiments, the polycistronic vector comprises an expression cassette that contains a nucleotide sequence encoding a commercially available embodiment of BCMA CAR, including, for example, idecabtagene vicleucel (ide-cel, also called bb2121). In some embodiments, the polycistronic vector comprises an expression cassette that contains a nucleotide sequence encoding idecabtagene vicleucel or portions thereof. Idecabtagene vicleucel comprises a BCMA CAR with the following components: the BB2121 binder, CD8α hinge domain, CD8α transmembrane domain, 4-1BB costimulatory domain, and CD3ζ signaling domain.

TABLE 14
Exemplary sequences of BCMA CARs

| SEQ ID | | |
|---|---|---|
| NO: | Sequence | Description |
| 127 | atggccttaccagtgaccgccttgctcctgccgctggccttgctg | Exemplary BCMA |
| | ctccacgccgccaggccggacatccagatgacccagtctccatcc | CAR nucleotide |
| | tccctgtctgcatctgtaggagacagagtcaccatcacttgccgg | sequence |
| | gcaagtcagagcattagcagctatttaaattggtatcagcagaaa | |
| | ccagggaaagcccctaagctcctgatctatgctgcatccagtttg | |
| | caaagtggggtcccatcaaggttcagtggcagtggatctgggaca | |
| | gatttcactctcaccatcagcagtctgcaacctgaagattttgca | |
| | acttactactgtcagcaaaaatacgacctcctcacttttggcgga | |
| | gggaccaaggttgagatcaaaggcagcaccagcggctccggcaag | |
| | cctggctctggcgagggcagcacaaagggacagctgcagctgcag | |
| | gagtcgggcccaggactggtgaagccttcggagaccctgtccctc | |
| | acctgcactgtctctggtggctccatcagcagtagtagttactac | |
| | tggggctggatccgccagcccccagggaaggggctggagtggatt | |
| | gggagtatctcctatagtgggagcacctactacaacccgtccctc | |
| | aagagtcgagtcaccatatccgtagacacgtccaagaaccagttc | |
| | tccctgaagctgagttctgtgaccgccgcagacacggcggtgtac | |
| | tactgcgccagagatcgtggagacaccatactagacgtatggggt | |
| | cagggtacaatggtcaccgtcagctcattcgtgcccgtgttcctg | |
| | cccgccaaacctaccaccacccctgcccctagacctcccacccca | |
| | gccccaacaatcgccagccagcctctgtctctgcggcccgaagcc | |
| | tgtagacctgctgccggcggagccgtgcacaccagaggcctggac | |
| | ttcgcctgcgacatctacatctgggcccctctggccggcacctgt | |
| | ggcgtgctgctgctgagcctggtgatcaccctgtactgcaaccac | |
| | cggaacaaacggggcagaaagaaactcctgtatatattcaaacaa | |
| | ccatttatgagaccagtacaaactactcaagaggaagatggctgt | |
| | agctgccgatttccagaagaagaagaaggaggatgtgaactgaga | |
| | gtgaagttcagcagatccgccgacgcccctgcctaccagcaggga | |
| | cagaaccagctgtacaacgagctgaacctgggcagacgggaagag | |
| | tacgacgtgctggacaagcggagaggccgggaccccgagatgggc | |
| | ggaaagcccagacggaagaacccccaggaaggcctgtataacgaa | |
| | ctgcagaaagacaagatggccgaggcctacagcgagatcggcatg | |
| | aagggcgagcggaggcgcggcaagggccacgatggcctgtaccag | |
| | ggcctgagcaccgccaccaaggacacctacgacgccctgcacatg | |
| | caggccctgccccccaga | |
| 128 | MALPVTALLLPLALLLHAARPDIQMTQSPSSLSASVGDRVTITCR | Exemplary BCMA |
| | ASQSISSYLNWYQQKPGKAPKLLIYAASSLQSGVPSRFSGSGSGT | CAR amino acid |
| | DFTLTISSLQPEDFATYYCQQKYDLLTFGGGTKVEIKGSTSGSGK | sequence |
| | PGSGEGSTKGQLQLQESGPGLVKPSETLSLTCTVSGGSISSSSYY | |
| | WGWIRQPPGKGLEWIGSISYSGSTYYNPSLKSRVTISVDTSKNQF | |
| | SLKLSSVTAADTAVYYCARDRGDTILDVWGQGTMVTVSSFVPVFL | |
| | PAKPTTTPAPRPPTPAPTIASQPLSLRPEACRPAAGGAVHTRGLD | |
| | FACDIYIWAPLAGTCGVLLLSLVITLYCNHRNKRGRKKLLYIFKQ | |
| | PFMRPVQTTQEEDGCSCRFPEEEEGGCELRVKFSRSADAPAYQQG | |
| | QNQLYNELNLGRREEYDVLDKRRGRDPEMGGKPRRKNPQEGLYNE | |
| | LQKDKMAEAYSEIGMKGERRRGKGHDGLYQGLSTATKDTYDALHM | |
| | QALPPR | |

### P. Characteristics of Hypoimmunogenic Cells

In some embodiments, the population of hypoimmunogenic stem cells retains pluripotency as compared to a control stem cell (e.g., a wild-type stem cell or immunogenic stem cell). In some embodiments, the population of hypoimmunogenic stem cells retains differentiation potential as compared to a control stem cell (e.g., a wild-type stem cell or immunogenic stem cell).

In some embodiments, the administered population of hypoimmunogenic cells such as hypoimmunogenic CAR-T cells elicits a decreased or lower level of immune activation in the subject or patient. In some instances, the level of immune activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation produced by the administration of immunogenic cells. In some embodiments, the administered population of hypoimmunogenic cells fails to elicit immune activation in the subject or patient.

In some embodiments, the administered population of hypoimmunogenic cells such as hypoimmunogenic CAR-T cells elicits a decreased or lower level of T cell response in the subject or patient. In some instances, the level of T cell response elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of T cell response produced by the administration of immunogenic cells. In some embodiments, the administered population of hypoimmunogenic cells fails to elicit a T cell response to the cells in the subject or patient.

In some embodiments, the administered population of hypoimmunogenic cells such as hypoimmunogenic CAR-T cells elicits a decreased or lower level of NK cell response in the subject or patient. In some instances, the level of NK cell response elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of NK cell response produced by the administration of immunogenic cells. In some embodiments, the administered population of hypoimmunogenic cells fails to elicit an NK cell response to the cells in the subject or patient.

In some embodiments, the administered population of hypoimmunogenic cells such as hypoimmunogenic CAR-T cells elicits a decreased or lower level of macrophage engulfment in the subject or patient. In some instances, the level of NK cell response elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of macrophage engulfment produced by the administration of immunogenic cells. In some embodiments, the administered population of hypoimmunogenic cells fails to elicit macrophage engulfment of the cells in the subject or patient.

In some embodiments, the administered population of hypoimmunogenic cells such as hypoimmunogenic CAR-T cells elicits a decreased or lower level of systemic TH1 activation in the subject or patient. In some instances, the level of systemic TH1 activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of systemic TH1 activation produced by the administration of immunogenic cells. In some embodiments, the administered population of hypoimmunogenic cells fails to elicit systemic TH1 activation in the subject or patient.

In some embodiments, the administered population of hypoimmunogenic cells such as hypoimmunogenic CAR-T cells elicits a decreased or lower level of NK cell killing in the subject or patient. In some instances, the level of NK cell killing elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of NK cell killing produced by the administration of immunogenic cells. In some embodiments, the administered population of hypoimmunogenic cells fails to elicit NK cell killing in the subject or patient.

In some embodiments, the administered population of hypoimmunogenic cells such as hypoimmunogenic CAR-T cells elicits a decreased or lower level of immune activation of peripheral blood mononuclear cells (PBMCs) in the subject or patient. In some instances, the level of immune activation of PBMCs elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation of PBMCs produced by the administration of immunogenic cells. In some embodiments, the administered population of hypoimmunogenic cells fails to elicit immune activation of PBMCs in the subject or patient.

In some embodiments, the administered population of hypoimmunogenic cells such as hypoimmunogenic CAR-T cells elicits a decreased or lower level of donor-specific IgG antibodies in the subject or patient. In some instances, the level of donor-specific IgG antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgG antibodies produced by the administration of immunogenic cells. In some embodiments, the administered population of hypoimmunogenic cells fails to elicit donor-specific IgG antibodies in the subject or patient.

In some embodiments, the administered population of hypoimmunogenic cells such as hypoimmunogenic CAR-T cells elicits a decreased or lower level of donor-specific IgM antibodies in the subject or patient. In some instances, the level of donor-specific IgM antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgM antibodies produced by the administration of immunogenic cells. In some embodiments, the administered population of hypoimmunogenic cells fails to elicit donor-specific IgM antibodies in the subject or patient.

In some embodiments, the administered population of hypoimmunogenic cells such as hypoimmunogenic CAR-T cells elicits a decreased or lower level of IgM and IgG antibody production in the subject or patient. In some instances, the level of IgM and IgG antibody production elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of IgM and IgG antibody production produced by the administration of immunogenic cells. In some embodiments, the administered population of hypoimmunogenic cells fails to elicit IgM and IgG antibody production in the subject or patient.

In some embodiments, the administered population of hypoimmunogenic cells such as hypoimmunogenic CAR-T cells elicits a decreased or lower level of cytotoxic T cell killing in the subject or patient. In some instances, the level of cytotoxic T cell killing elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of cytotoxic T cell killing produced by the administration of immunogenic cells. In some embodiments, the administered population of hypoimmunogenic cells fails to elicit cytotoxic T cell killing in the subject or patient.

In some embodiments, the administered population of hypoimmunogenic cells such as hypoimmunogenic CAR-T cells elicits a decreased or lower level of complement-dependent cytotoxicity (CDC) in the subject or patient. In some instances, the level of CDC elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of CDC produced by the administration of immunogenic cells. In some embodiments, the administered population of hypoimmunogenic cells fails to elicit CDC in the subject or patient.

Q. Therapeutic Cells from Primary T Cells

Provided herein are hypoimmunogenic cells including, but not limited to, primary T cells that evade immune recognition. In some embodiments, the hypoimmunogenic cells are produced (e.g., generated, cultured, or derived) from T cells such as primary T cells. In some instances, primary T cells are obtained (e.g., harvested, extracted, removed, or taken) from a subject or an individual. In some embodiments, primary T cells are produced from a pool of T cells such that the T cells are from one or more subjects (e.g., one or more human including one or more healthy humans). In some embodiments, the pool of primary T cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of T cells does not include cells from the patient. In some embodiments, one or more of the donor subjects from which the pool of T cells is obtained are different from the patient.

In some embodiments, the hypoimmunogenic cells do not activate an immune response in the patient (e.g., recipient upon administration). Provided are methods of treating a disorder by administering a population of hypoimmunogenic cells to a subject (e.g., recipient) or patient in need thereof. In some embodiments, the hypoimmunogenic cells described herein comprise T cells engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells comprise reduced expression of an endogenous T cell receptor.

In some embodiments, the present technology is directed to hypoimmunogenic primary T cells that overexpress an HLA-E variant, an HLA-G variant, and/or exogenous PD-L1 and CARs, and have reduced expression or lack expression of MHC class I and/or MHC class II human leukocyte antigens and have reduced expression or lack expression of TCR complex molecules. The cells outlined herein overexpress an HLA-E variant, an HLA-G variant, and/or exogenous PD-L1 and CARs and evade immune recognition. In some embodiments, the primary T cells display reduced levels or activity of MHC class I antigens, MHC class II antigens, and/or TCR complex molecules. In many embodiments, primary T cells overexpress an HLA-E variant, an HLA-G variant, and/or exogenous PD-L1 and CARs and harbor a genomic modification in the B2M gene. In some embodiments, T cells overexpress an HLA-E variant, an HLA-G variant, and/or exogenous PD-L1 and CARs and harbor a genomic modification in the CIITA gene. In some embodiments, primary T cells overexpress an HLA-E variant, an HLA-G variant, and/or exogenous PD-L1 and CARs and harbor a genomic modification in the TRAC gene. In some embodiments, primary T cells overexpress an HLA-E variant, an HLA-G variant, and/or exogenous PD-L1 and CARs and harbor a genomic modification in the TRB gene. In some embodiments, T cells overexpress an HLA-E variant, an HLA-G variant, and/or exogenous PD-L1 and CARs and harbor genomic modifications in one or more of the following genes: the B2M, CIITA, TRAC and TRB genes.

In some embodiments, primary T cells overexpress an HLA-E variant, an HLA-G variant, and/or an exogenous PD-L1 and CARs and harbor genomic modifications in one or more of the following genes: the HLA-A, HLA-B, HLA-C, CD155, B2M, CIITA, TRAC and TRB genes. In some embodiments, primary T cells overexpress an HLA-E variant, an HLA-G variant, and/or an exogenous PD-L1 and CARs and harbor genomic modifications in one or more of the following genes: the HLA-A, HLA-B, HLA-C, and CD155 genes. In some embodiments, primary T cells overexpress an HLA-E variant, an HLA-G variant, and/or an exogenous PD-L1 and CARs and harbor a genomic modification in the HLA-A and HLA-C genes. In some embodiments, primary T cells overexpress an HLA-E variant, an HLA-G variant, and/or an exogenous PD-L1 and CARs and harbor a genomic modification in the HLA-A, HLA-B and HLA-C genes. In some embodiments, primary T cells overexpress an HLA-E variant, an HLA-G variant, and/or an exogenous PD-L1 and CARs and harbor a genomic modification in the HLA-A, HLA-C, CD155 genes. In some embodiments, primary T cells overexpress an HLA-E variant, an HLA-G variant, and/or an exogenous PD-L1 and CARs and harbor a genomic modification in the HLA-A, HLA-B, HLA-C, and CD155 genes.

Exemplary T cells of the present disclosure are selected from the group consisting of cytotoxic T cells, helper T cells, memory T cells, central memory T cells, effector memory T cells, effector memory RA T cells, regulatory T cells, tissue infiltrating lymphocytes, and combinations thereof. In many embodiments, the T cells express CCR7, CD27, CD28, and CD45RA. In some embodiments, the central T cells express CCR7, CD27, CD28, and CD45RO. In other embodiments, the effector memory T cells express PD-1, CD27, CD28, and CD45RO. In other embodiments, the effector memory RA T cells express PD-1, CD57, and CD45RA.

In some embodiments, the T cell is a modified (e.g., an engineered) T cell. In some cases, the modified T cell comprise a modification causing the cell to express at least one chimeric antigen receptor that specifically binds to an antigen or epitope of interest expressed on the surface of at least one of a damaged cell, a dysplastic cell, an infected cell, an immunogenic cell, an inflamed cell, a malignant cell, a metaplastic cell, a mutant cell, and combinations thereof. In other cases, the modified T cell comprise a modification causing the cell to express at least one protein that modulates a biological effect of interest in an adjacent cell, tissue, or organ when the cell is in proximity to the adjacent cell, tissue, or organ. Useful modifications to primary T cells are described in detail in US2016/0348073 and WO2020/018620, the disclosures of which are incorporated herein in their entireties.

In some embodiments, the hypoimmunogenic cells described herein comprise T cells are engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells include reduced expression of an endogenous T cell receptor. In some embodiments, the T cells described herein such as the engineered or modified T cells include reduced expression of cytotoxic T-lymphocyte-associated protein 4 (CTLA-4). In other embodiments, the T cells described herein such as the engineered or modified T cells include reduced expression of programmed cell death (PD-1). In many embodiments, the T cells described herein such as the engineered or modified T cells include reduced expression of CTLA-4 and PD-1. Methods of reducing or eliminating expression of CTLA-4, PD-1 and both CTLA-4 and PD-1 can include any recognized by those skilled in the art, such as but not limited to, genetic modification technologies that utilize rare-cutting endonucleases and RNA silencing or RNA interference technologies. Non-limiting examples of a rare-cutting endonuclease include any Cas protein, TALEN, zinc finger nuclease, meganuclease, and homing endonuclease. In some embodiments, an exogenous nucleic acid encoding a polypeptide as disclosed herein (e.g., a chimeric antigen receptor, an HLA-E variant, an HLA-G variant, and/or anexogenous PD-L1, or another tolerogenic factor disclosed herein) is inserted at a CTLA-4 and/or PD-1 gene locus.

In some embodiments, the T cells described herein such as the engineered or modified T cells include enhanced expression of PD-L1.

In some embodiments, the hypoimmunogenic T cell includes a polynucleotide encoding a CAR, wherein the polynucleotide is inserted in a genomic locus. In some embodiments, the polynucleotide is inserted into a safe harbor locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus. In some embodiments, the polynucleotide is inserted in a B2M, CIITA, TRAC, TRB, PD-1, CTLA-4, HLA-A, HLA-B, HLA-C, or CD155 gene.

Hypoimmunogenic T cells provided herein are useful for the treatment of suitable cancers including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

R. Therapeutic Cells Differentiated from Hypoimmunogenic Pluripotent Stem Cells

Provided herein are hypoimmunogenic cells including, cells derived from pluripotent stem cells, that evade immune recognition. In some embodiments, the cells do not activate an immune response in the patient or subject (e.g., recipient upon administration). Provided are methods of treating a disorder comprising repeat dosing of a population of hypoimmunogenic cells to a recipient subject in need thereof.

In some embodiments, the pluripotent stem cell and any cell differentiated from such a pluripotent stem cell is modified to exhibit reduced expression of MHC class I human leukocyte antigens. In other embodiments, the pluripotent stem cell and any cell differentiated from such a pluripotent stem cell is modified to exhibit reduced expression of MHC class II human leukocyte antigens. In many embodiments, the pluripotent stem cell and any cell differentiated from such a pluripotent stem cell is modified to exhibit reduced expression of TCR complexes. In some embodiments, the pluripotent stem cell and any cell differentiated from such a pluripotent stem cell is modified to exhibit reduced expression of MHC class I and II human leukocyte antigens. In some embodiments, the pluripotent stem cell and any cell differentiated from such a pluripotent stem cell is modified to exhibit reduced expression of MHC class I and II human leukocyte antigens and TCR complexes.

In some embodiments, the pluripotent stem cell and any cell differentiated from such a pluripotent stem cell is modified to exhibit reduced expression of MHC class I human leukocyte antigens. In other embodiments, the pluripotent stem cell and any cell differentiated from such a pluripotent stem cell is modified to exhibit reduced expression of MHC class II human leukocyte antigens. In many embodiments, the pluripotent stem cell and any cell differentiated from such a pluripotent stem cell is modified to exhibit reduced expression of TCR complexes. In some embodiments, the pluripotent stem cell and any cell differentiated from such a pluripotent stem cell is modified to exhibit reduced expression of MHC class I and II human leukocyte antigens. In some embodiments, the pluripotent stem cell and any cell differentiated from such a pluripotent stem cell is modified to exhibit reduced expression of MHC class I and II human leukocyte antigens and TCR complexes.

In some embodiments, the pluripotent stem cell and any cell differentiated from such a pluripotent stem cell is modified to exhibit reduced expression of MHC class I and/or II human leukocyte antigens and exhibit increased HLA-E variant, HLA-G variant, and/or exogenous PD-L1 expression. In some instances, the cell overexpresses an HLA-E variant, an HLA-G variant, and/or an exogenous PD-L1 by harboring one or more HLA-E variant, HLA-G variant, and/or exogenous PD-L1 transgenes. In some embodiments, the pluripotent stem cell and any cell differentiated from such a pluripotent stem cell is modified to exhibit reduced expression of MHC class I and II human leukocyte antigens and exhibit increased HLA-E variant, HLA-G variant, and/or exogenous PD-L1 expression. In some embodiments, the pluripotent stem cell and any cell differentiated from such a pluripotent stem cell is modified to exhibit reduced expression of MHC class I and II human leukocyte antigens and TCR complexes and exhibit increased HLA-E variant, HLA-G variant, and/or exogenous PD-L1 expression.

In some embodiments, the pluripotent stem cell and any cell differentiated from such a pluripotent stem cell is modified to exhibit reduced expression of MHC class I and/or II human leukocyte antigens, to exhibit increased HLA-E variant, HLA-G variant, and/or exogenous PD-L1 expression, and to exogenously express a chimeric antigen receptor. In some instances, the cell overexpresses an HLA-E variant, an HLA-G variant, and/or an exogenous PD-L1 polypeptides by harboring one or more HLA-E variant HLA-G variant, and/or exogenous PD-L1 transgenes. In some instances, the cell overexpresses CAR polypeptides by harboring one or more CAR transgenes. In some embodiments, the pluripotent stem cell and any cell differentiated from such a pluripotent stem cell is modified to exhibit reduced expression of MHC class I and II human leukocyte antigens, exhibit increased HLA-E variant, HLA-G variant, and/or exogenous PD-L1 expression, and to exogenously express a chimeric antigen receptor. In some embodiments, the pluripotent stem cell and any cell differentiated from such a pluripotent stem cell is modified to exhibit reduced expression of MHC class I and II human leukocyte antigens and TCR complexes, to exhibit increased HLA-E variant, HLA-G variant, and/or exogenous PD-L1 expression, and to exogenously express a chimeric antigen receptor.

Such pluripotent stem cells are hypoimmunogenic stem cells. Such differentiated cells are hypoimmunogenic cells.

Any of the pluripotent stem cells described herein can be differentiated into any cells of an organism and tissue. In some embodiments, the cells exhibit reduced expression of MHC class I and/or II human leukocyte antigens and reduced expression of TCR complexes. In some instances, expression of MHC class I and/or II human leukocyte antigens is reduced compared to unmodified or wildtype cell of the same cell type. In some instances, expression of TCR complexes is reduced compared to unmodified or wildtype cell of the same cell type. In some embodiments, the cells exhibit increased HLA-E variant, HLA-G variant, and/or exogenous PD-L1 expression. In some instances, expression of an HLA-E variant, an HLA-G variant, and/or exogenous PD-L1 is increased in cells encompassed by the present technology as compared to unmodified or wildtype cells of the same cell type. In some embodiments, the cells exhibit exogenous CAR expression. Methods for reducing levels of MHC class I and/or II human leukocyte antigens and TCR complexes and increasing the expression of an HLA-E variant, an HLA-G variant, and/or exogenous PD-L1 and CARs are described herein.

In some embodiments, the cells used in the methods described herein evade immune recognition and responses when administered to a patient (e.g., recipient subject). The cells can evade killing by immune cells in vitro and in vivo. In some embodiments, the cells evade killing by macrophages and NK cells. In some embodiments, the cells are ignored by immune cells or a subject's immune system. In other words, the cells administered in accordance with the methods described herein are not detectable by immune cells of the immune system. In some embodiments, the cells are cloaked and therefore avoid immune rejection.

Methods of determining whether a pluripotent stem cell and any cell differentiated from such a pluripotent stem cell evades immune recognition include, but are not limited to, IFN-γ Elispot assays, microglia killing assays, cell engraftment animal models, cytokine release assays, ELISAs, killing assays using bioluminescence imaging or chromium release assay or a real-time, quantitative microelectronic biosensor system for cell analysis (xCELLigence® RTCA system, Agilent), mixed-lymphocyte reactions, immunofluorescence analysis, etc.

Therapeutic cells outlined herein are useful to treat a disorder such as, but not limited to, a cancer, a genetic disorder, a chronic infectious disease, an autoimmune disorder, a neurological disorder, and the like.

### 1. Cardiac Cells

Provided herein are cardiac cell types differentiated from hypoimmunogenic induced pluripotent (HIP) cells for subsequent transplantation or engraftment into subjects (e.g., recipients). As will be appreciated by those in the art, the methods for differentiation depend on the desired cell type using known techniques. Exemplary cardiac cell types include, but are not limited to, a cardiomyocyte, nodal cardiomyocyte, conducting cardiomyocyte, working cardiomyocyte, cardiomyocyte precursor cell, cardiomyocyte progenitor cell, cardiac stem cell, cardiac muscle cell, atrial cardiac stem cell, ventricular cardiac stem cell, epicardial cell, hematopoietic cell, vascular endothelial cell, endocardial endothelial cell, cardiac valve interstitial cell, cardiac pacemaker cell, and the like.

In some embodiments, cardiac cells described herein are administered to a recipient subject to treat a cardiac disorder selected from the group consisting of pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, idiopathic cardiomyopathy, other cardiomyopathy, myocardial ischemic reperfusion injury, ventricular dysfunction, heart failure, congestive heart failure, coronary artery disease, end-stage heart disease, atherosclerosis, ischemia, hypertension, restenosis, angina pectoris, rheumatic heart, arterial inflammation, cardiovascular disease, myocardial infarction, myocardial ischemia, congestive heart failure, myocardial infarction, cardiac ischemia, cardiac injury, myocardial ischemia, vascular disease, acquired heart disease, congenital heart disease, atherosclerosis, coronary artery disease, dysfunctional conduction systems, dysfunctional coronary arteries, pulmonary hypertension, cardiac arrhythmias, muscular dystrophy, muscle mass abnormality, muscle degeneration, myocarditis, infective myocarditis, drug- or toxin-induced muscle abnormalities, hypersensitivity myocarditis, and autoimmune endocarditis.

Accordingly, provided herein are methods for the treatment and prevention of a cardiac injury or a cardiac disease or disorder in a subject in need thereof. The methods described herein can be used to treat, ameliorate, prevent or slow the progression of a number of cardiac diseases or their symptoms, such as those resulting in pathological damage to the structure and/or function of the heart. The terms “cardiac disease,” “cardiac disorder,” and “cardiac injury,” are used interchangeably herein and refer to a condition and/or disorder relating to the heart, including the valves, endothelium, infarcted zones, or other components or structures of the heart. Such cardiac diseases or cardiac-related disease include, but are not limited to, myocardial infarction, heart failure, cardiomyopathy, congenital heart defect, heart valve disease or dysfunction, endocarditis, rheumatic fever, mitral valve prolapse, infective endocarditis, hypertrophic cardiomyopathy, dilated cardiomyopathy, myocarditis, cardiomegaly, and/or mitral insufficiency, among others.

In some embodiments, the cardiomyocyte precursor includes a cell that is capable giving rise to progeny that include mature (end-stage) cardiomyocytes. Cardiomyocyte precursor cells can often be identified using one or more markers selected from GATA-4, Nkx2.5, and the MEF-2 family of transcription factors. In some instances, cardiomyocytes refer to immature cardiomyocytes or mature cardiomyocytes that express one or more markers (sometimes at least 2, 3, 4 or 5 markers) from the following list: cardiac troponin I (cTnl), cardiac troponin T (cTnT), sarcomeric myosin heavy chain (MHC), GATA-4, Nkx2.5, N-cadherin, β2-adrenoceptor, ANF, the MEF-2 family of transcription factors, creatine kinase MB (CK-MB), myoglobin, and atrial natriuretic factor (ANF). In some embodiments, the cardiac cells demonstrate spontaneous periodic contractile activity. In some cases, when that cardiac cells are cultured in a suitable tissue culture environment with an appropriate Ca2+ concentration and electrolyte balance, the cells can be observed to contract in a periodic fashion across one axis of the cell, and then release from contraction, without having to add any additional components to the culture medium. In some embodiments, the cardiac cells are hypoimmunogenic cardiac cells.

In some embodiments, the method of producing a population of hypoimmunogenic cardiac cells from a population of hypoimmunogenic induced pluripotent stem cells by in vitro differentiation comprises: (a) culturing a population of hypoimmunogenic induced pluripotent stem cells in a culture medium comprising a GSK inhibitor; (b) culturing the population of hypoimmunogenic induced pluripotent stem cells in a culture medium comprising a WNT antagonist to produce a population of pre-cardiac cells; and (c) culturing the population of pre-cardiac cells in a culture medium comprising insulin to produce a population of hypoimmune cardiac cells. In some embodiments, the GSK inhibitor is CHIR-99021, a derivative thereof, or a variant thereof. In some instances, the GSK inhibitor is at a concentration ranging from about 2 mM to about 10 mM. In some embodiments, the WNT antagonist is IWR1, a derivative thereof, or a variant thereof. In some instances, the WNT antagonist is at a concentration ranging from about 2 mM to about 10 mM.

In some embodiments, the population of hypoimmunogenic cardiac cells is isolated from non-cardiac cells. In some embodiments, the isolated population of hypoimmunogenic cardiac cells are expanded prior to administration. In many embodiments, the isolated population of hypoimmunogenic cardiac cells are expanded and cryopreserved prior to administration.

Other useful methods for differentiating induced pluripotent stem cells or pluripotent stem cells into cardiac cells are described, for example, in US2017/0152485; US2017/0058263; US2017/0002325; US2016/0362661; US2016/0068814; U.S. Pat. Nos. 9,062,289; 7,897,389; and U.S. Pat. No. 7,452,718. Additional methods for producing cardiac cells from induced pluripotent stem cells or pluripotent stem cells are described in, for example, Xu et al, Stem Cells and Development, 2006, 15(5): 631-9, Burridge et al, Cell Stem Cell, 2012, 10: 16-28, and Chen et al, Stem Cell Res, 2015, 15(2):365-375.

In various embodiments, hypoimmunogenic cardiac cells can be cultured in culture medium comprising a BMP pathway inhibitor, a WNT signaling activator, a WNT signaling inhibitor, a WNT agonist, a WNT antagonist, a Src inhibitor, a EGFR inhibitor, a PCK activator, a cytokine, a growth factor, a cardiotropic agent, a compound, and the like.

The WNT signaling activator includes, but is not limited to, CHIR99021. The PCK activator includes, but is not limited to, PMA. The WNT signaling inhibitor includes, but is not limited to, a compound selected from KY02111, SO3031 (KY01-I), SO2031 (KY02-I), and SO3042 (KY03-I), and XAV939. The Src inhibitor includes, but is not limited to, A419259. The EGFR inhibitor includes, but is not limited to, AG1478.

Non-limiting examples of an agent for generating a cardiac cell from an iPSC include activin A, BMP4, Wnt3a, VEGF, soluble frizzled protein, cyclosporin A, angiotensin II, phenylephrine, ascorbic acid, dimethylsulfoxide, 5-aza-2′-deoxycytidine, and the like.

The cells provided herein can be cultured on a surface, such as a synthetic surface to support and/or promote differentiation of hypoimmunogenic pluripotent cells into cardiac cells. In some embodiments, the surface comprises a polymer material including, but not limited to, a homopolymer or copolymer of selected one or more acrylate monomers. Non-limiting examples of acrylate monomers and methacrylate monomers include tetra(ethylene glycol) diacrylate, glycerol dimethacrylate, 1,4-butanediol dimethacrylate, poly(ethylene glycol) diacrylate, di(ethylene glycol) dimethacrylate, tetra(ethyiene glycol) dimethacrylate, 1,6-hexanediol propoxylate diacrylate, neopentyl glycol diacrylate, trimethylolpropane benzoate diacrylate, trimethylolpropane eihoxylate (1 EO/QH) methyl, tricyclo[5.2.1.02,6]decane dimethanol diacrylate, neopentyl glycol ethoxylate diacrylate, and trimethylolpropane triacrylate. Acrylate synthesized as known in the art or obtained from a commercial vendor, such as Polysciences, Inc., Sigma Aldrich, Inc. and Sartomer, Inc.

The polymeric material can be dispersed on the surface of a support material. Useful support materials suitable for culturing cells include a ceramic substance, a glass, a plastic, a polymer or co-polymer, any combinations thereof, or a coating of one material on another. In some instances, a glass includes soda-lime glass, pyrex glass, vycor glass, quartz glass, silicon, or derivatives of these or the like.

In some instances, plastics or polymers including dendritic polymers include poly(vinyl chloride), poly(vinyl alcohol), poly(methyl methacrylate), poly(vinyl acetate-maleic anhydride), poly(dimethylsiloxane) monomethacrylate, cyclic olefin polymers, fluorocarbon polymers, polystyrenes, polypropylene, polyethyleneimine or derivatives of these or the like. In some instances, copolymers include poly(vinyl acetate-co-maleic anhydride), poly(styrene-co-maleic anhydride), poly(ethylene-co-acrylic acid) or derivatives of these or the like.

The efficacy of cardiac cells prepared as described herein can be assessed in animal models for cardiac cryoinjury, which causes 55% of the left ventricular wall tissue to become scar tissue without treatment (Li et al, Ann. Thorac. Surg. 62:654, 1996; Sakai et al, Ann. Thorac. Surg. 8:2074, 1999, Sakai et al., Thorac. Cardiovasc. Surg. 118:715, 1999). Successful treatment can reduce the area of the scar, limit scar expansion, and improve heart function as determined by systolic, diastolic, and developed pressure. Cardiac injury can also be modeled using an embolization coil in the distal portion of the left anterior descending artery (Watanabe et al., Cell Transplant. 7:239, 1998), and efficacy of treatment can be evaluated by histology and cardiac function.

In some embodiments, the administration comprises implantation into the subject's heart tissue, intravenous injection, intraarterial injection, intracoronary injection, intramuscular injection, intraperitoneal injection, intramyocardial injection, trans-endocardial injection, trans-epicardial injection, or infusion.

In some embodiments, the patient administered the engineered cardiac cells is also administered a cardiac drug. Illustrative examples of cardiac drugs that are suitable for use in combination therapy include, but are not limited to, growth factors, polynucleotides encoding growth factors, angiogenic agents, calcium channel blockers, antihypertensive agents, antimitotic agents, inotropic agents, anti-atherogenic agents, anti-coagulants, beta-blockers, anti-arhythmic agents, anti-inflammatory agents, vasodilators, thrombolytic agents, cardiac glycosides, antibiotics, antiviral agents, antifungal agents, agents that inhibit protozoans, nitrates, angiotensin converting enzyme (ACE) inhibitors, angiotensin II receptor antagonist, brain natriuretic peptide (BNP); antineoplastic agents, steroids, and the like.

The effects of therapy according to the methods provided herein can be monitored in a variety of ways. For instance, an electrocardiogram (ECG) or holier monitor can be utilized to determine the efficacy of treatment. An ECG is a measure of the heart rhythms and electrical impulses, and is a very effective and non-invasive way to determine if therapy has improved or maintained, prevented, or slowed degradation of the electrical conduction in a subject's heart. The use of a holier monitor, a portable ECG that can be worn for long periods of time to monitor heart abnormalities, arrhythmia disorders, and the like, is also a reliable method to assess the effectiveness of therapy. An ECG or nuclear study can be used to determine improvement in ventricular function.

### 2. Neural Cells

Provided herein are different neural cell types differentiated from hypoimmunogenic induced pluripotent stem (HIP) cells that are useful for subsequent transplantation or engraftment into recipient subjects. As will be appreciated by those in the art, the methods for differentiation depend on the desired cell type using known techniques. Exemplary neural cell types include, but are not limited to, cerebral endothelial cells, neurons (e.g., dopaminergic neurons), glial cells, and the like.

In some embodiments, differentiation of induced pluripotent stem cells is performed by exposing or contacting cells to specific factors which are known to produce a specific cell lineage(s), so as to target their differentiation to a specific, desired lineage and/or cell type of interest. In some embodiments, terminally differentiated cells display specialized phenotypic characteristics or features. In many embodiments, the stem cells described herein are differentiated into a neuroectodermal, neuronal, neuroendocrine, dopaminergic, cholinergic, serotonergic (5-HT), glutamatergic, GABAergic, adrenergic, noradrenergic, sympathetic neuronal, parasympathetic neuronal, sympathetic peripheral neuronal, or glial cell population. In some instances, the glial cell population includes a microglial (e.g., amoeboid, ramified, activated phagocytic, and activated non-phagocytic) cell population or a macroglial (central nervous system cell: astrocyte, oligodendrocyte, ependymal cell, and radial glia; and peripheral nervous system cell: Schwann cell and satellite cell) cell population, or the precursors and progenitors of any of the preceding cells.

Protocols for generating different types of neural cells are described in PCT Application No. WO2010144696, U.S. Pat. Nos. 9,057,053; 9,376,664; and 10,233,422. Additional descriptions of methods for differentiating hypoimmunogenic pluripotent cells can be found, for example, in Deuse et al., Nature Biotechnology, 2019, 37, 252-258 and Han et al., Proc Natl Acad Sci USA, 2019, 116(21), 10441-10446. Methods for determining the effect of neural cell transplantation in an animal model of a neurological disorder or condition are described in the following references: for spinal cord injury—Curtis et al., Cell Stem Cell, 2018, 22, 941-950; for Parkinson's disease—Kikuchi et al., Nature, 2017, 548:592-596; for ALS—Izrael et al., Stem Cell Research, 2018, 9(1):152 and Izrael et al., IntechOpen, DOI: 10.5772/intechopen. 72862; for epilepsy—Upadhya et al., PNAS, 2019, 116(1):287-296

### 3. Cerebral Endothelial Cells

In some embodiments, neural cells are administered to a subject to treat Parkinson's disease, Huntington disease, multiple sclerosis, other neurodegenerative disease or condition, attention deficit hyperactivity disorder (ADHD), Tourette Syndrome (TS), schizophrenia, psychosis, depression, other neuropsychiatric disorder. In some embodiments, neural cells described herein are administered to a subject to treat or ameliorate stroke. In some embodiments, the neurons and glial cells are administered to a subject with amyotrophic lateral sclerosis (ALS). In some embodiments, cerebral endothelial cells are administered to alleviate the symptoms or effects of cerebral hemorrhage. In some embodiments, dopaminergic neurons are administered to a patient with Parkinson's disease. In some embodiments, noradrenergic neurons, GABAergic interneurons are administered to a patient who has experienced an epileptic seizure. In some embodiments, motor neurons, interneurons, Schwann cells, oligodendrocytes, and microglia are administered to a patient who has experienced a spinal cord injury.

In some embodiments, cerebral endothelial cells (ECs), precursors, and progenitors thereof are differentiated from pluripotent stem cells (e.g., induced pluripotent stem cells) on a surface by culturing the cells in a medium comprising one or more factors that promote the generation of cerebral ECs or neural cell. In some instances, the medium includes one or more of the following: CHIR-99021, VEGF, basic FGF (bFGF), and Y-27632. In some embodiments, the medium includes a supplement designed to promote survival and functionality for neural cells.

In some embodiments, cerebral endothelial cells (ECs), precursors, and progenitors thereof are differentiated from pluripotent stem cells on a surface by culturing the cells in an unconditioned or conditioned medium. In some instances, the medium comprises factors or small molecules that promote or facilitate differentiation. In some embodiments, the medium comprises one or more factors or small molecules selected from the group consisting of VEGR, FGF, SDF-1, CHIR-99021, Y-27632, SB 431542, and any combination thereof. In some embodiments, the surface for differentiation comprises one or more extracellular matrix proteins. The surface can be coated with the one or more extracellular matrix proteins. The cells can be differentiated in suspension and then put into a gel matrix form, such as matrigel, gelatin, or fibrin/thrombin forms to facilitate cell survival. In some cases, differentiation is assayed as is known in the art, generally by evaluating the presence of cell-specific markers.

In some embodiments, the cerebral endothelial cells express or secrete a factor selected from the group consisting of CD31, VE cadherin, and a combination thereof. In many embodiments, the cerebral endothelial cells express or secrete one or more of the factors selected from the group consisting of CD31, CD34, CD45, CD117 (c-kit), CD146, CXCR4, VEGF, SDF-1, PDGF, GLUT-1, PECAM-1, eNOS, claudin-5, occludin, ZO-1, p-glycoprotein, von Willebrand factor, VE-cadherin, low density lipoprotein receptor LDLR, low density lipoprotein receptor-related protein 1 LRP1, insulin receptor INSR, leptin receptor LEPR, basal cell adhesion molecule BCAM, transferrin receptor TFRC, advanced glycation endproduct-specific receptor AGER, receptor for retinol uptake STRA6, large neutral amino acids transporter small subunit 1 SLC7A5, excitatory amino acid transporter 3 SLC1A1, sodium-coupled neutral amino acid transporter 5 SLC38A5, solute carrier family 16 member 1 SLC16A1, ATP-dependent translocase ABCB1, ATP-ABCC2-binding cassette transporter ABCG2, multidrug resistance-associated protein 1 ABCC1, canalicular multispecific organic anion transporter 1 ABCC2, multidrug resistance-associated protein 4 ABCC4, and multidrug resistance-associated protein 5 ABCC5.

In some embodiments, the cerebral ECs are characterized with one or more of the features selected from the group consisting of high expression of tight junctions, high electrical resistance, low fenestration, small perivascular space, high prevalence of insulin and transferrin receptors, and high number of mitochondria.

In some embodiments, cerebral ECs are selected or purified using a positive selection strategy. In some instances, the cerebral ECs are sorted against an endothelial cell marker such as, but not limited to, CD31. In other words, CD31 positive cerebral ECs are isolated. In some embodiments, cerebral ECs are selected or purified using a negative selection strategy. In some embodiments, undifferentiated or pluripotent stem cells are removed by selecting for cells that express a pluripotency marker including, but not limited to, TRA-1-60 and SSEA-1.

### 4. Dopaminergic Neurons

In some embodiments, hypoimmunogenic induced pluripotent stem (HIP)cells described herein are differentiated into dopaminergic neurons include neuronal stem cells, neuronal progenitor cells, immature dopaminergic neurons, and mature dopaminergic neurons.

In some cases, the term “dopaminergic neurons” includes neuronal cells which express tyrosine hydroxylase (TH), the rate-limiting enzyme for dopamine synthesis. In some embodiments, dopaminergic neurons secrete the neurotransmitter dopamine, and have little or no expression of dopamine hydroxylase. A dopaminergic (DA) neuron can express one or more of the following markers: neuron-specific enolase (NSE), 1-aromatic amino acid decarboxylase, vesicular monoamine transporter 2, dopamine transporter, Nurr-1, and dopamine-2 receptor (D2 receptor). In certain cases, the term “neural stem cells” includes a population of pluripotent cells that have partially differentiated along a neural cell pathway and express one or more neural markers including, for example, nestin. Neural stem cells may differentiate into neurons or glial cells (e.g., astrocytes and oligodendrocytes). The term “neural progenitor cells” includes cultured cells which express FOXA2 and low levels of b-tubulin, but not tyrosine hydroxylase. Such neural progenitor cells have the capacity to differentiate into a variety of neuronal subtypes; particularly a variety of dopaminergic neuronal subtypes, upon culturing the appropriate factors, such as those described herein.

In some embodiments, the DA neurons derived from hypoimmunogenic induced pluripotent stem (HIP) cells are administered to a patient, e.g., human patient to treat a neurodegenerative disease or condition. In some cases, the neurodegenerative disease or condition is selected from the group consisting of Parkinson's disease, Huntington disease, and multiple sclerosis. In other embodiments, the DA neurons are used to treat or ameliorate one or more symptoms of a neuropsychiatric disorder, such as attention deficit hyperactivity disorder (ADHD), Tourette Syndrome (TS), schizophrenia, psychosis, and depression. In yet other embodiments, the DA neurons are used to treat a patient with impaired DA neurons.

In some embodiments, DA neurons, precursors, and progenitors thereof are differentiated from pluripotent stem cells by culturing the stem cells in medium comprising one or more factors or additives. Useful factors and additives that promote differentiation, growth, expansion, maintenance, and/or maturation of DA neurons include, but are not limited to, Wntl, FGF2, FGF8, FGF8a, sonic hedgehog (SHH), brain derived neurotrophic factor (BDNF), transforming growth factor a (TGF-a), TGF-b, interleukin 1 beta, glial cell line-derived neurotrophic factor (GDNF), a GSK-3 inhibitor (e.g., CHIR-99021), a TGF-b inhibitor (e.g., SB-431542), B-27 supplement, dorsomorphin, purmorphamine, noggin, retinoic acid, cAMP, ascorbic acid, neurturin, knockout serum replacement, N-acetyl cysteine, c-kit ligand, modified forms thereof, mimics thereof, analogs thereof, and variants thereof. In some embodiments, the DA neurons are differentiated in the presence of one or more factors that activate or inhibit the WNT pathway, NOTCH pathway, SHH pathway, BMP pathway, FGF pathway, and the like. Differentiation protocols and detailed descriptions thereof are provided in, e.g., U.S. Pat. Nos. 9,968,637, 7,674,620, Kim et al, Nature, 2002, 418,50-56; Bjorklund et al, PNAS, 2002, 99(4), 2344-2349; Grow et al., Stem Cells Transl Med. 2016, 5(9): 1133-44, and Cho et al, PNAS, 2008, 105:3392-3397, the disclosures in their entirety including the detailed description of the examples, methods, figures, and results are herein incorporated by reference.

In some embodiments, the population of hypoimmunogenic dopaminergic neurons is isolated from non-neuronal cells. In some embodiments, the isolated population of hypoimmunogenic dopaminergic neurons are expanded prior to administration. In many embodiments, the isolated population of hypoimmunogenic dopaminergic neurons are expanded and cryopreserved prior to administration.

To characterize and monitor DA differentiation and assess the DA phenotype, expression of any number of molecular and genetic markers can be evaluated. For example, the presence of genetic markers can be determined by various methods known to those skilled in the art. Expression of molecular markers can be determined by quantifying methods such as, but not limited to, qPCR-based assays, immunoassays, immunocytochemistry assays, immunoblotting assays, and the like. Exemplary markers for DA neurons include, but are not limited to, TH, b-tubulin, paired box protein (Pax6), insulin gene enhancer protein (Isl1), nestin, diaminobenzidine (DAB), G protein-activated inward rectifier potassium channel 2 (GIRK2), microtubule-associated protein 2 (MAP-2), NURR1, dopamine transporter (DAT), forkhead box protein A2 (FOXA2), FOX3, doublecortin, and LIM homeobox transcription factor 1-beta (LMX1B), and the like. In some embodiments, the DA neurons express one or more of the markers selected from corin, FOXA2, TuJ1, NURR1, and any combination thereof.

In some embodiments, DA neurons are assessed according to cell electrophysiological activity. The electrophysiology of the cells can be evaluated by using assays knowns to those skilled in the art. For instance, whole-cell and perforated patch clamp, assays for detecting electrophysiological activity of cells, assays for measuring the magnitude and duration of action potential of cells, and functional assays for detecting dopamine production of DA cells.

In some embodiments, DA neuron differentiation is characterized by spontaneous rhythmic action potentials, and high-frequency action potentials with spike frequency adaption upon injection of depolarizing current. In other embodiments, DA differentiation is characterized by the production of dopamine. The level of dopamine produced is calculated by measuring the width of an action potential at the point at which it has reached half of its maximum amplitude (spike half-maximal width).

In some embodiments, the differentiated DA neurons are transplanted either intravenously or by injection at particular locations in the patient. In some embodiments, the differentiated DA cells are transplanted into the substantia nigra (particularly in or adjacent of the compact region), the ventral tegmental area (VTA), the caudate, the putamen, the nucleus accumbens, the subthalamic nucleus, or any combination thereof, of the brain to replace the DA neurons whose degeneration resulted in Parkinson's disease. The differentiated DA cells can be injected into the target area as a cell suspension. Alternatively, the differentiated DA cells can be embedded in a support matrix or scaffold when contained in such a delivery device. In some embodiments, the scaffold is biodegradable. In other embodiments, the scaffold is not biodegradable. The scaffold can comprise natural or synthetic (artificial) materials.

The delivery of the DA neurons can be achieved by using a suitable vehicle such as, but not limited to, liposomes, microparticles, or microcapsules. In other embodiments, the differentiated DA neurons are administered in a pharmaceutical composition comprising an isotonic excipient. The pharmaceutical composition is prepared under conditions that are sufficiently sterile for human administration. In some embodiments, the DA neurons differentiated from HIP cells are supplied in the form of a pharmaceutical composition. General principles of therapeutic formulations of cell compositions are found in Cell Therapy: Stem Cell Transplantation, Gene Therapy, and Cellular Immunotherapy, G. Morstyn & W. Sheridan eds, Cambridge University Press, 1996, and Hematopoietic Stem Cell Therapy, E. Ball, J. Lister & P. Law, Churchill Livingstone, 2000, the disclosures are incorporated herein by reference.

Useful descriptions of neurons derived from stem cells and methods of making thereof can be found, for example, in Kirkeby et al., Cell Rep, 2012, 1:703-714; Kriks et al., Nature, 2011, 480:547-551; Wang et al., Stem Cell Reports, 2018, 11(1): 171-182; Lorenz Studer, “Chapter 8—Strategies for Bringing Stem Cell-Derived Dopamine Neurons to the clinic—The NYSTEM Trial” in Progress in Brain Research, 2017, volume 230, pg. 191-212; Liu et al., Nat Protoc, 2013, 8:1670-1679; Upadhya et al., Curr Protoc Stem Cell Biol, 38, 2D.7.1-2D.7.47; US Publication Appl. No. 20160115448, and U.S. Pat. Nos. 8,252,586; 8,273,570; 9,487,752 and 10,093,897, the contents are incorporated herein by reference in their entirety.

In addition to DA neurons, other neuronal cells, precursors, and progenitors thereof can be differentiated from the HIP cells outlined herein by culturing the cells in medium comprising one or more factors or additive. Non-limiting examples of factors and additives include GDNF, BDNF, GM-CSF, B27, basic FGF, basic EGF, NGF, CNTF, SMAD inhibitor, Wnt antagonist, SHH signaling activator, and any combination thereof. In some embodiments, the SMAD inhibitor is selected from the group consisting of SB431542, LDN-193189, Noggin PD169316, SB203580, LY364947, A77-01, A-83-01, BMP4, GW788388, GW6604, SB-505124, lerdelimumab, metelimumab, GC-1008, AP-12009, AP-11014, LY550410, LY580276, LY364947, LY2109761, SB-505124, E-616452 (RepSox ALK inhibitor), SD-208, SMI6, NPC-30345, K 26894, SB-203580, SD-093, activin-M108A, P144, soluble TBR2-Fc, DMH-1, dorsomorphin dihydrochloride and derivatives thereof. In some embodiments, the Wnt antagonist is selected from the group consisting of XAV939, DKK1, DKK-2, DKK-3, DKK-4, SFRP-1, SFRP-2, SFRP-3, SFRP-4, SFRP-5, WIF-1, Soggy, IWP-2, IWR1, ICG-001, KY0211, Wnt-059, LGK974, IWP-L6 and derivatives thereof. In some embodiments, the SHH signaling activator is selected from the group consisting of Smoothened agonist (SAG), SAG analog, SHH, C25-SHH, C24-SHH, purmorphamine, Hg—Ag and/or derivatives thereof.

In some embodiments, the neurons express one or more of the markers selected from the group consisting of glutamate ionotropic receptor NMDA type subunit 1 GRIN1, glutamate decarboxylase 1 GAD1, gamma-aminobutyric acid GABA, tyrosine hydroxylase TH, LIM homeobox transcription factor 1-alpha LMX1A, Forkhead box protein O1 FOXO1, Forkhead box protein A2 FOXA2, Forkhead box protein O4 FOX04, FOXG1, 2′,3′-cyclic-nucleotide 3′-phosphodiesterase CNP, myelin basic protein MBP, tubulin beta chain 3 TUB3, tubulin beta chain 3 NEUN, solute carrier family 1 member 6 SLC1A6, SST, PV, calbindin, RAX, LHX6, LHX8, DLX1, DLX2, DLX5, DLX6, SOX6, MAFB, NPAS1, ASCL1, SIX6, OLIG2, NKX2.1, NKX2.2, NKX6.2, VGLUT1, MAP2, CTIP2, SATB2, TBR1, DLX2, ASCL1, ChAT, NGFI-B, c-fos, CRF, RAX, POMC, hypocretin, NADPH, NGF, Ach, VAChT, PAX6, EMX2p75, CORIN, TUJ1, NURR1, and/or any combination thereof.

### 5. Glial Cells

In some embodiments, the neural cells described include glial cells such as, but not limited to, microglia, astrocytes, oligodendrocytes, ependymal cells and Schwann cells, glial precursors, and glial progenitors thereof are produced by differentiating pluripotent stem cells into therapeutically effective glial cells and the like. Differentiation of hypoimmunogenic pluripotent stem cells produces hypoimmunogenic neural cells, such as hypoimmunogenic glial cells.

In some embodiments, glial cells, precursors, and progenitors thereof generated by culturing pluripotent stem cells in medium comprising one or more agents selected from the group consisting of retinoic acid, IL-34, M-CSF, FLT3 ligand, GM-CSF, CCL2, a TGFbeta inhibitor, a BMP signaling inhibitor, a SHH signaling activator, FGF, platelet derived growth factor PDGF, PDGFR-alpha, HGF, IGF1, noggin, SHH, dorsomorphin, noggin, and any combination thereof. In certain instances, the BMP signaling inhibitor is LDN193189, SB431542, or a combination thereof. In some embodiments, the glial cells express NKX2.2, PAX6, SOX10, brain derived neurotrophic factor BDNF, neutrotrophin-3 NT-3, NT-4, EGF, ciliary neurotrophic factor CNTF, nerve growth factor NGF, FGF8, EGFR, OLIG1, OLIG2, myelin basic protein MBP, GAP-43, LNGFR, nestin, GFAP, CD11b, CD11c, CX3CR1, P2RY12, IBA-1, TMEM119, CD45, and any combination thereof. Exemplary differentiation medium can include any specific factors and/or small molecules that may facilitate or enable the generation of a glial cell type as recognized by those skilled in the art.

To determine if the cells generated according to the in vitro differentiation protocol display glial cell characteristics and features, the cells can be transplanted into an animal model. In some embodiments, the glial cells are injected into an immunocompromised mouse, e.g., an immunocompromised shiverer mouse. The glial cells are administered to the brain of the mouse and after a pre-selected amount of time the engrafted cells are evaluated. In some instances, the engrafted cells in the brain are visualized by using immunostaining and imaging methods. In some embodiments, it is determined that the glial cells express known glial cell biomarkers.

Useful methods for generating glial cells, precursors, and progenitors thereof from stem cells are found, for example, in U.S. Pat. Nos. 7,579,188; 7,595,194; 8,263,402; 8,206,699; 8,252,586; 9,193,951; 9,862,925; 8,227,247; 9,709,553; US2018/0187148; US2017/0198255; US2017/0183627; US2017/0182097; US2017/253856; US2018/0236004; WO2017/172976; and WO2018/093681. Methods for differentiating pluripotent stem cells are described in, e.g., Kikuchi et al., Nature, 2017, 548, 592-596; Kriks et al., Nature, 2011, 547-551; Doi et al., Stem Cell Reports, 2014, 2, 337-50; Perrier et al., Proc Natl Acad Sci USA, 2004, 101, 12543-12548; Chambers et al., Nat Biotechnol, 2009, 27, 275-280; and Kirkeby et al., Cell Reports, 2012, 1, 703-714.

The efficacy of neural cell transplants for spinal cord injury can be assessed in, for example, a rat model for acutely injured spinal cord, as described by McDonald, et al., Nat. Med., 1999, 5:1410) and Kim, et al., Nature, 2002, 418:50. For instance, successful transplants may show transplant-derived cells present in the lesion 2-5 weeks later, differentiated into astrocytes, oligodendrocytes, and/or neurons, and migrating along the spinal cord from the lesioned end, and an improvement in gait, coordination, and weight-bearing. Specific animal models are selected based on the neural cell type and neurological disease or condition to be treated.

The neural cells can be administered in a manner that permits them to engraft to the intended tissue site and reconstitute or regenerate the functionally deficient area. For instance, neural cells can be transplanted directly into parenchymal or intrathecal sites of the central nervous system, according to the disease being treated. In some embodiments, any of the neural cells described herein including cerebral endothelial cells, neurons, dopaminergic neurons, ependymal cells, astrocytes, microglial cells, oligodendrocytes, and Schwann cells are injected into a patient by way of intravenous, intraspinal, intracerebroventricular, intrathecal, intraarterial, intramuscular, intraperitoneal, subcutaneous, intramuscular, intra-abdominal, intraocular, retrobulbar and combinations thereof. In some embodiments, the cells are injected or deposited in the form of a bolus injection or continuous infusion. In many embodiments, the neural cells are administered by injection into the brain, apposite the brain, and combinations thereof. The injection can be made, for example, through a burr hole made in the subject's skull. Suitable sites for administration of the neural cell to the brain include, but are not limited to, the cerebral ventricle, lateral ventricles, cisterna magna, putamen, nucleus basalis, hippocampus cortex, striatum, caudate regions of the brain and combinations thereof.

Additional descriptions of neural cells including dopaminergic neurons for use in the present technology are found in WO2020/018615, the disclosure is herein incorporated by reference in its entirety.

### 6. Endothelial Cells

Provided herein are hypoimmunogenic pluripotent cells that are differentiated into various endothelial cell types for subsequent transplantation or engraftment into subjects (e.g., recipients). As will be appreciated by those in the art, the methods for differentiation depend on the desired cell type using known techniques.

In some embodiments, the endothelial cells differentiated from the subject hypoimmunogenic pluripotent cells are administered to a patient, e.g., a human patient in need thereof. The endothelial cells can be administered to a patient suffering from a disease or condition such as, but not limited to, cardiovascular disease, vascular disease, peripheral vascular disease, ischemic disease, myocardial infarction, congestive heart failure, peripheral vascular obstructive disease, stroke, reperfusion injury, limb ischemia, neuropathy (e.g., peripheral neuropathy or diabetic neuropathy), organ failure (e.g., liver failure, kidney failure, and the like), diabetes, rheumatoid arthritis, osteoporosis, vascular injury, tissue injury, hypertension, angina pectoris and myocardial infarction due to coronary artery disease, renal vascular hypertension, renal failure due to renal artery stenosis, claudication of the lower extremities, and the like. In many embodiments, the patient has suffered from or is suffering from a transient ischemic attack or stroke, which in some cases, may be due to cerebrovascular disease. In some embodiments, the engineered endothelial cells are administered to treat tissue ischemia e.g., as occurs in atherosclerosis, myocardial infarction, and limb ischemia and to repair of injured blood vessels. In some instances, the cells are used in bioengineering of grafts.

For instance, the endothelial cells can be used in cell therapy for the repair of ischemic tissues, formation of blood vessels and heart valves, engineering of artificial vessels, repair of damaged vessels, and inducing the formation of blood vessels in engineered tissues (e.g., prior to transplantation). Additionally, the endothelial cells can be further modified to deliver agents to target and treat tumors.

In many embodiments, provided herein is a method of repair or replacement for tissue in need of vascular cells or vascularization. The method involves administering to a human patient in need of such treatment, a composition containing the isolated endothelial cells to promote vascularization in such tissue. The tissue in need of vascular cells or vascularization can be a cardiac tissue, liver tissue, pancreatic tissue, renal tissue, muscle tissue, neural tissue, bone tissue, among others, which can be a tissue damaged and characterized by excess cell death, a tissue at risk for damage, or an artificially engineered tissue.

In some embodiments, vascular diseases, which may be associated with cardiac diseases or disorders can be treated by administering endothelial cells, such as but not limited to, definitive vascular endothelial cells and endocardial endothelial cells derived as described herein. Such vascular diseases include, but are not limited to, coronary artery disease, cerebrovascular disease, aortic stenosis, aortic aneurysm, peripheral artery disease, atherosclerosis, varicose veins, angiopathy, infarcted area of heart lacking coronary perfusion, non-healing wounds, diabetic or non-diabetic ulcers, or any other disease or disorder in which it is desirable to induce formation of blood vessels.

In many embodiments, the endothelial cells are used for improving prosthetic implants (e.g., vessels made of synthetic materials such as Dacron and Gortex.) which are used in vascular reconstructive surgery. For example, prosthetic arterial grafts are often used to replace diseased arteries which perfuse vital organs or limbs. In other embodiments, the engineered endothelial cells are used to cover the surface of prosthetic heart valves to decrease the risk of the formation of emboli by making the valve surface less thrombogenic.

The endothelial cells outlined can be transplanted into the patient using well known surgical techniques for grafting tissue and/or isolated cells into a vessel. In some embodiments, the cells are introduced into the patient's heart tissue by injection (e.g., intramyocardial injection, intracoronary injection, trans-endocardial injection, trans-epicardial injection, percutaneous injection), infusion, grafting, and implantation.

Administration (delivery) of the endothelial cells includes, but is not limited to, subcutaneous or parenteral including intravenous, intraarterial (e.g., intracoronary), intramuscular, intraperitoneal, intramyocardial, trans-endocardial, trans-epicardial, intranasal administration as well as intrathecal, and infusion techniques.

As will be appreciated by those in the art, the HIP derivatives are transplanted using techniques known in the art that depends on both the cell type and the ultimate use of these cells. In some embodiments, the cells differentiated from the subject HIPs provided herein are transplanted either intravenously or by injection at particular locations in the patient. When transplanted at particular locations, the cells may be suspended in a gel matrix to prevent dispersion while they take hold.

Exemplary endothelial cell types include, but are not limited to, a capillary endothelial cell, vascular endothelial cell, aortic endothelial cell, arterial endothelial cell, venous endothelial cell, renal endothelial cell, brain endothelial cell, liver endothelial cell, and the like.

The endothelial cells outlined herein can express one or more endothelial cell markers. Non-limiting examples of such markers include VE-cadherin (CD 144), ACE (angiotensin-converting enzyme) (CD 143), BNH9/BNF13, CD31, CD34, CD54 (ICAM-1), CD62E (E-Selectin), CD105 (Endoglin), CD146, Endocan (ESM-1), Endoglyx-1, Endomucin, Eotaxin-3, EPAS1 (Endothelial PAS domain protein 1), Factor VIII related antigen, FLI-1, Flk-1 (KDR, VEGFR-2), FLT-1 (VEGFR-1), GATA2, GBP-1 (guanylate-binding protein-1), GRO-alpha, HEX, ICAM-2 (intercellular adhesion molecule 2), LM02, LYVE-1, MRB (magic roundabout), Nucleolin, PAL-E (pathologische anatomie Leiden-endothelium), RTKs, sVCAM-1, TALI, TEM1 (Tumor endothelial marker 1), TEM5 (Tumor endothelial marker 5), TEM7 (Tumor endothelial marker 7), thrombomodulin (TM, CD141), VCAM-1 (vascular cell adhesion molecule-1) (CD106), VEGF, vWF (von Willebrand factor), ZO-1, endothelial cell-selective adhesion molecule (ESAM), CD102, CD93, CD184, CD304, and DLL4.

In some embodiments, the endothelial cells are genetically modified to express an exogenous gene encoding a protein of interest such as but not limited to an enzyme, hormone, receptor, ligand, or drug that is useful for treating a disorder/condition or ameliorating symptoms of the disorder/condition. Standard methods for genetically modifying endothelial cells are described, e.g., in U.S. Pat. No. 5,674,722.

Such endothelial cells can be used to provide constitutive synthesis and delivery of polypeptides or proteins, which are useful in prevention or treatment of disease. In this way, the polypeptide is secreted directly into the bloodstream or other area of the body (e.g., central nervous system) of the individual. In some embodiments, the endothelial cells can be modified to secrete insulin, a blood clotting factor (e.g., Factor VIII or von Willebrand Factor), alpha-1 antitrypsin, adenosine deaminase, tissue plasminogen activator, interleukins (e.g., IL-1, IL-2, IL-3), and the like.

In many embodiments, the endothelial cells can be modified in a way that improves their performance in the context of an implanted graft. Non-limiting illustrative examples include secretion or expression of a thrombolytic agent to prevent intraluminal clot formation, secretion of an inhibitor of smooth muscle proliferation to prevent luminal stenosis due to smooth muscle hypertrophy, and expression and/or secretion of an endothelial cell mitogen or autocrine factor to stimulate endothelial cell proliferation and improve the extent or duration of the endothelial cell lining of the graft lumen.

In some embodiments, the engineered endothelial cells are utilized for delivery of therapeutic levels of a secreted product to a specific organ or limb. For example, a vascular implant lined with endothelial cells engineered (transduced) in vitro can be grafted into a specific organ or limb. The secreted product of the transduced endothelial cells will be delivered in high concentrations to the perfused tissue, thereby achieving a desired effect to a targeted anatomical location.

In other embodiments, the endothelial cells are genetically modified to contain a gene that disrupts or inhibits angiogenesis when expressed by endothelial cells in a vascularizing tumor. In some cases, the endothelial cells can also be genetically modified to express any one of the selectable suicide genes described herein which allows for negative selection of grafted endothelial cells upon completion of tumor treatment.

In some embodiments, endothelial cells described herein are administered to a recipient subject to treat a vascular disorder selected from the group consisting of vascular injury, cardiovascular disease, vascular disease, peripheral vascular disease, ischemic disease, myocardial infarction, congestive heart failure, peripheral vascular obstructive disease, hypertension, ischemic tissue injury, reperfusion injury, limb ischemia, stroke, neuropathy (e.g., peripheral neuropathy or diabetic neuropathy), organ failure (e.g., liver failure, kidney failure, and the like), diabetes, rheumatoid arthritis, osteoporosis, cerebrovascular disease, hypertension, angina pectoris and myocardial infarction due to coronary artery disease, renal vascular hypertension, renal failure due to renal artery stenosis, claudication of the lower extremities, other vascular condition or disease.

In some embodiments, the hypoimmunogenic pluripotent cells are differentiated into endothelial colony forming cells (ECFCs) to form new blood vessels to address peripheral arterial disease. Techniques to differentiate endothelial cells are known. See, e.g., Prasain et al., doi: 10.1038/nbt.3048, incorporated herein by reference in its entirety and specifically for the methods and reagents for the generation of endothelial cells from human pluripotent stem cells, and also for transplantation techniques. Differentiation can be assayed as is known in the art, generally by evaluating the presence of endothelial cell associated or specific markers or by measuring functionally.

In some embodiments, the method of producing a population of hypoimmunogenic endothelial cells from a population of hypoimmunogenic induced pluripotent stem (HIP) cells by in vitro differentiation comprises: (a) culturing a population of HIP cells in a first culture medium comprising a GSK inhibitor; (b) culturing the population of HIP cells in a second culture medium comprising VEGF and bFGF to produce a population of pre-endothelial cells; and (c) culturing the population of pre-endothelial cells in a third culture medium comprising a ROCK inhibitor and an ALK inhibitor to produce a population of hypoimmunogenic endothelial cells.

In some embodiments, the GSK inhibitor is CHIR-99021, a derivative thereof, or a variant thereof. In some instances, the GSK inhibitor is at a concentration ranging from about 1 mM to about 10 mM. In some embodiments, the ROCK inhibitor is Y-27632, a derivative thereof, or a variant thereof. In some instances, the ROCK inhibitor is at a concentration ranging from about 1 pM to about 20 pM. In some embodiments, the ALK inhibitor is SB-431542, a derivative thereof, or a variant thereof. In some instances, the ALK inhibitor is at a concentration ranging from about 0.5 pM to about 10 pM.

In some embodiments, the first culture medium comprises from 2 pM to about 10 pM of CHIR-99021. In some embodiments, the second culture medium comprises 50 ng/ml VEGF and 10 ng/ml bFGF. In other embodiments, the second culture medium further comprises Y-27632 and SB-431542. In various embodiments, the third culture medium comprises 10 pM Y-27632 and 1 pM SB-431542. In many embodiments, the third culture medium further comprises VEGF and bFGF. In particular instances, the first culture medium and/or the second medium is absent of insulin.

The cells provided herein can be cultured on a surface, such as a synthetic surface to support and/or promote differentiation of hypoimmunogenic pluripotent cells into cardiac cells. In some embodiments, the surface comprises a polymer material including, but not limited to, a homopolymer or copolymer of selected one or more acrylate monomers. Non-limiting examples of acrylate monomers and methacrylate monomers include tetra(ethylene glycol) diacrylate, glycerol dimethacrylate, 1,4-butanediol dimethacrylate, poly(ethylene glycol) diacrylate, di(ethylene glycol) dimethacrylate, tetra(ethyiene glycol) dimethacrylate, 1,6-hexanediol propoxylate diacrylate, neopentyl glycol diacrylate, trimethylolpropane benzoate diacrylate, trimethylolpropane eihoxylate (1 EO/QH) methyl, tricyclo[5.2.1.02,6]decane dimethanol diacrylate, neopentyl glycol exhoxylate diacrylate, and trimethylolpropane triacrylate. Acrylate synthesized as known in the art or obtained from a commercial vendor, such as Polysciences, Inc., Sigma Aldrich, Inc. and Sartomer, Inc.

In some embodiments, the endothelial cells may be seeded onto a polymer matrix. In some cases, the polymer matrix is biodegradable. Suitable biodegradable matrices are well known in the art and include collagen-GAG, collagen, fibrin, PLA, PGA, and PLA/PGA copolymers. Additional biodegradable materials include poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes and polysaccharides.

Non-biodegradable polymers may also be used as well. Other non-biodegradable, yet biocompatible polymers include polypyrrole, polyanibnes, polythiophene, polystyrene, polyesters, non-biodegradable polyurethanes, polyureas, poly(ethylene vinyl acetate), polypropylene, polymethacrylate, polyethylene, polycarbonates, and poly(ethylene oxide). The polymer matrix may be formed in any shape, for example, as particles, a sponge, a tube, a sphere, a strand, a coiled strand, a capillary network, a film, a fiber, a mesh, or a sheet. The polymer matrix can be modified to include natural or synthetic extracellular matrix materials and factors.

The polymeric material can be dispersed on the surface of a support material. Useful support materials suitable for culturing cells include a ceramic substance, a glass, a plastic, a polymer or co-polymer, any combinations thereof, or a coating of one material on another. In some instances, a glass includes soda-lime glass, pyrex glass, vycor glass, quartz glass, silicon, or derivatives of these or the like.

In some instances, plastics or polymers including dendritic polymers include poly(vinyl chloride), poly(vinyl alcohol), poly(methyl methacrylate), poly(vinyl acetate-maleic anhydride), poly(dimethylsiloxane) monomethacrylate, cyclic olefin polymers, fluorocarbon polymers, polystyrenes, polypropylene, polyethyleneimine or derivatives of these or the like. In some instances, copolymers include poly(vinyl acetate-co-maleic anhydride), poly(styrene-co-maleic anhydride), poly(ethylene-co-acrylic acid) or derivatives of these or the like.

In some embodiments, the population of hypoimmunogenic endothelial cells is isolated from non-endothelial cells. In some embodiments, the isolated population of hypoimmunogenic endothelial cells are expanded prior to administration. In many embodiments, the isolated population of hypoimmunogenic endothelial cells are expanded and cryopreserved prior to administration.

Additional descriptions of endothelial cells for use in the methods provided herein are found in WO2020/018615, the disclosure is herein incorporated by reference in its entirety.

### 7. Thyroid Cells

In some embodiments, the hypoimmunogenic pluripotent cells are differentiated into thyroid progenitor cells and thyroid follicular organoids that can secrete thyroid hormones to address autoimmune thyroiditis. Techniques to differentiate thyroid cells are known the art. See, e.g., Kurmann et al., Cell Stem Cell, Nov. 5, 2015; 17(5):527-42, incorporated herein by reference in its entirety and specifically for the methods and reagents for the generation of thyroid cells from human pluripotent stem cells, and also for transplantation techniques. Differentiation can be assayed as is known in the art, generally by evaluating the presence of thyroid cell associated or specific markers or by measuring functionally.

### 8. Hepatocytes

In some embodiments, the hypoimmunogenic induced pluripotent stem (HIP) cells are differentiated into hepatocytes to address loss of the hepatocyte functioning or cirrhosis of the liver. There are a number of techniques that can be used to differentiate HIP cells into hepatocytes; see for example, Pettinato et al., doi: 10.1038/spre32888, Snykers et al., Methods Mol Biol, 2011 698:305-314, Si-Tayeb et al., Hepatology, 2010, 51:297-305 and Asgari et al, Stem Cell Rev, 2013, 9(4):493-504, all of which are incorporated herein by reference in their entirety and specifically for the methodologies and reagents for differentiation. Differentiation can be assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake, ICG uptake and release, and glycogen storage.

### 9. Pancreatic Islet Cells

In some embodiments, pancreatic islet cells (also referred to as pancreatic beta cells) are derived from the hypoimmunogenic induced pluripotent stem (HIP) cells described herein. In some instances, hypoimmunogenic pluripotent cells that are differentiated into various pancreatic islet cell types are transplanted or engrafted into subjects (e.g., recipients). As will be appreciated by those in the art, the methods for differentiation depend on the desired cell type using known techniques. Exemplary pancreatic islet cell types include, but are not limited to, pancreatic islet progenitor cell, immature pancreatic islet cell, mature pancreatic islet cell, and the like. In some embodiments, pancreatic cells described herein are administered to a subject to treat diabetes.

In some embodiments, pancreatic islet cells are derived from the hypoimmunogenic pluripotent cells described herein. Useful method for differentiating pluripotent stem cells into pancreatic islet cells are described, for example, in U.S. Pat. Nos. 9,683,215; 9,157,062; and 8,927,280.

In some embodiments, the pancreatic islet cells produced by the methods as disclosed herein secretes insulin. In some embodiments, a pancreatic islet cell exhibits at least two characteristics of an endogenous pancreatic islet cell, for example, but not limited to, secretion of insulin in response to glucose, and expression of beta cell markers.

Exemplary beta cell markers or beta cell progenitor markers include, but are not limited to, c-peptide, Pdxl, glucose transporter 2 (Glut2), HNF6, VEGF, glucokinase (GCK), prohormone convertase (PC 1/3), Cdcpl, NeuroD, Ngn3, Nkx2.2, Nkx6.1, Nkx6.2, Pax4, Pax6, Ptfla, Isll, Sox9, Sox17, and FoxA2.

In some embodiments, the isolated pancreatic islet cells produce insulin in response to an increase in glucose. In various embodiments, the isolated pancreatic islet cells secrete insulin in response to an increase in glucose. In some embodiments, the cells have a distinct morphology such as a cobblestone cell morphology and/or a diameter of about 17 pm to about 25 pm.

In some embodiments, the hypoimmunogenic pluripotent cells are differentiated into beta-like cells or islet organoids for transplantation to address type I diabetes mellitus (T1DM). Cell systems are a promising way to address T1DM, see, e.g., Ellis et al, Nat Rev Gastroenterol Hepatol. 2017 October; 14(10):612-628, incorporated herein by reference. Additionally, Pagliuca et al. (Cell, 2014, 159(2):428-39) reports on the successful differentiation of B-cells from human iPSCs, the contents incorporated herein by reference in its entirety and in particular for the methods and reagents outlined there for the large-scale production of functional human β cells from human pluripotent stem cells). Furthermore, Vegas et al. shows the production of human β cells from human pluripotent stem cells followed by encapsulation to avoid immune rejection by the host; Vegas et al., Nat Med, 2016, 22(3):306-11, incorporated herein by reference in its entirety and in particular for the methods and reagents outlined there for the large-scale production of functional human β cells from human pluripotent stem cells.

In some embodiments, the method of producing a population of hypoimmunogenic pancreatic islet cells from a population of hypoimmunogenic induced pluripotent stem (HIP) cells by in vitro differentiation comprises: (a) culturing the population of HIP cells in a first culture medium comprising one or more factors selected from the group consisting insulin-like growth factor, transforming growth factor, FGF, EGF, HGF, SHH, VEGF, transforming growth factor-b superfamily, BMP2, BMP7, a GSK inhibitor, an ALK inhibitor, a BMP type 1 receptor inhibitor, and retinoic acid to produce a population of immature pancreatic islet cells; and (b) culturing the population of immature pancreatic islet cells in a second culture medium that is different than the first culture medium to produce a population of hypoimmune pancreatic islet cells. In some embodiments, the GSK inhibitor is CHIR-99021, a derivative thereof, or a variant thereof. In some instances, the GSK inhibitor is at a concentration ranging from about 2 mM to about 10 mM. In some embodiments, the ALK inhibitor is SB-431542, a derivative thereof, or a variant thereof. In some instances, the ALK inhibitor is at a concentration ranging from about 1 pM to about 10 pM. In some embodiments, the first culture medium and/or second culture medium are absent of animal serum.

In some embodiments, the population of hypoimmunogenic pancreatic islet cells is isolated from non-pancreatic islet cells. In some embodiments, the isolated population of hypoimmunogenic pancreatic islet cells are expanded prior to administration. In many embodiments, the isolated population of hypoimmunogenic pancreatic islet cells are expanded and cryopreserved prior to administration.

Differentiation is assayed as is known in the art, generally by evaluating the presence of β cell associated or specific markers, including but not limited to, insulin. Differentiation can also be measured functionally, such as measuring glucose metabolism, see generally Muraro et al., Cell Syst. Oct. 26, 2016; 3(4): 385-394.e3, hereby incorporated by reference in its entirety, and specifically for the biomarkers outlined there. Once the beta cells are generated, they can be transplanted (either as a cell suspension or within a gel matrix as discussed herein) into the portal vein/liver, the omentum, the gastrointestinal mucosa, the bone marrow, a muscle, or subcutaneous pouches.

Additional descriptions of pancreatic islet cells including dopaminergic neurons for use in the present technology are found in WO2020/018615, the disclosure is herein incorporated by reference in its entirety.

### 10. Retinal Pigmented Epithelium (RPE) Cells

Provided herein are retinal pigmented epithelium (RPE) cells derived from the hypoimmunogenic induced pluripotent stem (HIP) cells described. For instance, human RPE cells can be produced by differentiating human HIP cells. In some embodiments, hypoimmunogenic pluripotent cells that are differentiated into various RPE cell types are transplanted or engrafted into subjects (e.g., recipients). As will be appreciated by those in the art, the methods for differentiation depend on the desired cell type using known techniques.

The term “RPE” cells refers to pigmented retinal epithelial cells having a genetic expression profile similar or substantially similar to that of native RPE cells. Such RPE cells derived from pluripotent stem cells may possess the polygonal, planar sheet morphology of native RPE cells when grown to confluence on a planar substrate.

The RPE cells can be implanted into a patient suffering from macular degeneration or a patient having damaged RPE cells. In some embodiments, the patient has age-related macular degeneration (AMD), early AMD, intermediate AMD, late AMD, non-neovascular age-related macular degeneration, dry macular degeneration (dry age-related macular degeneration), wet macular degeneration (wet age-real ted macular degeneration), juvenile macular degeneration (JMD) (e.g., Stargardt disease, Best disease, and juvenile retinoschisis), Leber's Congenital Ameurosis, or retinitis pigmentosa. In other embodiments, the patient suffers from retinal detachment.

Exemplary RPE cell types include, but are not limited to, retinal pigmented epithelium (RPE) cell, RPE progenitor cell, immature RPE cell, mature RPE cell, functional RPE cell, and the like.

Useful methods for differentiating pluripotent stem cells into RPE cells are described in, for example, U.S. Pat. Nos. 9,458,428 and 9,850,463, the disclosures are herein incorporated by reference in their entirety, including the specifications. Additional methods for producing RPE cells from human induced pluripotent stem cells can be found in, for example, Lamba et al., PNAS, 2006, 103(34): 12769-12774; Mellough et al, Stem Cells, 2012, 30(4):673-686; Idelson et al, Cell Stem Cell, 2009, 5(4): 396-408; Rowland et al, Journal of Cellular Physiology, 2012, 227(2):457-466, Buchholz et al, Stem Cells Trans Med, 2013, 2(5): 384-393, and da Cruz et al, Nat Biotech, 2018, 36:328-337.

Human pluripotent stem cells have been differentiated into RPE cells using the techniques outlined in Kamao et al, Stem Cell Reports 2014:2:205-18, hereby incorporated by reference in its entirety and in particular for the methods and reagents outlined there for the differentiation techniques and reagents; see also Mandai et al., N Engl J Med, 2017, 376:1038-1046, the contents herein incorporated in its entirety for techniques for generating sheets of RPE cells and transplantation into patients. Differentiation can be assayed as is known in the art, generally by evaluating the presence of RPE associated and/or specific markers or by measuring functionally. See for example Kamao et al., Stem Cell Reports, 2014, 2(2):205-18, the contents incorporated herein by reference in its entirety and specifically for the markers outlined in the first paragraph of the results section.

In some embodiments, the method of producing a population of hypoimmunogenic retinal pigmented epithelium (RPE) cells from a population of hypoimmunogenic pluripotent cells by in vitro differentiation comprises: (a) culturing the population of hypoimmunogenic pluripotent cells in a first culture medium comprising any one of the factors selected from the group consisting of activin A, bFGF, BMP4/7, DKK1, IGF1, noggin, a BMP inhibitor, an ALK inhibitor, a ROCK inhibitor, and a VEGFR inhibitor to produce a population of pre-RPE cells; and (b) culturing the population of pre-RPE cells in a second culture medium that is different than the first culture medium to produce a population of hypoimmunogenic RPE cells. In some embodiments, the ALK inhibitor is SB-431542, a derivative thereof, or a variant thereof. In some instances, the ALK inhibitor is at a concentration ranging from about 2 mM to about 10 pM. In some embodiments, the ROCK inhibitor is Y-27632, a derivative thereof, or a variant thereof. In some instances, the ROCK inhibitor is at a concentration ranging from about 1 pM to about 10 pM. In some embodiments, the first culture medium and/or second culture medium are absent of animal serum.

Differentiation can be assayed as is known in the art, generally by evaluating the presence of RPE associated and/or specific markers or by measuring functionally. See for example Kamao et al., Stem Cell Reports, 2014, 2(2):205-18, the contents are herein incorporated by reference in its entirety and specifically for the results section.

Additional descriptions of RPE cells for use in the present technology are found in WO2020/018615, the disclosure is herein incorporated by reference in its entirety.

For therapeutic application, cells prepared according to the disclosed methods can typically be supplied in the form of a pharmaceutical composition comprising an isotonic excipient, and are prepared under conditions that are sufficiently sterile for human administration. For general principles in medicinal formulation of cell compositions, see “Cell Therapy: Stem Cell Transplantation, Gene Therapy, and Cellular Immunotherapy,” by Morstyn & Sheridan eds, Cambridge University Press, 1996; and “Hematopoietic Stem Cell Therapy,” E. D. Ball, J. Lister & P. Law, Churchill Livingstone, 2000. The cells can be packaged in a device or container suitable for distribution or clinical use.

### 11. T Lymphocytes

Provided herein, T lymphocytes (T cells) are derived from the hypoimmunogenic induced pluripotent stem (HIP) cells described. Methods for generating T cells, including CAR-T cells, from pluripotent stem cells (e.g., iPSCs) are described, for example, in Iriguchi et al., Nature Communications 12, 430 (2021); Themeli et al., Cell Stem Cell, 16(4):357-366 (2015); Themeli et al., Nature Biotechnology 31:928-933 (2013).

In some embodiments, the hypoimmunogenic induced pluripotent stem cell-derived T cell includes a chimeric antigen receptor (CAR). Any suitable CAR can be included in the hypoimmunogenic induced pluripotent stem cell-derived T cell, including the CARs described herein. In some embodiments, the hypoimmunogenic induced pluripotent stem cell-derived T cell includes a polynucleotide encoding a CAR, wherein the polynucleotide is inserted in a genomic locus. In some embodiments, the polynucleotide is inserted into a safe harbor locus. In some embodiments, the polynucleotide is inserted in a B2M, CIITA, TRAC, TRB, PD-1 or CTLA-4 gene. Any suitable method can be used to insert the CAR into the genomic locus of the hypoimmunogenic cell including the gene editing methods described herein (e.g., a CRISPR/Cas system).

HIP-derived T cells provided herein are useful for the treatment of suitable cancers including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

### S. Methods of Genetic Modifications

In some embodiments, the rare-cutting endonuclease is introduced into a cell containing the target polynucleotide sequence in the form of a nucleic acid encoding a rare-cutting endonuclease. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

The present technology contemplates altering target polynucleotide sequences in any manner which is available to the skilled artisan utilizing a CRISPR/Cas system of the present technology. Any CRISPR/Cas system that is capable of altering a target polynucleotide sequence in a cell can be used. Such CRISPR-Cas systems can employ a variety of Cas proteins (Haft et al. PLOS Comput Biol. 2005; 1(6)e60). The molecular machinery of such Cas proteins that allows the CRISPR/Cas system to alter target polynucleotide sequences in cells include RNA binding proteins, endo- and exo-nucleases, helicases, and polymerases. In some embodiments, the CRISPR/Cas system is a CRISPR type I system. In some embodiments, the CRISPR/Cas system is a CRISPR type II system. In some embodiments, the CRISPR/Cas system is a CRISPR type V system.

The CRISPR/Cas systems of the present technology can be used to alter any target polynucleotide sequence in a cell. Those skilled in the art will readily appreciate that desirable target polynucleotide sequences to be altered in any particular cell may correspond to any genomic sequence for which expression of the genomic sequence is associated with a disorder or otherwise facilitates entry of a pathogen into the cell. For example, a desirable target polynucleotide sequence to alter in a cell may be a polynucleotide sequence corresponding to a genomic sequence which contains a disease associated single polynucleotide polymorphism. In such example, the CRISPR/Cas systems of the present technology can be used to correct the disease associated SNP in a cell by replacing it with a wild-type allele. As another example, a polynucleotide sequence of a target gene which is responsible for entry or proliferation of a pathogen into a cell may be a suitable target for deletion or insertion to disrupt the function of the target gene to prevent the pathogen from entering the cell or proliferating inside the cell.

In some embodiments, the target polynucleotide sequence is a genomic sequence. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence.

In some embodiments, a CRISPR/Cas system of the present technology includes a Cas protein and at least one to two ribonucleic acids that are capable of directing the Cas protein to and hybridizing to a target motif of a target polynucleotide sequence. As used herein, “protein” and “polypeptide” are used interchangeably to refer to a series of amino acid residues joined by peptide bonds (i.e., a polymer of amino acids) and include modified amino acids (e.g., phosphorylated, glycated, glycosylated, etc.) and amino acid analogs. Exemplary polypeptides or proteins include gene products, naturally occurring proteins, homologs, paralogs, fragments and other equivalents, variants, and analogs of the above.

In some embodiments, a Cas protein comprises one or more amino acid substitutions or modifications. In some embodiments, the one or more amino acid substitutions comprises a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In some embodiments, a Cas protein comprises a core Cas protein. Exemplary Cas core proteins include, but are not limited to Cas1, Cas2, Cas3, Cas4, Cas5, Cas6, Cas7, Cas8 and Cas9. In some embodiments, a Cas protein comprises type V Cas protein. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include but are not limited to Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Casa. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6. See, e.g., Klompe et al., Nature 571, 219-225 (2019); Strecker et al., Science 365, 48-53 (2019).

In some embodiments, a Cas protein comprises any one of the Cas proteins described herein or a functional portion thereof. As used herein, “functional portion” refers to a portion of a peptide which retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cas12a (also known as Cpf1) protein functional domains selected from the group consisting of a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9 protein comprises a functional portion of the HNH nuclease domain. In some embodiments, a functional portion of the Cas12a protein comprises a functional portion of a RuvC-like domain.

In some embodiments, exogenous Cas protein can be introduced into the cell in polypeptide form. In many embodiments, Cas proteins can be conjugated to or fused to a cell-penetrating polypeptide or cell-penetrating peptide. As used herein, “cell-penetrating polypeptide” and “cell-penetrating peptide” refers to a polypeptide or peptide, respectively, which facilitates the uptake of molecule into a cell. The cell-penetrating polypeptides can contain a detectable label.

In many embodiments, Cas proteins can be conjugated to or fused to a charged protein (e.g., that carries a positive, negative or overall neutral electric charge). Such linkage may be covalent. In some embodiments, the Cas protein can be fused to a superpositively charged GFP to significantly increase the ability of the Cas protein to penetrate a cell (Cronican et al. ACS Chem Biol. 2010; 5(8):747-52). In many embodiments, the Cas protein can be fused to a protein transduction domain (PTD) to facilitate its entry into a cell. Exemplary PTDs include Tat, oligoarginine, and penetratin. In some embodiments, the Cas9 protein comprises a Cas9 polypeptide fused to a cell-penetrating peptide. In some embodiments, the Cas9 protein comprises a Cas9 polypeptide fused to a PTD. In some embodiments, the Cas9 protein comprises a Cas9 polypeptide fused to a tat domain. In some embodiments, the Cas9 protein comprises a Cas9 polypeptide fused to an oligoarginine domain. In some embodiments, the Cas9 protein comprises a Cas9 polypeptide fused to a penetratin domain. In some embodiments, the Cas9 protein comprises a Cas9 polypeptide fused to a superpositively charged GFP. In some embodiments, the Cas12a protein comprises a Cas12a polypeptide fused to a cell-penetrating peptide. In some embodiments, the Cas12a protein comprises a Cas12a polypeptide fused to a PTD. In some embodiments, the Cas12a protein comprises a Cas12a polypeptide fused to a tat domain. In some embodiments, the Cas12a protein comprises a Cas12a polypeptide fused to an oligoarginine domain. In some embodiments, the Cas12a protein comprises a Cas12a polypeptide fused to a penetratin domain. In some embodiments, the Cas12a protein comprises a Cas12a polypeptide fused to a superpositively charged GFP.

In some embodiments, the Cas protein can be introduced into a cell containing the target polynucleotide sequence in the form of a nucleic acid encoding the Cas protein. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

In some embodiments, the Cas protein is complexed with one to two ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

The methods of the present technology contemplate the use of any ribonucleic acid that is capable of directing a Cas protein to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, a single ribonucleic acid comprises a guide RNA that directs the Cas protein to and hybridizes to a target motif of the target polynucleotide sequence in a cell. In some embodiments, at least one of the ribonucleic acids comprises a guide RNA that directs the Cas protein to and hybridizes to a target motif of the target polynucleotide sequence in a cell. In some embodiments, both of the one to two ribonucleic acids comprise a guide RNA that directs the Cas protein to and hybridizes to a target motif of the target polynucleotide sequence in a cell. The ribonucleic acids of the present technology can be selected to hybridize to a variety of different target motifs, depending on the particular CRISPR/Cas system employed, and the sequence of the target polynucleotide, as will be appreciated by those skilled in the art. The one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs.

In some embodiments, each of the one to two ribonucleic acids comprises guide RNAs that directs the Cas protein to and hybridizes to a target motif of the target polynucleotide sequence in a cell.

In some embodiments, one or two ribonucleic acids (e.g., guide RNAs) are complementary to and/or hybridize to sequences on the same strand of a target polynucleotide sequence. In some embodiments, one or two ribonucleic acids (e.g., guide RNAs) are complementary to and/or hybridize to sequences on the opposite strands of a target polynucleotide sequence. In some embodiments, the one or two ribonucleic acids (e.g., guide RNAs) are not complementary to and/or do not hybridize to sequences on the opposite strands of a target polynucleotide sequence. In some embodiments, the one or two ribonucleic acids (e.g., guide RNAs) are complementary to and/or hybridize to overlapping target motifs of a target polynucleotide sequence. In some embodiments, the one or two ribonucleic acids (e.g., guide RNAs) are complementary to and/or hybridize to offset target motifs of a target polynucleotide sequence.

In some embodiments, nucleic acids encoding Cas protein and nucleic acids encoding the at least one to two ribonucleic acids are introduced into a cell via viral transduction (e.g., lentiviral transduction). In some embodiments, the Cas protein is complexed with 1-2 ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

Exemplary gRNA sequences useful for CRISPR/Cas-based targeting of genes described herein are provided in Table 15. The sequences can be found in WO2016183041 filed May 9, 2016, the disclosure including the Tables, Appendices, and Sequence Listing is incorporated herein by reference in its entirety.

TABLE 15
Exemplary gRNA sequences useful for targeting genes

| Gene Name | SEQ ID NO: | WO2016183041 |
|---|---|---|
| HLA-A | SEQ ID NOs: 2-1418 | Table 8, Appendix 1 |
| HLA-B | SEQ ID NOs: 1419-3277 | Table 9, Appendix 2 |
| HLA-C | SEQ ID NOS: 3278-5183 | Table 10, Appendix 3 |
| RFX-ANK | SEQ ID NOs: 95636-102318 | Table 11, Appendix 4 |
| NFY-A | SEQ ID NOs: 102319-121796 | Table 13, Appendix 6 |
| RFX5 | SEQ ID NOs: 85645-90115 | Table 16, Appendix 9 |
| RFX-AP | SEQ ID NOs: 90116-95635 | Table 17, Appendix 10 |
| NFY-B | SEQ ID NOs: 121797-135112 | Table 20, Appendix 13 |
| NFY-C | SEQ ID NOs: 135113-176601 | Table 22, Appendix 15 |
| IRF1 | SEQ ID NOs: 176602-182813 | Table 23, Appendix 16 |
| TAP1 | SEQ ID NOs: 182814-188371 | Table 24, Appendix 17 |
| CIITA | SEQ ID NOS: 5184-36352 | Table 12, Appendix 5 |
| B2M | SEQ ID NOS: 81240-85644 | Table 15, Appendix 8 |
| NLRC5 | SEQ ID NOS: 36353-81239 | Table 14, Appendix 7 |
| CD47 | SEQ ID NOS: 200784-231885 | Table 29, Appendix 22 |
| HLA-E | SEQ ID NOS: 189859-193183 | Table 19, Appendix 12 |
| HLA-F | SEQ ID NOS: 688808-699754 | Table 45, Appendix 38 |
| HLA-G | SEQ ID NOS: 188372-189858 | Table 18, Appendix 11 |
| PD-L1 | SEQ ID NOS: 193184-200783 | Table 21, Appendix 14 |
| Gene Name | SEQ ID NO: | US20160348073 |
| TRAC | SEQ ID NOS: 532-609 and | |
| | 9102-9797 | |
| TRB (also | SEQ ID NOS: 610-765 and | |
| TCRB and | 9798-10532 | |
| TRBC) | | |

In some embodiments, the cells of the technology are made using Transcription Activator-Like Effector Nucleases (TALEN) methodologies.

By a “TALE-nuclease” (TALEN) is intended a fusion protein consisting of a nucleic acid-binding domain typically derived from a Transcription Activator Like Effector (TALE) and one nuclease catalytic domain to cleave a nucleic acid target sequence. The catalytic domain is preferably a nuclease domain and more preferably a domain having endonuclease activity, like for instance I-TevI, ColE7, NucA and Fok-I. In numerous embodiments, the TALE domain can be fused to a meganuclease like for instance I-CreI and I-OnuI or functional variant thereof. In a more preferred embodiment, said nuclease is a monomeric TALE-Nuclease. A monomeric TALE-Nuclease is a TALE-Nuclease that does not require dimerization for specific recognition and cleavage, such as the fusions of engineered TAL repeats with the catalytic domain of I-TevI described in WO2012138927. Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. TALEN kits are sold commercially.

In some embodiments, the cells are manipulated using zinc finger nuclease (ZFN). A “zinc finger binding protein” is a protein or polypeptide that binds DNA, RNA and/or protein, preferably in a sequence-specific manner, as a result of stabilization of protein structure through coordination of a zinc ion. The term zinc finger binding protein is often abbreviated as zinc finger protein or ZFP. The individual DNA binding domains are typically referred to as “fingers.” A ZFP has least one finger, typically two fingers, three fingers, or six fingers. Each finger binds from two to four base pairs of DNA, typically three or four base pairs of DNA. A ZFP binds to a nucleic acid sequence called a target site or target segment. Each finger typically comprises an approximately 30 amino acid, zinc-chelating, DNA-binding subdomain. Studies have demonstrated that a single zinc finger of this class consists of an alpha helix containing the two invariant histidine residues co-ordinated with zinc along with the two cysteine residues of a single beta turn (see, e.g., Berg & Shi, Science 271:1081-1085 (1996)).

In some embodiments, the cells of the present technology are made using a homing endonuclease. Such homing endonucleases are well-known to the art (Stoddard 2005). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease according to the technology may for example correspond to a LAGLIDADG endonuclease, to a HNH endonuclease, or to a GIY-YIG endonuclease. Preferred homing endonuclease according to the present technology can be an I-CreI variant.

In some embodiments, the cells of the technology are made using a meganuclease. Meganucleases are by definition sequence-specific endonucleases recognizing large sequences (Chevalier, B. S. and B. L. Stoddard, Nucleic Acids Res., 2001, 29, 3757-3774). They can cleave unique sites in living cells, thereby enhancing gene targeting by 1000-fold or more in the vicinity of the cleavage site (Puchta et al., Nucleic Acids Res., 1993, 21, 5034-5040; Rouet et al., Mol. Cell. Biol., 1994, 14, 8096-8106; Choulika et al., Mol. Cell. Biol., 1995, 15, 1968-1973; Puchta et al., Proc. Natl. Acad. Sci. USA, 1996, 93, 5055-5060; Sargent et al., Mol. Cell. Biol., 1997, 17, 267-77; Donoho et al., Mol. Cell. Biol, 1998, 18, 4070-4078; Elliott et al., Mol. Cell. Biol., 1998, 18, 93-101; Cohen-Tannoudji et al., Mol. Cell. Biol., 1998, 18, 1444-1448).

In some embodiments, the cells of the technology are made using RNA silencing or RNA interference (RNAi) to knockdown (e.g., decrease, eliminate, or inhibit) the expression of a polypeptide such as a tolerogenic factor. Useful RNAi methods include those that utilize synthetic RNAi molecules, short interfering RNAs (siRNAs), PIWI-interacting NRAs (piRNAs), short hairpin RNAs (shRNAs), microRNAs (miRNAs), and other transient knockdown methods recognized by those skilled in the art. Reagents for RNAi including sequence specific shRNAs, siRNA, miRNAs and the like are commercially available. For instance, CIITA can be knocked down in a pluripotent stem cell by introducing a CIITA siRNA or transducing a CIITA shRNA-expressing virus into the cell. In some embodiments, RNA interference is employed to reduce or inhibit the expression of at least one selected from the group consisting of CIITA, B2M, NLRC5, TCR-alpha, and TCR-beta.

In some embodiments, the cells provided herein are genetically modified to reduce expression of one or more immune factors (including target polypeptides) to create immune-privileged or hypoimmunogenic cells. In many embodiments, the cells (e.g., stem cells, induced pluripotent stem cells, differentiated cells, hematopoietic stem cells, primary T cells and CAR-T cells) disclosed herein comprise one or more genetic modifications to reduce expression of one or more target polynucleotides. Non-limiting examples of such target polynucleotides and polypeptides include CIITA, B2M, NLRC5, CTLA-4, PD-1, HLA-A, HLA-BM, HLA-C, RFX-ANK, NFY-A, RFX5, RFX-AP, NFY-B, NFY-C, IRF1, and TAP1.

In some embodiments, the genetic modification occurs using a CRISPR/Cas system. By modulating (e.g., reducing or deleting) expression of one or a plurality of the target polynucleotides, such cells exhibit decreased immune activation when engrafted into a recipient subject. In some embodiments, the cell is considered hypoimmunogenic, e.g., in a recipient subject or patient upon administration.

a. Additional Descriptions of Gene Editing Systems

In some embodiments, the methods for genetically modifying cells to knock out, knock down, or otherwise modify one or more genes comprise using a site-directed nuclease, including, for example, zinc finger nucleases (ZFNs), transcription activator-like effector nucleases (TALENs), meganucleases, transposases, and clustered regularly interspaced short palindromic repeat (CRISPR)/Cas systems, as well as nickase systems, base editing systems, prime editing systems, and gene writing systems known in the art.

i. ZFNs

ZFNs are fusion proteins comprising an array of site-specific DNA binding domains adapted from zinc finger-containing transcription factors attached to the endonuclease domain of the bacterial FokI restriction enzyme. A ZFN may have one or more (e.g., 1, 2, 3, 4, 5, 6, 7, 8, 9, 10, or more) of the DNA binding domains or zinc finger domains. See, e.g., Carroll et al., Genetics Society of America (2011) 188:773-782; Kim et al., Proc. Natl. Acad. Sci. USA (1996) 93:1156-1160. Each zinc finger domain is a small protein structural motif stabilized by one or more zinc ions and usually recognizes a 3- to 4-bp DNA sequence. Tandem domains can thus potentially bind to an extended nucleotide sequence that is unique within a cell's genome.

Various zinc fingers of known specificity can be combined to produce multi-finger polypeptides which recognize about 6, 9, 12, 15, or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art. See, e.g., Sera et al., Biochemistry (2002) 41:7074-7081; Liu et al., Bioinformatics (2008) 24:1850-1857.

ZFNs containing FokI nuclease domains or other dimeric nuclease domains function as a dimer. Thus, a pair of ZFNs are required to target non-palindromic DNA sites. The two individual ZFNs must bind opposite strands of the DNA with their nucleases properly spaced apart. See Bitinaite et al., Proc. Natl. Acad. Sci. USA (1998) 95:10570-10575. To cleave a specific site in the genome, a pair of ZFNs are designed to recognize two sequences flanking the site, one on the forward strand and the other on the reverse strand. Upon binding of the ZFNs on either side of the site, the nuclease domains dimerize and cleave the DNA at the site, generating a DSB with 5′ overhangs. HDR can then be utilized to introduce a specific mutation, with the help of a repair template containing the desired mutation flanked by homology arms. The repair template is usually an exogenous double-stranded DNA vector introduced to the cell. See Miller et al., Nat. Biotechnol. (2011) 29:143-148; Hockemeyer et al., Nat. Biotechnol. (2011) 29:731-734.

ii. TALENS

TALENs are another example of an artificial nuclease which can be used to edit a target gene. TALENs are derived from DNA binding domains termed TALE repeats, which usually comprise tandem arrays with 10 to 30 repeats that bind and recognize extended DNA sequences. Each repeat is 33 to 35 amino acids in length, with two adjacent amino acids (termed the repeat-variable di-residue, or RVD) conferring specificity for one of the four DNA base pairs. Thus, there is a one-to-one correspondence between the repeats and the base pairs in the target DNA sequences.

TALENs are produced artificially by fusing one or more TALE DNA binding domains (e.g., 1, 2, 3, 4, 5, 6, 7, 8, 9, 10 or more) to a nuclease domain, for example, a FokI endonuclease domain. See Zhang, Nature Biotech. (2011) 29:149-153. Several mutations to FokI have been made for its use in TALENs; these, for example, improve cleavage specificity or activity. See Cermak et al., Nucl. Acids Res. (2011) 39: e82; Miller et al., Nature Biotech. (2011) 29:143-148; Hockemeyer et al., Nature Biotech. (2011) 29:731-734; Wood et al., Science (2011) 333:307; Doyon et al., Nature Methods (2010) 8:74-79; Szczepek et al., Nature Biotech (2007) 25:786-793; Guo et al., J. Mol. Biol. (2010) 200:96. The FokI domain functions as a dimer, requiring two constructs with unique DNA binding domains for sites in the target genome with proper orientation and spacing. Both the number of amino acid residues between the TALE DNA binding domain and the FokI nuclease domain and the number of bases between the two individual TALEN binding sites appear to be important parameters for achieving high levels of activity. Miller et al., Nature Biotech. (2011) 29:143-148.

By combining engineered TALE repeats with a nuclease domain, a site-specific nuclease can be produced specific to any desired DNA sequence. Similar to ZFNs, TALENs can be introduced into a cell to generate DSBs at a desired target site in the genome, and so can be used to knock out genes or knock in mutations in similar, HDR-mediated pathways. See Boch, Nature Biotech. (2011) 29:135-136; Boch et al., Science (2009) 326:1509-1512; Moscou et al., Science (2009) 326:3501.

iii. Meganucleases

Meganucleases are enzymes in the endonuclease family which are characterized by their capacity to recognize and cut large DNA sequences (from 14 to 40 base pairs). Meganucleases are grouped into families based on their structural motifs which affect nuclease activity and/or DNA recognition. The most widespread and best known meganucleases are the proteins in the LAGLIDADG family, which owe their name to a conserved amino acid sequence. See Chevalier et al., Nucleic Acids Res. (2001) 29(18): 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29(18):3757-3774. Members of the NHN family are defined by motifs containing two pairs of conserved histidines surrounded by asparagine residues. See Chevalier et al., Nucleic Acids Res. (2001) 29(18): 3757-3774.

Because the chance of identifying a natural meganuclease for a particular target DNA sequence is low due to the high specificity requirement, various methods including mutagenesis and high throughput screening methods have been used to create meganuclease variants that recognize unique sequences. Strategies for engineering a meganuclease with altered DNA-binding specificity, e.g., to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mol Biol (2004) 342:31-41; Doyon et al., J Am Chem Soc (2006) 128:2477-2484; Chen et al., Protein Eng Des Sel (2009) 22:249-256; Arnould et al., J Mol Biol. (2006) 355:443-458; Smith et al., Nucleic Acids Res. (2006) 363(2):283-294.

Like ZFNs and TALENs, Meganucleases can create DSBs in the genomic DNA, which can create a frame-shift mutation if improperly repaired, e.g., via NHEJ, leading to a decrease in the expression of a target gene in a cell. Alternatively, foreign DNA can be introduced into the cell along with the meganuclease. Depending on the sequences of the foreign DNA and chromosomal sequence, this process can be used to modify the target gene. See Silva et al., Current Gene Therapy (2011) 11:11-27.

iv. Transposases

Transposases are enzymes that bind to the end of a transposon and catalyze its movement to another part of the genome by a cut and paste mechanism or a replicative transposition mechanism. By linking transposases to other systems such as the CRISPER/Cas system, new gene editing tools can be developed to enable site specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

v. CRISPR Cas Systems

The CRISPR system was originally discovered in prokaryotic organisms (e.g., bacteria and archaea) as a system involved in defense against invading phages and plasmids that provides a form of acquired immunity. Now it has been adapted and used as a popular gene editing tool in research and clinical applications.

CRISPR/Cas systems generally comprise at least two components: one or more guide RNAs (gRNAs) and a Cas protein. The Cas protein is a nuclease that introduces a DSB into the target site. CRISPR-Cas systems fall into two major classes: class 1 systems use a complex of multiple Cas proteins to degrade nucleic acids; class 2 systems use a single large Cas protein for the same purpose. Class 1 is divided into types I, III, and IV; class 2 is divided into types II, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2cl), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. The most widely used Cas9 is described herein as illustrative. These Cas proteins may be originated from different source species. For example, Cas9 can be derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAs (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

Since its discovery, the CRISPR system has been adapted for inducing sequence specific DSBs and targeted genome editing in a wide range of cells and organisms spanning from bacteria to eukaryotic cells including human cells. In its use in gene editing applications, artificially designed, synthetic gRNAs have replaced the original crRNA:tracrRNA complex. For example, the gRNAs can be single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease derived from S. pyogenes recognizes a PAM sequence of 5′-NGG-3′ or, at less efficient rates, 5′-NAG-3′, where “N” can be any nucleotide. Other Cas nuclease variants with alternative PAMs have also been characterized and successfully used for genome editing, which are summarized in Table 16 below.

TABLE 16
Exemplary Cas nuclease variants and their PAM sequences

| CRISPR Nuclease | Source Organism | PAM Sequence (5′→3′) |
|---|---|---|
| SpCas9 | Streptococcus pyogenes | NGG or NAG |
| SaCas9 | Staphylococcus aureus | NGRRT or NGRRN |
| NmeCas9 | Neisseria meningitidis | NNNNGATT |
| CjCas9 | Campylobacter jejuni | NNNNRYAC |
| StCas9 | Streptococcus thermophilus | NNAGAAW |
| TdCas9 | Treponema denticola | NAAAAC |
| LbCas12a (Cpf1) | Lachnospiraceae bacterium | TTTV |
| AsCas12a (Cpf1) | Acidaminococcus sp. | TTTV |
| AacCas12b | Alicyclobacillus acidiphilus | TTN |
| BhCas12b v4 | Bacillus hisashii | ATTN, TTTN, or GTTN |

R = A or G;
Y = C or T;
W = A or T;
V = A or C or G;
N = any base

In some embodiments, Cas nucleases may comprise one or more mutations to alter their activity, specificity, recognition, and/or other characteristics. For example, the Cas nuclease may have one or more mutations that alter its fidelity to mitigate off-target effects (e.g., eSpCas9, SpCas9-HF1, HypaSpCas9, HeFSpCas9, and evoSpCas9 high-fidelity variants of SpCas9). For another example the Cas nuclease may have one or more mutations that alter its PAM specificity.

vi. Nickases

Nuclease domains of the Cas, in particular the Cas9, nuclease can be mutated independently to generate enzymes referered to as DNA “nickases”. Nickases are capable of introducing a single-strand cut with the same specificity as a regular CRISPR/Cas nucleas system, including for example CRISPR/Cas9. Nickases can be employed to generate double-strand breaks which can find use in gene editing systems (Mali et al., Nat Biotech, 31(9):833-838 (2013); Mali et al. Nature Methods, 10:957-963 (2013); Mali et al., Science, 339(6121):823-826 (2013)). In some instances, when two Cas nickases are used, long overhangs are produced on each of the cleaved ends instead of blunt ends which allows for additional control over precise gene integration and insertion (Mali et al., Nat Biotech, 31(9):833-838 (2013); Mali et al. Nature Methods, 10:957-963 (2013); Mali et al., Science, 339(6121):823-826 (2013)). As both nicking Cas enzymes must effectively nick their target DNA, paired nickases can have lower off-target effects compared to the double-strand-cleaving Cas-based systems (Ran et al., Cell, 155(2):479-480(2013); Mali et al., Nat Biotech, 31(9):833-838 (2013); Mali et al. Nature Methods, 10:957-963 (2013); Mali et al., Science, 339(6121):823-826 (2013)).

T. Overexpression of Tolerogenic Factors and/or Chimeric Antigen Receptors

For all of these technologies, well-known recombinant techniques are used, to generate recombinant nucleic acids as outlined herein. In many embodiments, the recombinant nucleic acids encoding a tolerogenic factor or a chimeric antigen receptor may be operably linked to one or more regulatory nucleotide sequences in an expression construct. Regulatory nucleotide sequences will generally be appropriate for the host cell and recipient subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, hybrid promoters that combine elements of more than one promoter, or synthetic promoters. An expression construct may be present in a cell on an episome, such as a plasmid, or the expression construct may be inserted in a chromosome such as in a gene locus. In some embodiment, the expression vector includes a selectable marker gene to allow the selection of transformed host cells. Some embodiments include an expression vector comprising a nucleotide sequence encoding a variant polypeptide operably linked to at least one regulatory sequence. Regulatory sequence for use herein include promoters, enhancers, and other expression control elements. In some embodiments, an expression vector is designed for the choice of the host cell to be transformed, the particular variant polypeptide desired to be expressed, the vector's copy number, the ability to control that copy number, and/or the expression of any other protein encoded by the vector, such as antibiotic markers.

Examples of suitable mammalian promoters include, for example, promoters from the following genes: elongation factor 1 alpha (EF1a) promoter, CAG promoter, ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication (Fiers et al, Nature 273: 113-120 (1978)). The immediate early promoter of the human cytomegalovirus is conveniently obtained as a HindIII restriction enzyme fragment (Greenaway et al, Gene 18: 355-360 (1982)). The foregoing references are incorporated by reference in their entirety.

In some embodiments, the expression vector is a bicistronic or multicistronic expression vector. Bicistronic or multicistronic expression vectors may include (1) multiple promoters fused to each of the open reading frames; (2) insertion of splicing signals between genes; (3) fusion of genes whose expressions are driven by a single promoter; and (4) insertion of proteolytic cleavage sites between genes (self-cleavage peptide) or insertion of internal ribosomal entry sites (IRESs) between genes.

The process of introducing the polynucleotides described herein into cells can be achieved by any suitable technique. Suitable techniques include calcium phosphate or lipid-mediated transfection, electroporation, fusogens, and transduction or infection using a viral vector. In some embodiments, the polynucleotides are introduced into a cell via viral transduction (e.g., lentiviral transduction) or otherwise delivered on a viral vector (e.g., fusogen-mediated delivery).

Unlike certain methods of introducing the polynucleotides described herein into cells which generally involve activating cells, such as activating T cells (e.g., CD8⁺ T cells), suitable techniques can be utilized to introduce polynucleotides into non-activated T cells. Suitable techniques include, but are not limited to, activation of T cells, such as CD8⁺ T cells, with one or more antibodies which bind to CD3, CD8, and/or CD28, or fragments or portions thereof (e.g., scFv and VHH) that may or may not be bound to beads. Surprisingly, fusogen-mediated introduction of polynucleotides into T cells is performed in non-activated T cells (e.g., CD8 T cells) that have not been previously contacted with one or more activating antibodies or fragments or portions thereof (e.g., CD3, CD8, and/or CD28). In some embodiments, fusogen-mediated introduction of polynucleotides into T cells is performed in vivo (e.g., after the T cells have been administered to a subject). In other embodiments, fusogen-mediated introduction of polynucleotides into T cells is performed in vitro (e.g., before the T cells are been administered to a subject).

Provided herein are non-activated T cells comprising reduced expression of HLA-A, HLA-B, HLA-C, CIITA, TCR-alpha, and/or TCR-beta relative to a wild-type T cell, wherein the activated T cell further comprises a first gene encoding a chimeric antigen receptor (CAR).

In some embodiments, the non-activated T cell has not been treated with an anti-CD3 antibody, an anti-CD28 antibody, a T cell activating cytokine, or a soluble T cell costimulatory molecule. In some embodiments, the non-activated T cell does not express activation markers. In some embodiments, the non-activated T cell expresses CD3 and CD28, and wherein the CD3 and/or CD28 are inactive.

In some embodiments, the anti-CD3 antibody is OKT3. In some embodiments, the anti-CD28 antibody is CD28.2. In some embodiments, the T cell activating cytokine is selected from the group of T cell activating cytokines consisting of IL-2, IL-7, IL-15, and IL-21. In some embodiments, the soluble T cell costimulatory molecule is selected from the group of soluble T cell costimulatory molecules consisting of an anti-CD28 antibody, an anti-CD80 antibody, an anti-CD86 antibody, an anti-CD137L antibody, and an anti-ICOS-L antibody.

In some embodiments, the non-activated T cell is a primary T cell. In other embodiments, the non-activated T cell is differentiated from the hypoimmunogenic cells of the present technology. In some embodiments, the T cell is a CD8+ T cell.

In some embodiments, the first gene is carried by a lentiviral vector that comprises a CD8 binding agent. In some embodiments, the first gene is a CAR is selected from the group consisting of a CD19-specific CAR and a CD22-specific CAR.

In some embodiments, the non-activated T cell further comprises a second gene as an HLA-E variant, an HLA-G variant, and/or exogenous PD-L1. In some embodiments, the first and/or second genes are inserted into a specific locus of at least one allele of the T cell. In some embodiments, the specific locus is selected from the group consisting of a safe harbor locus, an HLA-A locus, an HLA-B locus, an HLA-C locus, a CD155 locus, a B2M locus, a CIITA locus, a TRAC locus, and a TRB locus. In some embodiments, the second gene encoding an HLA-E variant, an HLA-G variant, and/or exogenous PD-L1 is inserted into the specific locus selected from the group consisting of a safe harbor locus, an HLA-A locus, an HLA-B locus, an HLA-C locus, a CD155 locus, a B2M locus, a CIITA locus, a TRAC locus and a TRB locus. In some embodiments, the first gene encoding the CAR is inserted into the specific locus selected from the group consisting of a safe harbor locus, an HLA-A locus, an HLA-B locus, an HLA-C locus, a CD155 locus, a B2M locus, a CIITA locus, a TRAC locus and a TRB locus. In some embodiments, the second gene encoding an HLA-E variant, an HLA-G variant, and/or exogenous PD-L1 and the first gene encoding the CAR are inserted into different loci. In some embodiments, the second gene encoding an HLA-E variant, an HLA-G variant, and/or exogenous PD-L1 and the first gene encoding the CAR are inserted into the same locus.

In some embodiments, the second gene encoding an HLA-E variant, an HLA-G variant, and/or exogenous PD-L1 and the first gene encoding the CAR are inserted into the HLA-A locus. In some embodiments, the second gene encoding an HLA-E variant, an HLA-G variant, and/or exogenous PD-L1 and the first gene encoding the CAR are inserted into the HLA-B locus. In some embodiments, the second gene encoding an HLA-E variant, an HLA-G variant, and/or exogenous PD-L1 and the first gene encoding the CAR are inserted into the HLA-C locus. In some embodiments, the second gene encoding an HLA-E variant, an HLA-G variant, and/or exogenous PD-L1 and the first gene encoding the CAR are inserted into the CD155 locus. In some embodiments, the second gene encoding an HLA-E variant, an HLA-G variant, and/or exogenous PD-L1 and the first gene encoding the CAR are inserted into the B2M locus. In some embodiments, the second gene encoding an HLA-E variant, an HLA-G variant, and/or exogenous PD-L1 and the first gene encoding the CAR are inserted into the CIITA locus. In some embodiments, the second gene encoding an HLA-E variant, an HLA-G variant, and/or exogenous PD-L1 and the first gene encoding the CAR are inserted into the TRAC locus. In some embodiments, the second gene encoding an HLA-E variant, an HLA-G variant, and/or exogenous PD-L1 and the first gene encoding the CAR are inserted into the TRB locus. In some embodiments, the second gene encoding an HLA-E variant, an HLA-G variant, and/or exogenous PD-L1 and the first gene encoding the CAR are inserted into the safe harbor locus. In some embodiments, the safe harbor locus is selected from the group consisting of a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, ad RHD gene locus, a FUT1 locus, and a KDM5D gene locus.

In some embodiments, the non-activated T cell does not express HLA-A, HLA-B, and/or HLA-C antigens. In some embodiments, the non-activated T cell does not express B2M. In some embodiments, the non-activated T cell does not express HLA-DP, HLA-DQ, and/or HLA-DR antigens. In some embodiments, the non-activated T cell does not express CIITA. In some embodiments, the non-activated T cell does not express TCR-alpha. In some embodiments, the non-activated T cell does not express TCR-beta. In some embodiments, the non-activated T cell does not express TCR-alpha and TCR-beta.

In some embodiments, the non-activated T cell is a HLA-A^{indel/indel}, HLA-B^{indel/indel}cell comprising second gene encoding an HLA-E variant, an HLA-G variant, and/or exogenous PD-L1 and/or the first gene encoding CAR inserted into a specific locus. In some embodiments, the non-activated T cell is a HLA-A^{indel/indel}, HLA-B^{indel/indel}, HLA-C^{indel/indel} cell comprising second gene encoding an HLA-E variant, an HLA-G variant, and/or exogenous PD-L1 and/or the first gene encoding CAR inserted into a specific locus. In some embodiments, the non-activated T cell is a HLA-A^{indel/indel}, HLA-B^{indel/indel}, CD155^{indel/indel} cell comprising second gene encoding an HLA-E variant, an HLA-G variant, and/or exogenous PD-L1 and/or the first gene encoding CAR inserted into a specific locus. In some embodiments, the non-activated T cell is a HLA-A indel/indel, HLA-B^{indel/indel}, HLA-C^{indel/indel}, CD155^{indel/indel} cell comprising second gene encoding an HLA-E variant, an HLA-G variant, and/or exogenous PD-L1 and/or the first gene encoding CAR inserted into a specific locus. In some embodiments, the specific locus is an HLA-A locus, an HLA-B locus, an HLA-C locus, a CD155 locus, a B2M locus, a CIITA locus, a TRAC locus or a TRB locus. In some embodiments, the specific locus is a safe harbor locus selected from the group consisting of a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, ad RHD gene locus, a FUT1 locus, and a KDM5D gene locus.

In some embodiments, the non-activated T cell is a B2M^{indel/indel} CIITA^{indel/indel}, TRAC^{indel/indel} cell comprising second gene encoding an HLA-E variant, an HLA-G variant, and/or exogenous PD-L1 and/or the first gene encoding CAR inserted into the TRAC locus. In some embodiments, the non-activated T cell is a B2M^{indel/indel}, CIITA^{indel/indel}, TRAC^{indel/indel} cell comprising the second gene encoding an HLA-E variant, an HLA-G variant, and/or exogenous PD-L1 and the first gene encoding CAR inserted into the TRAC locus. In some embodiments, the non-activated T cell is a B2M^{indel/indel}, CIITA^{indel/indel}, TRAC^{indel/indel} cell comprising second gene encoding an HLA-E variant, an HLA-G variant, and/or exogenous PD-L1 and/or the first gene encoding CAR inserted into the TRB locus. In some embodiments, the non-activated T cell is a B2M^{indel/indel} CIITA^{indel/indel}, TRAC^{indel/indel} cell comprising the second gene encoding an HLA-E variant, an HLA-G variant, and/or exogenous PD-L1 and the first gene encoding CAR inserted into the TRB locus. In some embodiments, the non-activated T cell is a B2M^{indel/indel} CIITA^{indel/indel}, TRAC^{indel/indel} cell comprising second gene encoding an HLA-E variant, an HLA-G variant, and/or exogenous PD-L1 and/or the first gene encoding CAR inserted into the B2M locus. In some embodiments, the non-activated T cell is a B2M^{indel/indel}, CIITA^{indel/indel}, TRAC^{indel/indel} cell comprising the second gene encoding an HLA-E variant, an HLA-G variant, and/or exogenous PD-L1 and the first gene encoding CAR inserted into a B2M locus. In some embodiments, the non-activated T cell is a B2M^{indel/indel}, CIITA^{indel/indel}, TRAC^{indel/indel} cell comprising second gene encoding an HLA-E variant, an HLA-G variant, and/or exogenous PD-L1 and/or the first gene encoding CAR inserted into the CIITA locus. In some embodiments, the non-activated T cell is a B2M^{indel/indel}, CIITA^{indel/indel}, TRAC^{indel/indel} cell comprising the second gene encoding an HLA-E variant, an HLA-G variant, and/or exogenous PD-L1 and the first gene encoding CAR inserted into a CIITA locus.

In some embodiments, the non-activated T cell is a B2M^{indel/indel}, CIITA^{indel/indel}, TRB^{indel/indel} cell comprising second gene encoding an HLA-E variant, an HLA-G variant, and/or exogenous PD-L1 and/or the first gene encoding CAR inserted into the TRAC locus. In some embodiments, the non-activated T cell is a B2M^{indel/indel}, CIITA^{indel/indel}, TRB^{indel/indel} cell comprising the second gene encoding an HLA-E variant, an HLA-G variant, and/or exogenous PD-L1 and the first gene encoding CAR inserted into the TRAC locus. In some embodiments, the non-activated T cell is a B2M^{indel/indel}, CIITA^{indel/indel}, TRB^{indel/indel} cell comprising second gene encoding an HLA-E variant, an HLA-G variant, and/or exogenous PD-L1 and/or the first gene encoding CAR inserted into the TRB locus. In some embodiments, the non-activated T cell is a B2M^{indel/indel}, CIITA^{indel/indel}, TRB^{indel/indel} cell comprising the second gene encoding an HLA-E variant, an HLA-G variant, and/or exogenous PD-L1 and the first gene encoding CAR inserted into the TRB locus. In some embodiments, the non-activated T cell is a B2M^{indel/indel}, CIITA^{indel/indel}, TRB^{indel/indel} cell comprising second gene encoding an HLA-E variant, an HLA-G variant, and/or exogenous PD-L1 and/or the first gene encoding CAR inserted into the B2M locus. In some embodiments, the non-activated T cell is a B2M^{indel/indel}, CIITA^{indel/indel}, TRB^{indel/indel} cell comprising the second gene encoding an HLA-E variant, an HLA-G variant, and/or exogenous PD-L1 and the first gene encoding CAR inserted into a B2M locus. In some embodiments, the non-activated T cell is a B2M^{indel/indel}, CIITA^{indel/indel}, TRB^{indel/indel} cell comprising second gene encoding an HLA-E variant, an HLA-G variant, and/or exogenous PD-L1 and/or the first gene encoding CAR inserted into the CIITA locus. In some embodiments, the non-activated T cell is a B2M^{indel/indel}, CIITA^{indel/indel}, TRB^{indel/indel} cell comprising the second gene encoding an HLA-E variant, an HLA-G variant, and/or exogenous PD-L1 and the first gene encoding CAR inserted into a CIITA locus.

Provided herein are engineered T cells comprising reduced expression of HLA-A, HLA-B, HLA-C, CIITA, TCR-alpha, and/or TCR-beta relative to a wild-type T cell, wherein the engineered T cell further comprises a first gene encoding a chimeric antigen receptor (CAR) carried by a lentiviral vector that comprises a CD8 binding agent.

In some embodiments, the engineered T cell is a primary T cell. In other embodiments, the engineered T cell is differentiated from the hypoimmunogenic cell of the present technology. In some embodiments, the T cell is a CD8″T cell. In some embodiments, the T cell is a CD4 T cell.

In some embodiments, the engineered T cell does not express activation markers. In some embodiments, the engineered T cell expresses CD3 and CD28, and wherein the CD3 and/or CD28 are inactive.

In some embodiments, the engineered T cell has not been treated with an anti-CD3 antibody, an anti-CD28 antibody, a T cell activating cytokine, or a soluble T cell costimulatory molecule. In some embodiments, the anti-CD3 antibody is OKT3, wherein the anti-CD28 antibody is CD28.2, wherein the T cell activating cytokine is selected from the group of T cell activating cytokines consisting of IL-2, IL-7, IL-15, and IL-21, and wherein soluble T cell costimulatory molecule is selected from the group of soluble T cell costimulatory molecules consisting of an anti-CD28 antibody, an anti-CD80 antibody, an anti-CD86 antibody, an anti-CD137L antibody, and an anti-ICOS-L antibody. In some embodiments, the engineered T cell has not been treated with one or more T cell activating cytokines selected from the group consisting of IL-2, IL-7, IL-15, and IL-21. In some instances, the cytokine is IL-2. In some embodiments, the one or more cytokines is IL-2 and another selected from the group consisting of IL-7, IL-15, and IL-21.

In some embodiments, the engineered T cell further comprises a second gene that is an HLA-E variant, an HLA-G variant, and/or exogenous PD-L1. In some embodiments, the first and/or second genes are inserted into a specific locus of at least one allele of the T cell. In some embodiments, the specific locus is selected from the group consisting of a safe harbor locus, an HLA-A locus, an HLA-B locus, an HLA-C locus, a CD155 locus, a B2M locus, a CIITA locus, a TRAC locus, and a TRB locus. In some embodiments, the second gene encoding an HLA-E variant, an HLA-G variant, and/or exogenous PD-L1 is inserted into the specific locus selected from the group consisting of a safe harbor locus, a B2M locus, a CIITA locus, a TRAC locus and a TRB locus. In some embodiments, the first gene encoding the CAR is inserted into the specific locus selected from the group consisting of a safe harbor locus, a B2M locus, a CIITA locus, a TRAC locus and a TRB locus. In some embodiments, the second gene encoding an HLA-E variant, an HLA-G variant, and/or exogenous PD-L1 and the first gene encoding the CAR are inserted into different loci. In some embodiments, the second gene encoding an HLA-E variant, an HLA-G variant, and/or exogenous PD-L1 and the first gene encoding the CAR are inserted into the same locus. In some embodiments, the second gene encoding an HLA-E variant, an HLA-G variant, and/or exogenous PD-L1 and the first gene encoding the CAR are inserted into the B2M locus, the CIITA locus, the TRAC locus, the TRB locus, or the safe harbor locus. In some embodiments, the safe harbor locus is selected from the group consisting of a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, ad RHD gene locus, a FUT1 locus, and a KDM5D gene locus.

In some embodiments, the CAR is selected from the group consisting of a CD19-specific CAR and a CD22-specific CAR. In some embodiments, the CAR is a CD19-specific CAR. In some embodiments, the CAR is a CD22-specific CAR. In some embodiments, the CAR comprises an antigen binding domain that binds to any one selected from the group consisting of CD19, CD22, CD38, CD123, CD138, and BCMA.

In some embodiments, the engineered T cell does not express HLA-A, HLA-B, and/or HLA-C antigens, wherein the engineered T cell does not express B2M, wherein the engineered T cell does not express HLA-DP, HLA-DQ, and/or HLA-DR antigens, wherein the engineered T cell does not express CIITA, and/or wherein the engineered T cell does not express TCR-alpha and TCR-beta.

In some embodiments, the engineered T cell is a B2M^{indel/indel}, CIITA^{indel/indel}, TRAC^{indel/indel} cell comprising the second gene encoding an HLA-E variant, an HLA-G variant, and/or exogenous PD-L1 and/or the first gene encoding CAR inserted into the TRAC locus, into the TRB locus, into the B2M locus, or into the CIITA locus. In some embodiments, the engineered T cell is a B2M^{indel/indel}, CIITA^{indel/indel}, TRB^{indel/indel} cell comprising the second gene encoding an HLA-E variant, an HLA-G variant, and/or exogenous PD-L1 and/or the first gene encoding CAR inserted into the TRAC locus, into the TRB locus, into the B2M locus, or into the CIITA locus.

In some embodiments, the non-activated T cell and/or the engineered T cell of the present technology are in a subject. In other embodiments, the non-activated T cell and/or the engineered T cell of the present technology are in vitro.

In some embodiments, the non-activated T cell and/or the engineered T cell of the present technology express a CD8 binding agent. In some embodiments, the CD8 binding agent is an anti-CD8 antibody. In some embodiments, the anti-CD8 antibody is selected from the group consisting of a mouse anti-CD8 antibody, a rabbit anti-CD8 antibody, a human anti-CD8 antibody, a humanized anti-CD8 antibody, a camelid (e.g., llama, alpaca, camel) anti-CD8 antibody, and a fragment thereof. In some embodiments, the fragment thereof is an scFv or a VHH. In some embodiments, the CD8 binding agent binds to a CD8 alpha chain and/or a CD8 beta chain.

In some embodiments, the CD8 binding agent is fused to a transmembrane domain incorporated in the viral envelope. In some embodiments, the lentivirus vector is pseudotyped with a viral fusion protein. In some embodiments, the viral fusion protein comprises one or more modifications to reduce binding to its native receptor.

In some embodiments, the viral fusion protein is fused to the CD8 binding agent. In some embodiments, the viral fusion protein comprises Nipah virus F glycoprotein and Nipah virus G glycoprotein fused to the CD8 binding agent. In some embodiments, the lentivirus vector does not comprise a T cell activating molecule or a T cell costimulatory molecule. In some embodiments, the lentivirus vector encodes the first gene and/or the second gene.

In some embodiments, following transfer into a first subject, the non-activated T cell or the engineered T cell exhibits one or more responses selected from the group consisting of (a) a T cell response, (b) an NK cell response, and (c) a macrophage response, that are reduced as compared to a wild-type cell following transfer into a second subject. In some embodiments, the first subject and the second subject are different subjects. In some embodiments, the macrophage response is engulfment.

In some embodiments, following transfer into a subject, the non-activated T cell or the engineered T cell exhibits one or more selected from the group consisting of (a) reduced TH1 activation in the subject, (b) reduced NK cell killing in the subject, and (c) reduced killing by whole PBMCs in the subject, as compared to a wild-type cell following transfer into the subject.

In some embodiments, following transfer into a subject, the non-activated T cell or the engineered T cell elicits one or more selected from the group consisting of (a) reduced donor specific antibodies in the subject, (b) reduced IgM or IgG antibodies in the subject, and (c) reduced complement-dependent cytotoxicity (CDC) in a subject, as compared to a wild-type cell following transfer into the subject.

In some embodiments, the non-activated T cell or the engineered T cell is transduced with a lentivirus vector comprising a CD8 binding agent within the subject. In some embodiments, the lentivirus vector carries a gene encoding the CAR and/or a HLA-E variant, a HLA-G variant, and/or an exogenous PD-L1.

Provided herein are pharmaceutical compositions comprising a population of the non-activated T cells and/or the engineered T cells of the present technology and a pharmaceutically acceptable additive, carrier, diluent or excipient.

Provided herein are methods comprising administering to a subject a composition comprising a population of the non-activated T cells and/or the engineered T cells of the present technology, or one or more the pharmaceutical compositions of the present technology.

In some embodiments, the subject is not administered a T cell activating treatment before, after, and/or concurrently with administration of the composition. In some embodiments, the T cell activating treatment comprises lymphodepletion.

Provided herein are methods of treating a subject suffering from cancer, comprising administering to a subject a composition comprising a population of the non-activated T cells and/or the engineered T cells of the present technology, or one or more the pharmaceutical compositions of the present technology, wherein the subject is not administered a T cell activating treatment before, after, and/or concurrently with administration of the composition. In some embodiments, the T cell activating treatment comprises lymphodepletion.

Provided herein are methods for expanding T cells capable of recognizing and killing tumor cells in a subject in need thereof within the subject, comprising administering to a subject a composition comprising a population of the non-activated T cells and/or the engineered T cells of the present technology, or one or more the pharmaceutical compositions of the present technology, wherein the subject is not administered a T cell activating treatment before, after, and/or concurrently with administration of the composition. In some embodiments, the T cell activating treatment comprises lymphodepletion.

Provided herein are dosage regimens for treating a condition, disease or disorder in a subject comprising administration of a pharmaceutical composition comprising a population of the non-activated T cells and/or the engineered T cells of the present technology, or one or more the pharmaceutical compositions of the present technology, and a pharmaceutically acceptable additive, carrier, diluent or excipient, wherein the pharmaceutical composition is administered in about 1-3 doses.

Once altered, the presence of expression of any of the molecule described herein can be assayed using known techniques, such as Western blots, ELISA assays, FACS assays, and the like.

### U. Generation of Induced Pluripotent Stem Cells

The technology provides methods of producing hypoimmunogenic pluripotent cells. In some embodiments, the method comprises generating pluripotent stem cells. The generation of mouse and human pluripotent stem cells (generally referred to as iPSCs; miPSCs for murine cells or hiPSCs for human cells) is generally known in the art. As will be appreciated by those in the art, there are a variety of different methods for the generation of iPCSs. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al, World J. Stem Cells 7(1): 116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogenous genes.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g., fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT; SOX2, OCT4 (POU5F1), KLF4, MYC, NANOG, LIN28, and SV40L T antigen. In general, these reprogramming factor genes are provided on episomal vectors such as are known in the art and commercially available.

In general, as is known in the art, iPSCs are made from non-pluripotent cells such as, but not limited to, blood cells, fibroblasts, etc., by transiently expressing the reprogramming factors as described herein.

### V. Assays for Hypoimmunogencity Phenotypes and Retention of Pluripotency

Once the hypoimmunogenic cells have been generated, they may be assayed for their hypoimmunogenicity and/or retention of pluripotency as is described in WO2016183041 and WO2018132783.

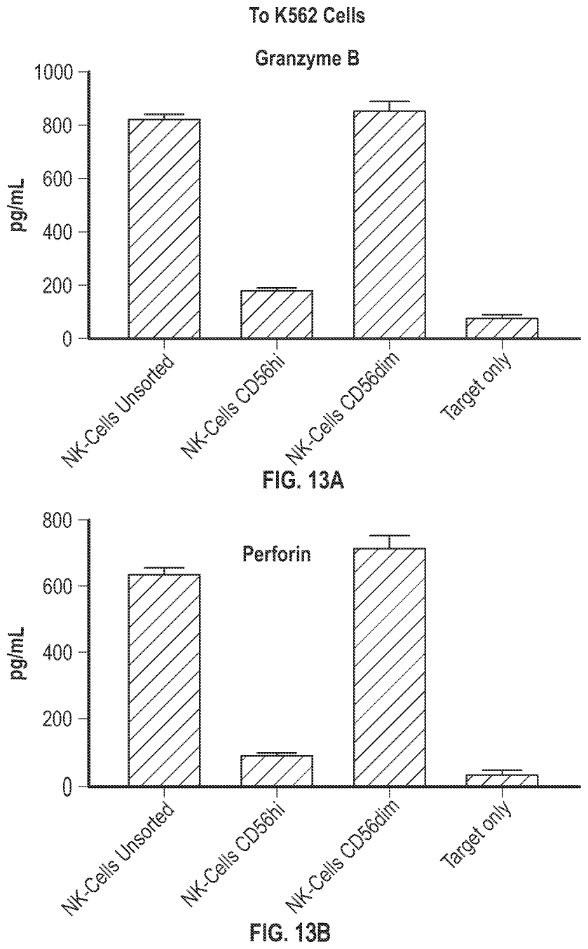

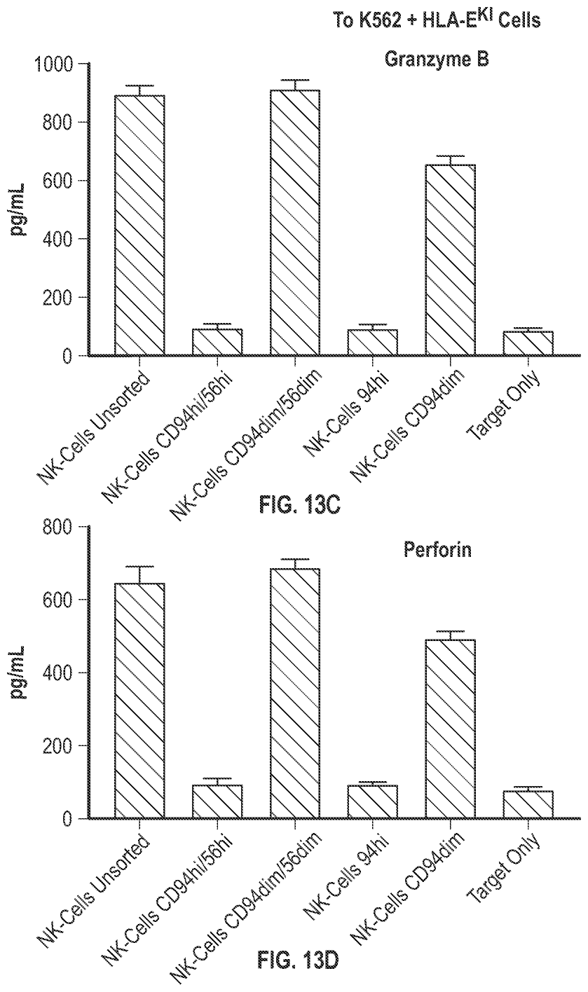

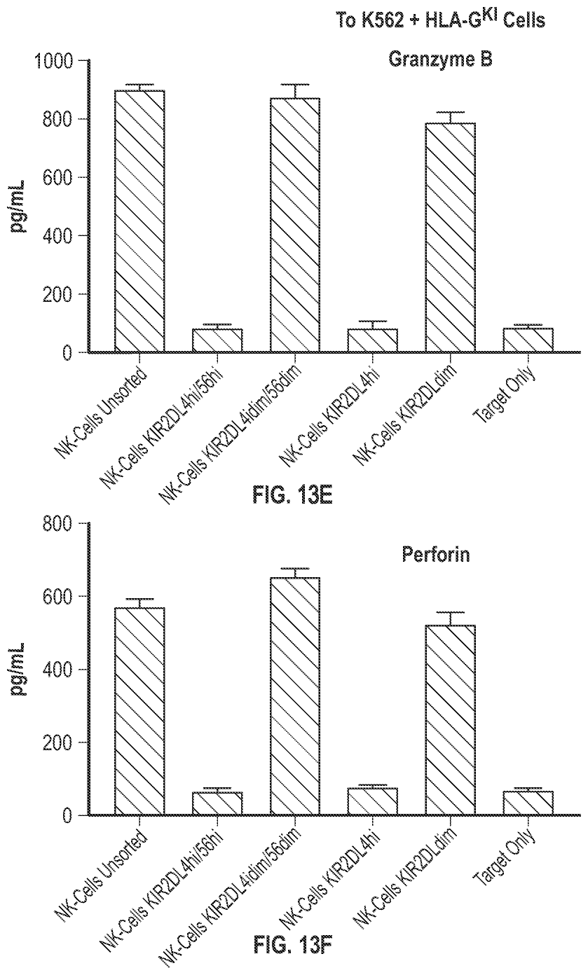

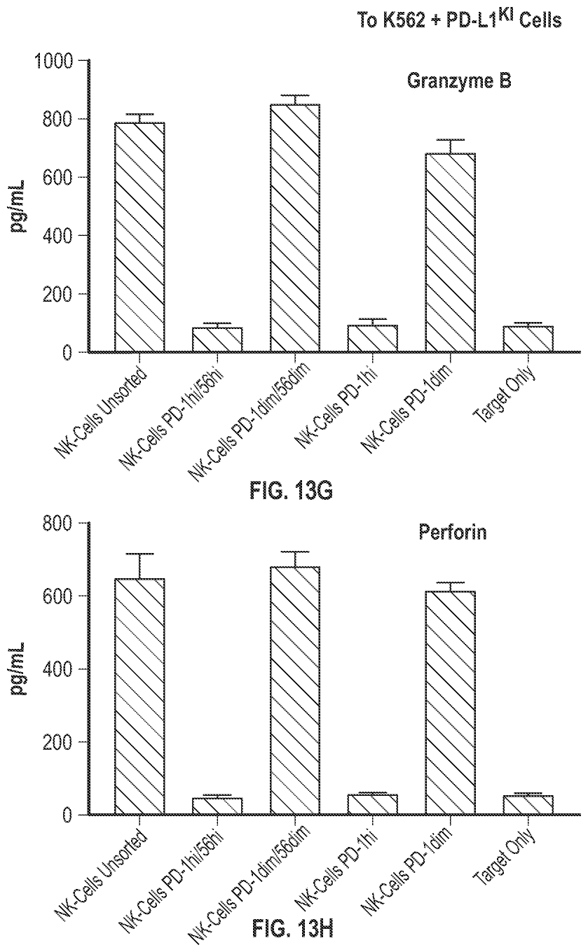

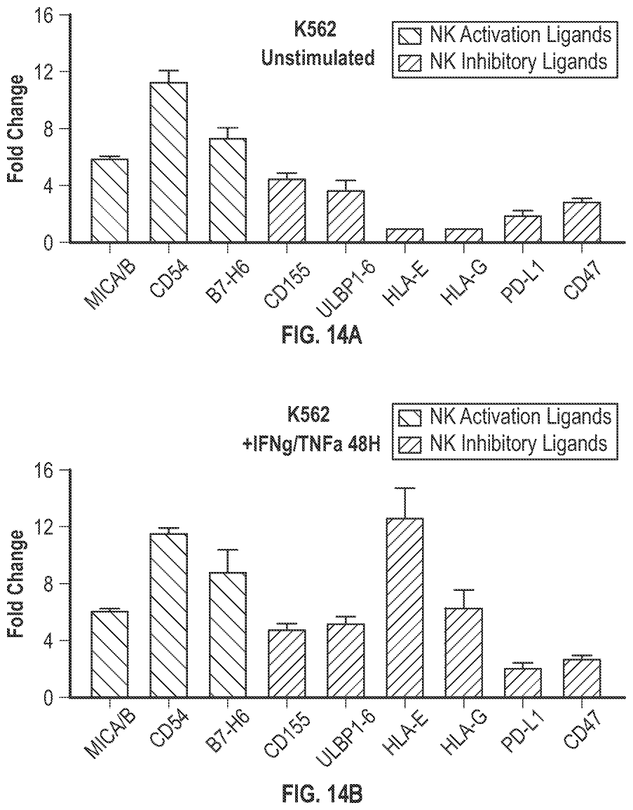

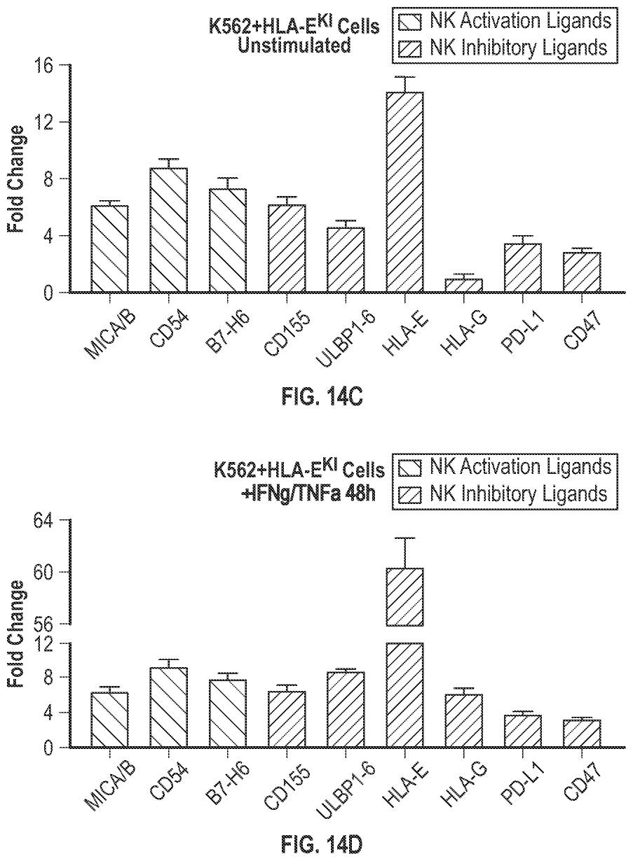

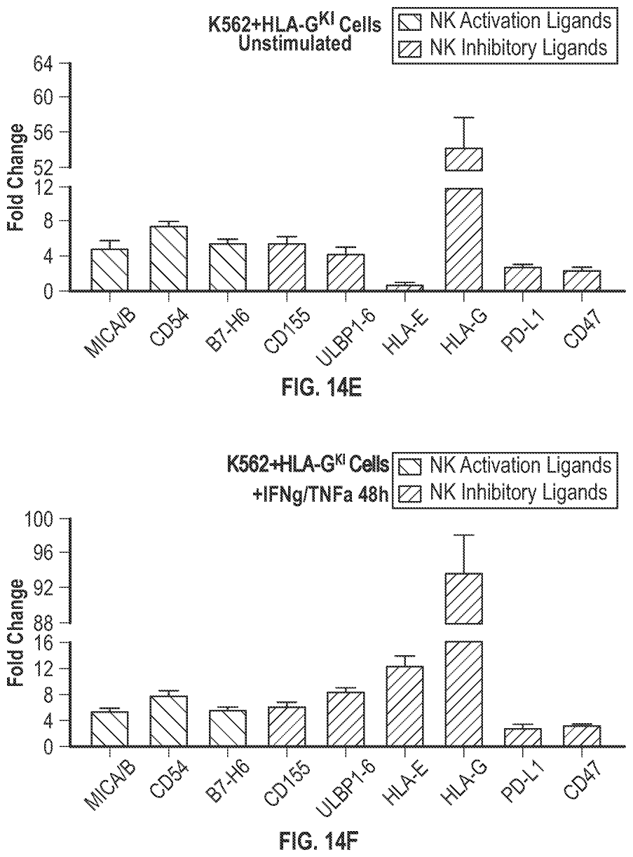

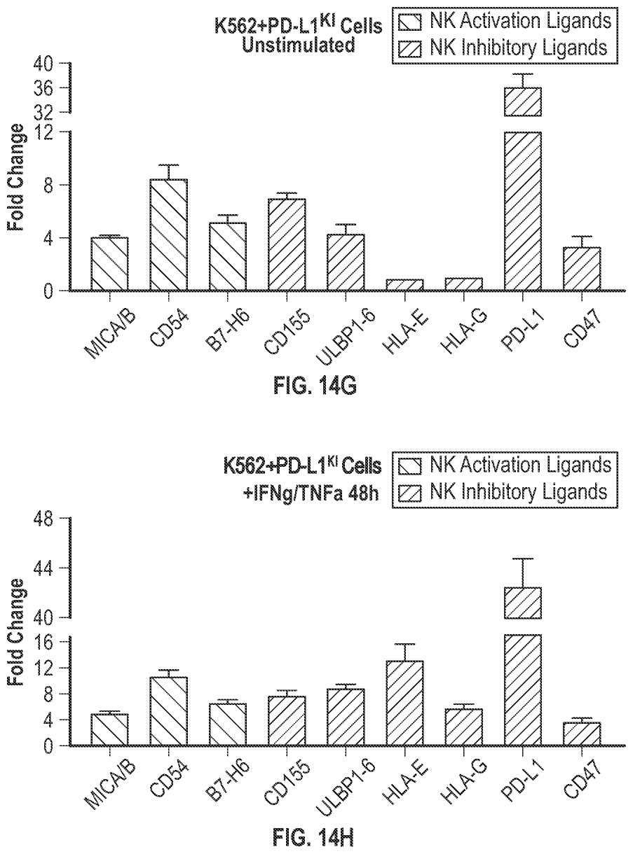

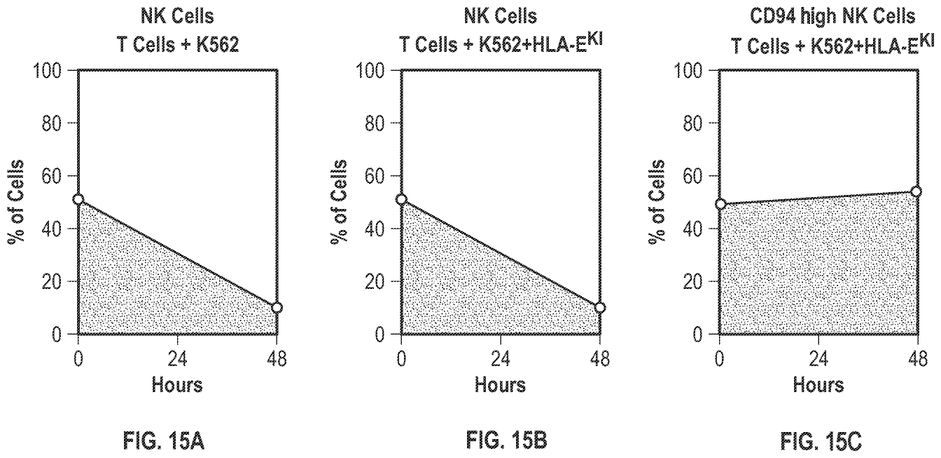

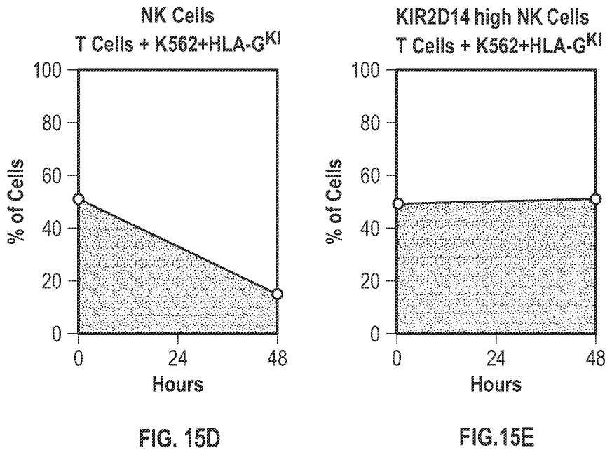

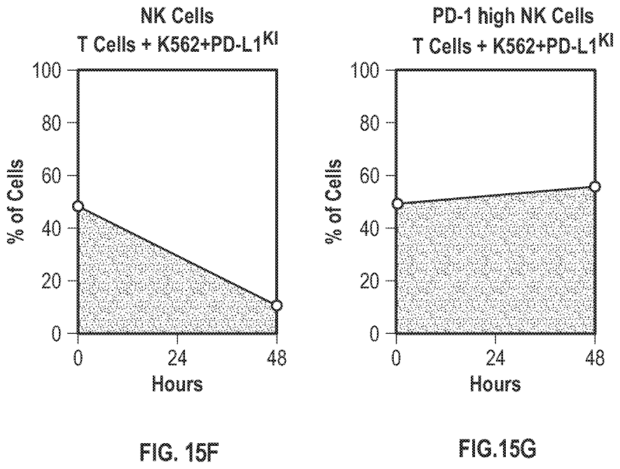

In some embodiments, hypoimmunogenicity is assayed using a number of techniques as exemplified in FIG. 13 and FIG. 15 of WO2018132783. These techniques include transplantation into allogeneic hosts and monitoring for hypoimmunogenic pluripotent cell growth (e.g., teratomas) that escape the host immune system. In some instances, hypoimmunogenic pluripotent cell derivatives are transduced to express luciferase and can then followed using bioluminescence imaging. Similarly, the T cell and/or B cell response of the host animal to such cells are tested to confirm that the cells do not cause an immune reaction in the host animal. T cell responses can be assessed by Elispot, ELISA, FACS, PCR, or mass cytometry (CYTOF). B cell responses or antibody responses are assessed using FACS or Luminex. Additionally or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of hypoimmunogenic cells is determined using an allogenic humanized immunodeficient mouse model. In some instances, the hypoimmunogenic pluripotent stem cells are transplanted into an allogenic humanized NSG-SGM3 mouse and assayed for cell rejection, cell survival, and teratoma formation. In some instances, grafted hypoimmunogenic pluripotent stem cells or differentiated cells thereof display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116(21), 10441-10446, the disclosures including the figures, figure legends, and description of methods are incorporated herein by reference in their entirety.

Similarly, the retention of pluripotency is tested in a number of ways. In some embodiments, pluripotency is assayed by the expression of certain pluripotency-specific factors as generally described herein and shown in FIG. 29 of WO2018132783. Additionally or alternatively, the pluripotent cells are differentiated into one or more cell types as an indication of pluripotency.

As will be appreciated by those in the art, the successful reduction of the MHC I function (HLA I when the cells are derived from human cells) in the pluripotent cells can be measured using techniques known in the art and as described below; for example, FACS techniques using labeled antibodies that bind the HLA complex; for example, using commercially available HLA-A, HLA-B, and HLA-C antibodies that bind to the alpha chain of the human major histocompatibility HLA Class I antigens.

In addition, the cells can be tested to confirm that the HLA I complex is not expressed on the cell surface. This may be assayed by FACS analysis using antibodies to one or more HLA cell surface components as discussed above.

The successful reduction of the MHC II function (HLA II when the cells are derived from human cells) in the pluripotent cells or their derivatives can be measured using techniques known in the art such as Western blotting using antibodies to the protein, FACS techniques, RT-PCR techniques, etc.

In addition, the cells can be tested to confirm that the HLA II complex is not expressed on the cell surface. Again, this assay is done as is known in the art (See FIG. 21 of WO2018132783, for example) and generally is done using either Western Blots or FACS analysis based on commercial antibodies that bind to human HLA Class II HLA-DR, DP and most DQ antigens.

In addition to the reduction of HLA I and II (or MHC I and II), the hypoimmunogenic cells of the technology have a reduced susceptibility to macrophage phagocytosis and NK cell killing. The resulting hypoimmunogenic cells “escape” the immune macrophage and innate pathways due to reduction or lack of the TCR complex and the expression of one or more HLA-E variant transgenes, HLA-G variant transgenes, and/or exogenous PD-L1 transgenes.

### W. Exogenous Polynucleotides

In some embodiments, the hypoimmunogenic cells provided herein are genetically modified to include one or more exogenous polynucleotides inserted into one or more genomic loci of the hypoimmunogenic cell. In some embodiments, the exogenous polynucleotide encodes a protein of interest, e.g., a chimeric antigen receptor. Any suitable method can be used to insert the exogenous polynucleotide into the genomic locus of the hypoimmunogenic cell including the gene editing methods described herein (e.g., a CRISPR/Cas system).

The exogenous polynucleotide can be inserted into any suitable genomic loci of the hypoimmunogenic cell. In some embodiments, the exogenous polynucleotide is inserted into a safe harbor locus as described herein. Suitable safe harbor loci include, but are not limited to, a CCR5 gene, a CXCR4 gene, a PPP1R12C (also known as AAVS1) gene, an albumin gene, a SHS231 locus, a CLYBL gene, a Rosa gene (e.g., ROSA26), an F3 gene (also known as CD142), a MICA gene, a MICB gene, a LRP1 gene (also known as CD91), a HMGB1 gene, an ABO gene, a RHD gene, a FUT1, and a KDM5D gene. In some embodiments, the exogenous polynucleotide is inserted into an endogenous gene wherein the insertion causes silencing or reduced expression of the endogenous gene. In some embodiments, the polynucleotide is inserted in a B2M, CIITA, TRAC, TRB, PD-1 or CTLA-4 gene. Exemplary genomic loci for insertion of an exogenous polynucleotide are depicted in Table 17.

TABLE 17
Exemplary genomic loci for insertion of exogenous polynucleotides

| | | | | Target region | Also |
|---|---|---|---|---|---|
| Number | species | name | Ensembl ID | for cleavage | known as |
| 1 | human | B2M | ENSG00000166710 | CDS | |
| 2 | human | CIITA | ENSG00000179583 | CDS | |
| 3 | human | TRAC | ENSG00000277734 | CDS | |
| 4 | human | PPP1R12C | ENSG00000125503 | Intron 1 and 2 | AAVS1 |
| 5 | human | CLYBL | ENSG00000125246 | Intron 2 | |
| 6 | human | CCR5 | ENSG00000160791 | Exons 1-3, | |
| | | | | introns 1-2, | |
| | | | | and CDS | |
| 7 | human | THUMPD3-AS1 | ENSG00000206573 | Intron 1 | ROSA26 |
| 8 | human | Ch-4: 58,976,613 | | 500 bp window | SHS231 |
| 9 | human | F3 | ENSG00000117525 | CDS | CD142 |
| 10 | human | MICA | ENSG00000204520 | CDS | |
| 11 | human | MICB | ENSG00000204516 | CDS | |
| 12 | human | LRP1 | ENSG00000123384 | CDS | |
| 13 | human | HMGB1 | ENSG00000189403 | CDS | |
| 14 | human | ABO | ENSG00000175164 | CDS | |
| 15 | human | RHD | ENSG00000187010 | CDS | |
| 16 | human | FUT1 | ENSG00000174951 | CDS | |
| 17 | human | KDM5D | ENSG00000012817 | CDS | HY |

In some embodiments, the hypoimmunogenic cell that includes the exogenous polynucleotide is derived from a hypoimmunogenic induced pluripotent cell (HIP), for example, as described herein. Such hypoimmunogenic cells include, for example, cardiac cells, neural cells, cerebral endothelial cells, dopaminergic neurons, glial cells, endothelial cells, thyroid cells, pancreatic islet cells (beta cells), retinal pigmented epithelium cells, and T cells. In some embodiments, the hypoimmunogenic cell that includes the exogenous polynucleotide is a pancreatic beta cell, a T cell (e.g., a primary T cell), or a glial progenitor cell.

In some embodiments, the hypoimmunogenic cell that includes the exogenous polynucleotide is a primary T cell or a T cell derived from a hypoimmunogenic induced pluripotent cell (e.g., a hypoimmunogenic iPSC). In exemplary embodiments, the exogenous polynucleotide is a chimeric antigen receptor (e.g., any of the CARs described herein). In some embodiments, the exogenous polynucleotide is operably linked to a promoter for expression of the exogenous polynucleotide in the hypoimmunogenic cell.

### X. Pharmaceutically Acceptable Carriers

In some embodiments, the pharmaceutical composition provided herein further include a pharmaceutically acceptable carrier. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polysorbates (TWEEN™), poloxamers (PLURONICS™) or polyethylene glycol (PEG). In some embodiments, the pharmaceutical composition includes a pharmaceutically acceptable buffer (e.g., neutral buffer saline or phosphate buffered saline).

In some embodiments, the pharmaceutical composition comprises hypoimmunogenic cells described herein and a pharmaceutically acceptable carrier comprising 31.25% (v/v) Plasma-Lyte A, 31.25% (v/v) of 5% dextrose/0.45% sodium chloride, 10% dextran 40 (LMD)/5% dextrose, 20% (v/v) of 25% human serum albumin (HSA), and 7.5% (v/v) dimethylsulfoxide (DMSO).

### Y. Formulations and Dosage Regimens

Any therapeutically effective amount of cells described herein can be included in the pharmaceutical composition, depending on the indication being treated. Non-limiting examples of the cells include primary T cells, T cells differentiated from hypoimmunogenic induced pluripotent stem cells, and other cells differentiated from hypoimmunogenic induced pluripotent stem cells described herein. In some embodiments, the pharmaceutical composition includes at least about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ cells. In some embodiments, the pharmaceutical composition includes up to about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ cells. In some embodiments, the pharmaceutical composition includes up to about 6.0×10⁸ cells. In some embodiments, the pharmaceutical composition includes up to about 8.0×10⁸ cells. In some embodiments, the pharmaceutical composition includes at least about 1×10²-5×10², 5×10²-1×10³, 1×10³-5×10³, 5×10³-1×10⁴, 1×10⁴-5×10⁴, 5×10⁴-1×10⁵, 1×10⁵-5×10⁵, 5×10⁵-1×10⁶, 1×10⁶-5×10⁶, 5×10⁶-1×10⁷, 1×10⁷-5×10⁷, 5×10⁷-1×10⁸, 1×10⁸-5×10⁸, 5×10⁸-1×10⁹, 1×10⁹-5×10⁹, 5×10⁹-1×10¹⁰, or 1×10¹⁰-5×10¹⁰ cells. In exemplary embodiments, the pharmaceutical composition includes from about 1.0×10⁶ to about 2.5×10⁸ cells. In many embodiments, the pharmaceutical composition includes from about 2.0×10⁶ to about 2.0×10⁸ cells, such as but not limited to, primary T cells, T cells differentiated from hypoimmunogenic induced pluripotent stem cells.

In some embodiments, the pharmaceutical composition has a volume of at least 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 ml. In exemplary embodiments, the pharmaceutical composition has a volume of up to about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 ml. In exemplary embodiments, the pharmaceutical composition has a volume of about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 ml. In some embodiments, the pharmaceutical composition has a volume of from about 1-50 ml, 50-100 ml, 100-150 ml, 150-200 ml, 200-250 ml, 250-300 ml, 300-350 ml, 350-400 ml, 400-450 ml, or 450-500 ml. In some embodiments, the pharmaceutical composition has a volume of from about 1-50 ml, 50-100 ml, 100-150 ml, 150-200 ml, 200-250 ml, 250-300 ml, 300-350 ml, 350-400 ml, 400-450 ml, or 450-500 ml. In some embodiments, the pharmaceutical composition has a volume of from about 1-10 ml, 10-20 ml, 20-30 ml, 30-40 ml, 40-50 ml, 50-60 ml, 60-70 ml, 70-80 ml, 70-80 ml, 80-90 ml, or 90-100 ml. In some embodiments, the pharmaceutical composition has a volume that ranges from about 5 ml to about 80 ml. In exemplary embodiments, the pharmaceutical composition has a volume that ranges from about 10 ml to about 70 ml. In many embodiments, the pharmaceutical composition has a volume that ranges from about 10 ml to about 50 ml.

The specific amount/dosage regimen will vary depending on the weight, gender, age and health of the individual; the formulation, the biochemical nature, bioactivity, bioavailability and the side effects of the cells and the number and identity of the cells in the complete therapeutic regimen.

In some embodiments, a dose of the pharmaceutical composition includes about 1.0×10⁵ to about 2.5×10⁸ cells at a volume of about 10 ml to 50 ml and the pharmaceutical composition is administered as a single dose. In some cases, the dose includes about 1.0×10⁵ to about 2.5×10⁸ primary T cells described herein at a volume of about 10 ml to 50 ml. In several cases, the dose includes about 1.0×10⁵ to about 2.5×10⁸ primary T cells that have been described above at a volume of about 10 ml to 50 ml. In various cases, the dose includes about 1.0×10⁵ to about 2.5×10⁸ T cells differentiated from hypoimmunogenic induced pluripotent stem cells described herein at a volume of about 10 ml to 50 ml. In other cases, the dose is at a range that is lower than about 1.0×10⁵ to about 2.5×10⁸ T cells, including primary T cells or T cells differentiated from hypoimmunogenic induced pluripotent stem cells. In yet other cases, the dose is at a range that is higher than about 1.0×10⁵ to about 2.5×10⁸ T cells, including primary T cells and T cells differentiated from hypoimmunogenic induced pluripotent stem cells.

In some embodiments, the pharmaceutical composition is administered as a single dose of from about 1.0×10⁵ to about 1.0×10⁷ cells (such as primary T cells and T cells differentiated from hypoimmunogenic induced pluripotent stem cells) per kg body weight for subjects 50 kg or less. In some embodiments, the pharmaceutical composition is administered as a single dose of from about 0.5×10⁵ to about 1.0×10⁷, about 1.0×10⁵ to about 1.0×10⁷, about 1.0×10⁵ to about 1.0×10⁷, about 5.0×10⁵ to about 1×10⁷, about 1.0×10⁶ to about 1×10⁷, about 5.0×10⁶ to about 1.0×10⁷, about 1.0×10⁵ to about 5.0×10⁶, about 1.0×10⁵ to about 1.0×10⁶, about 1.0×10⁵ to about 5.0×10⁵, about 1.0×10⁵ to about 5.0×10⁶, about 2.0×10⁵ to about 5.0×10⁶, about 3.0×10⁵ to about 5.0×10⁶, about 4.0×10⁵ to about 5.0×10⁶, about 5.0×10⁵ to about 5.0×10⁶, about 6.0×10⁵ to about 5.0×10⁶, about 7.0×10⁵ to about 5.0×10⁶, about 8.0×10⁵ to about 5.0×10⁶, or about 9.0×10⁵ to about 5.0×10⁶ cells per kg body weight for subjects 50 kg or less. In some embodiments, the dose is from about 0.2×10⁶ to about 5.0×10⁶ cells per kg body weight for subjects 50 kg or less. In many embodiments, the dose is at a range that is lower than from about 0.2×10⁶ to about 5.0×10⁶ cells per kg body weight for subjects 50 kg or less. In many embodiments, the dose is at a range that is higher than from about 0.2×10⁶ to about 5.0×10⁶ cells per kg body weight for subjects 50 kg or less. In exemplary embodiments, the single dose is at a volume of about 10 ml to 50 ml. In some embodiments, the dose is administered intravenously.

In exemplary embodiments, the cells are administered in a single dose of from about 1.0×10⁶ to about 5.0×10⁸ cells (such as primary T cells and T cells differentiated from hypoimmunogenic induced pluripotent stem cells) for subjects above 50 kg. In some embodiments, the pharmaceutical composition is administered as a single dose of from about 0.5×10⁶ to about 1.0×10⁹, about 1.0×10⁶ to about 1.0×10⁹, about 1.0×10⁶ to about 1.0×10⁹, about 5.0×10⁶ to about 1.0×10⁹, about 1.0×10⁷ to about 1.0×10⁹, about 5.0×10⁷ to about 1.0×10⁹, about 1.0×10⁶ to about 5.0×10⁷, about 1.0×10⁶ to about 1.0×10⁷, about 1.0×10⁶ to about 5.0×10⁷, about 1.0×10⁷ to about 5.0×10⁸, about 2.0×10⁷ to about 5.0×10⁸, about 3.0×10⁷ to about 5.0×10⁸, about 4.0×10⁷ to about 5.0×10⁸, about 5.0×10⁷ to about 5.0×10⁸, about 6.0×10⁷ to about 5.0×10⁸, about 7.0×10⁷ to about 5.0×10⁸, about 8.0×10⁷ to about 5.0×10⁸, or about 9.0×10⁷ to about 5.0×10⁸ cells per kg body weight for subjects 50 kg or less. In many embodiments, the cells are administered in a single dose of about 1.0×10⁷ to about 2.5×10⁸ cells for subjects above 50 kg. In some embodiments, the cells are administered in a single dose of a range that is less than about 1.0×10⁷ to about 2.5×10⁸ cells for subjects above 50 kg. In some embodiments, the cells are administered in a single dose of a range that is higher than about 1.0×10⁷ to about 2.5×10⁸ cells for subjects above 50 kg. In some embodiments, the dose is administered intravenously. In exemplary embodiments, the single dose is at a volume of about 10 ml to 50 ml. In some embodiments, the dose is administered intravenously.

In exemplary embodiments, the dose is administered intravenously at a rate of about 1 to 50 ml per minute, 1 to 40 ml per minute, 1 to 30 ml per minute, 1 to 20 ml per minute, 10 to 20 ml per minute, 10 to 30 ml per minute, 10 to 40 ml per minute, 10 to 50 ml per minute, 20 to 50 ml per minute, 30 to 50 ml per minute, 40 to 50 ml per minute. In numerous embodiments, the pharmaceutical composition is stored in one or more infusion bags for intravenous administration. In some embodiments, the dose is administered completely at no more than 10 minutes, 15 minutes, 20 minutes, 25 minutes, 30 minutes, 35 minutes, 40 minutes, 45 minutes, 50 minutes, 55 minutes, 60 minutes, 70 minutes, 80 minutes, 90 minutes, 120 minutes, 150 minutes, 180 minutes, 240 minutes, or 300 minutes.

In some embodiments, a single dose of the pharmaceutical composition is present in a single infusion bag. In other embodiments, a single dose of the pharmaceutical composition is divided into 2, 3, 4 or 5 separate infusion bags.

In some embodiments, the cells described herein are administered in a plurality of doses such as 2, 3, 4, 5, 6 or more doses. In some embodiments, each dose of the plurality of doses is administered to the subject ranging from 1 to 24 hours apart. In some instances, a subsequent dose is administered from about 1 hour to about 24 hours (e.g., about 1, 2, 3, 4, 5, 6, 7, 8, 9, 10, 11, 12, 13, 14, 15, 16, 17, 18, 19, 20, 21, 22, 23 or about 24 hours) after an initial or preceding dose. In some embodiments, each dose of the plurality of doses is administered to the subject ranging from about 1 day to 28 days apart. In some instances, a subsequent dose is administered from about 1 day to about 28 days (e.g., about 1, 2, 3, 4, 5, 6, 7, 8, 9, 10, 11, 12, 13, 14, 15, 16, 17, 18, 19, 20, 21, 22, 23, 24, 25, 26, 27, or about 28 days) after an initial or preceding dose. In many embodiments, each dose of the plurality of doses is administered to the subject ranging from 1 week to about 6 weeks apart. In certain instances, a subsequent dose is administered from about 1 week to about 6 weeks (e.g., about 1, 2, 3, 4, 5, or 6 weeks) after an initial or preceding dose. In several embodiments, each dose of the plurality of doses is administered to the subject ranging from about 1 month to about 12 months apart. In several instances, a subsequent dose is administered from about 1 month to about 12 months (e.g., about 1, 2, 3, 4, 5, 6, 7, 8, 9, 10, 11, or 12 months) after an initial or preceding dose.

In some embodiments, a subject is administered a first dosage regimen at a first timepoint, and then subsequently administered a second dosage regimen at a second timepoint. In some embodiments, the first dosage regimen is the same as the second dosage regimen. In other embodiments, the first dosage regimen is different than the second dosage regimen. In some instances, the number of cells in the first dosage regimen and the second dosage regimen are the same. In some instances, the number of cells in the first dosage regimen and the second dosage regimen are different. In some cases, the number of doses of the first dosage regimen and the second dosage regimen are the same. In some cases, the number of doses of the first dosage regimen and the second dosage regimen are different.

In some embodiments, the first dosage regimen includes hypoimmune T cells or primary T cells expressing a first CAR and the second dosage regimen includes hypoimmune T cells or primary T cells expressing a second CAR such that the first CAR and the second CAR are different. For instance, the first CAR and second CAR bind different target antigens. In some cases, the first CAR includes an scFv that binds an antigen and the second CAR includes an scFv that binds a different antigen. In some embodiments, the first dosage regimen includes hypoimmune T cell or primary T cells expressing a first CAR and the second dosage regimen includes hypoimmune T cell or primary T cells expressing a second CAR such that the first CAR and the second CAR are the same. The first dosage regimen can be administered to the subject at least 1 month, 2 months, 3 months, 4 months, 5 months, 6 months, 7 months, 8 months, 9 months, 10 months, 11 months, 12 months, 1-3 months, 1-6 months, 4-6 months, 3-9 months, 3-12 months, or more months apart from the second dosage regimen. In some embodiments, a subject is administered a plurality of dosage regimens during the course of a disease (e.g., cancer) and at least two of the dosage regimens comprise the same type of hypoimmune T cells or primary T cells described herein. In other embodiments, at least two of the plurality of dosage regimens comprise different types of hypoimmune T cells or primary T cells described herein.

### Z. Methods for Administering Hypoimmunogenic Cells Including T Cells

As is described in further detail herein, provided herein are methods for treating a patient with a condition, disorder, or disorder through administration of hypoimmunogenic cells, particularly hypoimmunogenic T cells. As will be appreciated, for all the multiple embodiments described herein related to the timing and/or combinations of therapies, the administration of the cells is accomplished by a method or route which results in at least partial localization of the introduced cells at a desired site. The cells can be infused, implanted, or transplanted directly to the desired site, or alternatively be administered by any appropriate route which results in delivery to a desired location in the subject where at least a portion of the implanted cells or components of the cells remain viable.

Provided herein are methods for treating a patient with a condition, disorder, or disorder includes administration of a population of hypoimmunogenic cells (e.g., primary T cells, T cells differentiated from hypoimmunogenic induced pluripotent stem cells, or other cells differentiated from hypoimmunogenic induced pluripotent stem cells described herein) to a subject, e.g., a human patient. For instance, a population of hypoimmunogenic primary T cells such as, but limited to, CD3+ T cells, CD4+ T cells, CD8+ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells that express CD45RA (TEMRA cells), tissue-resident memory (Trm) cells, virtual memory T cells, innate memory T cells, memory stem cell (Tsc), γδ T cells, and any other subtype of T cell is administered to a patient to treat a condition, disorder, or disorder. In some embodiments, an immunosuppressive and/or immunomodulatory agent (such as, but not limited to a lymphodepletion agent) is not administered to the patient before the administration of the population of hypoimmunogenic cells. In some embodiments, an immunosuppressive and/or immunomodulatory agent is administered at least 1, 2, 3, 4, 5, 6, 7, 8, 9, 10, 11, 12, 13, 14 days or more before the administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more before the administration of the cells. In numerous embodiments, an immunosuppressive and/or immunomodulatory agent is not administered to the patient after the administration of the cells, or is administered at least 1, 2, 3, 4, 5, 6, 7, 8, 9, 10, 11, 12, 13, 14 days or more after the administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more after the administration of the cells. In some embodiments where an immunosuppressive and/or immunomodulatory agent is administered to the patient before or after the administration of the cells, the administration is at a lower dosage than would be required for cells with MHC I and/or MHC II expression and without exogenous expression of one or more receptors selected from the group consisting of HLA-E, HLA-G, PD-L1, CD47, and the like.

Non-limiting examples of an immunosuppressive and/or immunomodulatory agent (such as, but not limited to a lymphodepletion agent) include cyclosporine, azathioprine, mycophenolic acid, mycophenolate mofetil, corticosteroids such as prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin, thymosin-α and similar agents. In some embodiments, the immunosuppressive and/or immunomodulatory agent is selected from a group of immunosuppressive antibodies consisting of antibodies binding to p75 of the IL-2 receptor, antibodies binding to, for instance, MHC, CD2, CD3, CD4, CD7, CD28, B7, CD40, CD45, IFN-gamma, TNF-alpha, IL-4, IL-5, IL-6R, IL-6, IGF, IGFR1, IL-7, IL-8, IL-10, CD11a, or CD58, and antibodies binding to any of their ligands. In some embodiments, such an immunosuppressive and/or immunomodulatory agent may be selected from soluble IL-15R, IL-10, B7 molecules (e.g., B7-1, B7-2, variants thereof, and fragments thereof), ICOS, and OX40, an inhibitor of a negative T cell regulator (such as an antibody against CTLA-4) and similar agents.

In some embodiments, where an immunosuppressive and/or immunomodulatory agent is administered to the patient before or after the administration of the cells, the administration is at a lower dosage than would be required for cells with MHC I and/or MHC II expression, TCR expression and without exogenous expression of CD47. In some embodiments, where an immunosuppressive and/or immunomodulatory agent is administered to the patient before or after the first administration of the cells, the administration is at a lower dosage than would be required for cells with MHC I and MHC II expression, TCR expression and without exogenous expression of one or more receptors selected from the group consisting of HLA-E, HLA-G, PD-L1, CD47, and the like.

In some embodiments, the cells described are co-administered with a therapeutic agent that that binds to and/or interacts with one or more receptors selected from the group consisting of CD94, KIR2DL4, PD-1, an inhibitory NK cell receptor, and an activating NK receptor. In some instances, the therapeutic agent binds to a receptor on the surface of an NK cell, including one or more subpopulations of NK cells. In some embodiments, the therapeutic agent is selected from the group consisting of an antibody and fragments and variants thereof, an antibody mimetic, a small molecule, a blocking peptide, and a receptor antagonist.

For therapeutic application, cells prepared according to the disclosed methods can typically be supplied in the form of a pharmaceutical composition comprising an isotonic excipient, and are prepared under conditions that are sufficiently sterile for human administration. For general principles in medicinal formulation of cell compositions, see “Cell Therapy: Stem Cell Transplantation, Gene Therapy, and Cellular Immunotherapy,” by Morstyn & Sheridan eds, Cambridge University Press, 1996; and “Hematopoietic Stem Cell Therapy,” E. D. Ball, J. Lister & P. Law, Churchill Livingstone, 2000. The cells can be packaged in a device or container suitable for distribution or clinical use.

### IV. EXAMPLES

### Example 1: HLA-E, HLA-G, or PD-L1 Overexpression in MHC I/II Knockout Cells

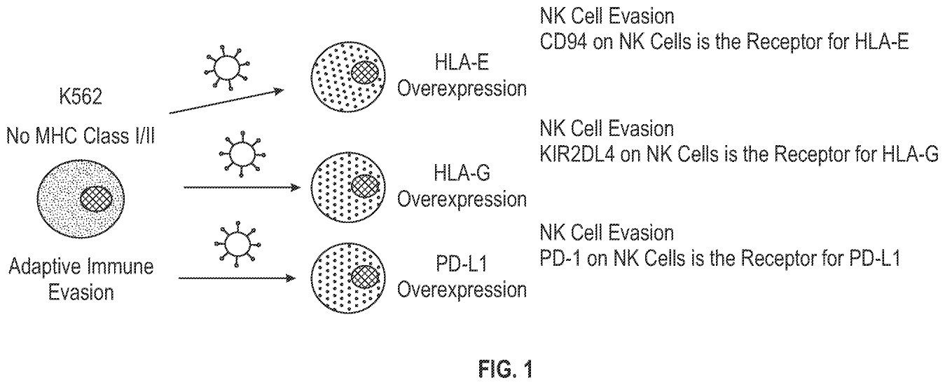

Experiments were performed to determine whether overexpression of various exemplary molecules could prevent activation of NK cell mediated innate immune responses. It is recognized in the art that HLA-I/HLA-II knock-out (MHC class I/II knock-out) cells such as K562 cells do not elicit an adaptive innate response in vitro and in vivo. Overexpression of various molecules such as HLA-E, HLA-G, and PD-L1 in K562 cells were investigated to prevent activation of NK cell mediated cytotoxicity. Briefly, K562 cells were engineered to overexpress either HLA-E, HLA-G, and PD-L1 by way of standard knock-in technology. The resulting modified K562 cells were analyzed to determine if they are able to inhibit HLA-I/II induced killing by NK cells. See FIG. 1.

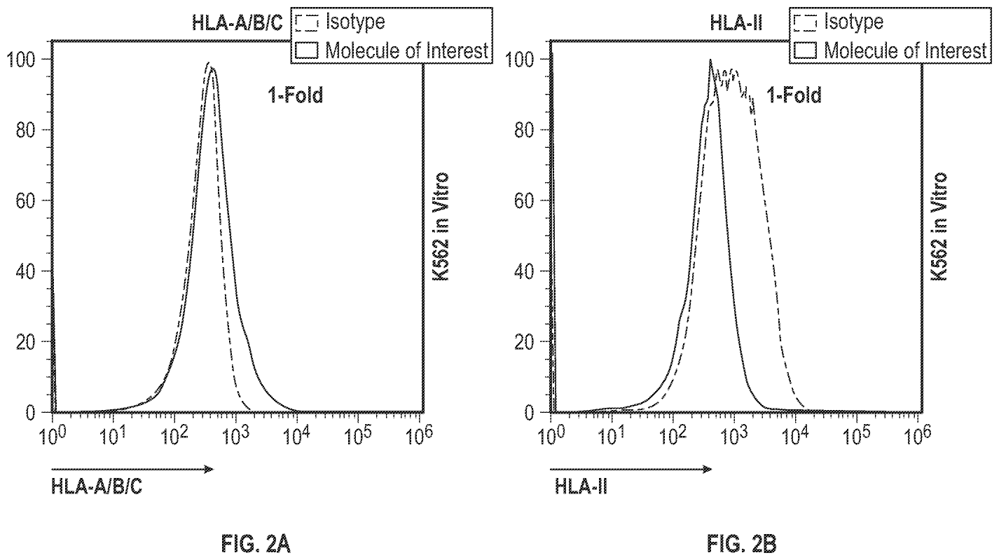

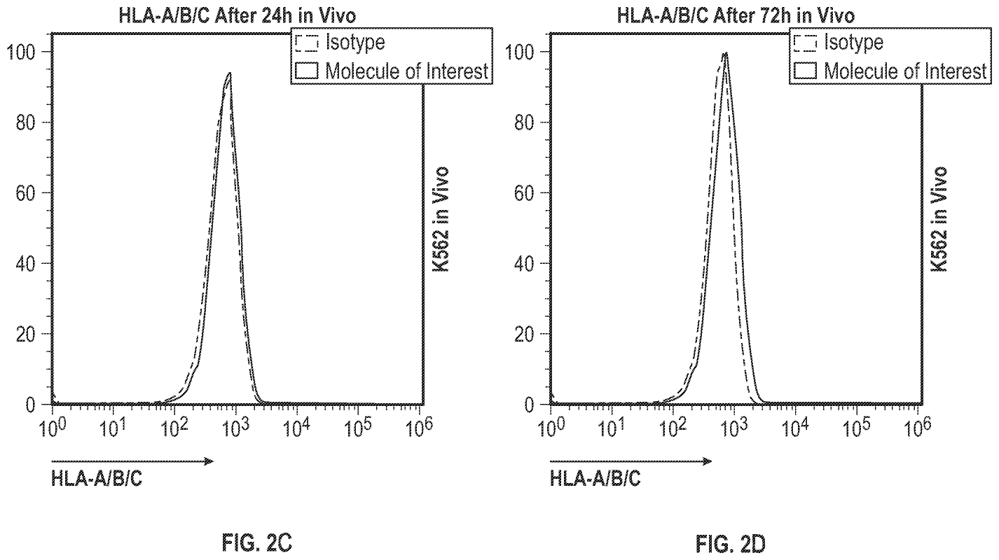

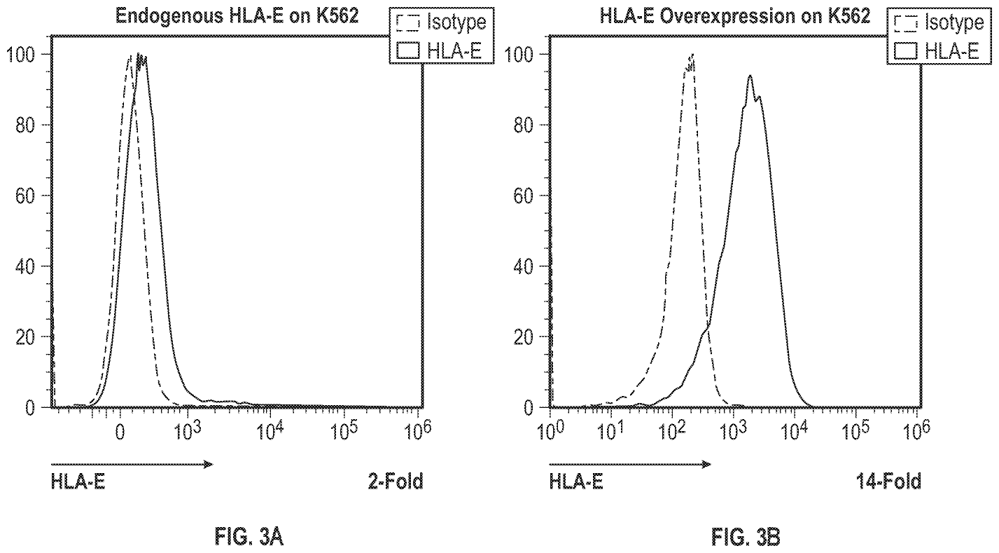

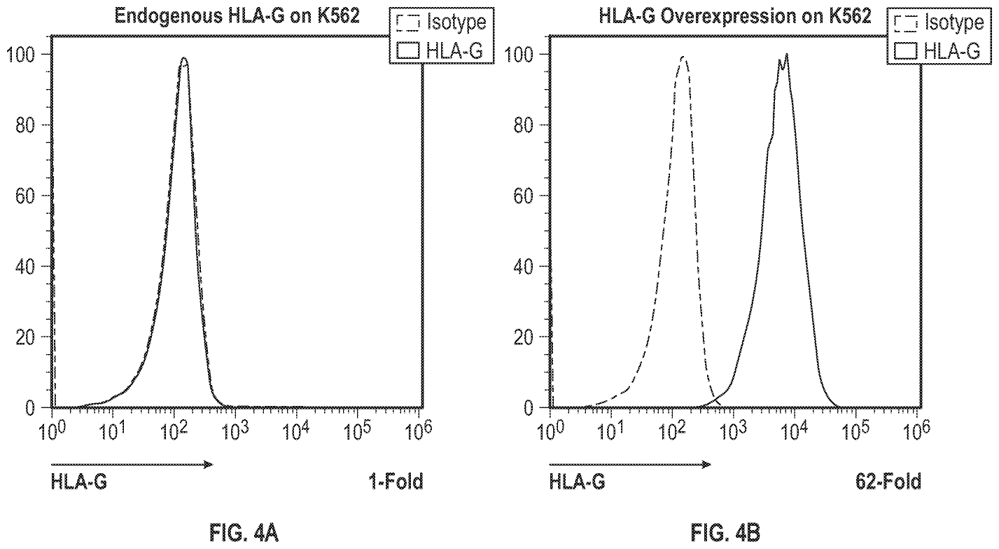

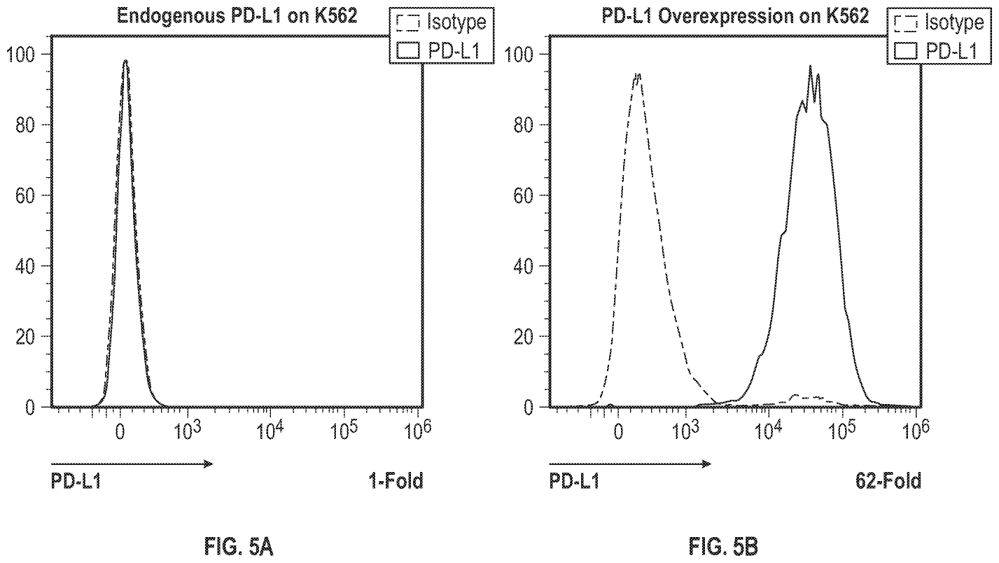

Surface expression of HLA-I, HLA-II, HLA-E, HLA-G, and PD-L1, on unmodified K562 cells and those overexpressing either HLA-E, HLA-G, or PD-L1 was measured using standard flow cytometry methods (FIGS. 2-5). Data showed that K562+HLA-E^{KI} cells expressed higher levels of HLA-E protein compared to unmodified K562 cells. Similar data was obtained for the K562+HLA-G^{KI} cells, K562+PD-L1^{KI} cells, and K562+CD47^{KI} cells.

It was also determined that HLA-I and/or HLA-II antigens are expressed on “in vivo” cells (FIGS. 2A-2D). To obtain the “in vivo” K562 cells, K562 cells were injected into humanized mice and the K562 cells were harvested 24 hours or 72 hours later. HLA-I and HLA-II antigens were not upregulated after in vivo transplantation into mice.

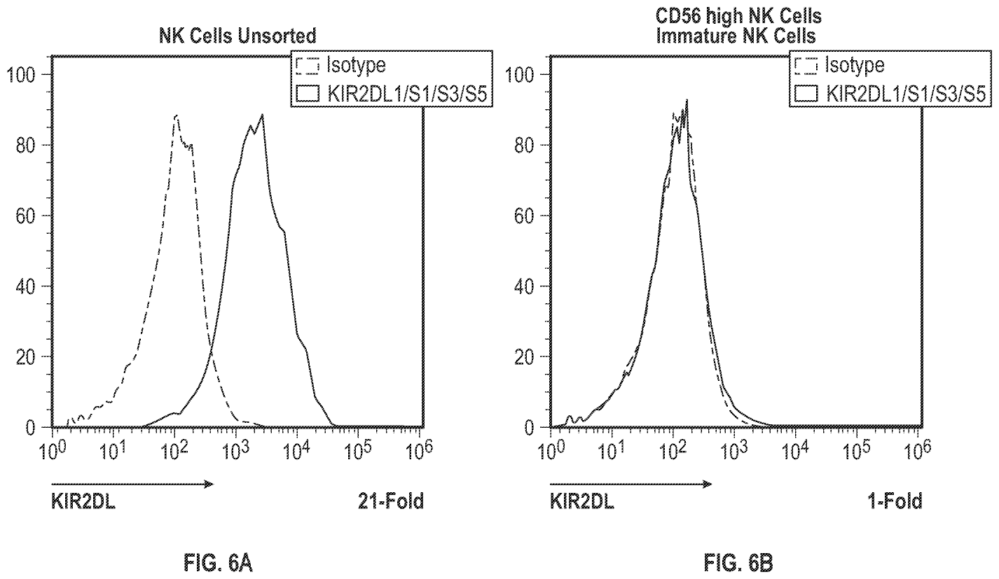

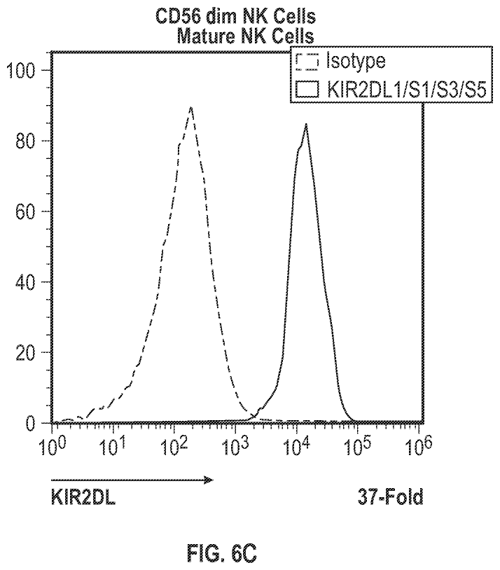

KIR receptor expression by NK cells was evaluated to confirm that those cells participate in the “missing-self” response. Immature NK cells such as CD56 high NK cells do not express KIR2DL receptors, and likely do not play a role in the “missing-self” response. Yet, mature NK cells such as CD56 dim NK cells express KIR2DL receptors and play a role in the “missing-self” response. Surface expression of KIR2DL on unsorted NK cells, CD56 high immature NK cells, and CD56 dim mature NK cells was measured using standard flow cytometry methods (FIGS. 6A-6C). Mature NK cells expressed KIR2DL at a higher level (37-fold higher) compared to an isotype control.

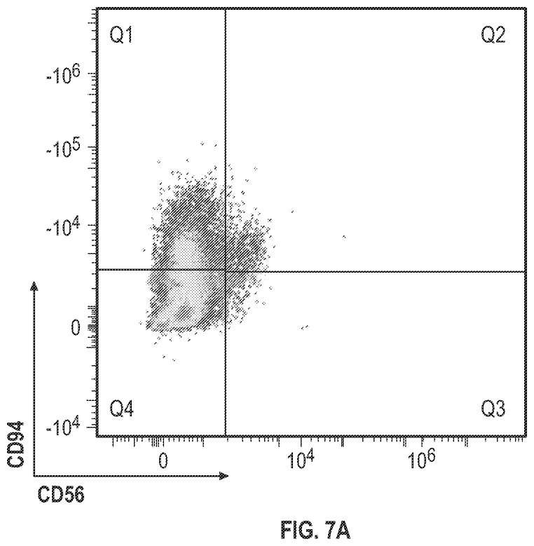

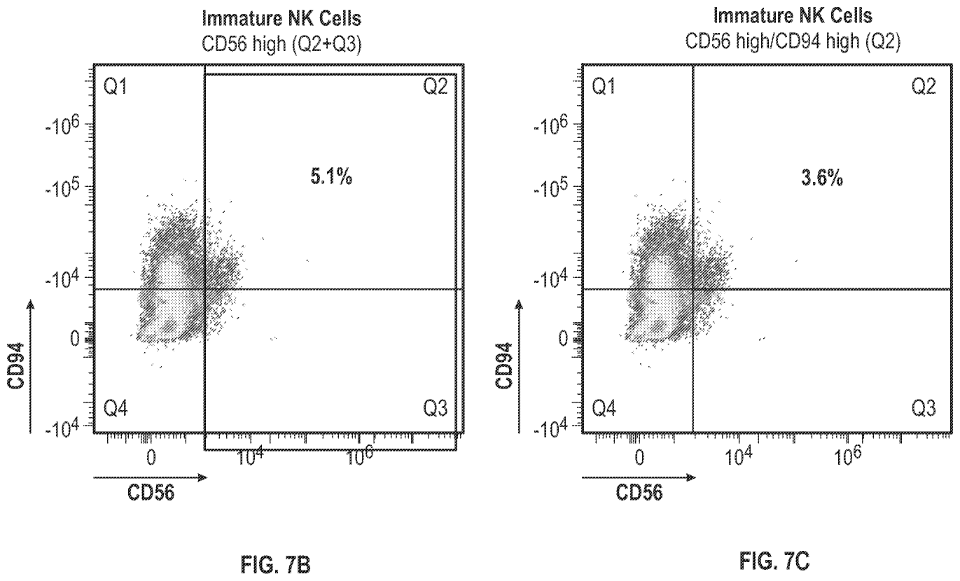

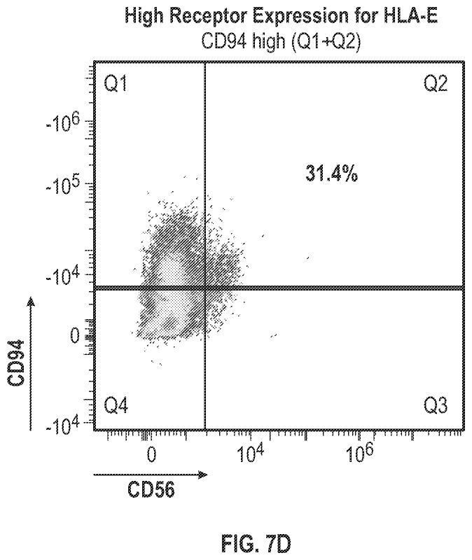

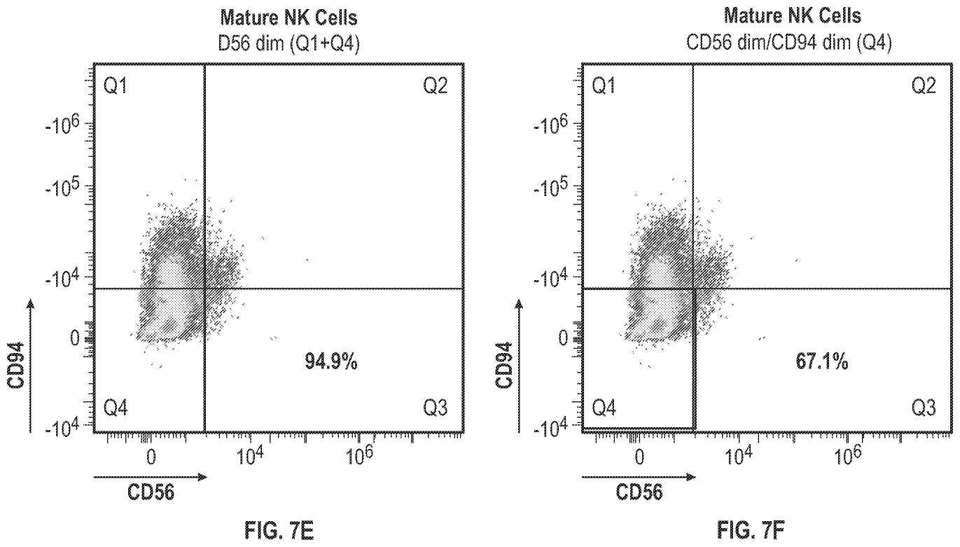

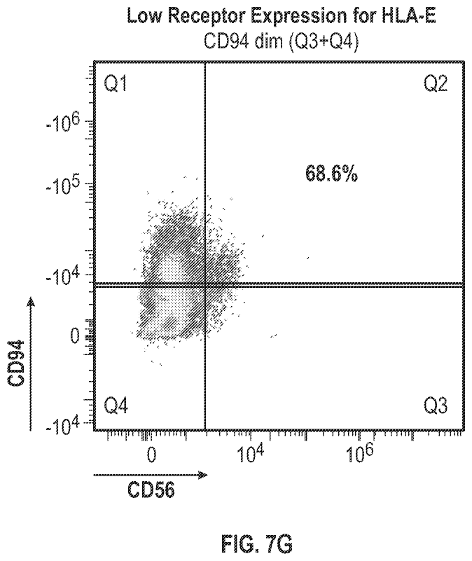

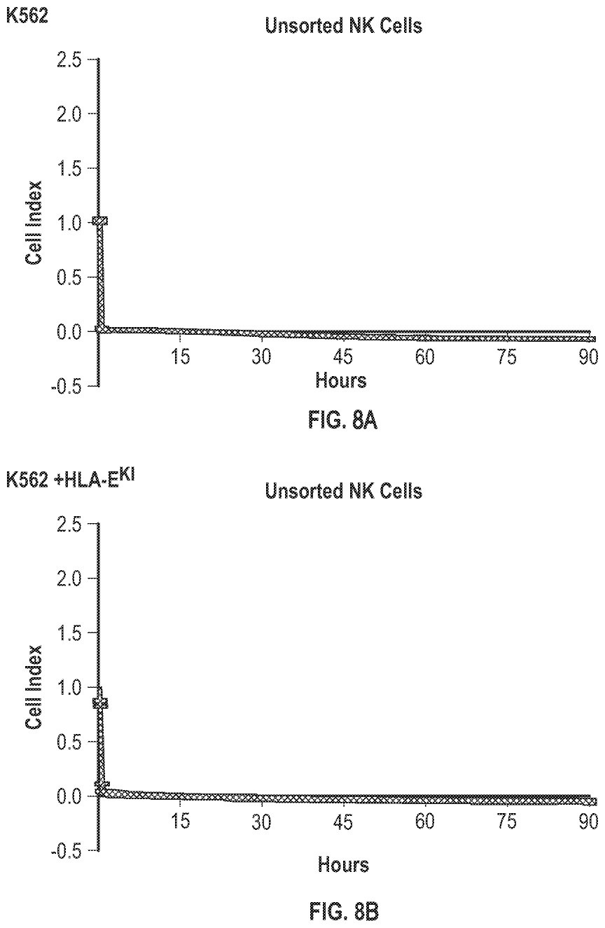

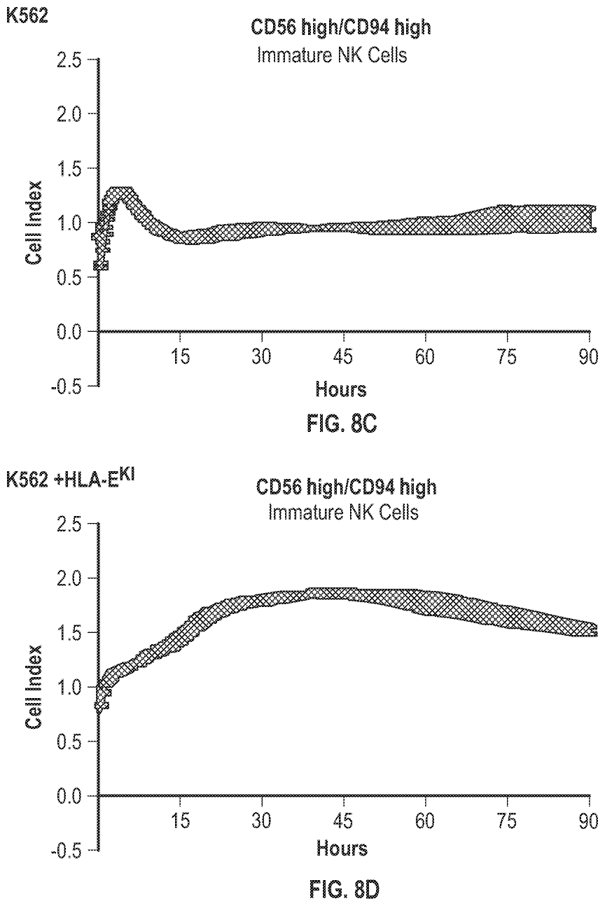

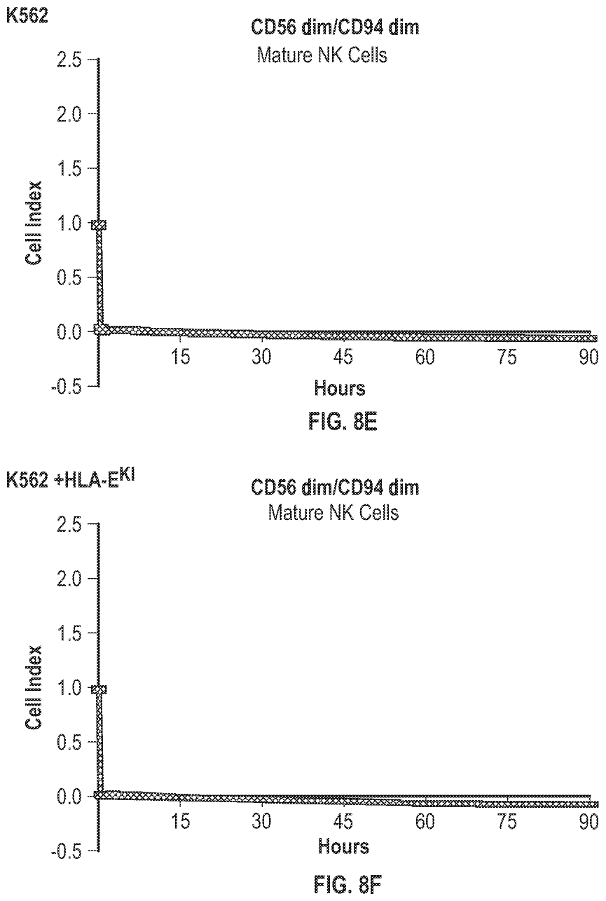

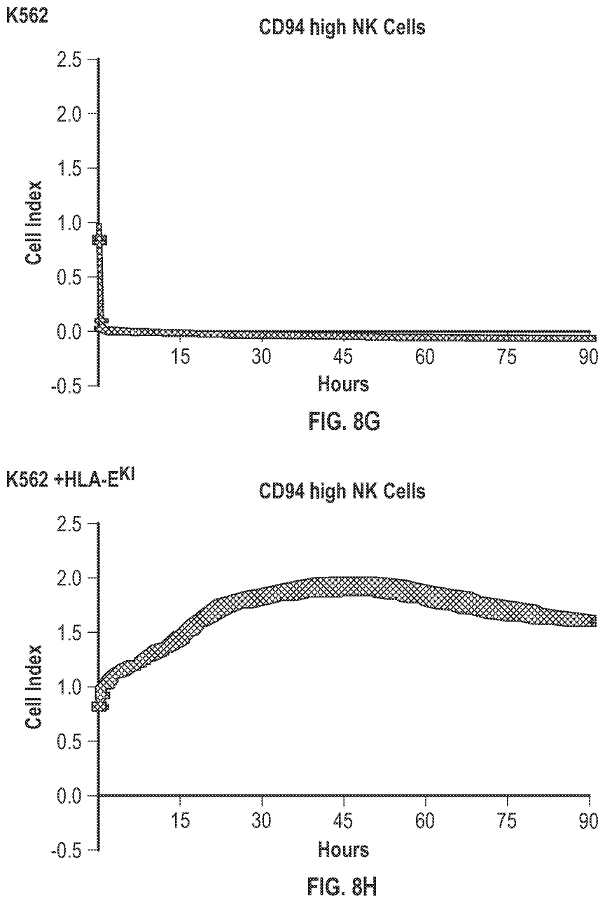

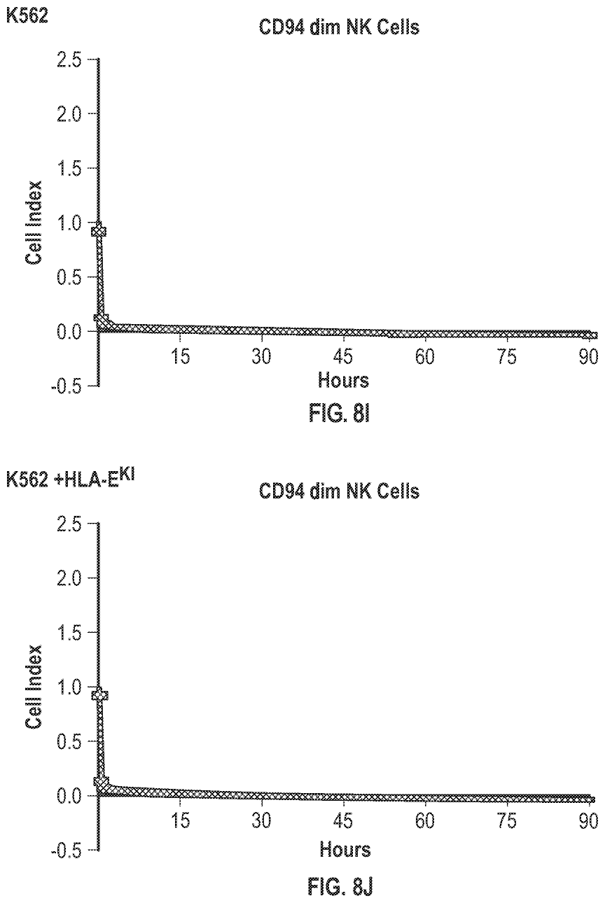

CD56 and CD94 expression was evaluated in stimulated NK cells (FIGS. 8A-8J) including various NK cell subpopulations. The percentages of various NK cell populations including immature NK cells, mature NK cells, CD94 high NK cells and CD94 dim NK cells were determined (FIGS. 7A-7E). It is recognized by those in the art that CD94 is a receptor for HLA-E.

Standard cell killing assays were performed to determine whether specific NK cell subpopulations can recognize and kill modified K562 cells overexpressing HLA-E (FIGS. 8A-8J). HLA-E knock-in K562 cells (“K562+HLA-E^{KI}” in FIGS. 8A-8J) were not protected from NK cell-mediated lysis by unsorted NK cells and mature NK cells (i.e., CD56 dim/CD94 dim NK cells). Immature NK cells (i.e., CD56 high/CD94 high NK cells) failed to recognize and kill unmodified K562 cells and HLA-E knock-in K562 cells. HLA-E overexpressing K562 cells were not killed by CD94 high NK cells, however they were killed by CD94 dim NK cells (FIGS. 8A-8J). In other words, HLA-E overexpressing K562 cells evaded NK cell mediated lysis by CD94 high NK cells.

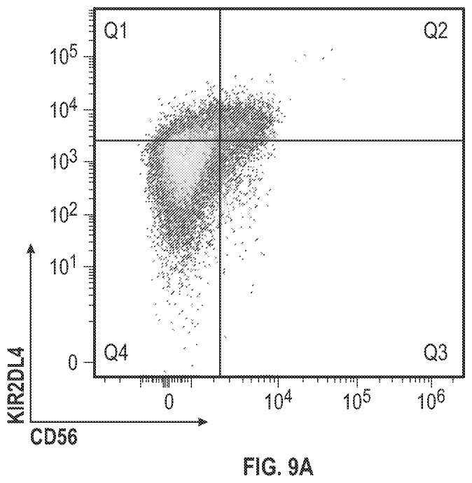

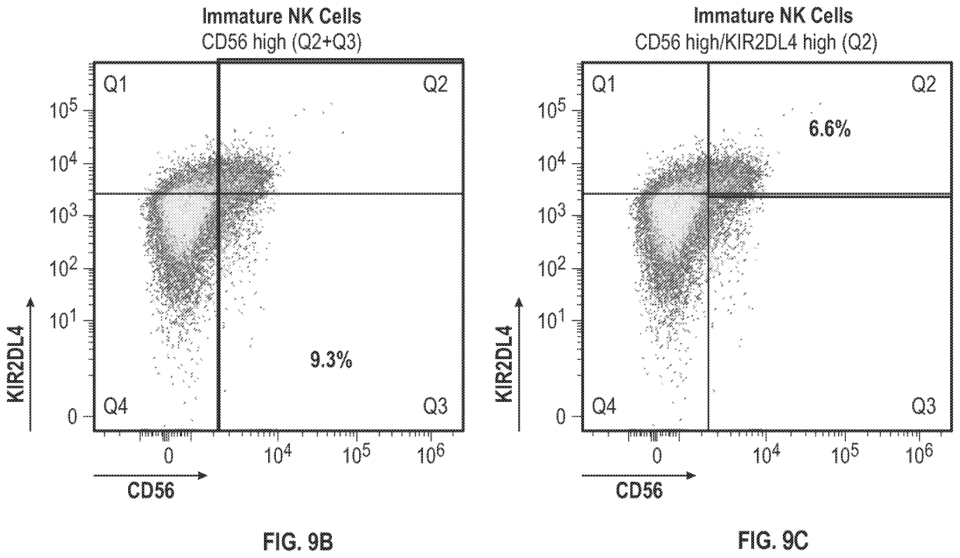

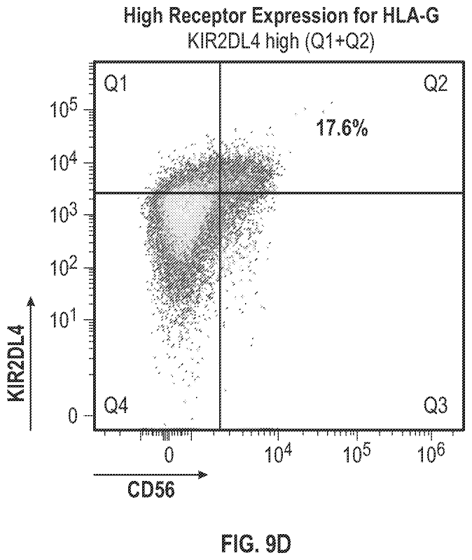

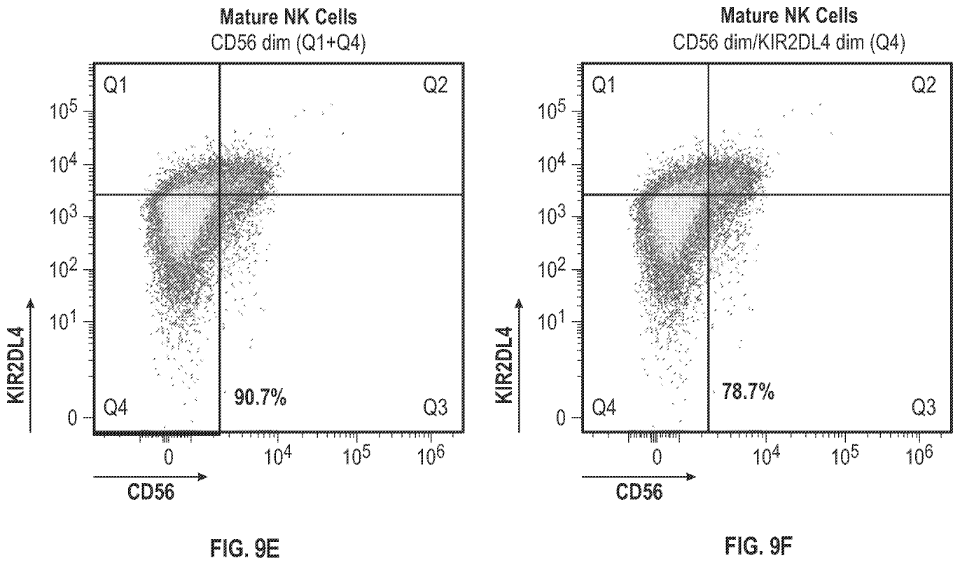

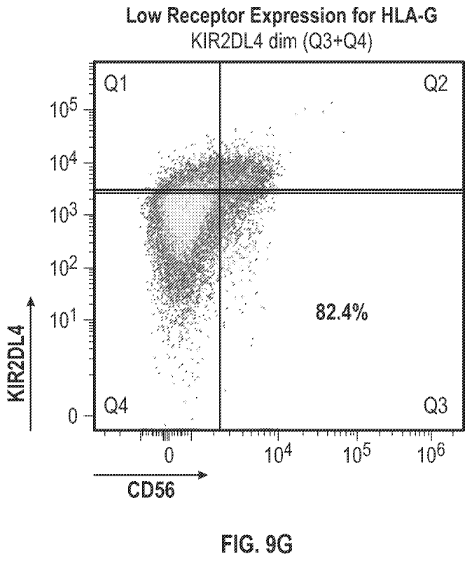

KIR2DL4 and CD56 expression was evaluated in stimulated NK cells (FIG. 9A) including the NK cell subpopulations. The percentages of various NK cell populations including immature NK cells, mature NK cells, CD56 high NK cells and KIR2DL4 high NK cells. (FIGS. 9B-9G). Less than 20% of the NK cells were KIR2DL4 high cells (FIGS. 9B-9D) and over 80% of the NK cells are KIR2DL4 dim cells (FIGS. 9E-9G). It is recognized by those in the art that KIR2DL4 is a receptor for HLA-G.

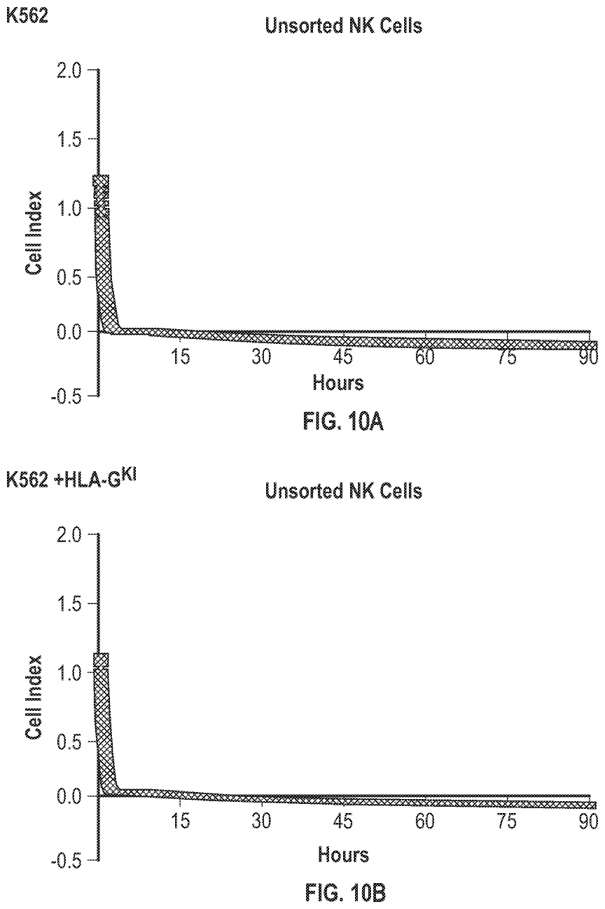

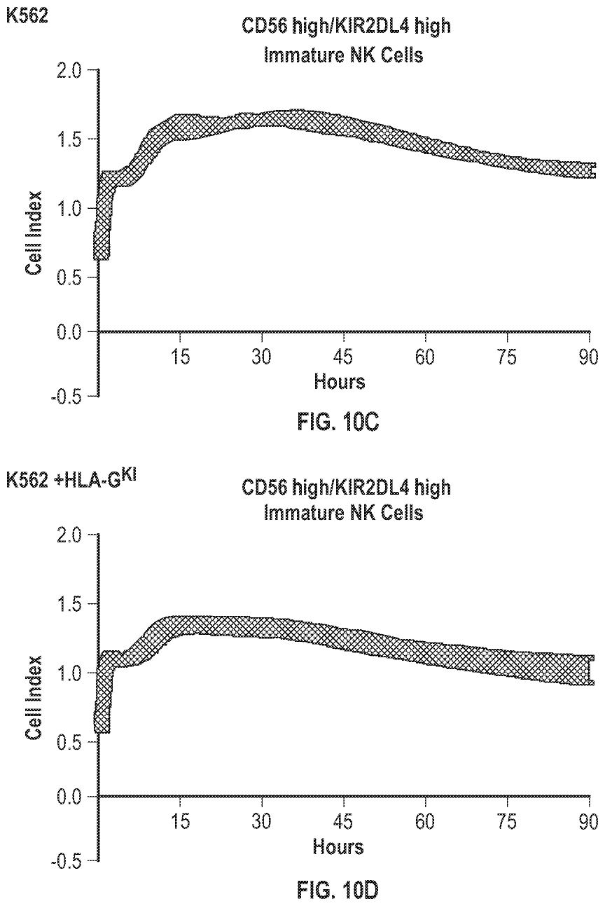

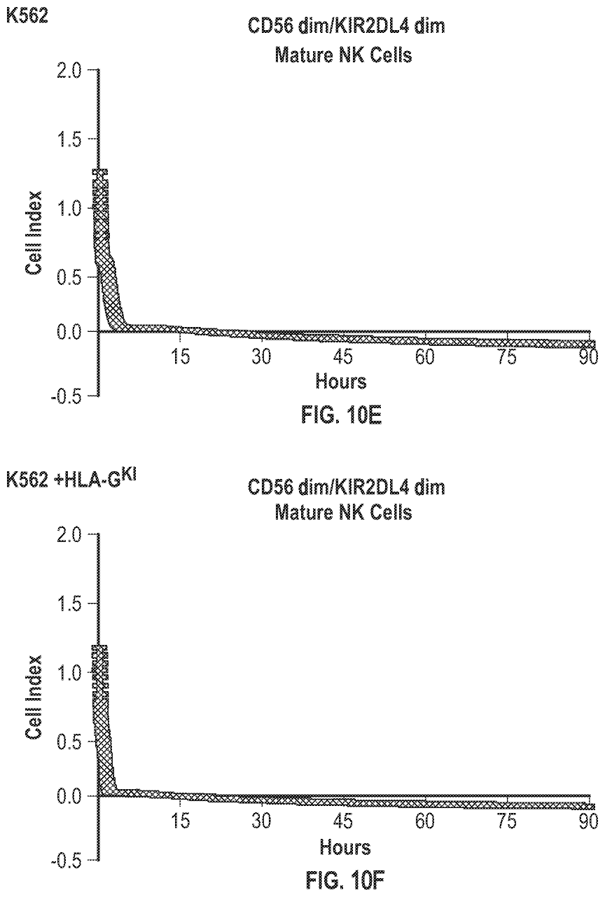

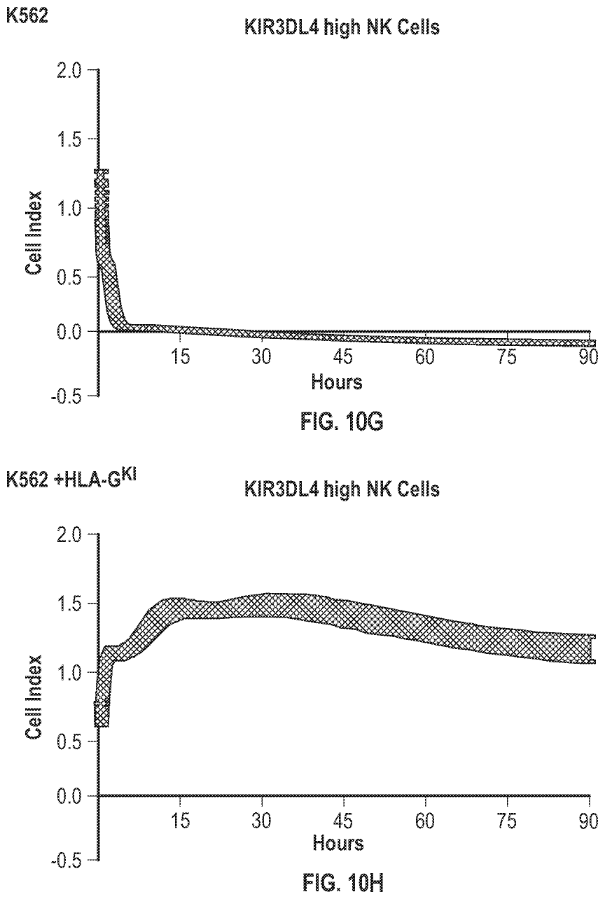

Standard cell killing assays were performed to determine whether specific NK cell subpopulations can recognize and kill modified K562 cells overexpressing HLA-G (FIGS. 10A-10J). HLA-G overexpressing K562 cells were not killed by KIR3DL4 high NK cells, however they were killed by KIR3DL4 dim NK cells (FIGS. 10D-10J). HLA-G overexpressing K562 cells evaded NK cell mediated lysis by KIR3DL4 high NK cells.

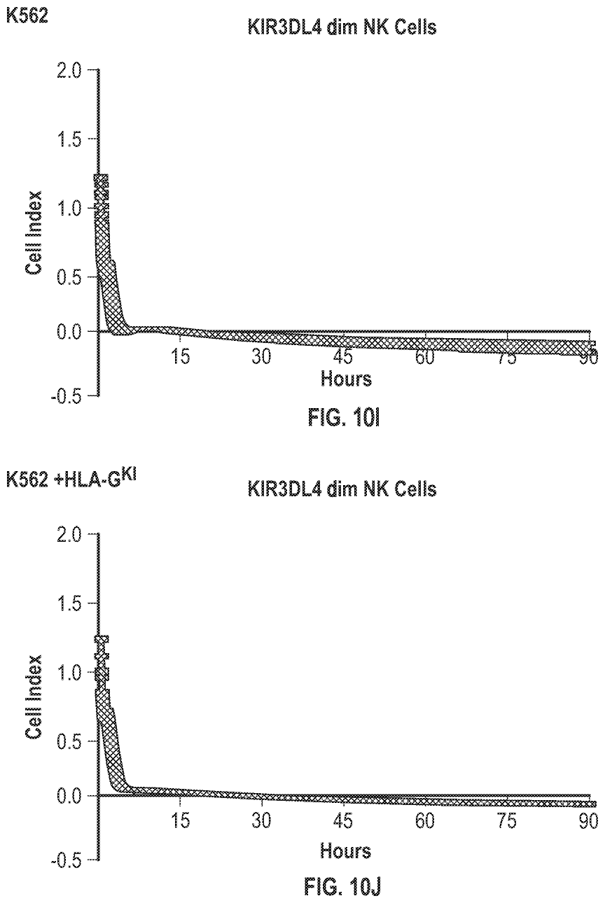

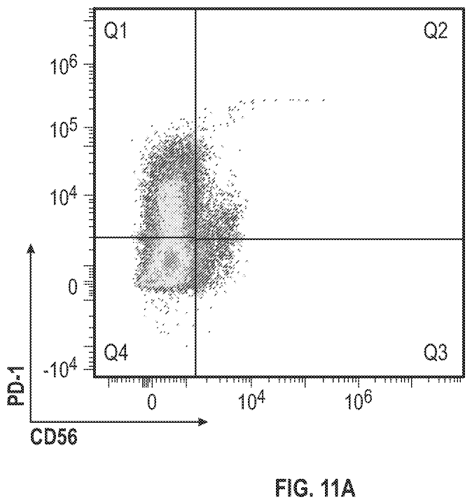

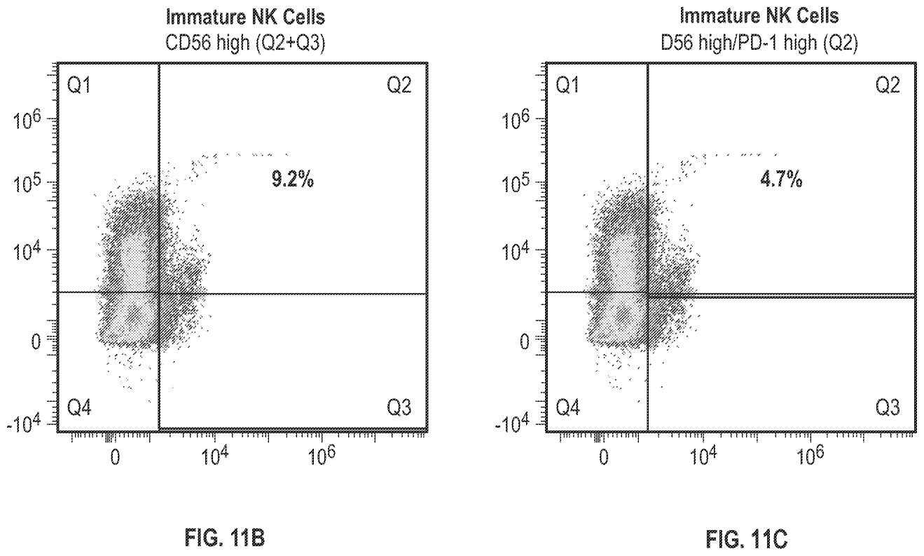

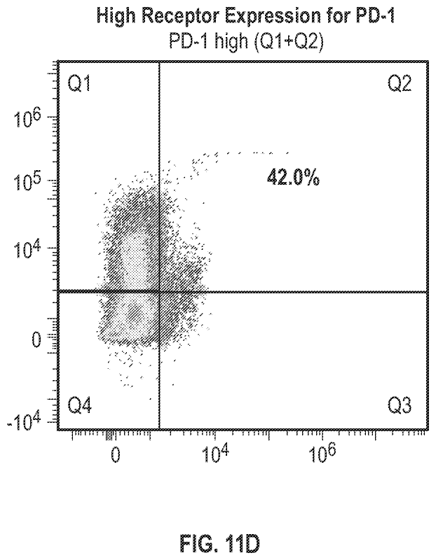

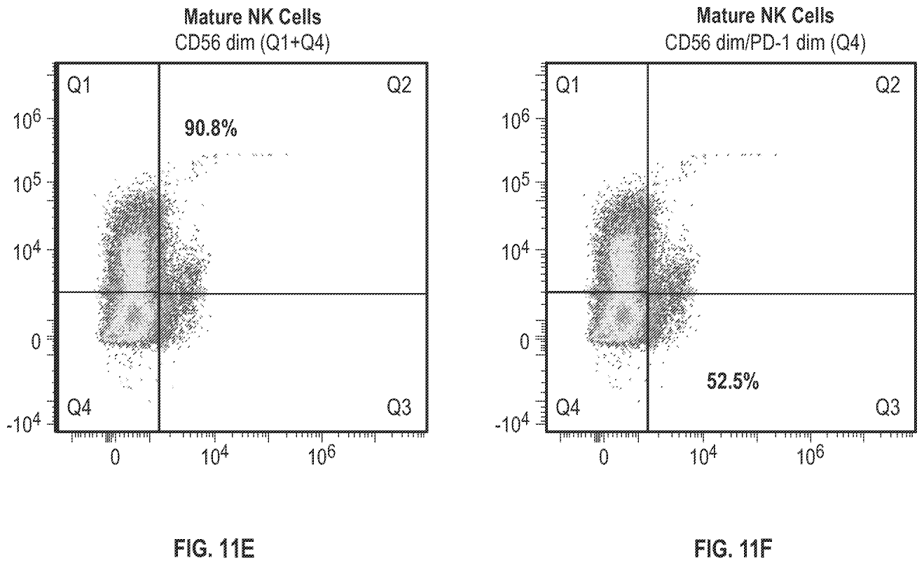

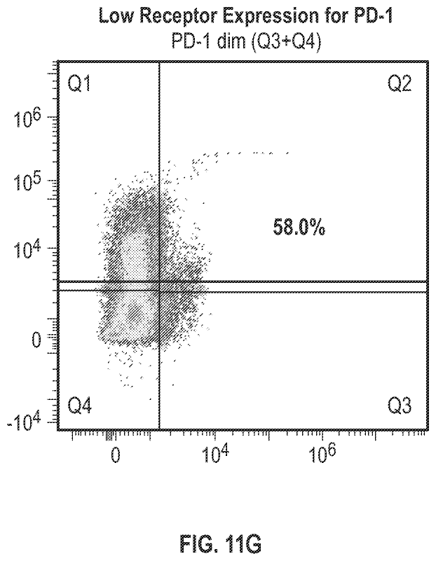

PD-1 and CD56 expression was evaluated in stimulated NK cells (FIG. 11A) including the NK cell subpopulations. The percentages of various NK cell populations including immature NK cells, mature NK cells, PD-1 high NK cells and PD-1 dim NK cells. (FIGS. 11B-11G).

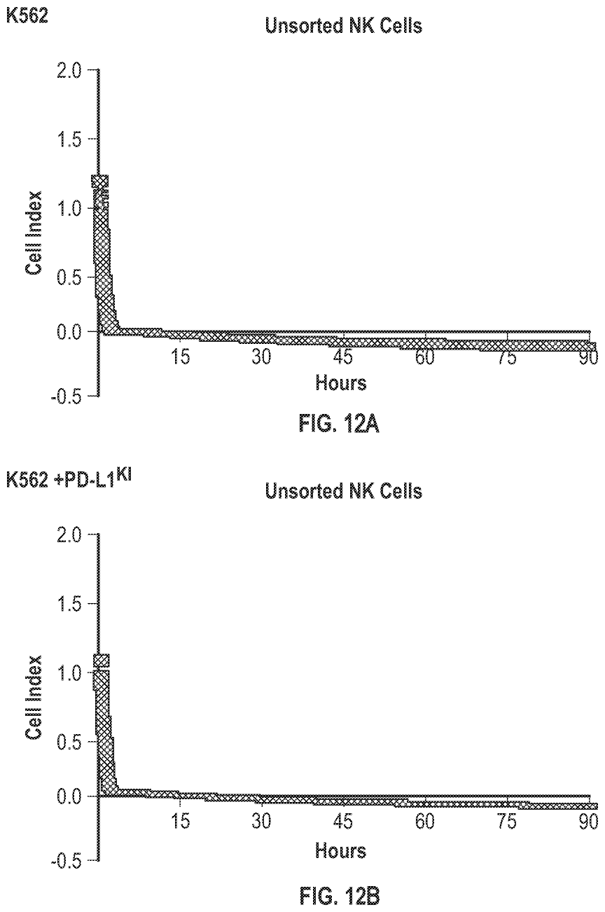

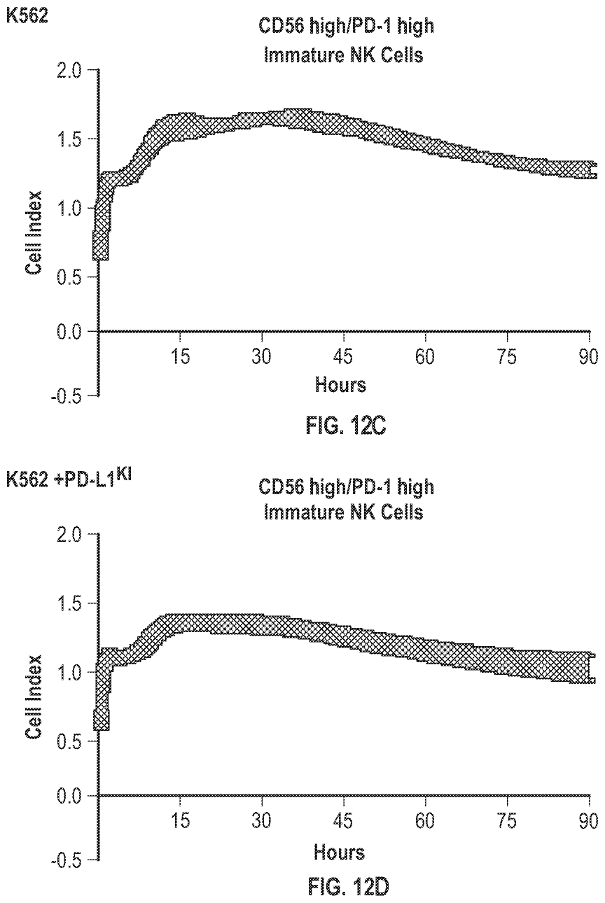

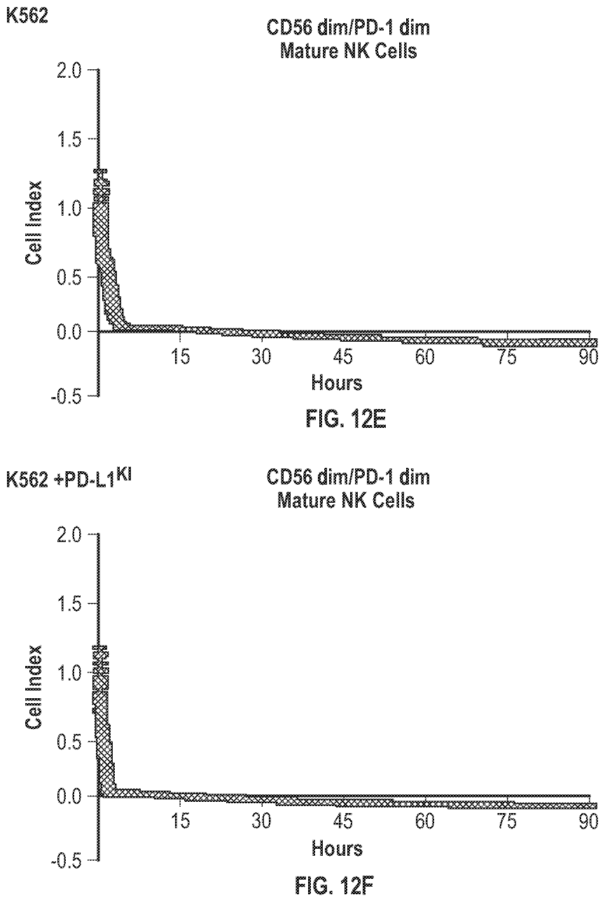

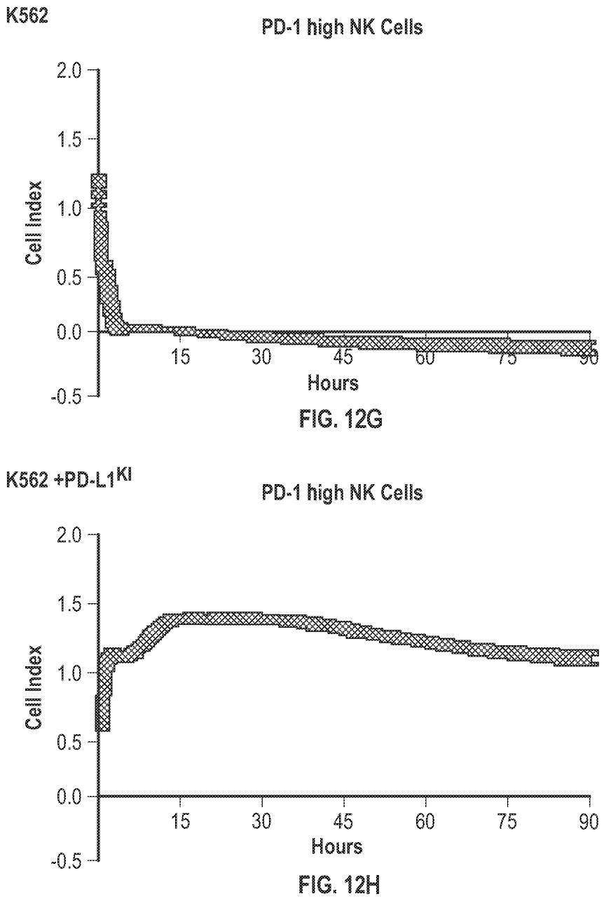

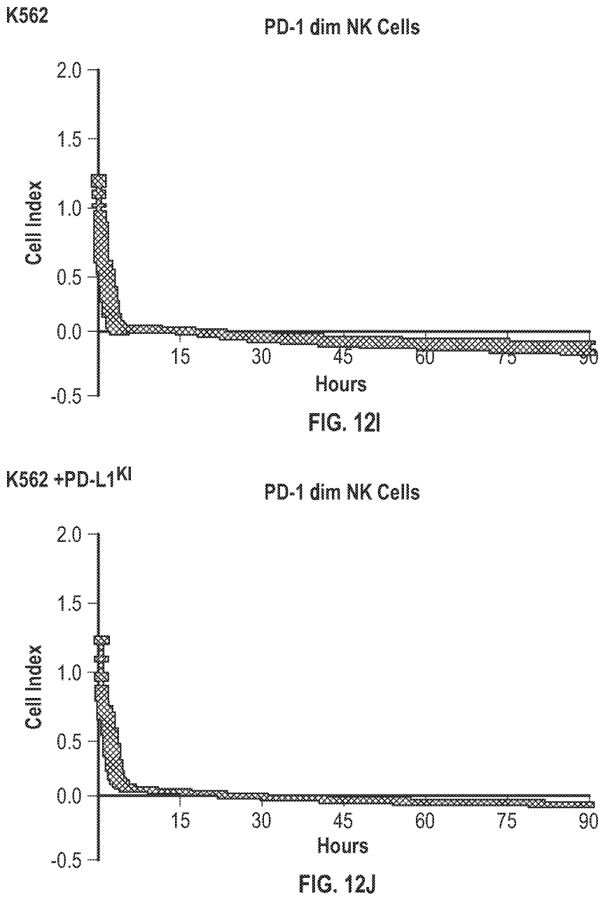

Standard cell killing assays were performed to determine whether specific NK cell subpopulations can recognize and kill modified K562 cells overexpressing PD-L1 (FIGS. 12A-12J). PD-L1 overexpressing K562 cells were not killed by PD-1 high NK cells, however they were killed by PD-1 dim NK cells (FIGS. 12A-12J).

To measure NK cell mediated killing, granzyme B and perforin release assays were performed using standard assays. It was determined that immature NK cells failed to recognize missing-self signals, and thus released only low levels of granzyme B and perforin (FIGS. 13A-13D). The data showed that unsorted primary NK cells were able to kill HLA-E knock-in K562 cells, HLA-G knock-in K562 cells and PD-L1 knock-in K562 cells. It was also determined that expression of NK cell stimulatory and inhibitory ligands was not affected in HLA-E knock-in K562 cells, HLA-G knock-in K562 cells, and PD-L1 knock-in K562 cells, in unstimulated and stimulated conditions (FIGS. 14A-14D).

In vivo killing assays were performed using either (i) a mixture of T cells and MHC I/II deficient cells or (ii) a mixture of T cells and HLA-I/-II deficient cells overexpressing HLA-E, HLA-G, or PD-L1. The mixture of cells was injected into the peritoneum of NSG mice, after adoptive transfer of human NK cells (such as unsorted or sorted for CD94). After 48 hours, peritoneal cells were recovered and sorted. The ratio of cells was calculated and plotted (FIGS. 15A-15D). K562 cells underwent in vivo killing and K562 cells overexpressing HLA-E were protected from killing by CD94 high NK cells (FIG. 15B). K562 cells overexpressing HLA-G were not protected by NK cell killing in vivo, yet the modified K562 cells were protected from NK cell killing by KIR2DL4 high NK cells (FIG. 15C). K562 cells overexpressing PD-L1 were not protected from NK cell killing in vivo, yet the modified K562 cells were protected from NK cell killing by PD-1 high NK cells (FIG. 15D).

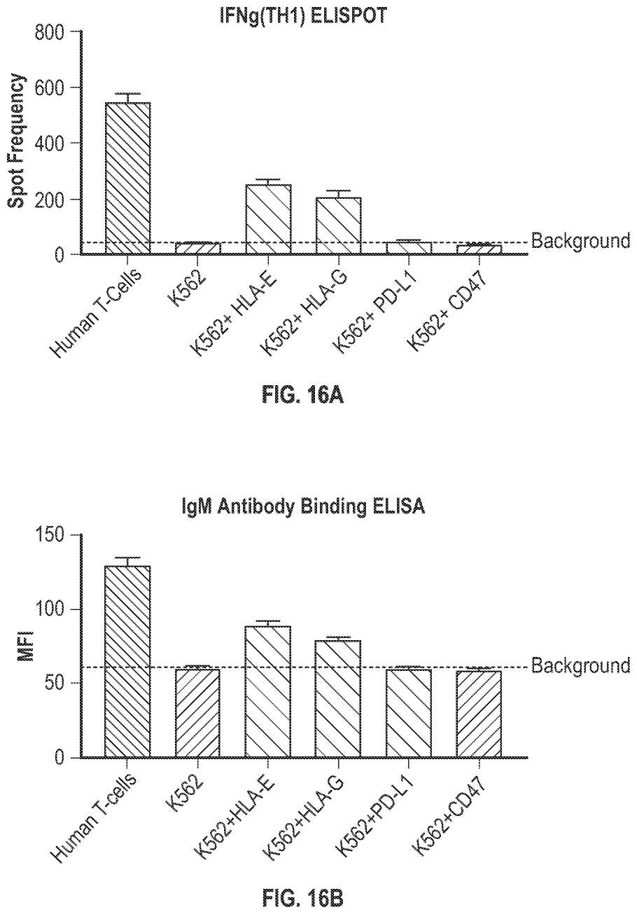

To determine T cell activation and donor-specific antibodies (DSA) in humanized mice, the mice were injected with either human T cells, K562 cells, HLA-E knock-in K562 cells, HLA-G knock-in K562 cells, or PD-L1 knock-in K562 cells. After 6 days, splenocytes were rechallenged in vitro with donor cells and human IFNg release were measured by spot frequency (indicating activation of T cells). See FIG. 16A. To analyze donor-specific antibodies (DSA), sera were incubated with injected cells in vitro and labeled with FITC IgM antibody. IgM were measured by flow (mean fluorescence intensity). See FIG. 16B. Overexpression of either HLA-E or HLA-G resulted in allo-peptide presentation, thereby leading to T cell and B cell activation.

### Results

The results of the experiments showed that overexpression of HLA-E by cells that do not express HLA-I/-II antigens (e.g., K562 cells) protected such cells from NK cell mediated cell lysis if the NK cells expressed CD94 (a receptor for HLA-E; see FIGS. 8A-8J). The flow cytometry data showed that a subpopulation of NK cells (about less than 40% of all NK cells) express CD94 (FIGS. 7A-7G). HLA-E overexpression was not sufficient to inhibit HLA-I/-II induced killing by primary NK cells in vitro and in vivo (FIGS. 13B, 14B and 15B).

Overexpression of HLA-G by cells that do not express HLA-I/-II antigens protected such cells from NK cell mediated cell lysis if the NK cells expressed KIR2DL4 (a receptor for HLA-G; see FIG. 10A-10J). A subpopulation of NK cells (about less than 20% of all NK cells) express high levels of KIR2DL4 (FIGS. 9A-9G). HLA-G overexpression was not sufficient to inhibit HLA-I/-II induced killing by primary NK cells in vitro and in vivo (FIGS. 13C, 14C and 15C).

Overexpression of PD-L1 by cells that do not express HLA-I/-II antigens protected such cells from NK cell mediated cell lysis if the NK cells expressed PD-1 (a receptor for PD-L1; see FIG. 11A). Only a subpopulation of all NK cells, for example, about less than 45%, express PD-1 (FIGS. 11A-11G). PD-L1 overexpression was not sufficient to inhibit HLA-I/-II induced killing by primary NK cells in vitro and in vivo (FIGS. 13, 14, and 15).

It was determined that HLA-E overexpression, HLA-G overexpression or PD-L1 overexpression does not affect the immune evasion concept to prevent allo-peptide presentation to the adaptive immune system.

All headings and section designations are used for clarity and reference purposes only and are not to be considered limiting in any way. For example, those of skill in the art will appreciate the usefulness of combining various embodiments from different headings and sections as appropriate according to the spirit and scope of the technology described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed is:

1. An engineered cell comprising one or more exogenous receptors selected from the group consisting of a human leukocyte antigen E (HLA-E) variant protein, a human leukocyte antigen G (HLA-G) variant protein, and an exogenous PD-L1 protein.

2. The engineered cell of claim 1, wherein the engineered cell comprises two or more exogenous receptors selected from the group consisting of a human leukocyte antigen E (HLA-E) variant protein, a human leukocyte antigen G (HLA-G) variant protein, and an exogenous PD-L1 protein.

3. The engineered cell of claim 1, further comprising reduced expression of MHC class I and/or MHC class II human leukocyte antigens relative to an unaltered or unmodified wild-type cell.

4. A hypoimmunogenic cell comprising: (i) reduced expression of MHC class I and/or MHC class II human leukocyte antigens relative to an unaltered or unmodified wild-type cell; and one or more exogenous receptors selected from the group consisting of an HLA-E variant protein, an HLA-G variant protein, and an exogenous PD-L1 protein.

5. The engineered cell or the hypoimmunogenic cell of claim 1-4, further comprising reduced expression and/or no expression of one or more receptors selected from the group consisting of HLA-A, HLA-B, HLA-C, and CD155.

6. The engineered cell or the hypoimmunogenic cell of claim 1-5, further comprising no expression of HLA-A and HLA-B.

7. The engineered cell or the hypoimmunogenic cell of claim 1-6, wherein the HLA-E variant protein comprises a modification in the antigen binding cleft and/or the HLA-G variant protein comprises a modification in the antigen binding cleft.

8. The engineered cell or the hypoimmunogenic cell of claim 1-7, wherein the HLA-E variant protein comprises a modification that increases protein stability compared to a wild-type HLA-E protein and/or the HLA-G variant protein comprises a modification that increases protein stability compared to a wild-type HLA-G protein.

9. The engineered cell or the hypoimmunogenic cell of claim 1-8, wherein i) the HLA-E variant protein comprises a modification that increases the recycling rate of the non-antigen bound HLA-E variant protein such that the HLA-E variant protein remains on the cell surface for a longer period of time compared to a wild-type HLA-E protein, and/or ii) the HLA-G variant protein comprises a modification that increases the recycling rate of the non-antigen bound HLA-G variant protein such that the HLA-G variant protein remains on the cell surface for a longer period of time compared to a wild-type HLA-G protein.

10. The engineered cell or the hypoimmunogenic cell of any one of claims 1-9, wherein the modification at the antigen binding cleft of the HLA-E variant protein prevents an antigen peptide from binding to the HLA-E variant protein and/or wherein the modification at the antigen binding cleft of the HLA-G variant protein prevents an antigen peptide from binding to the HLA-G variant protein

11. The engineered cell or the hypoimmunogenic cell of any one of claims 1-10, wherein the HLA-E variant protein comprises a modification such that the HLA-E variant protein binds a first decoy peptide and/or the HLA-G variant protein comprises a modification such that the HLA-G variant protein binds a second decoy peptide.

12. The engineered cell or the hypoimmunogenic cell of claim 11, wherein the first decoy peptide of the HLA-E variant protein is tethered to the HLA-E variant protein.

13. The engineered cell or the hypoimmunogenic cell of claim 11 or 12, wherein the first decoy peptide of the HLA-E variant protein binds the antigen binding cleft of the HLA-E variant protein.

14. The engineered cell or the hypoimmunogenic cell of claim 11, wherein the second decoy peptide of the HLA-G variant protein is tethered to the HLA-G variant protein.

15. The engineered cell or the hypoimmunogenic cell of claim 11 or 14, wherein the second decoy peptide of the HLA-G variant protein binds the antigen binding cleft of the HLA-G variant protein.

16. The engineered cell or the hypoimmunogenic cell of any one of claims 11-15, wherein the first decoy peptide and the second decoy peptide are different peptides.

17. The engineered cell or the hypoimmunogenic cell of any one of claims 11-16, wherein the HLA-E variant protein comprises a deletion in one or more of the intracellular domains and/or the HLA-G variant protein comprises a deletion in one or more of the intracellular domains.

18. The engineered cell or the hypoimmunogenic cell of claim 17, wherein the deletion in the one or more of the intracellular domains of HLA-E reduces or eliminates HLA-E signaling and/or the deletion in the one or more of the intracellular domains of HLA-G reduces or eliminates HLA-G signaling.

19. The engineered cell or the hypoimmunogenic cell of any one of claims 11-18, wherein i) the HLA-E variant protein comprises a deletion or other modification in the extracellular antigen binding domain region of the variant protein such that when the HLA-E variant protein is bound to an antigen peptide, the variant protein fails to recognize another binding partner, and/or ii) the HLA-G variant protein comprises a deletion or other modification in the extracellular antigen binding domain region of the variant protein such that when the HLA-G variant protein is bound to an antigen peptide, the variant protein fails to recognize another binding partner.

20. The engineered cell or the hypoimmunogenic cell of any one of claims 1-18, wherein the HLA-E variant protein comprises an HLA-E single chain dimer comprising an HLA-E heavy chain, a B2M subunit, and a linker, wherein the linker connects the HLA-E heavy chain and the B2M subunit.

21. The engineered cell or the hypoimmunogenic cell of any one of claims 1-18, wherein the HLA-E variant protein comprises an HLA-E single chain trimer comprising an HLA-E heavy chain, a B2M subunit, an antigen peptide, a first linker, and a second linker, wherein the first linker connects the HLA-E heavy chain and the B2M subunit and the second linker connects the B2M subunit to the antigen peptide.

22. The engineered cell or the hypoimmunogenic cell of any one of claims 1-21, wherein the engineered cell or the hypoimmunogenic cell does not express MHC class I and/or MHC class II human leukocyte antigens.

23. The engineered cell or the hypoimmunogenic cell of any one of claims 1-22, wherein the engineered cell or the hypoimmunogenic cell does not express HLA-DP, HLA-DQ, and/or HLA-DR antigens.

24. The engineered cell or the hypoimmunogenic cell of any one of claims 1-23, wherein the engineered cell or the hypoimmunogenic cell comprises reduced expression of beta-2-microglobulin (B2M) and/or MHC class II transactivator (CIITA) relative to an unaltered or unmodified wild-type cell.

25. The engineered cell or the hypoimmunogenic cell of any one of claims 1-24, wherein the engineered cell or the hypoimmunogenic cell does not express B2M and/or CIITA.

26. The engineered cell or the hypoimmunogenic cell of any one of claims 1-25, wherein the engineered cell or the hypoimmunogenic cell comprises one or more exogenous polynucleotides selected from the group consisting of a first polynucleotide encoding the HLA-E variant protein, a second polynucleotide encoding the HLA-G variant protein, and a third polynucleotide encoding the exogenous PD-L1 protein.

27. The engineered cell or the hypoimmunogenic cell of any one of claims 1-25, wherein the engineered cell or the hypoimmunogenic cell comprising two or more exogenous polynucleotides selected from the group consisting of a first polynucleotide encoding the HLA-E variant protein, a second polynucleotide encoding the HLA-G variant protein, and a third polynucleotide encoding the exogenous PD-L1 protein.

28. The engineered cell or the hypoimmunogenic cell of claim 26 or 27, wherein the first polynucleotide encoding the HLA-E variant protein is inserted into a first specific locus of at least one allele of the cell.

29. The engineered cell or the hypoimmunogenic cell of claim 26 or 27, wherein the second polynucleotide encoding the HLA-G variant protein is inserted into a second specific locus of at least one allele of the cell.

30. The engineered cell or the hypoimmunogenic cell of any one of claims 26-29, wherein the third polynucleotide encoding the exogenous PD-L1 protein is inserted into a third specific locus of at least one allele of the cell.

31. The engineered cell or the hypoimmunogenic cell of any one of claims 28-30, wherein the first, second and/or third specific loci are selected from the group consisting of a safe harbor locus, an RHD locus, a B2M locus, a CIITA locus, a TRAC locus, a TRB locus, an HLA-A locus, an HLA-B locus, an HLA-C locus, and a CD155 locus.

32. The engineered cell or the hypoimmunogenic cell of claim 31, wherein the safe harbor locus is selected from the group consisting of a CCR5 locus, a CXCR4 locus, a PPP1R12C locus, an ALB locus, a SHS231 locus, a CLYBL locus, a Rosa locus, an F3 (CD142) locus, a MICA locus, a MICB locus, a LRP1 (CD91) locus, a HMGB1 locus, an ABO locus, a FUT1 locus, and a KDM5D locus.

33. The engineered cell or the hypoimmunogenic cell of any one of claims 28-32, wherein any two of the first, second and third loci are the same locus.

34. The engineered cell or the hypoimmunogenic cell of any one of claims 28-32, wherein the first, second and third loci are the same locus.

35. The engineered cell or the hypoimmunogenic cell of any one of claims 28-32, wherein the first, second and third loci are different loci.

36. The engineered cell or the hypoimmunogenic cell of any one of claims 26-34, further comprising a single bicistronic polynucleotide comprising two polynucleotides selected from the group consisting of the first polynucleotide, the second polynucleotide and the third polynucleotide.

37. The engineered cell or the hypoimmunogenic cell of any one of claims 26-36, wherein the first polynucleotide, second polynucleotide and/or third polynucleotide are introduced into the engineered cell or the hypoimmunogenic cell using a lentiviral vector.

38. The engineered cell or the hypoimmunogenic cell of any one of claims 1-37, wherein the engineered cell or the hypoimmunogenic cell is derived from a human cell or an animal cell.

39. The engineered cell or the hypoimmunogenic cell of any one of claims 1-38, wherein the engineered cell or the hypoimmunogenic cell is a differentiated cell derived from an induced pluripotent stem cell or a progeny thereof.

40. The engineered cell or the hypoimmunogenic cell of claim 39, wherein the differentiated cell is selected from the group consisting of a T cell, a natural killer (NK) cell, and an endothelial cell.

41. The engineered cell or the hypoimmunogenic cell of any one of claims 1-38, wherein the engineered cell or the hypoimmunogenic cell is a primary immune cell or a progeny thereof.

42. The engineered cell or the hypoimmunogenic cell of claim 41, wherein the primary immune cell or a progeny thereof is a T cell or an NK cell.

43. The engineered cell or the hypoimmunogenic cell of claim 40 or 42, wherein the T cell comprises one or more one or more chimeric antigen receptors (CARs).

44. The engineered cell or the hypoimmunogenic cell of claim 43, wherein the one or more CARs are selected from the group consisting of a CD19-specific CAR, such that the T cell is a CD19 CAR T cell, a CD20-specific CAR, such that the T cell is a CD20 CAR T cell, a CD22-specific CAR, such that the T cell is a CD22 CAR T cell, and a BCMA-specific CAR such that the T cell is a BCMA CAR T cell, or a combination thereof.

45. The engineered cell or the hypoimmunogenic cell of claim 44, wherein the T cell comprises a CD19-specific CAR and a CD22-specific CAR such that the cell is a CD19/CD22 CAR T cell.

46. The engineered cell or the hypoimmunogenic cell of claim 45, wherein the CD19-specific CAR and a CD22-specific CAR are encoded by a single bicistronic polynucleotide.

47. The engineered cell or the hypoimmunogenic cell of claim 45, wherein the CD19-specific CAR and a CD22-specific CAR are encoded by two separate polynucleotides.

48. The engineered cell or the hypoimmunogenic cell of any one of claims 40 and 42-47, wherein the one or more CARs are introduced to the T cell using a lentiviral vector.

49. The engineered cell or the hypoimmunogenic cell of any one of claims 40 and 42-48, wherein the one or more CARs are introduced to the T cell in vivo in a recipient patient.

50. The engineered cell or the hypoimmunogenic cell of claim 49, wherein the one or more CARs are introduced to the T cell by contacting the recipient patient with a composition comprising one or more lentiviral vectors comprising (i) a CD4 binding agent or a CD8 binding agent, and (ii) one or more polynucleotides encoding the one or more CARs, wherein the T cell of the recipient patient is transduced with the one or more lentiviral vectors.

51. The engineered cell or the hypoimmunogenic cell of any one of claims 40 and 42-48, wherein the one or more CARs are introduced the T cell using CRISPR/Cas gene editing.

52. The engineered cell or the hypoimmunogenic cell of claim 51, wherein the CRISPR/Cas gene editing is carried out ex vivo from a donor subject.

53. The engineered cell or the hypoimmunogenic cell of claim 52, wherein the CRISPR/Cas gene editing is carried out using a lentiviral vector.

54. The engineered cell or the hypoimmunogenic cell of claim 53, wherein the CRISPR/Cas gene editing is carried out in vivo in a recipient patient.

55. The engineered cell or the hypoimmunogenic cell of claim 54, wherein the CRISPR/Cas gene editing is carried out by contacting the recipient patient with a composition comprising lentiviral vectors comprising (i) a CD4 binding agent or a CD8 binding agent, (ii) polynucleotides encoding CRISPR/Cas gene editing components, and (iii) one or more polynucleotides encoding the one or more CARs, wherein the T cell of the recipient patient is transduced with the lentiviral vectors.

56. The engineered cell or the hypoimmunogenic cell of any one of claims 39-55, wherein the differentiated cell or the progeny thereof, or the primary immune cell or the progeny thereof evades NK cell mediated cytotoxicity upon administration to a recipient patient.

57. The engineered cell or the hypoimmunogenic cell of any one of claims 39-56, wherein the differentiated cell or the progeny thereof, or the primary immune cell or the progeny thereof is protected from cell lysis by mature NK cells upon administration to a recipient patient.

58. The engineered cell or the hypoimmunogenic cell of any one of claims 39-57, wherein the differentiated cell or the progeny thereof, or the primary immune cell or the progeny thereof does not induce an immune response to the cell upon administration to a recipient patient.

59. A pharmaceutical composition comprising a population of the engineered cells of any one of claims 1-58 or a population of the hypoimmunogenic cells of any one of claims 4-58, and a pharmaceutically acceptable additive, carrier, diluent or excipient.

60. A method of treating a condition or disease in a patient in need thereof comprising administering a population of the differentiated cells of any one of claims 39-58 to the patient.

61. The method of claim 60, wherein the differentiated cells are selected from the group consisting of T cells, NK cells, and endothelial cells.

62. The method of claim 60, further administering a therapeutic agent that binds and/or interacts with one or more receptors on NK cells selected from the group consisting of CD94, KIR2DL4, PD-1, an inhibitory NK cell receptor, and an activating NK receptor.

63. The method of claim 60, wherein the therapeutic agent is selected from the group consisting of an antibody and fragments and variants thereof, an antibody mimetic, a small molecule, a blocking peptide, and a receptor antagonist.

64. The method of claim 60 or 61, wherein the condition or disease is selected from the group consisting of cancer, cardiovascular disease, stroke, peripheral artery disease (PAD), abdominal aortic aneurysm (AAA), carotid artery disease (CAD), arteriovenous malformation (AVM), critical limb-threatening ischemia (CLTI), pulmonary embolism (blood clots), deep vein thrombosis (DVT), chronic venous insufficiency (CVI), and any another vascular disorder/condition.

65. The method of any one of claims of 60-64, wherein the administration is selected from the group consisting of intravenous injection, intramuscular injection, intravascular injection, and transplantation.

66. A method of treating cancer in a patient in need thereof comprising administering a population of the primary immune cells of any one of claims 41-58 to the patient.

67. The method of claim 66, wherein the primary immune cells are selected from the group consisting of T cells and NK cells.

68. Use of a population of engineered T cells for treating a disorder or conditions in a recipient patient, wherein the engineered T cells comprise one or more exogenous receptors selected from the group consisting of an HLA-E variant protein, a HLA-G variant protein, and an exogenous PD-L1 protein and reduced expression of MHC class I and/or MHC class II human leukocyte antigens relative to an unaltered or unmodified wild-type cell, wherein the engineered T cells are propagated from a primary T cell or a progeny thereof, or are derived from an iPSC or a progeny thereof.

69. The use of claim 68, wherein the engineered T cell comprises two or more exogenous receptors selected from the group consisting of a HLA-E variant protein, a HLA-G variant protein, and an exogenous PD-L1 protein.

70. The use of claim 68 or 69, wherein the engineered T cell further comprises reduced expression and/or no expression of one or more receptors selected from the group consisting of HLA-A, HLA-B, HLA-C, and CD155.

71. The use of any one of claim 68-70, wherein the engineered T cell further comprises no expression of HLA-A and HLA-B.

72. The use of any one of claims 68-71, wherein the engineered T cells comprise an HLA-E variant protein and an HLA-G variant protein and reduced expression and/or no expression of one or more receptors selected from the group consisting of HLA-A, HLA-B, HLA-C, and CD155 relative to an unaltered or unmodified wild-type cell.

73. The use of any one of claims 68-72, wherein the engineered T cells comprise an HLA-E variant protein and an HLA-G variant protein and no expression of HLA-A and HLA-B.

74. The use of any one of claims 68-71, wherein the engineered T cells comprise an HLA-E variant protein and an exogenous PD-L1 protein and reduced expression and/or no expression of one or more receptors selected from the group consisting of HLA-A, HLA-B, HLA-C, and CD155 relative to an unaltered or unmodified wild-type cell.

75. The use of any one of claims 68-71 and 74, wherein the engineered T cells comprise an HLA-E variant protein and an exogenous PD-L1 protein and no expression of HLA-A and HLA-B.

76. The use of any one of claims 68-71, wherein the engineered T cells comprise an HLA-G variant protein and an exogenous PD-L1 protein and reduced expression and/or no expression of one or more receptors selected from the group consisting of HLA-A, HLA-B, HLA-C, and CD155 relative to an unaltered or unmodified wild-type cell.

77. The use of any one of claims 68-71 and 76, wherein the engineered T cells comprise an HLA-G variant protein and an exogenous PD-L1 protein and no expression of HLA-A and HLA-B.

78. The use of any one of claims 68-71, wherein the engineered T cells comprise an HLA-E variant protein and an HLA-G variant protein and reduced expression of MHC class I and MHC class II human leukocyte antigens relative to an unaltered or unmodified wild-type cell.

79. The use of any one of claims 68-71, wherein the engineered T cells comprise an HLA-E variant protein and an exogenous PD-L1 protein and reduced expression of MHC class I and MHC class II human leukocyte antigens relative to an unaltered or unmodified wild-type cell.

80. The use of any one of claims 68-71, wherein the engineered T cells comprise an HLA-G variant protein and an exogenous PD-L1 protein and reduced expression of MHC class I and MHC class II human leukocyte antigens relative to an unaltered or unmodified wild-type cell.

81. The use of any one of claims 68-71 and 78, wherein the engineered T cells comprise an HLA-E variant protein and an HLA-G variant protein and reduced expression of B2M and/or CIITA relative to an unaltered or unmodified wild-type cell.

82. The use of any one of claims 68-71 and 79, wherein the engineered T cells comprise an HLA-E variant protein and an exogenous PD-L1 protein and reduced expression of B2M and/or CIITA relative to an unaltered or unmodified wild-type cell.

83. The use of any one of claims 68-71 and 80, wherein the engineered T cells comprise an HLA-G variant protein and an exogenous PD-L1 protein and reduced expression of B2M and/or CIITA relative to an unaltered or unmodified wild-type cell.

84. The use of any one of claims 68-71, 78 and 81, wherein the engineered T cells comprise an HLA-E variant protein and an HLA-G variant protein and reduced expression of B2M and CIITA relative to an unaltered or unmodified wild-type cell.

85. The use of any one of claims 68-71, 79 and 82, wherein the engineered T cells comprise an HLA-E variant protein and an exogenous PD-L1 protein and reduced expression of B2M and CIITA relative to an unaltered or unmodified wild-type cell.

86. The use of any one of claims 68-71, 80 and 83, wherein the engineered T cells comprise an HLA-G variant protein and an exogenous PD-L1 protein and reduced expression of B2M and CIITA relative to an unaltered or unmodified wild-type cell.

87. The use of any one of claims 68-71, 78, 81 and 84, wherein the engineered T cells do not express MHC class I human leukocyte antigens, do not express MHC class II human leukocyte antigens and comprise an HLA-E variant protein and an HLA-G variant protein.

88. The use of any one of claims 68-71, 79, 82 and 85, wherein the engineered T cells do not express MHC class I human leukocyte antigens, do not express MHC class II human leukocyte antigens and comprise an HLA-E variant protein and an exogenous PD-L1 protein.

89. The use of any one of claims 68-71, 80, 83 and 86, wherein the engineered T cells do not express B2M, do not express CIITA and comprise an HLA-G variant protein and an exogenous PD-L1 protein.

90. The use of any one of claims 68-71, 78, 81, 84 and 87, wherein the engineered T cells do not express B2M, do not express CIITA and comprise an HLA-E variant protein and an HLA-G variant protein.

91. The use of any one of claims 68-71, 79, 82, 85 and 88, wherein the engineered T cells do not express B2M, do not express CIITA and comprise an HLA-E variant protein and an exogenous PD-L1 protein.

92. The use of any one of claims 68-71, 80, 83, 86 and 89, wherein the engineered T cells do not express B2M, do not express CIITA and comprise an HLA-G variant protein and an exogenous PD-L1 protein.

93. The use of any one of claims 68-92, wherein the HLA-E variant protein comprises a modification in the antigen binding cleft and/or the HLA-G variant protein comprises a modification in the antigen binding cleft.

94. The use of any one of claims 68-93, wherein the modification at the antigen binding cleft of the HLA-E variant protein prevents an antigen peptide from binding to the HLA-E variant protein and/or wherein the modification at the antigen binding cleft of the HLA-G variant protein prevents an antigen peptide from binding to the HLA-G variant protein.

95. The use of any one of claims 68-94, wherein the HLA-E variant protein comprises a modification such that the HLA-E variant protein binds a first decoy peptide and/or the HLA-G variant protein comprises a modification such that the HLA-G variant protein binds a second decoy peptide.

96. The use of any claim 95, wherein the first decoy peptide of the HLA-E variant protein is tethered to the HLA-E variant protein.

97. The use of claim 95 or 96, wherein the first decoy peptide of the HLA-E variant protein binds the antigen binding cleft of the HLA-E variant protein.

98. The use of claim 95, wherein the second decoy peptide of the HLA-G variant protein is tethered to the HLA-G variant protein.

99. The use of claim 95 or 98, wherein the second decoy peptide of the HLA-G variant protein binds the antigen binding cleft of the HLA-G variant protein.

100. The use of any one of claims 95-99, wherein the first decoy peptide and the second decoy peptide are different peptides.

101. The use of any one of claims 68-100, wherein the HLA-E variant protein comprises a deletion in one or more of the intracellular domains and/or the HLA-G variant protein comprises a deletion in one or more of the intracellular domains.

102. The use of any one of claims 68-101, wherein the deletion in the one or more of the intracellular domains of HLA-E reduces or eliminates HLA-E signaling and/or the deletion in the one or more of the intracellular domains of HLA-G reduces or eliminates HLA-G signaling.

103. The use of any one of claims 68-102, wherein i) the HLA-E variant protein comprises a deletion or other modification in the extracellular antigen binding domain region of the variant protein such that when the HLA-E variant protein is bound to an antigen peptide, the variant protein fails to recognize another binding partner, and/or ii) the HLA-G variant protein comprises a deletion or other modification in the extracellular antigen binding domain region of the variant protein such that when the HLA-G variant protein is bound to an antigen peptide, the variant protein fails to recognize another binding partner.

104. The use of any one of claims 68-81, 82, 84, 85, 87, 88, 90, 91, 93-97, and 100-103, wherein the HLA-E variant protein comprises an HLA-E single chain dimer comprising an HLA-E heavy chain, a B2M subunit, and a linker wherein the linker connects the HLA-E heavy chain and the B2M subunit.

105. The use of any one of claims 68-81, 82, 84, 85, 87, 88, 90, 91, 93-97 and 100-103, wherein the HLA-E variant protein comprises an HLA-E single chain trimer comprising an HLA-E heavy chain, a B2M subunit, an antigen peptide, a first linker, and a second linker, wherein the first linker connects the HLA-E heavy chain and the B2M subunit and the second linker connects the B2M subunit to the antigen peptide.

106. The use of any one of claims 68-105, wherein the engineered T cells comprise one or more exogenous polynucleotides selected from the group consisting of a first polynucleotide encoding the HLA-E variant protein, a second polynucleotide encoding the HLA-G variant protein, and a third polynucleotide encoding the exogenous PD-L1 protein.

107. The use of any one of claims 68-105, wherein the engineered T cells comprise two or more exogenous polynucleotides selected from the group consisting of a first polynucleotide encoding the HLA-E variant protein, a second polynucleotide encoding the HLA-G variant protein, and a third polynucleotide encoding the exogenous PD-L1 protein.

108. The use of claim 106 or 107, wherein the first polynucleotide encoding the HLA-E variant protein is inserted into a first specific locus of at least one allele of the cell, the second polynucleotide encoding the HLA-G variant protein is inserted into a second specific locus of at least one allele of the cell, and/or the third polynucleotide encoding the exogenous PD-L1 protein is inserted into a third specific locus of at least one allele of the cell.

109. The use of claim 108, wherein the first, second and/or third specific loci are selected from the group consisting of a safe harbor locus, an RHD locus, a B2M locus, a CIITA locus, a TRAC locus, a TRB locus, an HLA-A locus, an HLA-B locus, an HLA-C locus, and a CD155 locus.

110. The use of claim 109, wherein the safe harbor locus is selected from the group consisting of a CCR5 locus, a CXCR4 locus, a PPP1R12C locus, an ALB locus, a SHS231 locus, a CLYBL locus, a Rosa locus, an F3 (CD142) locus, a MICA locus, a MICB locus, a LRP1 (CD91) locus, a HMGB1 locus, an ABO locus, a FUT1 locus, and a KDM5D locus.

111. The use of any one of claims 108-110, wherein the any two of the first, second and third loci are the same locus.

112. The use of any one of claims 108-110, wherein the first, second and third loci are the same locus.

113. The use of any one of claims 108-110, wherein the first, second and third loci are different loci.

114. The use of any one of claims 108-112, wherein the engineered T cells further comprise a single bicistronic polynucleotide comprising two polynucleotides selected from the group consisting of the first polynucleotide, the second polynucleotide and the third polynucleotide.

115. The use of any one of claims 106-114, wherein the first polynucleotide, the second polynucleotide and/or the third polynucleotide are introduced into the engineered T cell using CRISPR/Cas gene editing.

116. The use of any one of claims 106-114, wherein the first polynucleotide, second polynucleotide and/or third polynucleotide are introduced into the engineered T cell using a lentiviral vector.

117. The use of any one of claims 106-116, wherein the engineered T cell comprises one or more one or more chimeric antigen receptors (CARs).

118. The use of claim 117, wherein the one or more CARs are selected from the group consisting of a CD19-specific CAR, such that the engineered T cell is a CD19 CAR T cell, a CD20-specific CAR, such that the engineered T cell is a CD20 CAR T cell, a CD22-specific CAR, such that the engineered T cell is a CD22 CAR T cell, and a BCMA-specific CAR such that the engineered T cell is a BCMA CAR T cell, or a combination thereof.

119. The use of claim 117 or 118, wherein the engineered T cell comprises a CD19-specific CAR and a CD22-specific CAR such that the cell is a CD19/CD22 CAR T cell.

120. The use of any one of claims 117-119, wherein the CD19-specific CAR and a CD22-specific CAR are encoded by a single bicistronic polynucleotide.

121. The use of any one of claims 117-119, wherein the CD19-specific CAR and a CD22-specific CAR are encoded by a two separate polynucleotides.

122. The use of any one of claims 117-121, wherein the one or more CARs are introduced to the engineered T cell using a lentiviral vector.

123. The use of any one of claims 117-122, wherein the one or more CARs are introduced to the engineered T cell in vivo in the recipient patient.

124. The use of claim 123, wherein the one or more CARs are introduced to the engineered T cell by contacting the recipient patient with a composition comprising one or more lentiviral vectors comprising (i) a CD4 binding agent or a CD8 binding agent, and (ii) one or more polynucleotides encoding the one or more CARs, wherein the engineered T cell of the recipient patient is transduced with the one or more lentiviral vectors.

125. The use of any one of claims 117-122, wherein the one or more CARs are introduced the engineered T cell using CRISPR/Cas gene editing.

126. The use of claim 125, wherein the CRISPR/Cas gene editing is carried out ex vivo from a donor subject.

127. The use of claim 125 or 126, wherein the CRISPR/Cas gene editing is carried out using a lentiviral vector.

128. The use of claim 125, wherein the CRISPR/Cas gene editing is carried out in vivo in the recipient patient.

129. The use of claim 128, wherein the CRISPR/Cas gene editing is carried out by contacting the recipient patient with a composition comprising one or more lentiviral vectors comprising (i) a CD4 binding agent or a CD8 binding agent, (ii) polynucleotides encoding CRISPR/Cas gene editing components, and (iii) one or more polynucleotides encoding the one or more CARs, wherein the T cell of the recipient patient is transduced with the one or more lentiviral vectors.

130. Use of a population of engineered differentiated cells for treating a disorder or conditions in a recipient patient, wherein the engineered differentiated cells comprise one or more exogenous receptors selected from the group consisting of an HLA-E variant protein, a HLA-G variant protein, and an exogenous PD-L1 protein and reduced expression of MHC class I and/or MHC class II human leukocyte antigens relative to an unaltered or unmodified wild-type cell, wherein the engineered differentiated cells are derived an iPSC or a progeny thereof.

131. The use of claim 130, wherein the engineered differentiated cells comprise two or more exogenous receptors selected from the group consisting of a HLA-E variant protein, a HLA-G variant protein, and an exogenous PD-L1 protein.

132. The use of claim 130 or 131, wherein the engineered differentiated cell further comprises reduced expression and/or no expression of one or more receptors selected from the group consisting of HLA-A, HLA-B, HLA-C, and CD155.

133. The use of any one of claims 130-132, wherein the engineered differentiated cell further comprises no expression of HLA-A and HLA-B.

134. The use of any one of claims 130-133, wherein the engineered differentiated cells comprise an HLA-E variant protein and an HLA-G variant protein and reduced expression and/or no expression of one or more receptors selected from the group consisting of HLA-A, HLA-B, HLA-C, and CD155 relative to an unaltered or unmodified wild-type cell.

135. The use of any one of claims 130-134, wherein the engineered differentiated cells comprise an HLA-E variant protein and an HLA-G variant protein and no expression of HLA-A and HLA-B.

136. The use of any one of claims 130-133, wherein the engineered differentiated cells comprise an HLA-E variant protein and an exogenous PD-L1 protein and reduced expression and/or no expression of one or more receptors selected from the group consisting of HLA-A, HLA-B, HLA-C, and CD155 relative to an unaltered or unmodified wild-type cell.

137. The use of any one of claims 130-133 and 136, wherein the engineered differentiated cells comprise an HLA-E variant protein and an exogenous PD-L1 protein and no expression of HLA-A and HLA-B.

138. The use of any one of claims 130-133, wherein the engineered differentiated cells comprise an HLA-G variant protein and an exogenous PD-L1 protein and reduced expression and/or no expression of one or more receptors selected from the group consisting of HLA-A, HLA-B, HLA-C, and CD155 relative to an unaltered or unmodified wild-type cell.

139. The use of any one of claims 130-133 and 138, wherein the engineered differentiated cells comprise an HLA-G variant protein and an exogenous PD-L1 protein and no expression of HLA-A and HLA-B.

140. The use of claim 130 or 131, wherein the engineered differentiated cells comprise an HLA-E variant protein and an HLA-G variant protein and reduced expression of MHC class I and MHC class II human leukocyte antigens relative to an unaltered or unmodified wild-type cell.

141. The use of claim 130 or 131, wherein the engineered differentiated cells comprise an HLA-E variant protein and an exogenous PD-L1 protein and reduced expression of MHC class I and MHC class II human leukocyte antigens relative to an unaltered or unmodified wild-type cell.

142. The use of claim 130 or 131, wherein the engineered differentiated cells comprise an HLA-G variant protein and an exogenous PD-L1 protein and reduced expression of MHC class I and MHC class II human leukocyte antigens relative to an unaltered or unmodified wild-type cell.

143. The use of any one of claims 130, 131 and 140, wherein the engineered differentiated cells comprise an HLA-E variant protein and an HLA-G variant protein and reduced expression of B2M and/or CIITA relative to an unaltered or unmodified wild-type cell.

144. The use of any one of claims 130, 131 and 141, wherein the engineered differentiated cells comprise an HLA-E variant protein and an exogenous PD-L1 protein and reduced expression of B2M and/or CIITA relative to an unaltered or unmodified wild-type cell.

145. The use of any one of claims 130, 131 and 142, wherein the engineered differentiated cells comprise an HLA-G variant protein and an exogenous PD-L1 protein and reduced expression of B2M and/or CIITA relative to an unaltered or unmodified wild-type cell.

146. The use of any one of claims 130, 131, 140 and 143, wherein the engineered differentiated cells comprise an HLA-E variant protein and an HLA-G variant protein and reduced expression of B2M and CIITA relative to an unaltered or unmodified wild-type cell.

147. The use of any one of claims 130, 131, 141 and 144, wherein the engineered differentiated cells comprise an HLA-E variant protein and an exogenous PD-L1 protein and reduced expression of B2M and CIITA relative to an unaltered or unmodified wild-type cell.

148. The use of any one of claims 130, 131, 142 and 145, wherein the engineered differentiated cells comprise an HLA-G variant protein and an exogenous PD-L1 protein and reduced expression of B2M and CIITA relative to an unaltered or unmodified wild-type cell.

149. The use of any one of claims 130, 131, 140, 143 and 146, wherein the engineered differentiated cells do not express MHC class I human leukocyte antigens, do not express MHC class II human leukocyte antigens and comprise an HLA-E variant protein and an HLA-G variant protein.

150. The use of any one of claims 130, 131, 142, 144 and 147, wherein the engineered differentiated cells do not express MHC class I human leukocyte antigens, do not express MHC class II human leukocyte antigens and comprise an HLA-E variant protein and an exogenous PD-L1 protein.

151. The use of any one of claims 130, 131, 142, 145 and 148, wherein the engineered differentiated cells do not express B2M, do not express CIITA and comprise an HLA-G variant protein and an exogenous PD-L1 protein.

152. The use of any one of claims 130, 131, 140, 143, 146 and 149, wherein the engineered differentiated cells do not express B2M, do not express CIITA and comprise an HLA-E variant protein and an HLA-G variant protein.

153. The use of any one of claims 130, 131, 142, 144, 147 and 150, wherein the engineered differentiated cells do not express B2M, do not express CIITA and comprise an HLA-E variant protein and an exogenous PD-L1 protein.

154. The use of any one of claims 130, 131, 142, 145, 148, and 151, wherein the engineered T cells do not express B2M, do not express CIITA and comprise an HLA-G variant protein and an exogenous PD-L1 protein.

155. The use of any one of claims 130-154, wherein the HLA-E variant protein comprises a modification in the antigen binding cleft and/or the HLA-G variant protein comprises a modification in the antigen binding cleft.

156. The use of any one of claims 130-155, wherein the modification at the antigen binding cleft of the HLA-E variant protein prevents an antigen peptide from binding to the HLA-E variant protein and/or wherein the modification at the antigen binding cleft of the HLA-G variant protein prevents an antigen peptide from binding to the HLA-G variant protein.

157. The use of any one of claims 130-156, wherein the HLA-E variant protein comprises a modification such that the HLA-E variant protein binds a first decoy peptide and/or the HLA-G variant protein comprises a modification such that the HLA-G variant protein binds a second decoy peptide.

158. The use of any claim 157, wherein the first decoy peptide of the HLA-E variant protein is tethered to the HLA-E variant protein.

159. The use of claim 157 or 158, wherein the first decoy peptide of the HLA-E variant protein binds the antigen binding cleft of the HLA-E variant protein.

160. The use of claim 157, wherein the second decoy peptide of the HLA-G variant protein is tethered to the HLA-G variant protein.

161. The use of claim 157 or 160, wherein the second decoy peptide of the HLA-G variant protein binds the antigen binding cleft of the HLA-G variant protein.

162. The use of any one of claims 157-161, wherein the first decoy peptide and the second decoy peptide are different peptides.

163. The use of any one of claims 130-162, wherein the HLA-E variant protein comprises a deletion in one or more of the intracellular domains and/or the HLA-G variant protein comprises a deletion in one or more of the intracellular domains.

164. The use of claim 163, wherein the deletion in the one or more of the intracellular domains of HLA-E reduces or eliminates HLA-E signaling and/or the deletion in the one or more of the intracellular domains of HLA-G reduces or eliminates HLA-G signaling.

165. The use of any one of claims 130-164, wherein i) the HLA-E variant protein comprises a deletion or other modification in the extracellular antigen binding domain region of the variant protein such that when the HLA-E variant protein is bound to an antigen peptide, the variant protein fails to recognize another binding partner, and/or ii) the HLA-G variant protein comprises a deletion or other modification in the extracellular antigen binding domain region of the variant protein such that when the HLA-G variant protein is bound to an antigen peptide, the variant protein fails to recognize another binding partner.

166. The use of any one of claims 130-137, 139-141, 143, 144, 146, 147 149, 150, 152, 153, 155-159, and 163-165, wherein the HLA-E variant protein comprises an HLA-E single chain dimer comprising an HLA-E heavy chain, a B2M subunit, and a linker wherein the linker connects the HLA-E heavy chain and the B2M subunit.

167. The use of any one of claims 130-137, 139-141, 143, 144, 146, 147 149, 150, 152, 153, 155-159, and 163-165, wherein the HLA-E variant protein comprises an HLA-E single chain trimer comprising an HLA-E heavy chain, a B2M subunit, an antigen peptide, a first linker, and a second linker, wherein the first linker connects the HLA-E heavy chain and the B2M subunit and the second linker connects the B2M subunit to the antigen peptide.

168. The use of any one of claims 130-167, wherein the engineered differentiated cells comprise one or more exogenous polynucleotides selected from the group consisting of a first polynucleotide encoding the HLA-E variant protein, a second polynucleotide encoding the HLA-G variant protein, and a third polynucleotide encoding the exogenous PD-L1 protein.

169. The use of any one of claims 130-167, wherein the engineered differentiated cells comprise two or more exogenous polynucleotides selected from the group consisting of a first polynucleotide encoding the HLA-E variant protein, a second polynucleotide encoding the HLA-G variant protein, and a third polynucleotide encoding the exogenous PD-L1 protein.

170. The use of claim 168 or 169, wherein the first polynucleotide encoding the HLA-E variant protein is inserted into a first specific locus of at least one allele of the cell, the second polynucleotide encoding the HLA-G variant protein is inserted into a second specific locus of at least one allele of the cell, and/or the third polynucleotide encoding the exogenous PD-L1 protein is inserted into a third specific locus of at least one allele of the cell.

171. The use of claim 170, wherein the first, second and/or third specific loci are selected from the group consisting of a safe harbor locus, an RHD locus, a B2M locus, a CIITA locus, a TRAC locus, a TRB locus, an HLA-A locus, an HLA-B locus, an HLA-C locus, and a CD155 locus.

172. The use of claim 171, wherein the safe harbor locus is selected from the group consisting of a CCR5 locus, a CXCR4 locus, a PPP1R12C locus, an ALB locus, a SHS231 locus, a CLYBL locus, a Rosa locus, an F3 (CD142) locus, a MICA locus, a MICB locus, a LRP1 (CD91) locus, a HMGB1 locus, an ABO locus, a FUT1 locus, and a KDM5D locus.

173. The use of any one of claims 170-172, wherein any two of the first, second and third loci are the same locus.

174. The use of any one of claims 170-173, wherein the first, second and third loci are the same locus.

175. The use of any one of claims 170-172, wherein the first, second and third loci are different loci.

176. The use of any one of claims 168-174, wherein the engineered differentiated cells further comprise a single bicistronic polynucleotide comprising two polynucleotides selected from the group consisting of the first polynucleotide, the second polynucleotide and the third polynucleotide.

177. The use of any one of claims 168-176, wherein the first polynucleotide, the second polynucleotide and/or the third polynucleotide are introduced the engineered differentiated cell using CRISPR/Cas gene editing.

178. The use of any one of claims 168-177, wherein the first polynucleotide, second polynucleotide and/or third polynucleotide are introduced into the engineered differentiated cell using a lentiviral vector.

179. A human leukocyte antigen E (HLA-E) variant protein comprising a modification at the antigen binding cleft.

180. The HLA-E variant protein of claim 179, wherein the modification at the antigen binding cleft of the HLA-E variant protein prevents an antigen peptide from binding to the variant protein.

181. The HLA-E variant protein of claim 179 or 180, wherein the HLA-E variant protein binds a decoy peptide.

182. The HLA-E variant protein of any one of claims 179-181, wherein the decoy peptide of the HLA-E variant protein is tethered to the HLA-E variant protein.

183. The HLA-E variant protein of any one of claims 179-182, wherein the decoy peptide of the HLA-E variant protein binds the antigen binding cleft of the HLA-E variant protein.

184. The HLA-E variant protein of any one of claims 179-183, wherein the HLA-E variant protein comprises a deletion in one or more of the intracellular domains.

185. The HLA-E variant protein of any one of claims 179-184, wherein the HLA-E variant protein comprises an HLA-E single chain dimer comprising an HLA-E heavy chain, a B2M subunit, and a linker wherein the linker connects the HLA-E heavy chain and the B2M subunit.

186. The HLA-E variant protein of any one of claims 179-184, wherein the HLA-E variant protein comprises an HLA-E single chain trimer comprising an HLA-E heavy chain, a B2M subunit, an antigen peptide, a first linker, and a second linker, wherein the first linker connects the HLA-E heavy chain and the B2M subunit and the second linker connects the B2M subunit to the antigen peptide.

187. A human leukocyte antigen G (HLA-G) variant protein comprising a modification in the antigen binding cleft.

188. The HLA-G variant protein of claim 187, wherein the modification at the antigen binding cleft of the HLA-G variant protein prevents an antigen peptide from binding to the variant protein.

189. The HLA-G variant protein of claim 187 or 188, wherein the HLA-G variant protein binds a decoy peptide.

190. The HLA-G variant protein of claim 189, wherein the decoy peptide of the HLA-E variant protein is tethered to the HLA-G variant protein.

191. The HLA-G variant protein of claim 189 or 190, wherein the decoy peptide of the HLA-G variant protein binds the antigen binding cleft of the HLA-G variant protein.

192. The HLA-G variant protein of any one of claims 187-191, wherein the HLA-G variant protein comprises a deletion in one or more of the intracellular domains.

193. A polynucleotide construct comprising a polynucleotide encoding the HLA-E variant protein of any one of claims 179-186.

194. A polynucleotide construct comprising a polynucleotide encoding the HLA-G variant protein of any one of claims 187-192.

195. The polynucleotide construct of claim 193 or 194, wherein polynucleotide construct further comprises one or more polynucleotides for CRISPR/Cas gene editing.

196. The polynucleotide construct of claim 195, wherein the polynucleotide construct further comprises one or more polynucleotides for CRISPR/Cas gene editing to insert the polynucleotide encoding the HLA-E variant protein into a specific locus of at least one allele of a cell.

197. The polynucleotide construct of claim 195, wherein the polynucleotide construct further comprises one or more polynucleotides for CRISPR/Cas gene editing to insert the polynucleotide encoding the HLA-G variant protein into a specific locus of at least one allele of a cell.

198. The polynucleotide construct of claim 196 or 197, wherein the specific locus is selected from the group consisting of a safe harbor locus, an RHD locus, a B2M locus, a CIITA locus, a TRAC locus, a TRB locus, an HLA-A locus, an HLA-B locus, an HLA-C locus, and a CD155 locus.

199. The polynucleotide construct of claim 198, wherein the safe harbor locus is selected from the group consisting of a CCR5 locus, a CXCR4 locus, a PPP1R12C locus, an ALB locus, a SHS231 locus, a CLYBL locus, a Rosa locus, an F3 (CD142) locus, a MICA locus, a MICB locus, a LRP1 (CD91) locus, a HMGB1 locus, an ABO locus, a FUT1 locus, and a KDM5D locus.

200. A single bicistronic polynucleotide construct comprising a first polynucleotide encoding the HLA-E variant protein of any one of claims 179-186 and a second polynucleotide encoding the HLA-G variant protein of any one of claims 187-192.

201. A single bicistronic polynucleotide construct comprising a first polynucleotide encoding the HLA-E variant protein of any one of claims 179-186 and a second polynucleotide encoding an PD-L1 protein.

202. A single bicistronic polynucleotide construct comprising a first polynucleotide encoding the HLA-G variant protein of any one of claims 187-192 and a second polynucleotide encoding an PD-L1 protein.

203. The nucleic acid construct of claims 179-199 or the single bicistronic polynucleotide construct of claims 200-202, further comprises a promoter.

204. The nucleic acid construct of claims 179-199 or the single bicistronic polynucleotide construct of claims 200-202, wherein the promoter is a constitutive promoter.

205. The nucleic acid construct of claims 179-199 or the single bicistronic polynucleotide construct of claims 200-202, wherein the promoter is a tissue-type specific promoter.


SEQUENCE LISTING (128 sequences)

SEQ ID NO: 1-5 - skipped (5 entries)

SEQ ID NO: 6 - protein, 21 aa, Artificial Sequence
  Synthetic peptide
MALPVTALLLPLALLLHAARP

SEQ ID NO: 7 - protein, 20 aa, Artificial Sequence
  Synthetic peptide
METDTLLLWVLLLWVPGSTG

SEQ ID NO: 8 - protein, 22 aa, Artificial Sequence
  Synthetic peptide
MLLLVTSLLLCELPHPAFLLIP

SEQ ID NO: 9 - protein, 45 aa, Artificial Sequence
  Synthetic hinge domain
TTTPAPRPPTPAPTIASQPLSLRPEACRPAAGGAVHTRGLDFACD

SEQ ID NO: 10 - protein, 39 aa, Artificial Sequence
  Synthetic hinge domain
IEVMYPPPYLDNEKSNGTIIHVKGKHLCPSPLFPGPSKP

SEQ ID NO: 11 - protein, 12 aa, Artificial Sequence
  Synthetic hinge domain
ESKYGPPCPPCP

SEQ ID NO: 12 - protein, 12 aa, Artificial Sequence
  Synthetic hinge domain
ESKYGPPCPSCP

SEQ ID NO: 13 - protein, 229 aa, Artificial Sequence
  Synthetic hinge domain
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 24 aa, Artificial Sequence
  Synthetic transmembrane domain
IYIWAPLAGTCGVLLLSLVITLYC

SEQ ID NO: 15 - protein, 27 aa, Artificial Sequence
  Synthetic transmembrane domain
FWVLVVVGGVLACYSLLVTVAFIIFWV

SEQ ID NO: 16 - protein, 42 aa, Artificial Sequence
  Synthetic costimulatory domain
KRGRKKLLYIFKQPFMRPVQTTQEEDGCSCRFPEEEEGGCEL

SEQ ID NO: 17 - protein, 41 aa, Artificial Sequence
  Synthetic costimulatory domain
RSKRSRLLHSDYMNMTPRRPGPTRKHYQPYAPPRDFAAYRS

SEQ ID NO: 18 - protein, 112 aa, Artificial Sequence
  Synthetic signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 245 aa, Artificial Sequence
  Synthetic amino acid
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 20 - protein, 107 aa, Artificial Sequence
  Synthetic amino acid
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 21 - protein, 6 aa, Artificial Sequence
  Synthetic amino acid
QDISKY

SEQ ID NO: 22 - protein, 3 aa, Artificial Sequence
  Synthetic amino acid
HTS

SEQ ID NO: 23 - protein, 9 aa, Artificial Sequence
  Synthetic amino acid
QQGNTLPYT

SEQ ID NO: 24 - protein, 18 aa, Artificial Sequence
  Synthetic amino acid linker
GSTSGSGKPGSGEGSTKG

SEQ ID NO: 25 - protein, 120 aa, Artificial Sequence
  Synthetic amino acid
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 26 - protein, 8 aa, Artificial Sequence
  Synthetic amino acid
GVSLPDYG

SEQ ID NO: 27 - protein, 7 aa, Artificial Sequence
  Synthetic amino acid
IWGSETT

SEQ ID NO: 28 - protein, 14 aa, Artificial Sequence
  Synthetic amino acid
AKHYYYGGSYAMDY

SEQ ID NO: 29 - protein, 242 aa, Artificial Sequence
  Synthetic amino acid
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 30 - protein, 15 aa, Artificial Sequence
  Synthetic amino acid linker
GGGGSGGGGSGGGGS

SEQ ID NO: 31 - DNA, 1458 bp, Artificial Sequence
  Synthetic nucleotide sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 32 - protein, 486 aa, Artificial Sequence
  Synthetic amino acid
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - DNA, 1383 bp, Artificial Sequence
  Synthetic nucleotide sequence
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 34 - protein, 461 aa, Artificial Sequence
  Synthetic amino acid
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - DNA, 1467 bp, Artificial Sequence
  Synthetic nucleotide sequence
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 36 - protein, 489 aa, Artificial Sequence
  Synthetic amino acid
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 246 aa, Artificial Sequence
  Synthetic amino acid
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGSGKPGSGEGSTKGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 38 - protein, 106 aa, Artificial Sequence
  Synthetic amino acid
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 39 - protein, 10 aa, Artificial Sequence
  Synthetic amino acid
RASSSVNYMD

SEQ ID NO: 40 - protein, 7 aa, Artificial Sequence
  Synthetic amino acid
ATSNLAS

SEQ ID NO: 41 - protein, 9 aa, Artificial Sequence
  Synthetic amino acid
QQWSFNPPT

SEQ ID NO: 42 - protein, 122 aa, Artificial Sequence
  Synthetic amino acid
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 43 - protein, 5 aa, Artificial Sequence
  Synthetic amino acid
SYNMH

SEQ ID NO: 44 - protein, 17 aa, Artificial Sequence
  Synthetic amino acid
AIYPGNGDTSYNQKFKG

SEQ ID NO: 45 - protein, 246 aa, Artificial Sequence
  Synthetic amino acid
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 46 - protein, 124 aa, Artificial Sequence
  Synthetic amino acid
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 47 - protein, 10 aa, Artificial Sequence
  Synthetic amino acid
GDSVSSNSAA

SEQ ID NO: 48 - protein, 9 aa, Artificial Sequence
  Synthetic amino acid
TYYRSKWYN

SEQ ID NO: 49 - protein, 14 aa, Artificial Sequence
  Synthetic amino acid
AREVTGDLEDAFDI

SEQ ID NO: 50 - protein, 107 aa, Artificial Sequence
  Synthetic amino acid
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 51 - protein, 6 aa, Artificial Sequence
  Synthetic amino acid
QTIWSY

SEQ ID NO: 52 - protein, 3 aa, Artificial Sequence
  Synthetic amino acid
AAS

SEQ ID NO: 53 - protein, 9 aa, Artificial Sequence
  Synthetic amino acid
QQSYSIPQT

SEQ ID NO: 54 - protein, 246 aa, Artificial Sequence
  Synthetic amino acid
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 55 - protein, 124 aa, Artificial Sequence
  Synthetic amino acid
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSS

SEQ ID NO: 56 - protein, 10 aa, Artificial Sequence
  Synthetic amino acid
GDSVSSNSVA

SEQ ID NO: 57 - protein, 9 aa, Artificial Sequence
  Synthetic amino acid
TYYRSTWYN

SEQ ID NO: 58 - protein, 14 aa, Artificial Sequence
  Synthetic amino acid
AREVTGDLEDAFDI

SEQ ID NO: 59 - protein, 107 aa, Artificial Sequence
  Synthetic amino acid
DIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 60 - protein, 6 aa, Artificial Sequence
  Synthetic amino acid
QTIWSY

SEQ ID NO: 61 - protein, 3 aa, Artificial Sequence
  Synthetic amino acid
AAS

SEQ ID NO: 62 - protein, 9 aa, Artificial Sequence
  Synthetic amino acid
QQSYSIPQT

SEQ ID NO: 63 - protein, 246 aa, Artificial Sequence
  Synthetic amino acid
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 64 - protein, 111 aa, Artificial Sequence
  Synthetic amino acid
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 65 - protein, 15 aa, Artificial Sequence
  Synthetic amino acid
RASESVSVIGAHLIH

SEQ ID NO: 66 - protein, 7 aa, Artificial Sequence
  Synthetic amino acid
LASNLET

SEQ ID NO: 67 - protein, 9 aa, Artificial Sequence
  Synthetic amino acid
LQSRIFPRT

SEQ ID NO: 68 - protein, 117 aa, Artificial Sequence
  Synthetic amino acid
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 69 - protein, 5 aa, Artificial Sequence
  Synthetic amino acid
DYSIN

SEQ ID NO: 70 - protein, 17 aa, Artificial Sequence
  Synthetic amino acid
WINTETREPAYAYDFRG

SEQ ID NO: 71 - protein, 8 aa, Artificial Sequence
  Synthetic amino acid
DYSYAMDY

SEQ ID NO: 72 - protein, 246 aa, Artificial Sequence
  Synthetic amino acid
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 73 - protein, 111 aa, Artificial Sequence
  Synthetic amino acid
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 74 - protein, 15 aa, Artificial Sequence
  Synthetic amino acid
RASESVTILGSHLIY

SEQ ID NO: 75 - protein, 7 aa, Artificial Sequence
  Synthetic amino acid
LASNVQT

SEQ ID NO: 76 - protein, 9 aa, Artificial Sequence
  Synthetic amino acid
LQSRTIPRT

SEQ ID NO: 77 - protein, 117 aa, Artificial Sequence
  Synthetic amino acid
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 78 - protein, 5 aa, Artificial Sequence
  Synthetic amino acid
HYSMN

SEQ ID NO: 79 - protein, 17 aa, Artificial Sequence
  Synthetic amino acid
RINTESGVPIYADDFKG

SEQ ID NO: 80 - protein, 8 aa, Artificial Sequence
  Synthetic amino acid
DYLYSLDF

SEQ ID NO: 81 - protein, 121 aa, Artificial Sequence
  Synthetic amino acid
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 82 - protein, 8 aa, Artificial Sequence
  Synthetic amino acid
GFTFSSYA

SEQ ID NO: 83 - protein, 8 aa, Artificial Sequence
  Synthetic amino acid
ISGSGDYI

SEQ ID NO: 84 - protein, 14 aa, Artificial Sequence
  Synthetic amino acid
AKEGTGANSSLADY

SEQ ID NO: 85-112 - skipped (28 entries)

SEQ ID NO: 113 - protein, 42 aa, Artificial Sequence
  Synthetic hinge domain
AAAIEVMYPPPYLDNEKSNGTIIHVKGKHLCPSPLFPGPSKP

SEQ ID NO: 114 - protein, 28 aa, Artificial Sequence
  Synthetic transmembrane domain
MFWVLVVVGGVLACYSLLVTVAFIIFWV

SEQ ID NO: 115 - protein, 112 aa, Artificial Sequence
  Synthetic signaling domain
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - DNA, 1467 bp, Artificial Sequence
  Synthetic nucleotide sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 117 - protein, 489 aa, Artificial Sequence
  Synthetic amino acid
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - protein, 243 aa, Artificial Sequence
  Synthetic amino acid
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 119 - protein, 106 aa, Artificial Sequence
  Synthetic amino acid
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIK

SEQ ID NO: 120 - protein, 6 aa, Artificial Sequence
  Synthetic amino acid
QSISSY

SEQ ID NO: 121 - protein, 3 aa, Artificial Sequence
  Synthetic amino acid
AAS

SEQ ID NO: 122 - protein, 8 aa, Artificial Sequence
  Synthetic amino acid
QQKYDLLT

SEQ ID NO: 123 - protein, 119 aa, Artificial Sequence
  Synthetic amino acid
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 124 - protein, 10 aa, Artificial Sequence
  Synthetic amino acid
GGSISSSSYY

SEQ ID NO: 125 - protein, 7 aa, Artificial Sequence
  Synthetic amino acid
ISYSGST

SEQ ID NO: 126 - protein, 11 aa, Artificial Sequence
  Synthetic amino acid
ARDRGDTILDV

SEQ ID NO: 127 - DNA, 1503 bp, Artificial Sequence
  Synthetic nucleotide sequence
atggccttaccagtgaccgccttgctcctgccgctggccttgctgctccacgccgccaggccggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaaaatacgacctcctcacttttggcggagggaccaaggttgagatcaaaggcagcaccagcggctccggcaagcctggctctggcgagggcagcacaaagggacagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctcctatagtgggagcacctactacaacccgtccctcaagagtcgagtcaccatatccgtagacacgtccaagaaccagttctccctgaagctgagttctgtgaccgccgcagacacggcggtgtactactgcgccagagatcgtggagacaccatactagacgtatggggtcagggtacaatggtcaccgtcagctcattcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 128 - protein, 501 aa, Artificial Sequence
  Synthetic amino acid
MALPVTALLLPLALLLHAARPDIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR